(19) United States
(12) Patent Application Publication
(10) Pub. No.: US 2024/0293364 A1
(43) Pub. Date: Sep. 5, 2024

(54) BIOMARKERS FOR CANCER THERAPY USING MDM2 ANTAGONISTS

(71) Applicant: OTSUKA PHARMACEUTICAL CO., LTD., Tokyo (JP)

(72) Inventors: Nicola FERRARI, Cambridge (GB); Harpreet Kaur SAINI, Cambridge (GB); Jong Sook AHN, Cambridge (GB); Jessica Laura BROTHWOOD, Cambridge (GB)

(73) Assignee: OTSUKA PHARMACEUTICAL CO., LTD., Tokyo (JP)

(21) Appl. No.: 18/548,522

(22) PCT Filed: Mar. 4, 2022
(86) PCT No.: PCT/IB2022/051906

§ 371 Date: Aug. 31, 2023

(30) Foreign Application Priority Data
Mar. 4, 2021 (GB) 2103080.4

(51) Int. Cl. A61K 31/4035; A61K 31/502; A61K 31/5025; A61P 35/00; G01N 33/574

(52) U.S. Cl. CPC A61K 31/4035; A61K 31/502; A61K 31/5025; A61P 35/00; G01N 33/57484; G01N 2333/9108

(57) ABSTRACT

The invention provides DNA damage response (DDR) pathway genes and their gene products as biomarkers to predict effective treatment of cancer using an MDM2 antagonist. Identifying one or more DDR pathway biomarkers in a cancer patient allows a determination to be made whether the patient's cancer is likely to be successfully treated using an MDM2 antagonist. Accordingly, the invention relates generally to a companion diagnostic for MDM2 antagonist therapy. In particular, the DDR pathway comprises one or more genes from: the homologous recombination repair (HRR) pathway; the non-homologous end joining (NHEJ) pathway; the mismatch repair (MMR) pathway; the Fanconi

### FIELD OF THE INVENTION

This invention relates to biomarkers for cancer therapy. In particular, the invention provides biological markers that identify a cancer cell as likely to be sensitive to an MDM2 antagonist. These biomarkers can be incorporated into methods, systems and kits for predicting response to treatment, and into personalised treatments for cancer.

### BACKGROUND TO THE INVENTION

Precision medicine, or personalised medicine, is an emerging approach for disease treatment and prevention that takes into account individual variability in genes, environment and lifestyle for each patient. It is often said to be the practice of administering the right dosage of the right drug at the right time.

A particular focus of precision medicine is the need to predict whether a given patient will respond to a specific drug. A test that is able to predict whether a particular drug will effectively treat an individual patient is often referred to as a companion diagnostic. Effective companion diagnostics are very desirable because of their ability to improve treatment outcomes for patients while also saving the significant economic cost of providing ineffective treatments. An effective companion diagnostic for a new therapeutic agent can also increase the chances of that therapy being trialled in the correct population and ultimately being approved.

Precision medicines and companion diagnostics often rely on biomarkers that are able to predict reliably whether a patient is likely to respond to a specific treatment. Identifying reliable biomarkers for every therapy and disease is a very significant challenge.

WO-A-2016/056673 describes complex gene signatures that are said to provide predictive molecular tools for clinical application. The disclosure also relates to methods of predicting the sensitivity of cancers or tumors to anticancer drugs that can influence the treatment of the cancers or tumors, particularly inhibitors of MDM2 activity and antagonists of the interaction of MDM2 and p53 proteins.

US-A-2015/0211073 also describes a gene panel, typically comprising at least four genes, as a biomarker for predicting the response of a cancer to an MDM2 antagonist

lorio et al (Cell. 2016 Jul. 28; 166(3):740-75) “A Landscape of Pharmacogenomic Interactions in Cancer” report how cancer-driven alterations identified in 11,289 tumours from 29 tissues (integrating somatic mutations, copy number alterations, DNA methylation, and gene expression) can be mapped onto 1,001 molecularly annotated human cancer cell lines and correlated with sensitivity to 265 drugs. While such studies provide a resource to link genotypes with cellular phenotypes and to identify therapeutic options for selected cancer sub-populations, the development of clinically-relevant molecularly-targeted cancer therapies remains a formidable challenge.

There remains a need to identify reliable biomarkers for use in precision medicine.

### SUMMARY OF THE INVENTION

The invention is based on the identification of biomarkers that can be used to predict effective treatment of cancer using an MDM2 antagonist. Identifying one or more of these biomarkers in a cancer patient allows a determination to be made whether the patient's cancer is likely to be treated or likely to be successfully treated using an MDM2 antagonist. Accordingly, in certain aspects the invention relates generally to a companion diagnostic for MDM2 antagonist therapy.

The biomarkers identified in the present invention are DNA damage response (DDR) pathway genes and their gene products. These proteins and the genes encoding them are all known in the art. As used herein, these biomarkers are referred to as the “biomarkers of the invention” and/or “DDR biomarkers”. Reduced DDR function in a cancer cell indicates sensitivity to MDM2 antagonism.

Depletion, loss, or reduced function of one or more DDR genes or gene products therefore indicates sensitivity to MDM2 antagonism.

In particular, in one aspect the invention provides an MDM2 antagonist for use in a method of treating cancer, wherein the cancer is depleted of one or more DDR genes, gene products or activities. The depletion may be of the gene itself, of the gene product (i.e. reduced expression), or a mutation causing reduced activity (i.e. a loss of function mutation). The depletion may be due to an aberration causing reduced activity, for example copy number loss, or epigenetic silencing.

There is a broad group of aberrations that induce loss/reduction of activity such as loss of activity by copy number loss or loss of function mutation. A loss of function mutation may be viewed as the depletion of the wild type gene product. Accordingly, when depletion of a biomarker is described, this includes a mutation in that biomarker such that the wild type is reduced or no longer detectable.

In one aspect, the invention provides an MDM2 antagonist for use in a method of treating cancer, wherein the cancer is depleted of one or more genes, gene products or activities in the homologous recombination (HR) pathway (which is also referred to as the Homologous Recombination Repair (HRR) pathway). These cancer cells have reduced HR function and can therefore be characterised as HR deficient. In one embodiment, the cancer is BRCA1 depleted. Human BRCA1 has Entrez gene ID 672.

In one embodiment the cancer is BRCA2 depleted. Human BRCA2 has Entrez gene ID 675. In one embodiment, the cancer is ATM depleted. ATM is the Ataxia Telangiectasia Mutated protein. Human wild-type ATM has Entrez gene ID 472. One embodiment of the invention relates to depletion of any two of BRCA1, BRCA2 and ATM. One embodiment of the invention relates to depletion of all three of BRCA1, BRCA2 and ATM. One embodiment of the invention relates to a loss of function mutation in BRCA1, BRCA2 or ATM compared to WT. One embodiment of the invention relates to a loss of function mutation in any two of BRCA1, BRCA2 and ATM compared to WT. One embodiment of the invention relates to a loss of function mutation in all three of BRCA1, BRCA2 and ATM compared to WT. One embodiment of the invention relates to a loss of copy number, or epigenetic silencing, in 1, 2 or 3 of BRCA1, BRCA2 or ATM compared to WT. One embodiment of the invention relates to a loss of function mutation in BRCA1, and/or BRCA2. One embodiment of the invention relates to a loss of copy number, or epigenetic silencing, in BRCA1 and/or BRCA2. In one embodiment the defect in homologous recombination repair mimics BRCA1 or BRCA2 loss.

In one embodiment the HR pathway gene(s) are selected from one or more of the following genes: LIG1, MRE11A, NBN, PARG, PARP1, PARPBP, RAD50, TP53BP1, XRCC2, XRCC3, EXO1, PCNA, POLD1, POLD2, POLD3, POLD4, RFC1, RFC2, RFC3, RFC4, RFC5, RPA1, RPA2, RPA3, RPA4, BARD1, BLM, BRCA1, BRCA2, BRIP1, DMC1, DNA2, EID3, EME1, EME2, ERCC1, H2AFX, HELQ, HFM1, IN080, KAT5, MUS81, NFATC2IP, NSMCE1, NSMCE2, NSMCE3, NSMCE4A, PALB2, PARP2, PAXIP1, POLH, POLQ, PPP4C, PPP4R1, PPP4R2, PPP4R4, RAD51, RAD51B, RAD51C, RAD51D, RAD52, RAD54B, RAD54L, RBBP8, RDM1, RECQL, RECQL4, RECQL5, RM11, RMI2, RTEL1, SHFM1, SLX1A, SLX1B, SLX4, SMARCAD1, SMC5, SMC6, SPO11, SWSAP1, TOP3A, TOP3B, UIMC1, WRN, and/or ZSWIM7. These HR pathway genes are all known in the art, for example as described by Knijnenburg et al Cell Rep. 2018 Apr. 3; 23(1):239-254.e6.

In some embodiments, the HR deficient cancer cells are not ATM deficient, i.e. wild-type ATM is expressed at a normal (or high) level in the cancer cells. In these cells, the HR deficiency is provided by a loss of function in a different HR gene or genes for example BRCA1 and/or BRCA2. These cancer cells can, in some embodiments, comprise HR depletion that consists of genes, gene products or activities in the HR pathway that are not ATM.

In some embodiments, the HR deficient cancer cells are not ATR deficient, i.e. wild-type ATR is expressed at a normal (or high) level in the cancer cells. In these cells, the HR deficiency is provided by a loss of function in a different HR gene or genes for example BRCA1 and/or BRCA2. These cancer cells can, in some embodiments, comprise HR depletion that consists of genes, gene products or activities in the HR pathway that are not ATR. ATR is “Ataxia Telangiectasia and Rad3-related protein”. An exemplary sequence of human ATR is available in UniProtKB database under the accession number of Q13535 (ATR-HUMAN), in the GenBank database under the NCBI accession number of AAK26749.1, and is also published in literatures such as Bentley et al., EMBO J., 15: 6641-6651 (1996) and Cimprich et al., Proc. Natl. Acad. Sci. USA 107: 18575-18480 (1996).

In some embodiments, the cancer cells comprise HR depletion that consists of genes, gene products or activities in the HR pathway that are not ATR.

In some embodiments, the HR-deficient cancer cells are not ATM deficient and are not ATR deficient, i.e. wild-type ATR and wild-type ATM are expressed at a normal (or high) levels in the cancer cells. In these cells, the HR deficiency is provided by a loss of function in a different HR gene or genes for example BRCA1 and/or BRCA2.

In some embodiments cancer cells comprise HR depletion that consists of genes, gene products or activities in the HR pathway that are not ATM and are not ATR.

In some embodiments, the cancer is determined to be depleted of one or more genes or gene products in one or more DNA damage repair (DDR) pathways, or the cancer has at least one loss of function mutation in at least one DDR pathway gene, wherein the one or more DDR genes or gene products do not consist of ATM and/or ATR. This means that the genes or gene products can comprise ATM and/or ATR and one or more other DDR pathway biomarkers, or if there is a sole biomarker then it is not ATM or ATR, and if there is a pair of biomarkers then they are not ATM and ATR but may comprise ATM or ATR and another DDR pathway biomarker. In some embodiments, the cancer may be depleted of one gene or gene product in one DNA damage repair (DDR) pathway, or the cancer has at least one loss of function mutation in one DDR pathway gene, and the DDR pathway gene or gene product is not ATM or ATR.

Accordingly, in some embodiments when there is a single biomarker according to the invention, it is not ATM or ATR. In some embodiments when there is a single biomarker according to the invention, it is not ATM. In some embodiments when there is a single biomarker according to the invention, it is not ATR.

In some embodiments, the cancer may be depleted of two genes or gene products in one or more DNA damage repair (DDR) pathways, or the cancer has at least one loss of function mutation in at least two DDR pathway genes, and the DDR pathway genes or gene products are not ATM and ATR.

In some embodiments, the cancer may be depleted of two genes or gene products in one or more DNA damage repair (DDR) pathways, or the cancer has at least one loss of function mutation in at least two DDR pathway genes, and the DDR pathway genes or gene products include one or more of ATM and/or ATR, and one or more DDR pathway genes or gene products that are not ATM and ATR. In another aspect, the cancer is depleted of one or more genes, gene products or activities in the Fanconi Anemia (FA) pathway. This pathway includes at least the genes FANCA (Entrez gene ID 2175), FANCB (gene ID 2187), FANCC (gene ID 2176), FANCD1 (also known as BRCA2, gene ID 675), FANCD2 (gene ID 2177), FANCE (gene ID 2178), FANCF (gene ID 2188), FANCG (gene ID 2189), FANCI (gene ID 55215), FANCJ (gene ID 83990), FANCL (gene ID 55120), FANCM (gene ID 57697), FANCN (gene ID 79728), FANCO (gene ID 889), FANCP (gene ID 84464), FANCQ (gene ID 2072), FANCR (gene ID 5888), FANCS (also known as BRCA1, gene ID 672), FANCT (gene ID 29089), FANCU (gene ID 7516), FANCV (gene ID 10459), and FANCW (gene ID 55159). One embodiment of the invention relates to a loss of function mutation in one or more FA pathway genes compared to WT.

In another aspect, the invention provides an MDM2 antagonist for use in a method of treating cancer, wherein the cancer is depleted of one or more genes, gene products or activities in the non-homologous end joining (NHEJ) pathway. These cancer cells can therefore be characterised as NHEJ deficient. In one embodiment, the cancer is ATRX depleted. ATRX is Alpha thalassemia/mental retardation syndrome X-linked. Human wild-type ATRX has Entrez gene ID 546. One embodiment of the invention relates to a loss of function mutation in ATRX compared to WT.

In a further aspect, the invention provides an MDM2 antagonist for use in a method of treating cancer, wherein the cancer is depleted of one or more genes, gene products or activities in the mismatch repair (MMR) pathway. These cancer cells can therefore be characterised as MMR deficient. In one embodiment, the cancer is depleted of one or more of MSH2 (Entrez gene ID 4436), MSH3 (gene ID 4437), MSH6 (gene ID 2956), MLH1 (gene ID 4292), PMS2 (gene ID 5395), and/or MLH3 (gene ID 27030). In a further embodiment, the cancer is depleted of POLD1 (Entrez gene ID 5424) or POLE (gene ID 5426), for example has one or more POLD1 and/or POLE mutations. One embodiment of the invention relates to a loss of function mutation in one or more MMR pathway genes compared to WT.

In some embodiments, the cancer comprises mutational signature SBS6 and/or SBS26 associated with defects in DNA mismatch repair, and/or the POLD1 mutational signature SBS20. These single base substitution (SBS) signatures are known from the Catalogue of Somatic Mutations in Cancer (COSMIC), GRCh37 v91. The COSMIC SBS signatures can be accessed at cancer.sanger.ac.uk/cosmic and were prepared as described by Alexandrov et al Nature volume 578, pages 94-101 (2020).

In a further aspect, the invention provides an MDM2 antagonist for use in a method of treating cancer, wherein the cancer is depleted of one or more genes, gene products or activities in the base excision repair (BER) pathway. These cancer cells can therefore be characterised as BER deficient. One embodiment of the invention relates to a loss of function mutation in one or more BER pathway genes compared to WT.

In one embodiment of all aspects, the loss or depletion of a DDR gene or functional gene product in a cancer is indicated by microsatellite instability (MSI) and/or the tumour mutational burden of the cancer, typically “MSI-High” and/or a “high tumour mutational burden”.

For some biomarkers, protein is typically measured. This can be achieved using, for example, immunohistochemistry (IHC). In some embodiments mutational analysis (e.g. DNA sequencing) may be used to detect biomarker status.

In some embodiments, the invention provides an MDM2 antagonist for use in a method of treating cancer, wherein the cancer is depleted of one or more DDR biomarkers and optionally has increased expression of one or more interferon signature (IFN signature) genes. The IFN signature genes comprises CXCL10, CXCL11, RSAD2, MX1, BATF2, IFI44L, IFITM1, ISG15, CMPK2, IF127, CD74, IFIH1, CCRL2, IFI44, HERC6, ISG20, IFIT3, HLA-C, OAS1, IFI35, IRF9, EPSTI1, USP18, BST2, CSF1, C1S, DHX58, TRIM14, OASL, IRF7, LGALS3BP, DDX60, LAP3, LAMP3, PARP12, PARP9, SP110, PLSCR1, WARS, STAT1, IRF3, IRF5, MSC, JUN, SP11, IRF1, COMMD3-BM11, STAT2, RUNX3, SREBF1, and FLI1. These biomarkers are collectively referred to herein as the “interferon signature”. Typically the IFN signature biomarkers will be detected as mRNA.

Measurement techniques for one or more nucleic acid biomarkers, for example the IFN signature biomarkers, can include quantitative techniques such as RT-PCR or Nanostring analysis, as are known in the art. DNA can also be measured. In some embodiments copy number variation (CNV) analysis and/or mutational analysis (e.g. DNA sequencing) may be used to detect biomarker gene status.

In some embodiments, the invention provides an MDM2 antagonist for use in a method of treating cancer, wherein the cancer is depleted of one or more DDR biomarkers and optionally also has decreased expression of one, two or three of CDKN2A, BAP1 and SKP2. Optionally, the cancer is depleted of one or more DDR biomarkers and is depleted of one, two or three of CDKN2A, BAP1 and SKP2. Such a cancer may further have increased expression of one or more interferon signature (IFN signature) genes described herein. In one embodiment, an MDM2 antagonist is provided for use in a method of treating cancer, wherein the cancer is depleted of one or more DDR biomarkers and optionally also be CDKN2A depleted; BAP1 depleted; and/or show increased expression of one, two, three, four, five or more of the interferon signature genes.

SKP2 depletion may mean loss or complete loss of the SKP2 gene, mutation of the SKP2 gene and loss of function, or it may mean low gene expression and low protein expression and function, which result from the loss or mutation of the gene or otherwise. In one embodiment, an MDM2 antagonist is provided for use in a method of treating cancer, wherein the cancer is depleted of one or more DDR biomarkers and optionally also has decreased, reduced, low or no SKP2 expression.

The CDKN2A gene encodes the p16(INK4A) and the p14(ARF) proteins, and references to the gene CDKN2A includes the proteins encoded by CDKN2A. The CDKN2A loss can be measured by low protein expression product levels i.e. an expression level that is lower than a control expression level, of p16(INK4A) and/or the p14(ARF) i.e. a consequence of the CDKN2A gene loss is loss of p16 and/or P14.

For CDKN2A, protein is typically measured. This can be achieved using, for example, immunohistochemistry (IHC). In some embodiments mutational analysis (e.g. DNA sequencing) may be used to detect CDKN2A status.

For BAP1, protein may typically be measured. This can be achieved using, for example, immunohistochemistry (IHC). Cellular location may also be measured in some embodiments. In some embodiments mutational analysis (e.g. DNA sequencing) may be used to detect BAP1 status. The biomarkers identified herein as having increased expression are sometimes referred to as the interferon signature, or IFN signature, biomarkers. They are also referred to by the term Type 1 interferon pathway genes. Typically, these biomarkers will be detected as mRNA. Measurement techniques for one or more IFN signature biomarkers can therefore include quantitative techniques such as RT-PCR or Nanostring analysis, as are known in the art. DNA can also be measured. In some embodiments copy number variation (CNV) analysis and/or mutational analysis (e.g. DNA sequencing) may be used to detect biomarker gene status.

The biomarkers of the invention may be measured directly or indirectly. Indirect measurement typically involves detection of a molecule that is functionally upstream or downstream of the biomarker and the level of which correlates with the level of the biomarker. For example, a substrate upon which the biomarker acts can be used as an indirect measurement of the biomarker. In one embodiment, BAP1 levels may be measured by detecting the level of histone H2A ubiquitination, with increased H2A ubiquitination typically reflecting decreased BAP1. In another embodiment, BAP1 depletion can be assessed by determining increased EZH2 expression or activity. In one embodiment, SKP2 can be detected indirectly through the detection of one or more SKP2 substrates. A typical SKP2 substrate is p27. In one embodiment of the invention the SKP2 level is assessed by measuring the levels of one or more of p27, p21, p57, E2F-1, MEF, P130, Tob1, cyclin D, cyclin E, Smad4, Myc, Mcb, RASSF1A, Foxo1, Orc1p, Cdt1, Rag2, Brca2, CDK9, MPK1, and/or UBP43.

Another indirect measurement of a DDR biomarker is a downstream readout of DDR defects. One such readout is the tumour mutational burden (TMB). Another downstream readout is Microsatellite instability (MSI). Clinical assays are already used in the clinic to stratify patients based on those features. For example, MSI-high (or MSI-H) is a well-known clinical definition of cancer that may be determined using techniques including next generation sequencing, fluorescent multiplex PCR and capillary electrophoreses, immunohistochemistry, or single-molecule molecular inversion probes. In addition, TMB can be measured using NGS methods. Tumor mutation burden (TMB) can be used as a marker of genomic instability and MSI. MSI testing methods (MSK-Impact and F1CDx) include both microsatellite instability (MSI) and tumor mutational burden (TMB) measures.

The data in the Examples below indicate that depletion, for example loss (also known as total or complete loss) of one or more of the DDR biomarker activities is predictive of sensitivity of cancer cells to an MDM2 antagonist. Accordingly, low levels of one or more of the DDR biomarkers can be used to identify a cancer suitable for treatment with an MDM2 antagonist. In certain embodiments, the DDR biomarker(s) may be from one or more of the following pathways: HR, NHEJ, FA, MMR and/or BER.

Multiple DDR biomarkers measured may all be in the same pathway, or may be from different pathways.

In one embodiment, the DDR biomarker or biomarkers measured comprises ATM, BRCA1 and/or BRCA2, typically a loss of function ATM, BRCA1 and/or BRCA2 mutation. In another embodiment the DDR biomarker or biomarkers measured comprises ATRX, typically a loss of function ATRX mutation.

In another embodiment, one or more of the DDR biomarkers is within the FA pathway. In a further embodiment, the MMR biomarker depletion is identified by MSI (e.g. MSI-High) and/or increased tumour mutational burden compared to a non-sensitive cell. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE.

In some embodiments, the DDR biomarker or biomarkers measured comprises the mutational signature SBS6 and/or SBS26 associated with defects in DNA mismatch repair, and/or the POLD1 mutational signature SBS20.

In some embodiments, decreased levels of the biomarker or biomarkers of the invention are determined relative to a non-cancer cell. This cancer:non-cancer comparison may be particularly useful. The non-cancer cell will typically be a cell of the same type as the cancer cell. The non-cancer cell may be from the same patient, or may be from a different patient, or may be a value known for a non-cancer cell of that type. In this way, the biomarker level e.g. expression or activity can be compared relative to control levels determined in healthy individuals or relative to control levels determined in normal non-proliferative tissue.

In some other embodiments, a decreased level e.g. expression of the biomarker or biomarkers of the invention are determined relative to cancer cell samples from MDM2 inhibitor non-responsive subjects, or in a sample of cancer cells from an MDM2 inhibitor non-responsive subject. The non-responsive cancer cells will typically be a cell of the same cancer type as the tested cancer cell. The non-responsive cancer cells will typically be from a different patient or patients from the tested sample, or may be a value known for a non-responsive cancer cell of that cancer type.

In some embodiments, the patient can be identified as a candidate for treatment with an MDM2 antagonist when the expression or activity level of the one or more DDR biomarkers is low relative to the upper limit of normal (ULN). In one embodiment, the one or more DDR biomarkers are from one or more of the following pathways: HR, NHEJ, FA, MMR and/or BER. The DDR biomarker or biomarkers measured may all be in the same pathway, or may be from different pathways.

In another embodiment, the DDR biomarker or biomarkers measured comprises ATM. In some embodiments, the presence of ATM with a loss of function mutation compared to wild type predicts sensitivity to MDM2 antagonism. In another embodiment, the DDR biomarker or biomarkers measured comprises BRCA1. In some embodiments, the presence of BRCA1 with a loss of function mutation compared to wild type predicts sensitivity to MDM2 antagonism. In another embodiment, the DDR biomarker or biomarkers measured comprises BRCA2. In some embodiments, the presence of BRCA2 with a loss of function mutation compared to wild type predicts sensitivity to MDM2 antagonism.

In another embodiment the DDR biomarker or biomarkers measured comprises ATRX. In some embodiments, the presence of ATRX with a loss of function mutation compared to wild type predicts sensitivity to MDM2 antagonism. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE. In some embodiments, the DDR biomarker or biomarkers measured comprises the mutational signature SBS6 and/or SBS26 associated with defects in DNA mismatch repair, and/or the POLD1 mutational signature SBS20.

In another embodiment, one or more of the DDR biomarkers is within the FA pathway.

In a further embodiment, the DDR biomarker, for example MMR biomarker, depletion is identified by MSI (e.g. MSI-High) and/or increased tumour mutational burden (e.g. high TMB, or increased relative to a non-sensitive cancer cell).

Optionally, the method may comprise the step of administering a therapeutically effective amount of an MDM2 antagonist to the patient.

In all aspects and embodiments described herein, the cancer is typically a p53-wild-type cancer.

In one embodiment, the invention provides an MDM2 antagonist for use in the treatment of cancer, in particular a p53 wild type cancer, wherein the cancer is characterised by one or more of the biomarkers of the invention within a biological sample obtained from the patient.

According to another embodiment of the invention, there is provided a method of treating cancer in a patient wherein said method comprises the steps of selecting a patient based on the expression profile of one more of the biomarkers of the invention. In certain embodiments, the patient is selected based on:
having decreased expression or activity of one or more DDR biomarkers within a biological sample obtained from said patient;
and optionally then administering a therapeutically effective amount of a MDM2 antagonist to said patient. In one embodiment, the one or more DDR biomarkers are from one or more of the following pathways: HR, NHEJ, FA, MMR and/or BER. The DDR biomarker or biomarkers measured may all be in the same pathway, or may be from different pathways. In a further embodiment, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. In another embodiment the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. In another embodiment, one or more of the DDR biomarkers is within the FA pathway. In a further embodiment, the biomarker depletion is identified by increased MSI and/or increased tumour mutational burden.

According to a further embodiment of the invention, there is provided an MDM2 antagonist for use in the treatment of cancer in a patient, characterised in that said patient has been selected for having decreased or low expression of one or more DDR biomarkers within a biological sample obtained from said patient. In one embodiment, the one or more DDR biomarkers are from one or more of the following pathways: HR, NHEJ, FA, MMR and/or BER. The DDR biomarker or biomarkers measured may all be in the same pathway, or may be from different pathways. In a further embodiment, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. In another embodiment the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE. In some embodiments, the DDR biomarker or biomarkers measured comprises measuring the mutational signature SBS6 and/or SBS26 associated with defects in DNA mismatch repair, and/or the POLD1 mutational signature SBS20. In another embodiment, one or more of the DDR biomarkers is within the FA pathway. In a further embodiment, the biomarker depletion is identified by increased MSI (e.g. MSI-High) and/or high tumour mutational burden (TMB).

In certain embodiments a sample of patient tissue is tested prior to treatment, to determine the cancer biomarker expression profile. The sample may typically comprise one or more cancer cells, cancer DNA, or circulating tumour DNA. The sample may be a blood sample. The sample may be a tumour sample, for example a tumour biopsy. The testing may comprise an assay to detect protein, mRNA, DNA and/or ctDNA.

In another aspect, the invention provides the use of the expression levels of one or more biomarkers of the invention in a cancer cell sample of a human patient, as biomarkers for assessing whether the cancer is susceptible to treatment with an MDM2 antagonist.

In a further aspect, the invention provides a method for prognosing or assessing the responsiveness of a human cancer patient to treatment with an MDM2 antagonist, comprising assessing the expression level in a sample from a cancer patient of one or more biomarkers of the invention and determining whether the tested expression level indicates that the cancer should be treated with an MDM2 antagonist.

In some embodiments, the one or more biomarkers of the invention indicate that the cancer is likely to be apoptosed effectively. Therefore, in some embodiments the invention is able to identify those patients for whom treatment will be particularly effective.

In some embodiments, the assessment step comprises an in vitro assay to determine the expression level of the biomarker or biomarkers.

In some embodiments, the assessment step comprises comparing the expression level with the expression level known to be associated with responsiveness or non-responsiveness to treatment with an MDM2 antagonist. In some embodiments, the assessment step comprises comparing the observed expression level with a threshold value reflecting in the same manner the expression level associated with susceptibility to treatment with an MDM2 antagonist, to assess whether the tested expression level indicates that the cancer can be treated with an MDM2 antagonist.

In some embodiments, the patient is classified into a group based on the biomarker profile. This may include classifying the patient as likely to respond well (or strongly), or not, to treatment with an MDM2 antagonist.

In a further aspect, the invention provides a method of determining whether a human cancer patient is suitable for treatment with an MDM2 antagonist, comprising detecting in a sample of cancer cells from the patient the expression or activity of one or more biomarkers of the invention; and assessing whether the cancer in the patient is likely to be treated with a MDM2 antagonist on the basis of the expression or activity level of the biomarkers in the sample. Optionally, the method of this aspect comprises the further step of treating the cancer in the patient using an MDM2 antagonist.

In a further embodiment the invention provides an MDM2 antagonist for use in the treatment of cancer in a patient in combination with an anticancer compound, characterised in that said cancer in said patient is a p53 wild type cancer, which has been selected for having one or more biomarkers of the invention.

In a further embodiment the invention provides a method of treating cancer in a patient, wherein said cancer in said patient is optionally a p53 wild type cancer, and wherein the patient has been selected as having one or more biomarkers of the invention at a level that indicates that MDM2 antagonist treatment will be effective; and administering a therapeutically effective amount of a MDM2 antagonist and optionally another anticancer agent to the selected patient.

In a further embodiment the invention provides a method of identifying a patient suffering from cancer suitable for treatment with an MDM2 antagonist wherein said method comprises detecting, and optionally quantifying, the expression of one or more biomarkers of the invention.

In a further embodiment the invention provides a method of selecting a patient (e.g. suffering from cancer) wherein said method comprises the steps of selecting a patient by detecting, and optionally quantifying, the expression of one or more biomarkers of the invention.

In a further embodiment the invention provides a method of determining the likelihood that a cancer patient will respond to therapy with an MDM2 antagonist, the method comprising:
obtaining a measurement of decreased expression of one or more of the DDR biomarkers in a cancer cell sample from the patient, compared to a corresponding non-cancer cell;
and determining that the patient is likely to respond to therapy with an MDM2 antagonist on the basis of that measurement. The one or more DDR biomarkers are optionally from one or more of the following pathways: HR, NHEJ, MMR, FA and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment the invention provides a drug administration process comprising:
determining one or more biomarkers of the invention
administering a therapeutically effective amount of an MDM2 antagonist to a patient with one or more biomarkers of the invention.

In yet a further aspect, the invention provides a method of detecting the expression of one or more biomarkers of the invention in a human patient suffering from cancer. This method typically comprises:
(a) obtaining a sample of cancer cells from a human patient; and
(b) detecting whether said biomarkers are expressed in the sampled cancer cells by contacting the sample with one or more reagents for detecting expression of the biomarkers.

In a still further aspect, the invention provides a kit or device for detecting the expression level of at least one biomarker for sensitivity to MDM2 antagonism in a sample from a human patient, said kit or device comprising a detection reagent or detection reagents for detecting one or more biomarkers of the invention

In a further aspect, the invention resides in a system for assessing whether a human cancer patient is susceptible to treatment with an MDM2 antagonist, the system comprising:
detection means able and adapted to detect in a sample of from the human patient one or more biomarkers of the invention
a processor able and adapted to determine from the determined biomarker or biomarkers an indication of the likelihood of the patient being treatable with an MDM2 antagonist.

The system optionally contains a data connection to an interface, particularly a graphical user interface, capable of presenting information, preferably also capable of putting in information such as the age of the subject, as well as optionally other patient information such as sex and/or medical history information, said interface being either a part of the system or a remote interface. Optionally one or more of the foregoing items, particularly the processor, are enabled to function “in the cloud”, i.e., not on a fixed machine, but by means of an internet-based application.

The invention also provides methods of identifying and screening patients, combinations, and kits.

In a further embodiment, the invention provides a method of screening or identifying a patient for treatment with an MDM2 antagonist comprising determining whether said patient has:
decreased expression of one or more DDR biomarkers within a biological sample obtained from said patient. In one embodiment, the one or more DDR biomarkers are from one or more of the following pathways: HR, NHEJ, MMR, FA and/or BER. The DDR biomarker or biomarkers measured may all be in the same pathway, or may be from different pathways. In a further embodiment, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. In another embodiment the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE. In some embodiments, the DDR biomarker or biomarkers measured comprises mutational signature SBS6 and/or SBS26 associated with defects in DNA mismatch repair, and/or the POLD1 mutational signature SBS20. In another embodiment, one or more of the DDR biomarkers is within the FA pathway. In a further embodiment, the MMR biomarker depletion is identified by MSI and/or high tumour mutational burden.

In a further embodiment, the invention provides a method of identifying a patient responder comprising testing a patient for:
decreased expression of one or more DDR biomarkers within a biological sample obtained from said patient. In one embodiment, the one or more DDR biomarkers are from one or more of the following pathways: HR, NHEJ, MMR, FA and/or BER. The DDR biomarker or biomarkers measured may all be in the same pathway, or may be from different pathways. In a further embodiment, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. In another embodiment the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. In another embodiment, one or more of the DDR biomarkers is within the FA pathway. In a further embodiment, the MMR biomarker depletion is identified by MSI and/or high tumour mutational burden.

In a further embodiment, the invention provides a method of treatment comprising:
(a) identifying a patient in need of treatment for cancer, optionally a p53 wild type cancer such as mesothelioma;
(b) determining that the patient has decreased expression of one or more DDR biomarkers within a biological sample obtained from said patient; and treating the patient with a therapeutically effective amount of an MDM2 antagonist.

In a further embodiment, the invention provides a method of treatment comprising:
(a) identifying a patient in need of treatment for cancer, optionally breast, ovarian, prostate or pancreatic cancer;
(b) determining that the patient has decreased expression of one or more DDR biomarkers within a biological sample obtained from said patient; and treating the patient with a therapeutically effective amount of an MDM2 antagonist.

Optionally, the one or more DDR biomarkers are from one or more of the following pathways: HR, NHEJ, MMR, FA and/or BER. The DDR biomarker or biomarkers measured may all be in the same pathway, or may be from different pathways. Optionally, the DDR biomarker or biomarkers measured comprises ATM. Optionally, the DDR biomarker or biomarkers measured comprises BRCA1. Optionally, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment, the invention provides a method of treatment comprising:
(a) identifying a patient in need of treatment for cancer, optionally mesothelioma;
(b) determining one or more biomarkers of the invention in the patient, optionally, one or more DDR biomarkers from one or more of the HR, NHEJ, FA, MMR and/or BER pathways;
   a. optionally, the DDR biomarker or biomarkers measured comprises 1, 2, 3 or 4 of BRCA1, BRCA2, ATM and ATRX; and/or
   b. optionally, one or more of the DDR biomarkers is within the FA pathway; and/or
   c. optionally, one or more of the DDR biomarkers is within the MMR pathway, for example one or more of MSH2, MSH3, MSH6, MLH1, PMS2, MLH3, POLE and/or POLD1, and/or mutational signature SBS6, SBS26 and/or SBS20, wherein optionally depletion of said one or more MMR pathway DDR biomarkers is identified by MSI and/or high tumour mutational burden;
(c) selecting an MDM2 antagonist as a treatment for the patient, based on the recognition that MDM2 antagonists are effective in patients who have one or more biomarkers of the invention;
(d) treating the patient with a therapeutically effective amount of an MDM2 antagonist.

In a further embodiment, the invention provides a method of treatment comprising:
(a) identifying a patient in need of treatment for cancer, optionally breast, ovarian, prostate or pancreatic cancer;
(b) determining one or more biomarkers of the invention in the patient, optionally, one or more DDR biomarkers from one or more of the HR, NHEJ, FA, MMR and/or BER pathways;
   a. optionally, the DDR biomarker or biomarkers measured comprises or consists of 1, 2, 3 or 4 of BRCA1, BRCA2, ATM and ATRX; and/or
   b. optionally, one or more of the DDR biomarkers is within the FA pathway; and/or
   c. optionally, one or more of the DDR biomarkers is within the MMR pathway, for example one or more of MSH2, MSH3, MSH6, MLH1, PMS2, MLH3, POLE and/or POLD1, and/or mutational signature SBS6, SBS26 and/or SBS20, wherein optionally depletion of said one or more MMR pathway DDR biomarkers is identified by MSI and/or high tumour mutational burden;
(c) selecting an MDM2 antagonist as a treatment for the patient, based on the recognition that MDM2 antagonists are effective in patients who have one or more biomarkers of the invention;
(d) treating the patient with a therapeutically effective amount of an MDM2 antagonist.

In a further embodiment, the invention provides a method of selecting a treatment for a cancer patient comprising:
(a) assaying one or more biological samples thereby determining one or more biomarkers of the invention in the patient;
(b) based on that determination selecting that patient for treatment with a therapeutically effective amount of an MDM2 antagonist.

In one embodiment, DDR biomarker is from the HR pathway and is one or more HR gene(s) selected from: LIG1, MRE11A, NBN, PARG, PARP1, PARPBP, RAD50, TP53BP1, XRCC2, XRCC3, EXO1, PCNA, POLD1, POLD2, POLD3, POLD4, RFC1, RFC2, RFC3, RFC4, RFC5, RPA1, RPA2, RPA3, RPA4, BARD1, BLM, BRCA1, BRCA2, BRIP1, DMC1, DNA2, EID3, EME1, EME2, ERCC1, H2AFX, HELQ, HFM1, INO80, KAT5, MUS81, NFATC2IP, NSMCE1, NSMCE2, NSMCE3, NSMCE4A, PALB2, PARP2, PAXIP1, POLH, POLQ, PPP4C, PPP4R1, PPP4R2, PPP4R4, RAD51, RAD51B, RAD51C, RAD51D, RAD52, RAD54B, RAD54L, RBBP8, RDM1, RECQL, RECQL4, RECQL5, RM11, RMI2, RTEL1, SHFM1, SLX1A, SLX1B, SLX4, SMARCAD1, SMC5, SMC6, SPO11, SWSAP1, TOP3A, TOP3B, UIMC1, WRN, and/or ZSWIM7.

In a further embodiment, the invention provides a process for selecting a patient (e.g. suffering from cancer) for treatment with an MDM2 antagonist, characterised in that said patient has been selected for having:
decreased or low expression of one or more DDR biomarkers within a biological sample obtained from said patient. Optionally, the one or more DDR biomarkers are from one or more of the following pathways: HR, NHEJ, FA, MMR and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment, the invention provides an MDM2 antagonist for use in the treatment of cancer in a patient, characterised in that said patient is known to have decreased expression or activity of one or more DDR biomarkers within a biological sample obtained from said patient. Optionally, the one or more DDR biomarkers are from one or more of the following pathways: HR, NHEJ, FA, MMR and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden. In a further embodiment, the invention provides a kit for treating cancer in a patient, wherein said kit comprises a biosensor for detection and/or quantification of one or more biomarkers of the invention, and/or reagents for the detection of one or more biomarkers of the invention, optionally together with instructions for use of the kit in accordance with the methods as defined herein.

In a further embodiment, the invention provides a method of determining responsiveness of an individual with cancer to treatment with an MDM2 antagonist comprising detecting decreased expression or activity of one or more DDR biomarkers within a biological sample obtained from said patient. Optionally, the one or more DDR biomarkers are from one or more of the following pathways: HR, NHEJ, FA, MMR and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2.

Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment, the invention provides a method of determining responsiveness of an individual with cancer to treatment with an MDM2 antagonist comprising identifying a patient:
having decreased expression or activity of one or more DDR biomarkers within a biological sample obtained from said patient; and then administering a therapeutically effective amount of an MDM2 antagonist to said patient.

Said one or more DDR biomarkers are optionally from one or more of the following pathways: HR, NHEJ, MMR, FA and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment, the invention provides a method of treating cancer in a patient wherein said method comprises the steps of selecting a patient having decreased expression or activity of one or more DDR biomarkers within a biological sample obtained from said patient. Optionally, the one or more DDR biomarkers are from one or more of the following pathways: HR, NHEJ, MMR and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment, the invention provides a drug administration process comprising:
(i) ordering determination of expression or activity of one or more DDR biomarkers;
   a. optionally, the one or more DDR biomarkers are from one or more of the following pathways: HR, NHEJ, FA, MMR and/or BER; and
(ii) administering a therapeutically effective amount of an MDM2 antagonist to a patient with decreased levels of one or more DDR biomarkers.

In a further embodiment, the invention provides a packaged pharmaceutical product comprising:
(i) an MDM2 antagonist;
(ii) patient insert detailing instructions for use of the MDM2 antagonist in the treatment of patients identified using the biomarker profile described herein.

In a further embodiment, the invention provides a method of treating cancer in a patient wherein said method comprises:
(i) contacting a sample from a patient with a primer, antibody, substrate or probe, to determine the expression or activity levels of one or more DDR biomarkers;
   a. optionally the one or more DDR biomarkers are from one or more of the following pathways: HR, FA, NHEJ, MMR and/or BER;
   b. optionally, the DDR biomarker or biomarkers measured comprises ATM, BRCA1 BRCA2 and/or ATRX;
   c. optionally, the DDR biomarker or biomarkers measured comprises MSH2, MSH3, MSH6, MLH1, MLH3, PMS2, POLE and/or POLD1 mutations, optionally wherein the cancer comprises mutational signature SBS6, SBS26 and/or SBS20;
   d. optionally, one or more of the DDR biomarkers is within the FA pathway; and/or
   e. optionally, where depletion of said one or more biomarkers may identified by MSI and/or high tumour mutational burden.
(ii) selecting a patient having decreased levels of one or more DDR biomarkers in a biological sample obtained from said patient;
(iii) followed by administering a therapeutically effective amount of an MDM2 antagonist to said patient selected in step (ii).

In a further embodiment, the invention provides a method for identifying a patient for treatment with an MDM2 antagonist, the method comprising:
(a) contacting a sample from the patient with a plurality of oligonucleotide primers, said plurality of primers comprising at least one pair of oligonucleotide primers for any one or more DDR biomarkers;
(b) performing PCR on said sample to amplify gene expression products/transcripts in the sample;
(c) determining the level of an expression product of at least one of said genes; and
(d) identifying the patient as a candidate for treatment with an MDM2 antagonist when the expression level of said at least one gene is low relative to the upper limit of normal (ULN).

The patient may optionally be identified as a candidate for treatment with an MDM2 antagonist when the expression level of one of more DDR biomarkers is low relative to (e.g. below) the upper limit of normal (ULN). Optionally, the one or more DDR biomarkers are from one or more of the following pathways: HR, NHEJ, FA, MMR and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises one or more POLD1 and/or POLE mutations. In some embodiments, the DDR biomarker or biomarkers measured comprises mutational signature SBS6 and/or SBS26 associated with defects in DNA mismatch repair, and/or the POLD1 mutational signature SBS20. Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In a further embodiment, the invention provides a method for identifying a patient for treatment with an MDM2 antagonist, the method comprising:
(a) contacting a sample from the patient with an antibody against one or more biomarkers of the invention;
(b) performing an assay on said sample;
(c) determining the level of one or more biomarkers of the invention; and
(d) identifying the patient as a candidate for treatment with an MDM2 antagonist when the level of one or more biomarkers of the invention is elevated or reduced relative to the upper limit of normal (ULN).

The assay in part (b) may be or comprise an immunohistochemical assay. In some embodiments, the assay may be or comprise an ELISA. When the sample from the patient is contacted with an antibody against one or more DDR biomarkers, an immunohistochemical assay is typically performed on said sample, and the patient is identified as a candidate for treatment with an MDM2 antagonist when the level of one or more DDR biomarkers is low (or absent) relative to the upper limit of normal (ULN).

Once a patient has been identified for treatment, the methods described herein can further comprise treating cancer in the patient with an MDM2 antagonist.

In a further embodiment, the invention provides a method of selecting a cancer patient for receiving an MDM2 antagonist therapy for a cancer, comprising:
(a) determining the level of one or more DDR biomarkers in a biological sample from the patient;
   the one or more DDR biomarkers are optionally from one or more of the HR, FA, NHEJ, MMR and/or BER pathways; and/or
   the DDR biomarker or biomarkers measured optionally comprise ATM, BRCA1, BRCA2 and/or ATRX; and/or
   the DDR biomarker or biomarkers measured optionally comprises MSH2, MSH3, MSH6, MLH1, PMS2, MLH3, POLE and/or POLD1, optionally wherein the cancer comprises mutational signature SBS6, SBS26 and/or SBS20; and/or one or more of the DDR biomarkers is optionally within the FA pathway; and/or where one or more of the DDR biomarkers is in the MMR pathway, depletion of said one or more biomarkers is optionally identified by MSI and/or high tumour mutational burden;
and
(b) selecting the patient who has a level of one or more DDR biomarkers in the biological sample from the patient that is lower than a predetermined value in the biological sample from the patient that is equal to or greater than a predetermined value.

In a further embodiment, the invention provides a method for predicting efficacy of MDM2 antagonist for a cancer in a patient, or for predicting response of a cancer patient to an MDM2 antagonist for a cancer, comprising determining the level of one or more DDR biomarker in a biological sample from the patient, where a biological sample level of the one or more DDR biomarker equal to or typically less than a predetermined value is predictive of efficacy in the patient. Optionally, the one or more DDR biomarkers are from one or more of the following pathways: HR, NHEJ, MMR and/or BER. The DDR biomarker or biomarkers measured may all be in the same pathway, or may be from different pathways. Optionally, the DDR biomarker or biomarkers measured comprises ATM. In a further embodiment, the DDR biomarker or biomarkers measured comprises BRCA1. In a further embodiment, the DDR biomarker or biomarkers measured comprises BRCA2. Optionally, the DDR biomarker or biomarkers measured comprises ATRX. In one embodiment, the DDR biomarker or biomarkers measured comprises one or more of MSH2, MSH3, MSH6, MLH1, PMS2 and/or MLH3. In a further embodiment, the DDR biomarker or biomarkers measured comprises POLD1 and/or POLE. In some embodiments, the DDR biomarker or biomarkers measured comprises mutational signature SBS6 and/or SBS26 associated with defects in DNA mismatch repair, and/or the POLD1 mutational signature SBS20.

Optionally, one or more of the DDR biomarkers is within the FA pathway. Optionally, where one or more of the DDR biomarkers is in the MMR pathway, depletion of said one or more biomarkers is identified by MSI and/or high tumour mutational burden.

In one embodiment the patient is selected for MDM2 antagonist treatment on the basis of measuring one more HR pathway biomarker genes selected from: LIG1, MRE11A, NBN, PARG, PARP1, PARPBP, RAD50, TP53BP1, XRCC2, XRCC3, EXO1, PCNA, POLD1, POLD2, POLD3, POLD4, RFC1, RFC2, RFC3, RFC4, RFC5, RPA1, RPA2, RPA3, RPA4, BARD1, BLM, BRCA1, BRCA2, BRIP1, DMC1, DNA2, EID3, EME1, EME2, ERCC1, H2AFX, HELQ, HFM1, INO80, KAT5, MUS81, NFATC2IP, NSMCE1, NSMCE2, NSMCE3, NSMCE4A, PALB2, PARP2, PAXIP1, POLH, POLQ, PPP4C, PPP4R1, PPP4R2, PPP4R4, RAD51, RAD51B, RAD51C, RAD51D, RAD52, RAD54B, RAD54L, RBBP8, RDM1, RECQL, RECQL4, RECQL5, RM11, RMI2, RTEL1, SHFM1, SLX1A, SLX1B, SLX4, SMARCAD1, SMC5, SMC6, SPO11, SWSAP1, TOP3A, TOP3B, UIMC1, WRN, and/or ZSWIM7.

In a further embodiment, the invention provides a method of selecting a patient having cancer in need of treatment with an MDM2 antagonist which comprises testing a tumour sample obtained from the patient for low level of one or more DDR biomarkers.

In a further embodiment, the invention provides a method of treating cancer comprising (i) testing a tumour sample obtained from a patient suffering from or likely to suffer from cancer for loss of one or more DDR biomarkers and (ii) administering an MDM2 antagonist to the patient from which the sample was taken. Optionally, the one or more DDR biomarkers in (i) are from one or more of the HR, NHEJ, MMR and/or BER pathways. Optionally, the DDR biomarker or biomarkers measured comprise ATM, BRCA1, BRCA2 and/or ATRX. Optionally, one or more of the DDR biomarkers is within the FA pathway.

Optionally, where one or more of the DDR biomarkers is in the MMR pathway, depletion of said one or more biomarkers is identified by MSI and/or high tumour mutational burden. Optionally, the DDR biomarker or biomarkers measured comprise one or more of MSH2, MSH3, MSH6, MLH1, PMS2 MLH3,POLD1 and/or POLE. Optionally, the DDR biomarker or biomarkers measured comprises mutational signature SBS6 and/or SBS26 associated with defects in DNA mismatch repair, and/or the POLD1 mutational signature SBS20.

In a further embodiment, the invention provides a method of identifying a patient having cancer most likely to benefit from treatment with an MDM2 antagonist comprising measuring the level of one or more of the biomarkers of the invention in a tumour sample obtained from the patient and identifying whether or not the patient is likely to benefit from treatment with an MDM2 antagonist according to the levels present.

Some embodiments of the invention comprise detecting the presence of mutation of one or more DDR biomarkers indicative of loss of said one or more DDR biomarkers. These mutations may be compared to control levels determined in normal non-proliferative tissue or absence of mutation.

The invention variously provides: a method of determining if a cancer patient is amenable to treatment with an MDM2 antagonist; a method of predicting the sensitivity of tumour cell growth to inhibition by a MDM2 antagonist; a method of predicting responsiveness of a cancer in a subject to a cancer therapy including an MDM2 antagonist; a method of developing a treatment plan for a subject with cancer; an in vitro method for the identification of a patient responsive to or sensitive to treatment with an MDM2 antagonist regimen. The methods typically comprise comparing the levels of one or more biomarkers of the invention in the sample, typically a tumour sample, to a reference level and predicting the responsiveness of the cancer to treatment with the cancer therapy including an MDM2 antagonist. In one embodiment the methods comprise analysing one or more, for example, two or more, or three or more, or four or more, or five or more, or six or more, or seven or more, or eight or more, or nine or more, or ten or more, or fifteen or more biomarkers described herein. In one embodiment the one or more biomarkers include ATM. In one embodiment the one or more biomarkers do not include ATM.

In one embodiment the one or more biomarkers do not include ATR. In one embodiment the one or more biomarkers include BRCA1 or BRAC2. In one embodiment the two or more biomarkers include BRCA1 and BRAC2. In one embodiment the two or more biomarkers include BAP1 and CDKN2A. In one embodiment the two or more biomarkers include ATM. In one embodiment the two or more biomarkers include ATR.

In a further embodiment, the invention provides an in vitro method for predicting the likelihood that a patient suffering from a tumour, who is a candidate for treatment with an MDM2 antagonist, will respond to the treatment with the compound, comprising the step of: (a) determining the levels of one or more DDR biomarkers in one or more tissue samples taken from the patient, wherein (i) loss of one or more DDR biomarkers (e.g compared to a reference value of at least one normal non-proliferative tissue) indicates that the patient is likely to respond to the treatment and/or (ii) normal or high levels of one or more DDR biomarkers indicates that the patient is less likely to respond to the treatment.

In a further embodiment, the invention provides an assay comprising: (a) measuring or quantifying the level of one or more DDR biomarkers; (b) comparing the level of said one or more DDR biomarkers (e.g. relative to control levels determined in healthy individuals) (e.g. relative to control levels determined in normal non-proliferative tissue), and if there is loss one or more DDR biomarkers (e.g. relative to control levels determined in normal non-proliferative tissue) identifying the patient as suitable for treatment with an MDM2 antagonist.

In a further embodiment, the invention provides an assay comprising:
(i) contacting a biological sample obtained from a patient with an antibody (e.g. antibody specific against one or more DDR biomarker);
(ii) washing the sample to remove unbound antibody;
(iii) measuring the intensity of the signal from the bound antibody;
(iv) comparing the measured intensity of the signal with a reference value and if the measured intensity is increased relative to the reference value;
(v) contacting a biological sample obtained from a patient with
   a. a primer (e.g. at least one oligonucleotide primer pairs for any one or more DDR biomarker),
   b. an antibody (e.g. antibody specific against one or more DDR biomarker), and/or
   c. a primer for a gene or mutant indicative of loss of one or more DDR biomarker;
(vi) performing PCR, RT-PCR or next generation sequencing on said sample to amplify gene expression products/transcripts in the sample;
(vii) determining the level of an expression product of at least one of said genes; and
(viii) identifying the subject as having an increased probability of being suitable for treatment with an MDM2 antagonist.

In a further embodiment, the invention provides a method of treating cancer comprising administering an MDM2 antagonist to a subject with loss of one or more DDR biomarkers in a tumour sample as determined by sequencing or immunoassay.

In a further embodiment, the invention provides a method of administering an MDM2 antagonist to a patient in need thereof comprising:
(1) determining the patient levels of one or more DDR biomarkers;
(2) assigning a phenotype to the patient based on the levels of the genes listed above and genotype of the tumour as determined in (1), wherein the phenotype is selected from poor (P), intermediate (I), and sensitive (S), and said phenotype is assigned based upon the level of the genes in the tumour; and
(3) administering to the patient with phenotype S an MDM2 antagonist.

In one embodiment the homologous recombination deficiency (HRD) score from Myriad MyChoice CDx (a FDA-approved tumor test from Myriad Genetics Inc., Salt Lake City, Utah, USA) is used to determine homologous recombination deficiency status by looking at BRCA1 and BRCA2 variants and assessing genomic instability using three biomarkers: loss of heterozygosity, telomeric allelic imbalance and large-scale state transitions. HRD positive is defined as HRD score>=42 and HRD negative is <42.

Myriad myChoice® CDx next generation sequencing-based in vitro diagnostic test assesses the qualitative detection and classification of single nucleotide variants, insertions and deletions, and large rearrangement variants in protein coding regions and intron/exon boundaries of the BRCA1 and BRCA2 genes and the determination of Genomic Instability Score (GIS) which is an algorithmic measurement of Loss of Heterozygosity (LOH), Telomeric Allelic Imbalance (TAI), and Large-scale State Transitions (LST) using DNA isolated from formalin-fixed paraffin embedded (FFPE) tumor tissue specimens.

The results of the test can be used as an aid in identifying cancer patients with positive homologous recombination deficiency (HRD) status, who are eligible, because of a positive test result for deleterious or suspected deleterious mutations in BRCA1 or BRCA2 genes, or may become eligible, because of a positive test result for deleterious or suspected deleterious mutations in BRCA1 or BRCA2 genes or a positive Genomic Instability Score, for treatment with the MDM2 antagonist (once approved in accordance with the approved therapeutic product labelling).

In one embodiment MSI testing is performed. MSI testing can be performed on fresh, frozen or paraffin-embedded tumor tissue using a PCR-based assay for detection of instability.

The National Cancer Institute Workshop agreed on five microsatellite markers necessary to determine MSI that include two mononucleotide—BAT25/26 and three dinucleotide markers—D2S123, D5S346, and D17S250. Interpretation of the profiles requires a comparison with normal DNA from each patient. An alternative molecular method based exclusively on quasi-monomorphic mononucleotide markers was developed to avoid the analysis of matching normal DNA. This method has been proven to be more specific and sensitive than the original NCI panel (J Clin Oncol. 2006 Jan. 10; 24(2):241-51.).

On the basis of the MSI status, cancers e.g. CRC can be classified into three groups: MSI-H, if two or more of the five microsatellite markers show instability; MSI-L (low-frequency MSI), if only one of five markers shows instability; and microsatellite stable (MSS) if none of the markers show instability (Cancer Res. 1998 Nov. 15; 58(22):5248-57.).

IHC is an alternative test that is widely available with the advantage of not requiring a molecular laboratory, and the ability to identify the affected gene by detecting loss of its protein product. Another advantage of IHC testing is that loss of a specific mismatch gene product (MLH1, MSH2, MSH6, and PMS2) can direct germline testing to that specific gene, and assists in the identification of patients.

Deficient mismatch repair (dMMR), is associated with high-frequency microsatellite instability (H-MSI); dMMR testing can also be done via IHC.

Cancer Cell Int 20, 16 (2020) contains a summary of microsatellite instability detection methods including NGS (next-generation sequencing), PCR (polymerase chain reaction), CE (capillary electrophoresis), IHC (immunohistochemistry), smMIPs (single-molecule molecular inversion probes).

| Detection | | | |
|---|---|---|---|
| method | Test items | Accuracy | Refs. |
| NGS | Nearly 100 MS loci | IMPACT ™: 92% | Hempelmann et |
| | | F1CDx: 94.6% | al. [symbol] |
| Fluorescent | 5 MS sites: BAT-26, | Gold standard, | Arulananda et |
| multiplex | NR-21, BAT-25, | 100% | al. [symbol] |
| PCR and | MONO-27 and NR-24 | | |
| CE | | | |
| IHC | The MMR protein: | 89-95% | Cheah et al. [symbol] |
| | hMLH1, hPMS2, | | |
| | hMSH2, hMSH6 | | |
| smMIPs | DNA from tumor tissue | 95.80% | Waalkes et al. |
| | | | [symbol] |

In a further embodiment, the invention provides use of an MDM2 antagonist in the manufacture of a medicament for use in the treatment of cancer in a patient wherein the cancer tumour has loss of one or more DDR biomarkers.

In a further embodiment, the invention provides use of an MDM2 antagonist in the manufacture of a medicament for use in the treatment of cancer in a patient identified as likely to be responsive to treatment with an MDM2 antagonist according to the method described herein.

In a further embodiment, the invention provides an article of manufacture comprising, packaged together, an MDM2 antagonist medicament in a pharmaceutically acceptable carrier and a package insert indicating that the cancer (e.g. mesothelioma, renal, or glioblastoma) medicament is for treating a patient with cancer based on levels of a biomarker or biomarkers identified herein as determined by an assay method used to measure the levels.

In a further embodiment, the invention provides a method for advertising an MDM2 antagonist medicament comprising promoting, to a target audience, the use of the MDM2 antagonist medicament for treating a cancer patient with loss of one or more DDR biomarkers.

In a further embodiment, the invention provides apparatus configured to identify a tumour (e.g. mesothelioma) of a cancer patient as being likely to benefit from treatment with a therapeutic agent or a combination of therapeutic agents targeting MDM2 or not likely to benefit from treatment with the therapeutic agent or combination of therapeutic agents. The apparatus may comprise a storage device storing sequencing data or immunoassay data from tumour or blood-based samples for the levels of one or more DDR biomarker genes, and/or loss of one or more DDR biomarkers to identify the patient as being either likely or not likely to benefit from the therapeutic agent or a combination of therapeutic agents targeting MDM2.

In one embodiment of the method described here when the levels of one or more DDR biomarkers is are low or absent (e.g. loss) then the patient is administered an MDM2 antagonist.

In another embodiment of the method described here when the levels of one or more DDR biomarkers are high (or present) then the patient is not administered an MDM2 antagonist.

In certain embodiments, an MDM2 antagonist may be administered to a patient in combination with an additional cancer treatment that is not an MDM2 antagonist. In one embodiment the at least one biomarker of the invention can be used to select a patient to treat with an MDM2 antagonist in combination with an agent described in (i)-(xlix) below.

In certain embodiments, an MDM2 antagonist may be administered to a patient in combination with an agent to induce sensitivity to an MDM2 antagonist for example to lower the levels of one or more genes or gene products in a DNA damage repair (DDR) pathway.

In certain embodiments, a method of treating cancer in a patient comprises the steps of selecting a patient:
(a) having normal or high levels of DDR pathway genes or gene products, within a biological sample obtained from said patient; and
(b) administering a therapeutically effective amount of an MDM2 antagonist and an agent to induce sensitivity to an MDM2 antagonist for example by lowering the levels of one or more genes or gene products in a DNA damage repair (DDR) pathway, to said patient selected in step (a).

In one embodiment the agent to lower the levels of one or more DDR pathway gene products is an inhibitor of the DDR pathway gene or gene product.

In one embodiment the agent to lower the levels of one or more DDR pathway gene products is an inhibitor of BRCA1, BRCA2, ATM and/or ATRX.

### BRIEF DESCRIPTION OF THE FIGURES

FIG. 1: Dual Loss of Function CRISPR screen identified DDR as the top sensitizing pathway to Compound 1. (A) Horizon CRISPR data: Network analysis of CRISPR hits showed enrichment of Fanconi Anemia pathway (Horizon's bioinformatics analysis). (B) Gene set enrichment analyses (GSEA) showed enrichment of other DDR pathways (in-house bioinformatics analysis). (C) Replication stress gene expression signature in mesothelioma apoptotic cell lines.

FIG. 2: ATM mutations are linked to increased sensitivity to Compound 1. (A) ATM mutant cancer cell lines are significantly dependent on MDM2 as compared to ATM wild cell lines based on public DepMAP RNAi data (version 20Q4). (B) ATM mutation status in apoptotic and non-apoptotic mesothelioma cell lines. (C) Reduction of proliferation in ATM mutant cell lines. (D) Apoptosis induction in ATM mutant cell lines. (E) Modulation of DDR signaling in ATM mutant cell lines. (F) Table summarizing the reduction of proliferation in BRCA1, BRCA2 and ATM mutant patient-derived organoids. (G) Reduction of proliferation in a breast cancer derived BRCA2 mutant compared to BRCA2 wild-type patient-derived organoid.

FIG. 3: ATRX is an additional hit from cell panel analysis. Bioinformatic analysis on the cell panel data, considering only P53-wild cell lines using all indications altogether. ANOVA method used to look for association of genomic features of Compound 1 sensitivity. ATRX loss predicted as significantly associated with sensitivity. (A) Volcano plot (ANOVA results of predictive biomarkers of sensitivity/resistance). (B) Boxplot of activity areas of ATRX-loss and ATRX-wild cell lines.

FIG. 4: Microsatellite instability (MSI) status and sensitivity. (A) Table listing activity areas and tumour mutation burden of MSI-H cancer cell lines in the cell panel. (B) Reduction of proliferation in MSI-H cancer cell lines from various indications. (C) Reduction of proliferation in six MSI-H colorectal cancer-derived patient-derived organoids. (D) In vivo efficacy experiment showing tumour growth reduction in an MSI-H xenograft model of colorectal cancer.

FIG. 5: Summary of the major DDR pathways, modified from Razqallah Hakem, EMBO J (2008) 27:589-605. Highlighted in boxes are DNA repair pathways where specific biomarkers have been identified by the inventors as sensitivity markers to MDM2 antagonism.

FIG. 6: X-ray powder diffractogram of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid.

FIG. 7: DSC scan of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid.

### DEFINITIONS

The term “MDM2 inhibitor” and “MDM2 antagonist” are used as synonyms and define MDM2 compounds or analogues of MDM2 compounds as described herein, including the ionic, salt, solvate, isomers, tautomers, N-oxides, ester, prodrugs, isotopes and protected forms thereof (preferably the salts or tautomers or isomers or N-oxides or solvates thereof, and more preferably, the salts or tautomers or N-oxides or solvates thereof), as described herein and above.

“MDM2 antagonist” means an antagonist of one or more MDM2 family members in particular MDM2 and MDM4 (also called MDMx). The term “antagonist” refers to a type of receptor ligand or drug that blocks or dampens agonist-mediated biological responses. Antagonists have affinity but no agonistic efficacy for their cognate receptors, and binding will disrupt the interaction and inhibit the function of any ligand (e.g. endogenous ligands or substrates, an agonist or inverse agonist) at receptors. The antagonism may arise directly or indirectly, and may be mediated by any mechanism and at any physiological level. As a result, antagonism of ligands may under different circumstances manifest itself in functionally different ways. Antagonists mediate their effects by binding to the active site or to allosteric sites on receptors, or they may interact at unique binding sites not normally involved in the biological regulation of the receptor's activity. Antagonist activity may be reversible or irreversible depending on the longevity of the antagonist-receptor complex, which, in turn, depends on the nature of antagonist receptor binding.

“Potency” is a measure of drug activity expressed in terms of the amount required to produce an effect of given intensity. A highly potent drug evokes a larger response at low concentrations. Potency is proportional to affinity and efficacy. Affinity is the ability of the drug to bind to a receptor. Efficacy is the relationship between receptor occupancy and the ability to initiate a response at the molecular, cellular, tissue or system level.

As used herein, the term “mediated”, as used e.g. in conjunction with MDM2/p53 as described herein (and applied for example to various physiological processes, diseases, states, conditions, therapies, treatments or interventions) is intended to operate limitatively so that the various processes, diseases, states, conditions, treatments and interventions to which the term is applied are those in which the protein plays a biological role. In cases where the term is applied to a disease, state or condition, the biological role played by the protein may be direct or indirect and may be necessary and/or sufficient for the manifestation of the symptoms of the disease, state or condition (or its aetiology or progression).

Thus, the protein function (and in particular aberrant levels of function, e.g. over- or under-expression) need not necessarily be the proximal cause of the disease, state or condition: rather, it is contemplated that the mediated diseases, states or conditions include those having multifactorial aetiologies and complex progressions in which the protein in question is only partially involved. In cases where the term is applied to treatment, prophylaxis or intervention, the role played by the protein may be direct or indirect and may be necessary and/or sufficient for the operation of the treatment, prophylaxis or outcome of the intervention. Thus, a disease state or condition mediated by a protein includes the development of resistance to any particular cancer drug or treatment.

The term “treatment” as used herein in the context of treating a condition i.e. state, disorder or disease, pertains generally to treatment and therapy, whether for a human or an animal (e.g. in veterinary applications), in which some desired therapeutic effect is achieved, for example, the inhibition of the progress of the condition, and includes a reduction in the rate of progress, a halt in the rate of progress, amelioration of the condition, diminishment or alleviation of at least one symptom associated or caused by the condition being treated and cure of the condition. For example, treatment can be diminishment of one or several symptoms of a disorder or complete eradication of a disorder.

The term “prophylaxis” (i.e. use of a compound as prophylactic measure) as used herein in the context of treating a condition i.e. state, disorder or disease, pertains generally to the prophylaxis or prevention, whether for a human or an animal (e.g. in veterinary applications), in which some desired preventative effect is achieved, for example, in preventing occurrence of a disease or guarding from a disease.

Prophylaxis includes complete and total blocking of all symptoms of a disorder for an indefinite period of time, the mere slowing of the onset of one or several symptoms of the disease, or making the disease less likely to occur.

References to the prophylaxis or treatment of a disease state or condition such as cancer include within their scope alleviating or reducing the incidence e.g. of cancer.

The combinations of the invention may produce a therapeutically efficacious effect relative to the therapeutic effect of the individual compounds/agents when administered separately.

The term ‘efficacious’ includes advantageous effects such as additivity, synergism, reduced side effects, reduced toxicity, increased time to disease progression, increased time of survival, sensitization or resensitization of one agent to another, or improved response rate. Advantageously, an efficacious effect may allow for lower doses of each or either component to be administered to a patient, thereby decreasing the toxicity of chemotherapy, whilst producing and/or maintaining the same therapeutic effect. A “synergistic” effect in the present context refers to a therapeutic effect produced by the combination which is larger than the sum of the therapeutic effects of the agents of the combination when presented individually. An “additive” effect in the present context refers to a therapeutic effect produced by the combination which is larger than the therapeutic effect of any of the agents of the combination when presented individually. The term “response rate” as used herein refers, in the case of a solid tumour, to the extent of reduction in the size of the tumour at a given time point, for example 12 weeks. Thus, for example, a 50% response rate means a reduction in tumour size of 50%. References herein to a “clinical response” refer to response rates of 50% or greater. A “partial response” is defined herein as being a response rate of less than 50%.

As used herein, the term “combination”, as applied to two or more compounds and/or agents, is intended to define material in which the two or more agents are associated. The terms “combined” and “combining” in this context are to be interpreted accordingly.

The association of the two or more compounds/agents in a combination may be physical or non-physical. Examples of physically associated combined compounds/agents include:
compositions (e.g. unitary formulations) comprising the two or more compounds/agents in admixture (for example within the same unit dose);
compositions comprising material in which the two or more compounds/agents are chemically/physicochemically linked (for example by crosslinking, molecular agglomeration or binding to a common vehicle moiety);
compositions comprising material in which the two or more compounds/agents are chemically/physicochemically co-packaged (for example, disposed on or within lipid vesicles, particles (e.g. micro- or nanoparticles) or emulsion droplets);
pharmaceutical kits, pharmaceutical packs or patient packs in which the two or more compounds/agents are co-packaged or co-presented (e.g. as part of an array of unit doses);

Examples of non-physically associated combined compounds/agents include:
material (e.g. a non-unitary formulation) comprising at least one of the two or more compounds/agents together with instructions for the extemporaneous association of the at least one compound to form a physical association of the two or more compounds/agents;
material (e.g. a non-unitary formulation) comprising at least one of the two or more compounds/agents together with instructions for combination therapy with the two or more compounds/agents;
material comprising at least one of the two or more compounds/agents together with instructions for administration to a patient population in which the other(s) of the two or more compounds/agents have been (or are being) administered;
material comprising at least one of the two or more compounds/agents in an amount or in a form which is specifically adapted for use in combination with the other(s) of the two or more compounds/agents.

As used herein, the term “combination therapy” is intended to define therapies which comprise the use of a combination of two or more compounds/agents (as defined above). Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologies of each of the two or more compounds/agents in a combination therapy may also differ with respect to the route of administration.

As used herein, the term “pharmaceutical kit” defines an array of one or more unit doses of a pharmaceutical composition together with dosing means (e.g. measuring device) and/or delivery means (e.g. inhaler or syringe), optionally all contained within common outer packaging. In pharmaceutical kits comprising a combination of two or more compounds/agents, the individual compounds/agents may unitary or non-unitary formulations. The unit dose(s) may be contained within a blister pack. The pharmaceutical kit may optionally further comprise instructions for use.

As used herein, the term “pharmaceutical pack” defines an array of one or more unit doses of a pharmaceutical composition, optionally contained within common outer packaging. In pharmaceutical packs comprising a combination of two or more compounds/agents, the individual compounds/agents may unitary or non-unitary formulations. The unit dose(s) may be contained within a blister pack. The pharmaceutical pack may optionally further comprise instructions for use.

The term ‘optionally substituted’ as used herein refers to a group which may be unsubstituted or substituted by a substituent as herein defined.

### DETAILED DESCRIPTION OF THE INVENTION

The invention is based on the identification of biomarkers that allow the determination of a cancer patient's likely response to MDM2 antagonist therapy. This provides for precision therapy of cancer using an MDM2 antagonist.

In certain embodiments, the invention provides a companion diagnostic for treatment of cancer using an MDM2 antagonist. As used herein, the term companion diagnostic is used to refer both to a test that is required to determine whether or not a patient will respond to a drug (i.e. a necessary companion diagnostic) and a test that is intended to identify whether the patient will respond favourably or optimally (which is sometimes referred to as a complementary diagnostic). In certain embodiments, the biomarkers identify a patient that will respond, and so discriminates responders from non-responders. In another embodiment, the biomarkers identify patients that will respond optimally, whereby the physician can then select the optimal treatment for that patient.

In some embodiments, the invention provides assays for determining the expression or activity level of 1, 2, 3, 4, 5, 6, 7, 8, 9, 10, 20, 25 or more of the biomarkers identified herein. This may be determined directly or indirectly, as discussed above. This assay may or may not include a step of deducing a prognostic outcome. The assay is typically an in vitro assay carried out on a sample from the patient, such as a cancer biopsy or a blood sample (whether or not the cancer is a blood cancer).

### Biomarkers for Effective Cancer Treatment

The present disclosure provides biomarkers that indicate increased sensitivity of cancer cells to treatment with an MDM2 antagonist. The identification of one or more of the identified biomarkers therefore allows a cancer patient to be selected for MDM2 antagonist treatment.

The biomarkers are DNA-damage response (DDR) pathway genes. Optionally, the one or more DDR biomarker genes are from one or more of the following pathways: HR, NHEJ, MMR, FA and/or BER. Optionally, the DDR biomarker or biomarkers measured comprises BRCA1. Optionally, the DDR biomarker or biomarkers measured comprises BRCA2. Optionally, the DDR biomarker or biomarkers measured comprises ATM. Optionally, the DDR biomarker or biomarkers measured comprises ATRX. Optionally, one or more of the DDR biomarkers is within the FA pathway. Optionally, where one or more of the DDR biomarkers is in the MMR pathway, depletion of said one or more biomarkers is identified by MSI (typically MSI-High) and/or high tumour mutational burden. Optionally, the DDR biomarker or biomarkers measured comprise MSH2, MSH3, MSH6, MLH1, PMS2 MLH3, POLE and/or POLD1. Optionally, the cancer comprises mutational signature SBS6 and/or SBS26 associated with defects in DNA mismatch repair, and/or the POLD1 mutational signature SBS20.

### DNA Damage Response (DDR)

Cells are continuously challenged by a variety of genotoxic attacks and can revert the large variety of DNA lesions via several DNA-repair machineries, many of which involve p53. Erroneous repair of the DNA can lead to mutations and chromosomal aberrations that can alter the functions of tumour suppressor genes or oncogenes, thus causing cancer development.

As a central tumour suppressor, p53 guards the genome by orchestrating a variety of DNA-damage-response (DDR) mechanisms (as covered by Williams and Schumacher, p53 in the DNA-Damage-Repair Process, Cold Spring Harbor Perspectives in Medicine, 2016,1-15). Importantly, p53 response is key in determining if a cell will undergo apoptosis or cell cycle arrest after DNA damage.

Nucleotide excision repair (NER) removes a variety of helix-distorting lesions commonly induced by UV irradiation, whereas base excision repair (BER) targets oxidative base modifications. Mismatch repair (MMR) corrects for nucleotides that have been erroneously inserted during replication. DNA double strand breaks that are typically induced by radiation are instead resolved either by nonhomologous end joining (NHEJ) or by homologous recombination (HR).

Brown et aL. Targeting DNA repair in cancer: beyond PARP inhibitors, Cancer Discovery, 2017, 20-37 reviews the DDR pathways. The NER pathway removes helix-distorting lesions from DNA, in particular UV-induced photo lesions (Brown et aL. 2017). It involves removal of a short oligonucleotide, including the damaged lesion using structure-specific endonucleases and subsequent restoration of the DNA sequence by DNA polymerases. Genes involved in the NER pathway include XPC, DDB2, CSA, XPA, RPA, XPG, ERCC1, POLE, POLD1, LIG1 and LIG3.

DNA glycosylases in the BER pathway recognise and remove damaged bases leading to basic sites that are processed by APE1. The BER pathway results in a single-strand break (SSB), that is repaired using the SSB repair pathway. PARP1 Is the DNA-damage sensor protein for DNA strand breaks in the SSB repair pathway. PARP1 localises to the sites of DNA damage, generating extensive poly ADP ribose chains. Ribosylated PARP1 promotes recruitment of SSB-repair proteins to DNA-damage sites.

Genes involved in the BER and SSB repair pathways include DNA glycosylases, APE1, PARP, XRCC1, PNKP, POLβ, FEN1, TDP1, Aprataxin, LIG1 and LIG3A.

MSH2, MSH3 and MSH6 recognise base-base mismatches and insertion/deletion loops (Brown et aL. 2017). MSH2, MSH3 and MSH6 recruit MLH1 and PMS2 to damaged sites. The concerted actions of the MMR proteins engage EXO1 to remove the mismatch and then POLD and LIG1 to fill the gap and seal the nick, respectively. Genes involved in MMR include MSH2, MSH3, MSH6, MLH1, PMS2, EXO1, POLD and LIG1. Deficient mismatch repair (dMMR), is associated with high-frequency microsatellite instability (H-MSI); dMMR testing can be done via IHC.

Classic NHEJ (c-NHEJ) is the predominant DNA DSB repair pathway in human cells, functioning throughout the cell cycle (Brown et aL. 2017). It involves the relatively rapid ligation of broken DNA ends, mediated by the core NHEJ complex, including DNA-PK, XRCC4, LIG4, XLF and PAXX. DNA end processing and DNA polymerase action may be required before ligation can occur, making NHEJ inherently error prone. NHEJ maintains genome stability by rapidly repairing DSBs in circumstances where recombinogenic events would likely result in gross chromosomal rearrangements, such as in noncycling or G1 cells. Genes involved in NHEJ include Ku70/Ku80, DNA-PK, XRCC4, XLF, LIG4, APLF, Artemis, PAXX, WRN and ATRX.

Alternative NHEJ (Alt-NHEJ or MMEJ) is a ligation pathway for DSBs when c-NHEJ is genetically compromised (Brown et aL. 2017). It occurs following limited DNA end resection, and contributes to the excessive genomic deletions and chromosomal translocations seen in tumours. It may also provide a back-up repair pathway in HR-deficient cells. Genes involved in Alt-NHEJ include PARP, XRCC1, LIG3, LIG1, CtIP and POLQ.

HR is relatively slow and restricted to late-S phase/G2, because it generally relies on a homologous sister chromatid DNA strand for repair (Brown et aL. 2017). Extensive DNA end resection by helicases and exonucleases, such as DNA2, BLM, WRN and EXO1 results in a 3′-ssDNA overhang, committing the break to repair by HR. Replication protein A (RPA), with the help of BRCA1 and PALB2, loads RAD51 onto the RPA-coated ssDNA, leading to strand invasion, with a number of factors negatively regulating this process to prevent hyper-recombination such as POLQ, PARI, RECQL5, FANCJ, and BLM. Genes involved in HR include MRN, ATM, ATR, MK2, CtIP, BRCA1, BARD1, BRCA2, PALB2, RPA, RAD51, MUS81/EME1, SLX1/SLX4, RTEL1, BLM, TOPOIII, POLQ, PARI, RECQL5, FANCJ, BLM.

The Fanconi anemia (FA) pathway is involved in repairing interstrand cross-links. The molecular details of the FA pathway are described in Niraj et aL. The Fanconi anemia pathway in cancer, Annu. Rev. Cancer Biol. 2019. 3:457-78). The FA core complex comprises FANCA, FANCG, FANCB, FANCL, UBE2T (FANCT) FANCF, FANCC, FANCE, FANCM, REV1, REV7 and REV3. It also includes a number of Fanconi-anemia-associated proteins (FAAPs). Mutations or deletions in the core complex genes or in a FAAP are within the scope of the invention.

DDR deficiency is found in ˜13% of all cancers, with higher incidences in certain tumour types including cancer of the pancreas (>35%), bladder (35%), prostate (33%), ovarian (24%) and Triple negative breast cancer (16%).

### A Loss of Function CRISPR Screen Identified DDR as the Top Sensitising Pathway to an MDM2 Antagonist

The Examples below describe a dual CRISPR screen in a panel of three P53-wild lung cancer cell lines, in the presence or absence of Compound 1, to identify novel predictive biomarkers of MDM2 antagonist sensitivity.

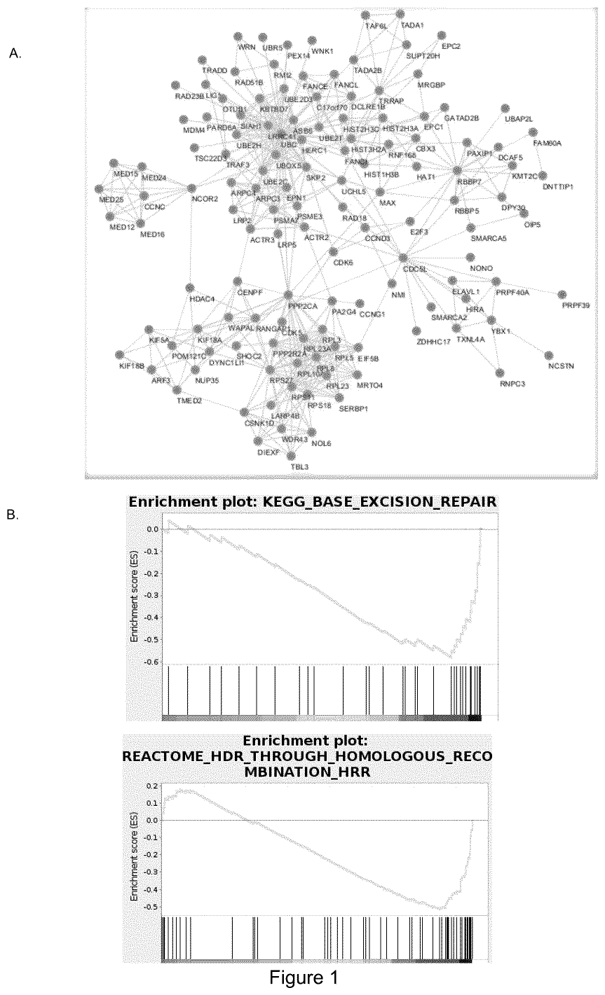

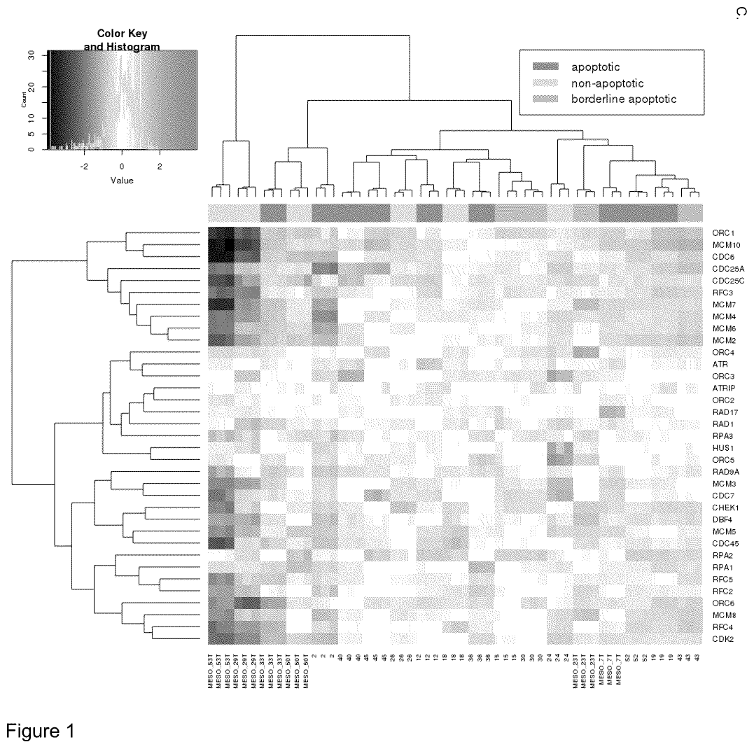

Several DNA-damage-response (DDR) related genes were identified as the top hits (FIG. 1A-B). Interestingly these genes are involved in several DDR pathways such as Homologous recombination, Fanconi anemia (FA), Base Excision Repair and Replication Stress pathways. Of note, the Replication Stress pathways is a readout of genomic instability and is caused by multiple defects in DDR pathways leading to high levels of DNA damage (which in turn affect the process of DNA replication). These data suggest that tumours with defects in their DDR machinery may generally be more sensitive to MDM2 antagonist treatment.

To confirm this result, early passages human mesothelioma cell lines, previously characterized for their sensitivity to Compound 1, were used to identify transcriptomic signatures differentially expressed between apoptotic and non-apoptotic samples. A “Replication stress” signature was strongly enriched in mesothelioma apoptotic cell lines confirming the link between Compound 1 sensitivity and activated DDR pathways (FIG. 1C).

The link between MDM2 antagonist and defects in multiple DDR pathways was identified through the dual CRISPR screen. Following the CRISPR screen, bioinformatics and wet lab analysis on multiple systems have facilitated the identification of specific biomarkers within the DDR. This additional validation of the results confirms the DDR deficiencies that indicate sensitivity to MDM2 antagonist treatment.

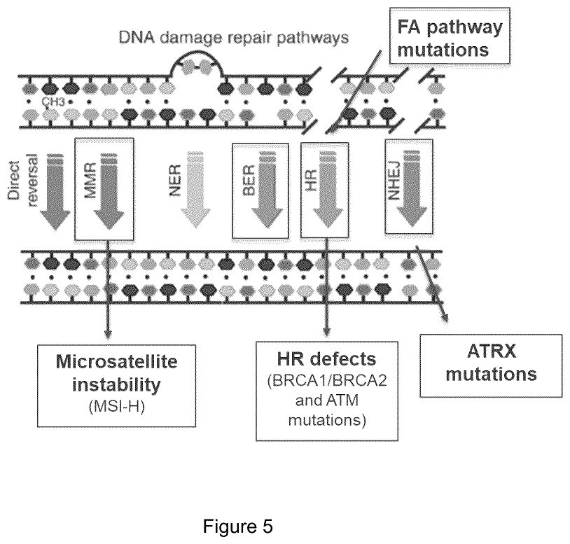

As a visual summary, FIG. 5 lists the major DDR pathways. Highlighted in boxes are DNA repair pathways where specific biomarkers have been identified by the inventors as sensitivity markers to treatment with an MDM2 antagonist.

### HR Pathway: BRCA1, BRCA2 and ATM Alterations

Genes involved in the homologous recombination pathway were identified in the CRISPR screen. Homologous recombination (HR) is an error-free DSB repair pathway that is largely restricted to the S—and G2-phases of the cell cycle. The enormous importance of the HR pathway for genome maintenance is shown by the identification of several cancer disabling mutations in BRCA1, BRCA2, ATM, CHEK2, RAD50, RAD51C in numerous cancers.

One of the central components of the HR is the serine-threonine kinase Ataxia Telangiectasia Mutated protein (ATM), which phosphorylates numerous key players in various branches of the DDR. Somatic ATM mutations or deletions are commonly found in lymphoid malignancies, as well as several solid tumors leading to loss of protein expression and impairment of DNA double strand breaks repair in the genome.

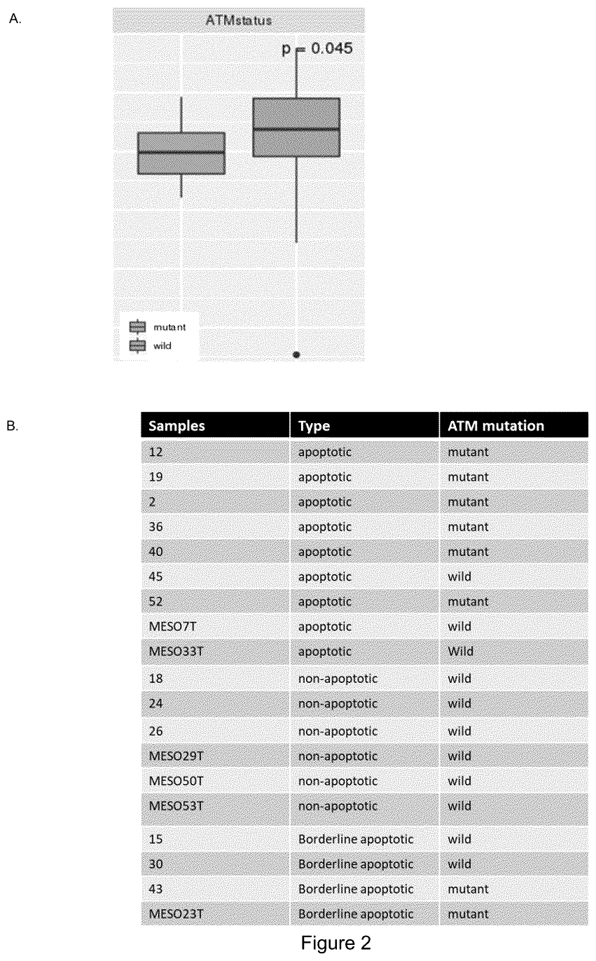

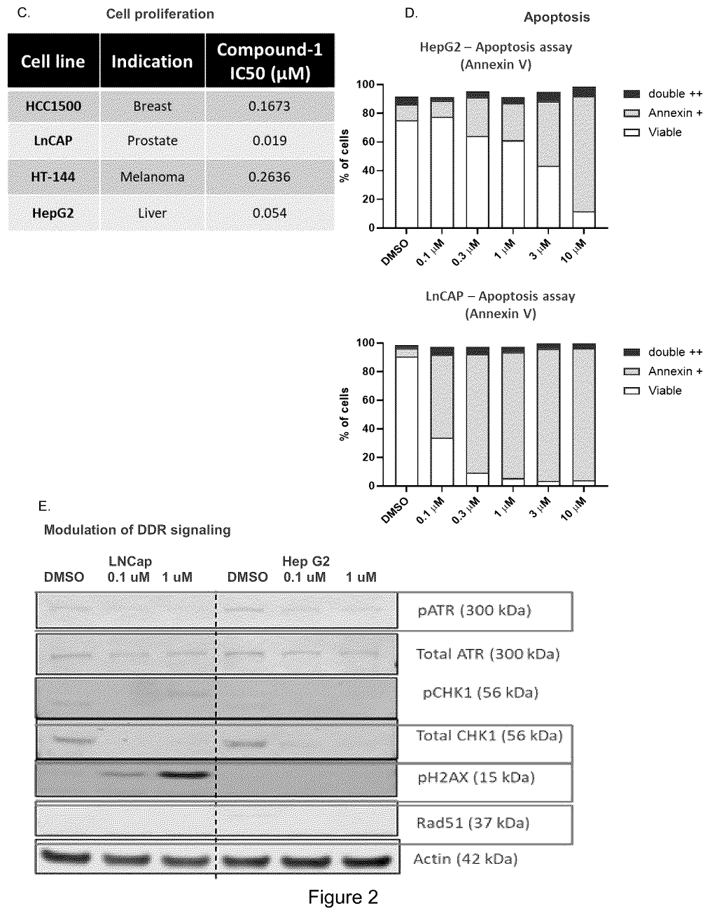

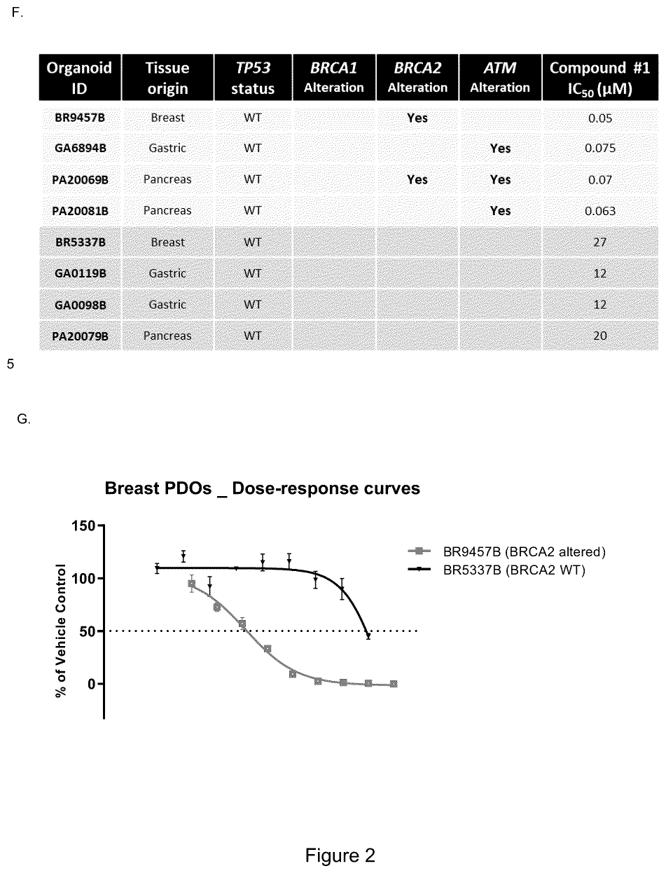

Bioinformatics analysis of publicly available DepMAP RNAi data (version 20Q4) for MDM2 predicted that ATM mutant cell lines are significantly more dependent on MDM2 as compared to ATM wild cell lines (FIG. 2A). In addition, a strong enrichment of ATM mutation was detected in apoptotic patient-derived mesothelioma lines (6/9) compared to non-apoptotic lines which are all ATM wild-type (6/6) (FIG. 2B).

Further, in vitro validation on four ATM mutant cell lines from different indications (HCC1500—breast, LNCap—prostate, HT-144—melanoma, HepG2—liver) showed sensitivity to Compound 1 as measured by reduction in cell proliferation (FIG. 2C), while data on LNCap—prostate and HepG2—liver showed sensitivity to Compound 1 as measured by increased apoptosis (FIG. 2D). Furthermore, western blot analysis showed a clear modulation of the DDR signalling pathway upon Compound 1 treatment (FIG. 2E).

Together with the identification of ATM mutations as a biomarker for MDM2 antagonist sensitivity additional bioinformatic analysis indicate that loss or mutation of other HR pathway genes may act as biomarkers for MDM2 antagonist sensitivity. HR pathway genes that may act as biomarkers for MDM2 antagonist therapy include but are not limited to BRCA1 and/or BRCA2.

In one embodiment the defect in homologous recombination repair includes BRCA1 or BRCA2 loss. In one embodiment the cancer displays BRCAness. Tumours with loss of canonical HRR, other than BRCA1/2 loss, display BRCAness (Trends in Cell Biology, September 2019, Vol. 29, No. 9, pg 740).

To confirm if ATM, BRCA1 and/or BRCA2 mutations (or loss of expression) could be linked to MDM2 antagonist sensitivity further in vitro validation on patient-derived organoids (PDOs) was carried out. 4 PDOs from various indications with alterations in either ATM, BRCA1 and/or BRCA2 showed sensitivity to Compound 1 as measured by reduction in cell proliferation (FIG. 2F-G). Of note 4 additional PDOs from the same indications but with no alterations in ATM, BRCA1 and/or BRCA2 were resistant to Compound 1.

### FA Pathway

FA pathway genes are also provided as MDM2 antagonist therapy biomarkers.

The dual CRISPR screen (CRISPR knock-out and CRISPRi) identified genes are involved in the Fanconi Anemia (FA) pathway. FIG. 1A shows enrichment of the Fanconi Anemia pathway in the CRISPR hits.

The CRISPR screen data indicate that tumours with defects in the FA pathway are generally sensitive to Compound 1 treatment.

These data therefore demonstrate the link between MDM2 antagonist sensitivity and defects in the Fanconi Anemia pathway. Loss of function in the FA pathway is therefore a biomarker for MDM2 antagonist sensitivity.

### NHEJ Pathway: ATRX Loss

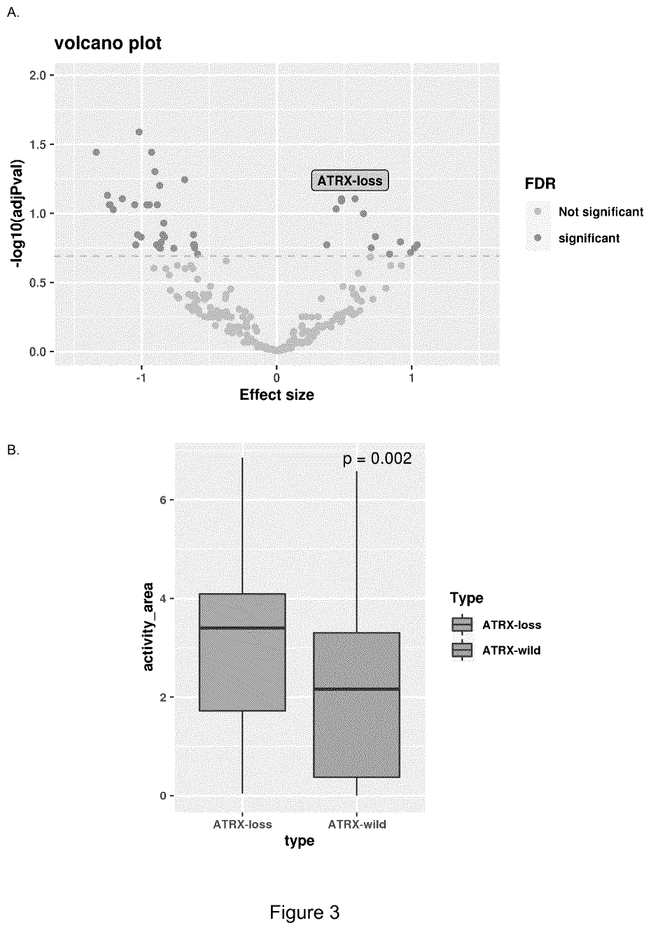

Additionally, bioinformatics analysis of the cell panel data predicted loss of ATRX as a significant biomarker of sensitivity to MDM2 Compound 1 (FIG. 3). ATRX is also been implicated in the regulation of DDR both by non-homologous end joining (NHEJ) and homologous recombination repair (HRR).

The identification of ATRX loss as a biomarker for MDM2 antagonist therapy and bioinformatics analysis demonstrate that loss or mutation of other NHEJ or HRR pathway genes may act as biomarkers for MDM2 antagonist sensitivity.

### MMR Pathway: Microsatellite Instability (MSI)

Microsatellites are regions that contain multiple repeats of 1 to 5 base pair motifs which are widely dispersed throughout the human genome. In normal cells, repeat count of microsatellites is verified and maintained during cell division by the mismatch repair (MMR). Impairment of the MMR system can render cells unable to regulate the lengths of their microsatellites during cell division, termed MSI (for microsatellite instability). MSI has been frequently observed within several types of cancer (colorectal, endometrial, and gastric adenocarcinomas) and MSI-High colorectal tumors have been shown to be more susceptible to immune-enhancing therapies.

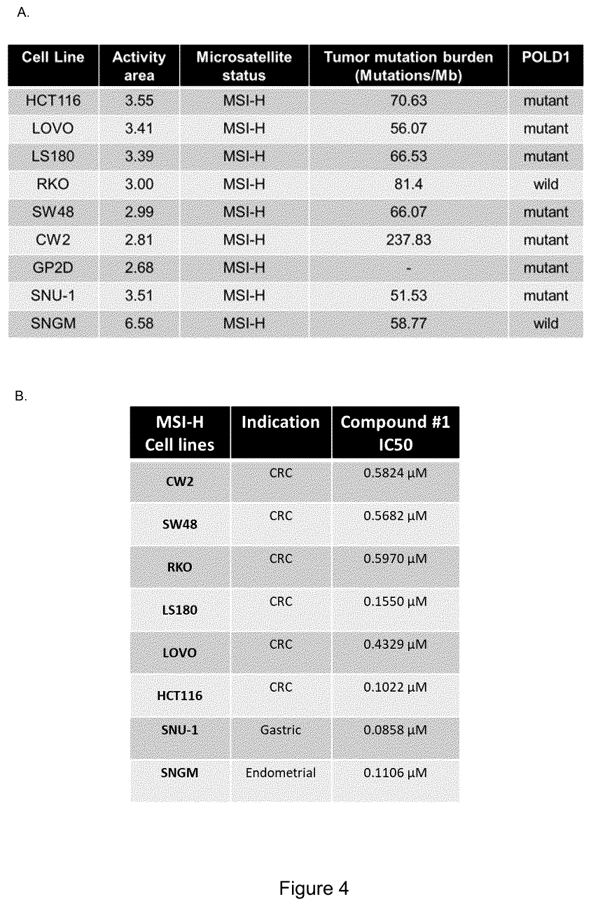

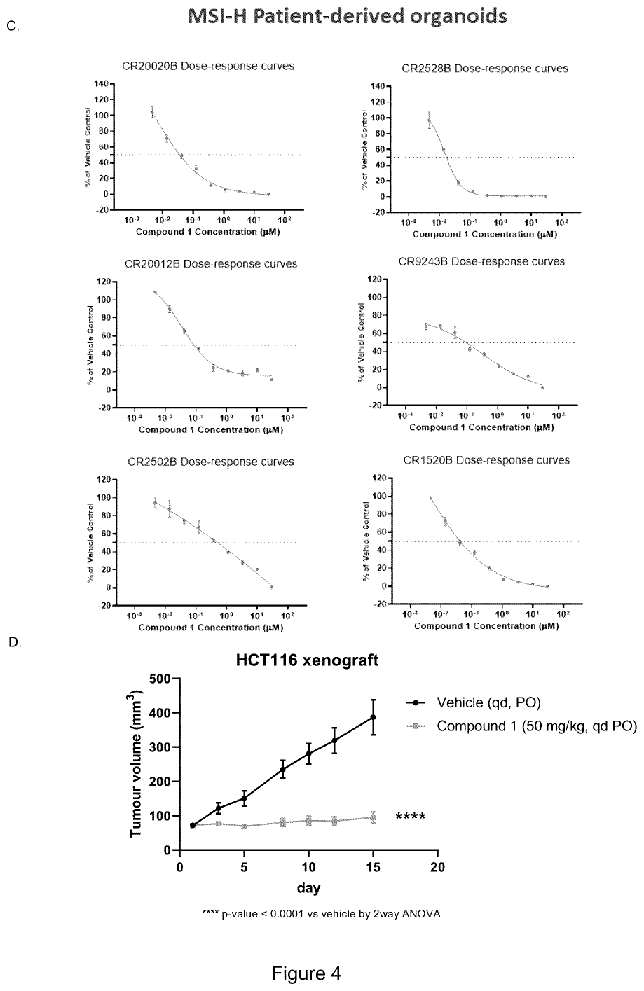

Information on microsatellite stability and tumour mutation burden for cell lines was obtained from Sanger Cell Models Passport database. We found that MSI-H cell lines exhibited a high tumour mutation burden (mutations/Mb) and were enriched in mutations related to DNA mismatch repair pathway (eg. MSH2, MSH3, MSH6, MLH1, MLH3, PMS2). Further, MSI-H cell lines showed strong enrichment of mutational signatures associated with defects in DNA mismatch repair and POLD1 and/or POLE mutations (FIG. 4A). Together, these findings were consistent and suggested that MSI tumours such as colorectal, endometroid and gastric would be sensitive to MDM2 antagonists. Further, in vitro validation on eight MSI-H cell lines from different indications showed sensitivity to Compound 1 as measured by reduction in cell proliferation (FIG. 4B). To confirm if MSI-H status could be linked to MDM2 antagonist sensitivity further in vitro validation on patient-derived organoids (PDOs) was carried out. 6 MSI-H colorectal cancer PDOs showed sensitivity to Compound 1 as measured by reduction in cell proliferation (FIG. 4C).In vivo efficacy data confirmed that Compound 1 significantly inhibits tumour growth in a xenograft model of MSI-H colorectal cancer (HCT-116) (FIG. 4D).

### Base Excision Repair (BER) Pathway

The CRISPR screen analysis identifies a strong correlation between the BER pathway and MDM2 antagonist sensitivity (FIG. 1). The DDR biomarker may comprise one or more BER pathway genes.

### Biomarkers and Combinations

For ease of reference, the biomarkers of the disclosure may be characterised into five groups:
a. HR Pathway, e.g. BRCA1, BRCA2 and/or ATM depletions.
b. NHEJ Pathway, e.g. ATRX loss.
c. MMR pathway, e.g. MSI-H (Microsatellite instable tumours and characterised by high tumor mutation burden).
d. FA pathway.
e. BER pathway.

In some embodiments, one biomarker is determined. This may be from any of groups a), b), c), d) or e).

In some embodiments, multiple biomarkers are determined, for example 2, 3, 4, 5, 6, 7, 8, 9, 10 or more biomarkers. These may comprise or consist of multiple biomarkers from a single group (i.e. group b) or group c)), or may comprise or consist of one more biomarkers from different groups, for example:
0, 1, 2 or more from group a; and 0, 1, 2 or more from group b); and 0, 1, 2 or more from group c); and with or group d) or group e); or
0, 1, 2 or more from group b); and 0, 1, 2 or more from group c), and with group d; or
2 or more from group b); 2 or more from group c); with or without group d; or
0, 1, 2 or more from group a); 0, 1, 2 or more from group b); 0, 1, 2 or more from group c), 0, 1, 2 or more from group d); and 0, 1, 2 or more from group e).

When multiple biomarkers are determined, the combination of biomarkers may be referred to as a biomarker panel. The biomarker panel can comprise or consist of the identified biomarkers.

In addition to the biomarkers of the invention, other biomarkers and or data, such as demographic data (e.g., age, sex) can be included in a set of data applied for the determination of suitability for MDM2 inhibition. When other biomarkers are optionally included, the total number of biomarkers (i.e. the biomarker panel of the invention plus other biomarkers) may be 3, 4, 5, 6 or more. In some embodiments, a predictive biomarker panel with fewer components can simplify the testing required.

The terms “loss” and “decreased” as used herein are to be given their usual meanings. The terms “increased” and “enhanced” as used herein are to be given their usual meanings.

The biomarkers can be determined by appropriate techniques that will be apparent to one skilled in the art. The biomarkers can be determined by direct or indirect techniques. Gene expression can be detected by detecting mRNA transcripts. Protein biomarkers can be detected by immunohistochemistry.

In some embodiments, depletion of one or more of the biomarkers of the invention may be determined by evaluating the function of the one or more biomarkers. The biomarker expression level may be directly proportional to the level of function. The function of the one or more biomarkers may be determined directly or indirectly.

In some embodiments, the expression or activity level can be compared to a threshold value reflecting in the same manner the expression or activity level known to be associated with sensitivity to treatment, to assess whether the tested value is indicative of sensitivity to MDM2 inhibition treatment in the patient.

A patient that is assessed according to the present disclosure is known or suspected to have a cancer. The sample that is tested may be known or suspected to comprise cancer cells. In typical embodiments, the sample that is tested will be a biopsy of cancer tissue. The biopsy may be a liquid biopsy or a solid tissue (e.g. solid tumour) biopsy.

### Biomarker Levels

The invention provides one or more biomarkers at decreased level. Typically, the comparison will be made to relative to normal healthy individuals, more typically to non-cancer cells of the same type as the cancer cell.

The reduced biomarker level may be the depletion of the gene itself, for example by a gross chromosomal rearrangement or other genetic abnormality that results in loss of the DDR pathway gene in the genome of the cancer cell. This will, of course, also deplete the gene product and activity.

The reduced biomarker level may be reduced expression of the gene product.

The reduced biomarker level may also be reduced activity, for example caused by a loss of function mutation. A loss of function mutation may be viewed as the depletion of the wild type gene product.

In some embodiments, increased or decreased biomarker levels are determined relative to non-cancerous cells from the same individual, typically non-cancerous cells of the same type, from the same individual.

In further embodiments, increased or decreased biomarker levels are determined relative to laboratory standards and values based on a known normal population value. Typically, the known levels are taken from a non-cancer cell.

In other embodiments the increased or decreased biomarker levels are relative to known values from normal (non-cancerous) individuals. For example, GTEx is a data resource of gene expression of normal healthy individuals from 44 different tissues, as discussed elsewhere herein.

In some other embodiments, increased or decreased biomarker levels are assessed relative to the level determined in cancer samples from MDM2 inhibitor non-responsive subjects, or in a cancer sample from an MDM2 inhibitor non-responsive subject. This may be particularly useful for the one or more IFN signature biomarkers.

In one embodiment, the RNA level of one or more DDR biomarker is decreased relative to the amount of said RNA in a control sample obtained from a normal subject not suffering from cancer.

In an alternative embodiment, the RNA level of one or more DDR biomarker is decreased relative to the amount of said RNA in an earlier sample obtained from the same patient when that patient did not have the cancer.

In one embodiment it is decreased relative to normal levels (e.g. “Upper limit of Normal” or ULN).

In one embodiment, the level of at least one of the biomarkers has an area under the curve (AUC) in cancer vs. a control sample of greater than (for increased biomarkers) or less than (for depleted biomarkers)0.5 relative to (a) the level of at least one of the biomarkers in a sample from a tissue or person not having cancer, or (b) the level of one or more control proteins in a sample from the subject. Optionally the AUC is less than or greater than 0.6, 0.7, 0.8, 0.9, 0.95, 0.975 or 0.99.

In some embodiments, the level of at least one of the biomarkers is at least one standard deviation from the control relative to (a) the level of the one or more biomarkers in a sample from a tissue or person not having cancer, or (b) the level of one or more control proteins in a sample from the cancer subject.

In some embodiments, the control for comparison is a sample obtained from a healthy patient or a non-cancerous tissue sample obtained from a patient diagnosed with cancer, such as a non-cancerous tissue sample from the same organ in which the tumour resides (e.g., non-cancerous colon tissue can serve as a control for a colon cancer). In some embodiments, the control is a historical control or standard value (i.e., a previously tested control sample or group of samples that represent baseline or normal values).

Controls or standards for comparison to a sample, for the determination of differential expression, include samples believed to be normal (in that they are not altered for the desired characteristic, for example a sample from a subject who does not have colon cancer) as well as laboratory values, even though possibly arbitrarily set. Laboratory standards and values may be set based on a known or determined population value and can be supplied in the format of a graph or table that permits comparison of measured, experimentally determined values.

In such embodiments, a reference score for biomarker or biomarkers is based on normal healthy individuals.

### Cancers

A cancer presenting one or more of the identified biomarkers has an increased likelihood of successful treatment with an MDM2 antagonist. The cancer to be treated is not particularly limited, provided that it presents one or more of the biomarkers.

The cancer is typically p53 wild-type. As is recognized in the art, p53 wild-type cancer cells express the tumour suppressor p53 at wild-type levels and with wild-type function. Wild-type p53 cells do not contain a mutation in the p53 gene that leads to decreased p53 tumour suppressor function.

The data provided in the Examples were generated from a range of cancerous tissues including colon, blood, breast, lung, prostate, liver, skin, ovary and pancreas. In one embodiment, the cancer is a lung cancer. In one embodiment, the cancer is a colon cancer. In another embodiment the cancer is a blood cancer. In a further embodiment the cancer is a breast cancer. In another embodiment the cancer is a lung cancer. In yet another embodiment the cancer is a skin cancer, for example a melanoma or a carcinoma. In another embodiment the cancer is an ovarian cancer. In a different embodiment the cancer is a pancreatic cancer. In certain embodiments, the cancer is a brain cancer, a clear cell renal cell carcinoma (ccRCC), an oesophageal cancer, or melanoma.

Particular cancers that can be assessed for treatment according to the invention include but are not limited to mesothelioma, non-small cell lung carcinoma (NSCLC), glioblastoma (e.g. GBM) and renal cancer (e.g. KIRC).

Particular cancers that can be assessed for treatment according to the invention include but are not limited to acute myeloid leukemia (AML), squamous cell carcinoma or tumors of the head, neck, skin, gastrointestinal system, or genital tract.

Particular cancers that can be assessed for treatment according to the invention include but are not limited to prostate, ovarian, breast and gynaecological cancers.

Particular cancers that can be assessed for treatment according to the invention include but are not limited to colorectal, gastric and gynaecological cancers.

Particular cancers that can be assessed for treatment according to the invention include Breast, ovarian, prostate and pancreas, in particular DDR defective Breast, ovarian, prostate and pancreas.

In certain embodiments, the proliferation of cancer cells is inhibited by an MDM2 antagonist with an IC₅₀ value in the nanomolar range. In certain embodiments, the IC₅₀ value is less than 500 nM, less than 400 nM, less than 300 nM, or less than 200 nM. In some embodiments, the IC₅₀ value is less than 100 nM. IC₅₀ values can be calculated, for example, using GraphPad Prism software as shown in the Examples.

In certain embodiments, the MDM2 antagonist induces apoptosis of the cancer cell. Apoptosis may typically be mediated via activated caspase-3. Induction of apoptosis can be determined by detecting cells that are positive for activated caspase-3 following 72-hour treatment with 1 μM of the MDM2 antagonist. Other assay concentrations and/or treatment lengths may be used, as will be apparent to the skilled person, for example 48 hours with 1 μM or 48 hours with 5 μM of the MDM2 antagonist. In certain embodiments, at least 10%, at least 20% or at least 30% of cells staining positive for activated caspase-3 is an indicator of induced apoptosis. In certain embodiments, 40% is a reliable level to identify strong induction of apoptosis wherein >40% of cells in a population, staining positive for activated caspase-3, can be deemed as apoptotic. Other levels may be used as appropriate to the cells and assay, as will be apparent to the skilled person, for example 10%, 20%, 30%, 50%, 60%, 70%, 75% or more. Active caspase-3 staining kits are commercially-available, for example the “Cleaved Caspase-3 Staining Kit (Red)” available from Abcam (Cambridge, UK) as catalogue number ab65617.

The Invitrogen Cell Event dye (C10423) may also be used.

Annexin V dye can also be used for detecting apoptosis. This was used in the Examples and is well known in the art as a useful dye for detecting apoptosis.

### MDM2 Antagonists

The transformation-related protein 53 (TP53) gene encodes a 53 KDa protein—p53. The tumour suppressor protein p53 reacts to cellular stresses, such as hypoxia, DNA damage and oncogenic activation, via a number of posttranslational modifications including phosphorylation, acetylation and methylation, and acts as a signalling node in the diverse pathways that become activated. p53 has additional roles in other physiological processes, including autophagy, cell adhesion, cell metabolism, fertility, and stem cell aging and development. Phosphorylation of p53, resulting from activation of kinases including ATM, CHK1 and 2, and DNA-PK, results in a stabilised and transcriptionally active form of the protein, thus producing a range of gene products. The responses to p53 activation include apoptosis, survival, cell-cycle arrest, DNA-repair, angiogenesis, invasion and autoregulation. The specific combination of which, in concert with the cell's genetic background, gives rise to the observed cellular effect i.e. apoptosis, cell-cycle arrest or senescence. For tumour cells, the apoptotic pathway may be favoured due to the loss of tumour suppressor proteins and associated cell cycle checkpoint controls, coupled with oncogenic stress.

Under conditions of stress such as hypoxia and DNA damage it is known that the cellular level of the protein p53 increases. p53 is known to initiate transcription of a number of genes which govern progression through the cell cycle, the initiation of DNA repair and programmed cell death. This provides a mechanism for the tumour suppressor role of p53 evidenced through genetic studies.

The activity of p53 is negatively and tightly regulated by a binding interaction with the MDM2 protein, the transcription of which is itself directly regulated by p53. p53 is inactivated when its transactivation domain is bound by the MDM2 protein. Once inactivated the functions of p53 are repressed and the p53-MDM2 complex becomes a target for ubiquitinylation.

In normal cells the balance between active p53 and inactive MDM2-bound p53 is maintained in an autoregulatory negative feedback loop. That is to say that p53 can activate MDM2 expression, which in turn leads to the repression of p53.

It has been found that inactivation of p53 by mutation is common in around half of all common adult sporadic cancers. Furthermore, in around 10% of tumours, gene amplification and over-expression of MDM2 results in the loss of functional p53, thereby allowing malignant transformation and uncontrolled tumour growth.

Inactivation of p53 by a range of mechanisms is a frequent causal event in the development and progression of cancer. These include inactivation by mutation, targeting by oncogenic viruses and, in a significant proportion of cases, amplification and/or an elevated rate of transcription of the MDM2 gene resulting in overexpression or increased activation of the MDM2 protein. Gene amplification of MDM2 giving rise to overexpression of MDM2 protein has been observed in tumour samples taken from common sporadic cancers. Overall, around 10% of tumours had MDM2 amplification, with the highest incidence found in hepatocellular carcinoma (44%), lung (15%), sarcomas and osteosarcomas (28%), and Hodgkin disease (67%) (Danovi et al., Mol. Cell. Biol. 2004, 24, 5835-5843, Toledo et al., Nat Rev Cancer 2006, 6, 909-923, Gembarska et al., Nat Med 2012, 18, 1239-1247). Normally, transcriptional activation of MDM2 by activated p53 results in increased MDM2 protein levels, forming a negative feedback loop. The essential nature of p53 regulation by MDM2 and MDMX is demonstrated by gene knockout mouse models. MDM2−/− knockout mice are embryonically lethal around the time of implantation. Lethality is rescued in the double knockout for MDM2 and TP53. MDM2 inhibits the activity of p53 directly, by binding to and occluding the p53 transactivation domain, and by promoting the proteosomal destruction of the complex, through its E3-ubiquitin ligase activity. In addition, MDM2 is a transcriptional target of p53, and so the two proteins are linked in an autoregulatory feedback loop, ensuring that p53 activation is transient.

Although MDMX shows strong amino acid sequence and structural homology to MDM2, neither protein can substitute for loss of the other; MDMX null mice die in utero, whereas MDM2 knockout is lethal during early embryogenesis, however both can be rescued by p53 knockout, demonstrating p53-dependence of the lethality. MDMX also binds p53 and inhibits p53-dependent transcription, but unlike MDM2 it is not transcriptionally activated by p53 and so does not form the same autoregulatory loop.

Furthermore, MDMX has neither E3 ubiquitin ligase activity nor a nuclear localisation signal, however it is believed to contribute to p53 degradation by forming heterodimers with MDM2 and contributing to MDM2 stabilisation.

The therapeutic rationale for MDM2-p53 inhibition is that a potent antagonist of the protein-protein interaction will liberate p53 from the repressive control of MDM2 and activate p53 mediated cell death in the tumour. In tumours, selectivity is envisioned to result from p53 sensing preexisting DNA-damage or oncogenic activation signals that had previously been blocked by the action of MDM2 at normal or overexpressed levels. In normal cells, p53 activation is anticipated to result in activation of non-apoptotic pathways and if anything a protective growth inhibition response. In addition due to the non-genotoxic mechanism of action for MDM2-p53 antagonists they are suitable for the treatment of cancer in particular in the pediatric population. MDM4 is also an important negative regulator of p53.

About 50% of cancers harbour cells in which TP53, the gene that encodes for p53, is mutated resulting in a loss of the protein's tumour suppressor function and sometimes even in p53 protein versions that gain novel oncogenic functions.

Cancers where there is a high level of MDM2 amplification include liposarcoma (88%), soft tissue sarcoma (20%), osteosarcoma (16%) oesophageal cancer (13%), and certain paediatric malignancies including B-cell malignancies.

### Examples of MDM2 Antagonists

In one embodiment the MDM2 antagonist is an agent that modulates MDM2, for example, a small molecule, antisense nucleic acid, antibody or nucleic acid that inhibits expression of MDM2. In one embodiment the MDM2 agent is a small molecule. In one embodiment the MDM2 agent is a small molecule as detailed herein.

Idasanutlin (RG-7388), a small molecule antagonist of MDM2 from Roche has been reported to be in Phase I-III clinical trials for solid and haematological tumours, AML, diffuse large B-cell lymphoma, essential thrombocythemia, polycythemia vera and follicular lymphoma. Idasanutlin (RG-7388) has the structure below:

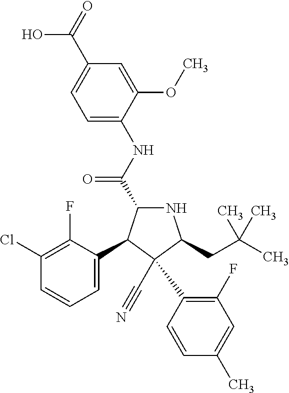 [SMILES: COc1cc(C(=O)O)ccc1NC(=O)[C@@H]1N[C@@H](CC(C)(C)C)[C@](C#N)(c2ccc(C)cc2F)[C@H]1c1cccc(Cl)c1F]

Idasanutlin (RG-7388) is commercially available or may be prepared for example as described in PCT Patent application WO 2014/128094 or by processes analogous thereto.

HDM-201 (NVP-HDM201, siremadlin) is being developed by Novartis in Phase I/II clinical trials for wild type TP53 characterised advanced/metastatic solid tumours, haematological tumours including ALL, AML, MS, metastatic uveal melanoma, dedifferentiated liposarcoma and well differentiated liposarcoma. Antagonist HDM-201 (NVP-HDM201) has the chemical structure below:

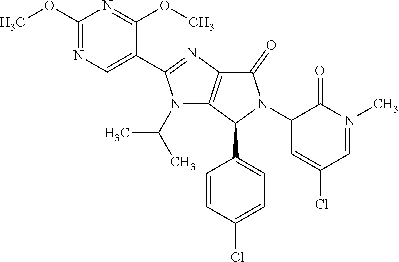

HDM-201 (NVP-HDM201) is commercially available or may be prepared for example as described in PCT Patent application WO 2013/111105 or by processes analogous thereto.

KRT-232 (AMG-232, navtemadlin) a small molecule antagonist of MDM2 is being developed by NCI/Amgen/GSK in Phase I-I/II clinical trials for solid tumours, soft tissue sarcomas such as liposarcoma, recurrent or newly diagnosed glioblastoma, metastatic breast cancer, refractory MM, metastatic cutaneous melanoma and relapsed/refractory AML. KRT-232 (AMG-232) has the chemical structure below:

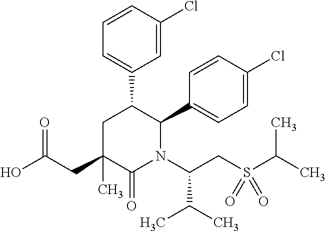 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

KRT-232 (AMG-232) is commercially available or may be prepared for example as described in PCT Patent application WO 2011/153509 or by processes analogous thereto.

ALRN-6924 (SP-315), a peptide dual antagonist of MDM2 and MDM4 is being developed by Aileron Therapeutics and Roche in Phase II clinical trials for intravenous treatment of solid tumours, small cell lung cancer and pediatric tumours including lymphomas, acute myeloid leukemia acute lymphocytic leukemia, retinoblastoma, hepatoblastoma, brain tumour, liposarcoma and metastatic breast cancer.

ALRN-6924 (SP-315) is a synthetic peptide which is developed based on stapled peptide technology that locks the peptides into certain folded shapes (biologically active shape), that are resistant to proteases. ALRN-6924 (SP-315) has the structure below:

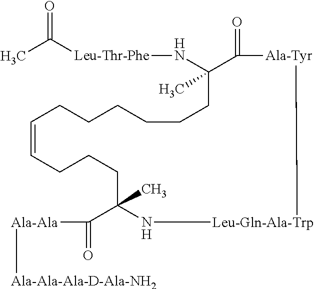

ALRN-6924 (SP-315) is commercially available or may be prepared for example as described in PCT Patent application WO2017205786 or by processes analogous thereto.

CGM-097 (NVP-CGM-097) a small molecule antagonist of MDM2 is being developed by Novartis in Phase I clinical trials for advanced solid tumours and acute lymphoblastic leukaemia (B-ALL). CGM-097 (NVP-CGM-097) has the chemical the structure below:

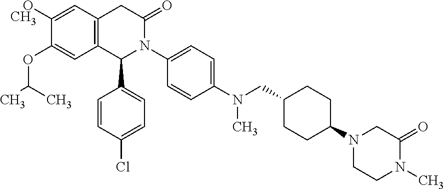

CGM-097 (NVP-CGM-097) is commercially available or may be prepared for example as described in PCT Patent application WO2011076786 or by processes analogous thereto.

Milademetan tosylate (DS-3032), licensed to Rain Therapeutics and renamed by research code RAIN-32, a small molecule antagonist of MDM2 is being developed by Daiichi Sankyo in Phase I clinical trials for advanced solid tumours, lymphomas, melanoma, refractory or relapsed AML, ALL, multiple myeloma, CML in blast phase, or high risk MDS and diffuse large B-cell lymphoma. Milademetan tosylate (DS-3032) has the chemical the structure below

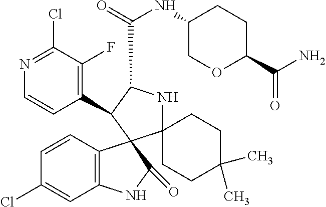 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

Milademetan tosylate (DS-3032) is commercially available or may be prepared for example as described in PCT Patent application WO 2015/033974 or by processes analogous thereto.

APG-115 (AAA-115; alrizomadlin, NCT-02935907) a small molecule antagonist of MDM2 is being developed by Ascentage Pharma in Phase I clinical trials for the treatment of solid tumours and lymphomas, AML, adenoid cystic carcinoma (ACC). APG-115 (AAA-115; NCT-02935907) has the chemical the structure below

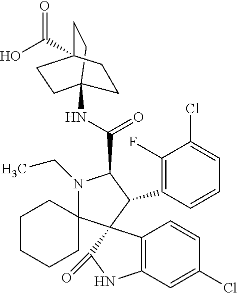 [SMILES: CCN1[C@@H](C(=O)N[C@]23CC[C@](C(=O)O)(CC2)CC3)[C@H](c2cccc(Cl)c2F)[C@]2(C(=O)Nc3cc(Cl)ccc32)C12CCCCC2]

APG-115 (AAA-115; NCT-02935907) is commercially available or may be prepared for example as described in PCT Patent application WO 2015/161032 or by processes analogous thereto.

BI-907828 an antagonist of MDM2 is being developed by BI in Phase I clinical trials for the treatment of GBM, metastatic brain tumour, NSCLC, soft tissue sarcoma and transitional cell carcinoma (urothelial cell carcinoma).

BI-907828 is commercially available or may be prepared for example as described in PCT Patent application WO 2015/161032 or by processes analogous thereto.

The University of Michigan is developing LE-004 a PROTAC of MI-1061 and a thalidomide conjugate, which showed that it efficiently inhibited growth in human leukaemia models in mice, by inducing MDM2 degradation. The structure is below and may be prepared for example as described in PCT Patent application WO 2017/176957 or WO 2017/176958 or by processes analogous thereto. LE-004 has the chemical the structure below

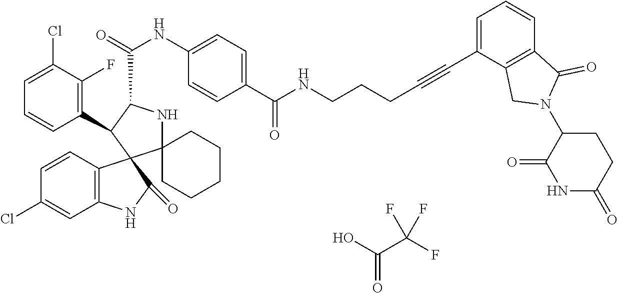 [SMILES: O=C1CCC(N2Cc3c(C#CCCCNC(=O)c4ccc(NC(=O)[C@@H]5NC6(CCCCC6)[C@@]6(C(=O)Nc7cc(Cl)ccc76)[C@H]5c5cccc(Cl)c5F)cc4)cccc3C2=O)C(=O)N1 ; O=C(O)C(F)(F)F]

MI-773 (SAR405838) is a highly potent and selective MDM2 inhibitor, binds to MDM2 with high specificity over other proteins and potently inhibits cell growth in cancer cell lines. SAR405838 effectively induces apoptosis and potently inhibits cell growth and induces dose-dependent apoptosis and is being investigated in clinical trials. The structure is:

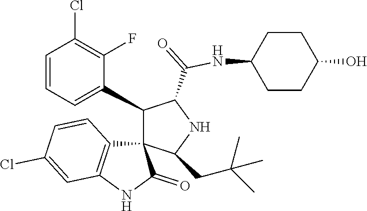 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

SAR405838 can be prepared for example as described in WO-A-2011/060049. DS-5272 is an antagonist of MDM2 is being developed by Daiichi Sankyo for Oral Dosing. The structure is:

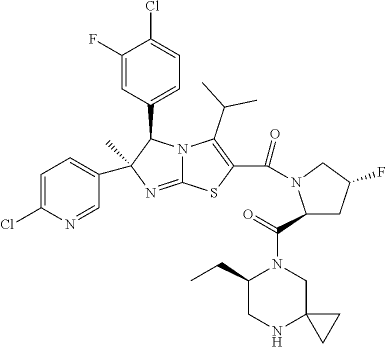 [SMILES: CC[C@@H]1CNC2(CC2)CN1C(=O)[C@@H]1C[C@@H](F)CN1C(=O)C1=C(C(C)C)N2C(=N[C@@](C)(c3ccc(Cl)nc3)[C@H]2c2ccc(Cl)c(F)c2)S1]

DS-5272 may be prepared for example as described in PCT Patent application WO 2015/033974 or by processes analogous thereto.

SJ-0211 is an antagonist of MDM2 is being developed by University of Tennessee, University of Kentucky and St Jude Children's Research Hospital for treatment of Retinotherapy. The structure is a Nutlin-3 analogue.

BI-0252 is an antagonist of MDM2 is being developed by BI for Oral Dosing. BI-0252 inhibits MDM2 and p53 interactions. The structure is:

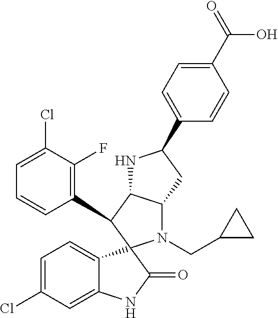

AM-7209 is an antagonist of MDM2 is being developed by Amgen as a back up for AMG-232. The structure is:

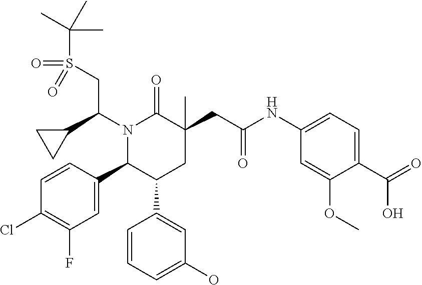 [SMILES: COc1cc(NC(=O)C[C@@]2(C)C[C@H](c3cccc(O)c3)[C@@H](c3ccc(Cl)c(F)c3)N([C@H](CS(=O)(=O)C(C)(C)C)C3CC3)C2=O)ccc1C(=O)O]

AM-7209 may be prepared for example as described in PCT Patent application WO 2014/200937 or by processes analogous thereto.

SP-141 (JapA) is a direct antagonist of MDM2 and is being developed by Texas Tech University. The structure is:

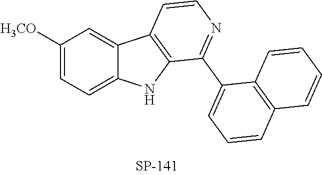 [SMILES: COc1ccc2[nH]c3c(-c4cccc5ccccc45)nccc3c2c1]

SCH-1450206 is an antagonist of MDM2 that is being developed by Schering-Plough & Merck for Oral Dosing. One example structure is:

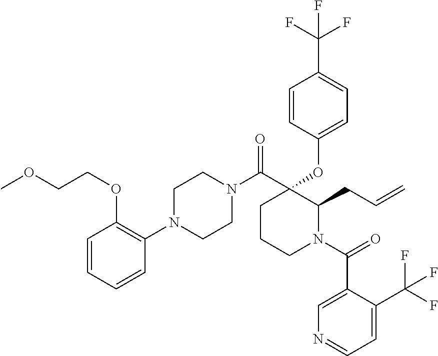 [SMILES: C=CC[C@H]1N(C(=O)c2cnccc2C(F)(F)F)CCC[C@@]1(Oc1ccc(C(F)(F)F)cc1)C(=O)N1CCN(c2ccccc2OCCOC)CC1]

Cytarabine, also known as MK-8242 and SCH-900242, is an antimetabolite analogue of cytidine with a modified sugar moiety (arabinose instead of ribose). An orally bioavailable inhibitor of human homolog of double minute 2 (HDM2) with potential antineoplastic activity, upon oral administration, HDM2 inhibitor MK-8242 inhibits the binding of the HDM2 protein to the transcriptional activation domain of the tumor suppressor protein p53. By preventing this HDM2-p53 interaction, the degradation of p53 is inhibited, which may result in the restoration of p53 signaling. This induces p53-mediated tumor cell apoptosis.

Nutlin-3a is an antagonist or inhibitor of MDM2 (human homolog of murine double minute 2), which disrupts its interaction with p53, leading to the stabilization and activation of p53. The structure is:

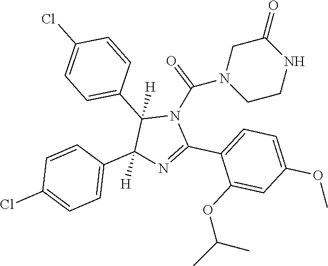 [SMILES: COc1ccc(C2=N[C@@H](c3ccc(Cl)cc3)[C@@H](c3ccc(Cl)cc3)N2C(=O)N2CCNC(=O)C2)c(OC(C)C)c1]

NXN-6 (NXN-7; NXN-552; NXN-561; NXN-11) is an antagonist of MDM2 being developed by Nexus, Priaxon and BI for Oral Dosing. An example structure is:

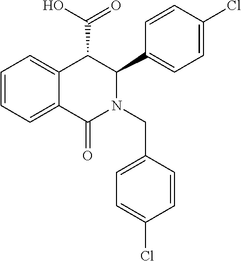 [SMILES: O=C(O)[C@H]1c2ccccc2C(=O)N(Cc2ccc(Cl)cc2)[C@@H]1c1ccc(Cl)cc1]

ADO-21 is an antagonist of MDM2 being developed by Adamed Group.

CTX-50—CTX-1 is a small molecule MDM2 antagonist being developed by MiRx Pharmaceuticals, CRC.

ISA-27 is a small molecule MDM2 antagonist being developed by the University of Napoli and the University of Salerno. The structure is:

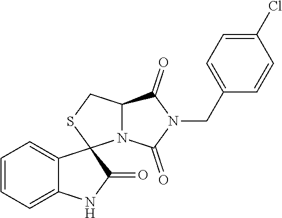 [SMILES: O=C1[C@@H]2CS[C@]3(C(=O)Nc4ccccc43)N2C(=O)N1Cc1ccc(Cl)cc1]

RG-7112 (R5045337) is a potent, selective, first clinical, orally active and blood-brain barrier crossed MDM2-p53 inhibitor. The structure is:

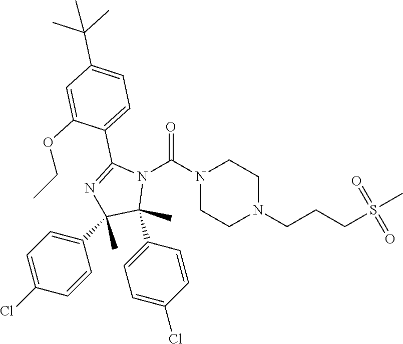 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

RO-8994 is a small molecule MDM2 antagonist being developed by Roche. RO-8994 has been shown to inhibit tumour growth inducing mitochondrial effects of p53. The structure is:

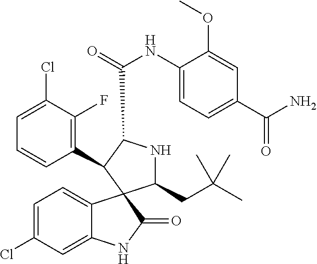 [SMILES: COc1cc(C(N)=O)ccc1NC(=O)[C@@H]1N[C@@H](CC(C)(C)C)[C@@]2(C(=O)Nc3cc(Cl)ccc32)[C@H]1c1cccc(Cl)c1F]

RO-8994 is commercially available or may be prepared for example as described in PCT Patent application WO 2011/067185 or by processes analogous thereto.

RO-6839921 (RG-7775) is a small molecule MDM2 antagonist being developed by Roche for IV administration. The structure is:

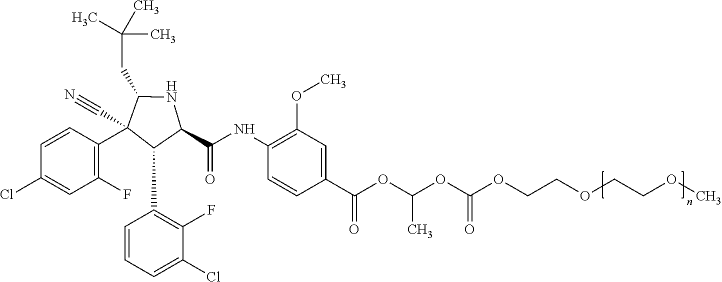

RO-6839921 (RG-7775) may be prepared for example as described in PCT Patent application WO 2014/206866 or by processes analogous thereto.

JNJ 26854165 (Serdemetan) has the structure below, as is an oral HDM2 inhibitor (or antagonist), which showed potent activity against multiple myeloma (MM) cells in vitro and ex vivo; potential agent to restore p53 function and to potentially impact other HDM2 dependent pathways.

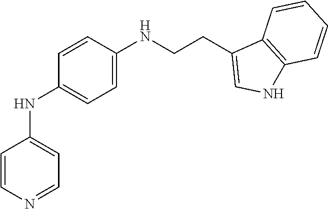 [SMILES: c1ccc2c(CCNc3ccc(Nc4ccncc4)cc3)c[nH]c2c1]

ATSP-7041 (SP-154), a stapled synthetic peptide dual antagonist of MDM2 and MDM4 is being developed by Aileron Therapeutics and Roche and is in Preclinical development. ATSP-7041 (SP-154) has the structure below:

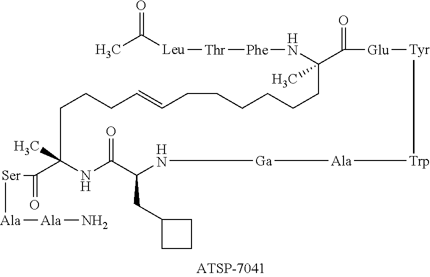

SAH-p53-8 is a stapled synthetic peptide antagonist of MDM4, Hdm2 and Caspase 3 is being developed by Harvard College and Dana-Faber in is in Preclinical development. SAH-p53-8 has the structure below:

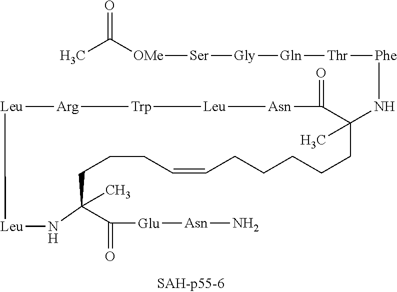

PM-2 (sMTide-02) is a stapled synthetic peptide antagonist of MDM4, Hdm2 and Caspase 3 is being developed by Harvard College and Dana-Faber and is in Preclinical development. PM-2 (sMTide-02) has the structure below:

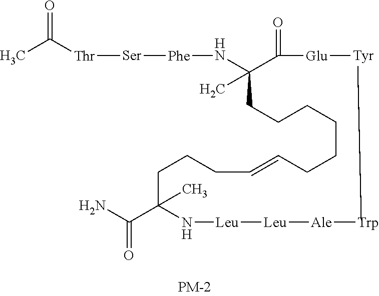

K-178 is a small molecule antagonist of MDM4 that is being developed by Kansai Medical University and is in Preclinical development. K-178 has the chemical the structure below:

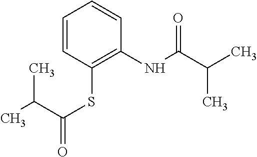 [SMILES: CC(C)C(=O)Nc1ccccc1SC(=O)C(C)C]

MMRi-64 is a small molecule antagonist of MDM2 and MDM4 that is being developed by Roswell Park Cancer Institute and is in the discovery phase. MMRi-64 has the chemical the structure below:

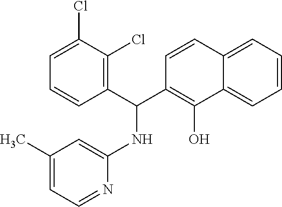 [SMILES: Cc1ccnc(NC(c2cccc(Cl)c2Cl)c2ccc3ccccc3c2O)c1]

Small molecule antagonists of MDM2 and MDM4 are also being developed by Jagiellonian University and the Second Military Medical University. On example has the chemical the structure below:

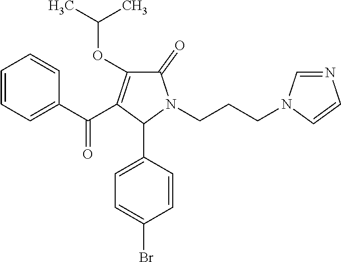 [SMILES: CC(C)OC1=C(C(=O)c2ccccc2)C(c2ccc(Br)cc2)N(CCCn2ccnc2)C1=O]

Small molecule antagonists of MDM2 and MDM4 are being developed by Emory and Georgia State University and are in Preclinical development for the treatment of acute lymphoblastic leukemia.

Small molecule antagonists of MDM2 and MDM4 are being developed by Adamed and are in the discovery phase.

In one embodiment of the invention, the MDM2 antagonist is selected from the group consisting of idasanutlin, HDM-201, KRT-232, ALRN-6924, ALRN-6924, CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

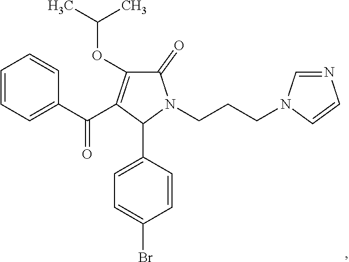 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.

In one embodiment of the invention, the MDM2 antagonist is selected from the group consisting of idasanutlin, HDM-201, KRT-232 (AMG-232), ALRN-6924, CGM-097, milademetan tosylate (DS-3032b), APG-115, BI-907828, LE-004, DS-5272, SJ-0211, APG-155, RG-7112, RG7388 (idasanutlin), SAR405939, Cytarabine (also known as MK-8242 and SCH-900242), BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

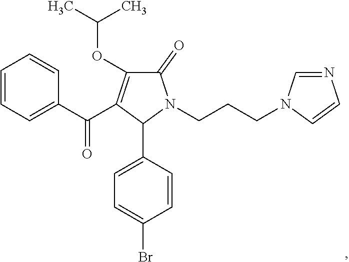 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.

In one embodiment of the invention, the MDM2 antagonist is selected from the group consisting of idasanutlin, HDM-201, KRT-232 (AMG-232), ALRN-6924, CGM-097, milademetan tosylate (DS-3032b), APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

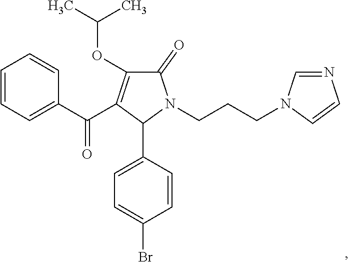 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.

In one embodiment of the invention, the MDM2 antagonist is selected from the group consisting of idasanutlin (RG-7388), HDM-201, KRT-232 (AMG-232), ALRN-6924, MI-773 (SAR405838), milademetan (DS-3032b), APG-115, BI-907828, or a tautomer or a solvate or a pharmaceutically acceptable salt thereof.

In one embodiment of the invention, the MDM2 antagonist is selected from the group consisting of idasanutlin (RG-7388), HDM-201, KRT-232 (AMG-232), ALRN-6924, MI-773 (SAR405838), milademetan (DS-3032b), APG-115, BI-907828, or a compound of formula 1°, or a tautomer or a solvate or a pharmaceutically acceptable salt thereof.

Compounds of formula 1° Particular MDM2 antagonists are isoindoline compounds which are disclosed in our earlier international patent applications PCT/GB2016/053042 and PCT/GB2016/053041 filed 29 Sep. 2016 claiming priority from United Kingdom patent application numbers 1517216.6 and 1517217.4 filed 29 Sep. 2015, the contents of all of which are incorporated herein by reference in their entirety. In particular, the compound (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid (“Compound 1”) is disclosed in our earlier international patent application PCT/GB2016/053042.

In one embodiment, the MDM2 antagonist is a compound of formula 1°:

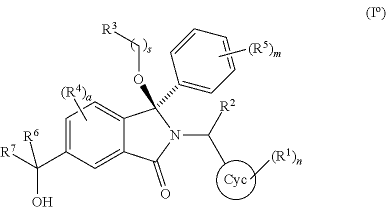

or a tautomer or a solvate or a pharmaceutically acceptable salt thereof, wherein:

wherein cyc is phenyl or a heterocyclic group Het which is pyridinyl, pyrimidinyl, pyrazinyl or pyridazinyl, or an N-oxide thereof;
R¹ is independently selected from hydroxy, halogen, nitro, nitrile, C₁₋₄alkyl, haloC₁₋₄alkyl, hydroxyC₁₋₄alkyl, C₂₋₆alkenyl, C₁₋₄alkoxy, haloC₁₋₄alkoxy, C₂₋₄alkynyl, —O_{0,1}—(CR^{x}R^{y})ᵥ—CO₂H, —(CR^{x}R^{y})ᵥ—CO₂C₁₋₄alkyl, —(CR^{x}R^{y})ᵥ—CON(C₁₋₄alkyl)₂, —P(═O)(R^{x})₂, —S(O)_{d}—R^{x}, —S(O)_{d}-heterocyclic group with 3 to 6 ring members and —S(O)_{d}—N(R⁸)₂, wherein when cyc is Het then R¹ is attached to a carbon atom;

R² is selected from hydrogen, C₁₋₄ alkyl, C₂₋₆alkenyl, hydroxyC₁₋₄alkyl, —(CR^{x}R^{y})ᵤ—CO₂H, —(CR^{x}R^{y})ᵤ—CO₂C₁₋₄alkyl, and —(CR^{x}R^{y})ᵤ—CONR^{x}R^{y};
s is selected from 0 and 1;
R³ is hydrogen or —(A)ₜ—(CR^{x}R^{y})_{q}—X;
t is selected from 0 and 1;
q is selected from 0, 1 and 2;
wherein when R³ is —(A)ₜ—(CR^{x}R^{y})_{q}—X then (i) at least one of s, t and q is other than 0 and (ii) when
is 0 then s is 1 and q is other than 0;
A is a C₃₋₆cycloalkyl group or a heterocyclic group with 3 to 6 ring members, wherein the heterocyclic group comprises one or more (e.g. 1, 2, or 3) heteroatoms selected from N, O, S and oxidised forms thereof;
X is selected from hydrogen, halogen, —CN, —OR⁹, —(CH₂)ᵥ—CO₂H, —(CH₂)ᵥ—CO₂C₁₋₄alkyl, —S(O)_{d}—R^{x}, —C(═O)—C₁₋₄alkyl, —S(O)_{d}—N(H)ₑ(C₁₋₄alkyl)₂₋ₑ, —NR^{x}R_{y}, —NHSO₂R^{x}, —NR^{x}COR^{Y}, and —C(═O)NR^{x}R^{y};
R⁴ and R⁵ are independently selected from halogen, nitrile, C₁₋₄ alkyl, haloC₁₋₄alkyl, C₁₋₄alkoxy and haloC₁₋₄alkoxy;
R⁶ and R⁷ are independently selected from hydrogen, C₁₋₆alkyl, haloC₁₋₆alkyl, C₂₋₆alkenyl, C₂₋₆alkynyl, hydroxy, hydroxyC₁₋₆alkyl, —COOC₁₋₆alkyl, —(CH₂)—O—C₁₋₆alkyl, —(CH₂)—O—(hydroxyC₁₋₆alkyl), —C₁₋₆alkyl-NR^{x}R_{y}, —(CR^{x}R^{y})ₚ—CONR^{x}R_{y}, —(CR^{x}R^{y})ₚ—NR^{x}COR^{Y}, —(CR^{x}R^{y})ₚ—O—CH₂—CONR^{x}R_{y}, heterocyclic group with 3 to 7 ring members, —CH₂-heterocyclic group with 3 to 7 ring members, —CH₂—O-heterocyclic group with 3 to 7 ring members, —CH₂—NH-heterocyclic group with 3 to 7 ring members, —CH₂—N(C₁₋₆alkyl)-heterocyclic group with 3 to 7 ring members, —C(═O)NH-heterocyclic group with 3 to 7 ring members, C₃₋₈cycloalkyl, —CH₂—C₃₋₈cycloalkyl, —CH₂—O—C₃₋₈cycloalkyl, and C₃₋₈cycloalkenyl, wherein said cycloalkyl, cycloalkenyl or heterocyclic groups may be optionally substituted by one or more R^{Z}groups, and wherein in each instance the heterocyclic group comprises one or more (e.g. 1, 2, or 3) heteroatoms selected from N, O, S and oxidised forms thereof;
or the R⁶ and R⁷ groups, together with the carbon atom to which they are attached, can join to form a C₃₋₆cycloalkyl or heterocyclyl group with 3 to 6 ring members, wherein the heterocyclic group comprises one or more (e.g. 1, 2, or 3) heteroatoms selected from N, O, S and oxidised forms thereof, and wherein said C₃₋₆cycloalkyl and heterocyclyl groups may be optionally substituted by one or more R^{z} groups;
R⁸ and R⁹ are independently selected from hydrogen, C₁₋₆alkyl, haloC₁₋₆alkyl, hydroxyC₁₋₆alkyl, —(CH₂)ₖ—O—C₁₋₆alkyl, —(CH₂)ₖ—O—(hydroxyC₁₋₆alkyl), hydroxyC₁₋₆alkoxy, —(CH₂)ₖ—CO₂C₁₋₆alkyl, —(CH₂)ₖ—CO₂H, —C₁₋₆ alkyl—N(H)ₑ(C₁₋₄alkyl)₂₋ₑ, —(CH₂)—C₃₋₈cycloalkyl and —(CH₂)—C₃₋₈cycloalkenyl;
R^{x} and R^{y} are independently selected from hydrogen, halogen, nitro, nitrile, C₁₋₆alkyl, haloC₁₋₆alkyl, C₂₋₆alkenyl, C₂₋₆alkynyl, hydroxy, hydroxyC₁₋₆alkyl, C₁₋₆alkoxy, —(CH₂)ₖ—O—C₁₋₆alkyl, hydroxyC₁₋₆alkoxy, —COOC₁₋₆alkyl, —N(H)ₑ(C₁₋₄alkyl)₂₋ₑ, —C₁₋₆alkyl—N(H)ₑ(C₁₋₄alkyl)₂₋ₑ, —(CH₂)ₖ—C(═O)N(H)ₑ(C₁₋₄alkyl)₂₋ₑ, C₃₋₈cycloalkyl and C₃₋₈cycloalkenyl;
or the R^{x} and R^{y} groups, together with the carbon or nitrogen atom to which they are attached, can join to form a C₃₋₆cycloalkyl or saturated heterocyclyl group with 3 to 6 ring members which may be optionally fused to an aromatic heterocyclyl group of 3 to 5 ring members; or when on a carbon atom the R^{x} and R^{y} groups can join together to form a ═CH₂ group;
R^{Z} is independently selected from halogen, nitro, nitrile, C₁₋₆alkyl, haloC₁₋₆alkyl, C₂₋₆alkenyl, C₂₋₆alkynyl, ═O, hydroxy, hydroxyC₁₋₆alkyl, C₁₋₆alkoxy, —(CH₂)ₖ—O—C₁₋₆alkyl, hydroxyC₁₋₆alkoxy, —C(═O)C₁₋₆alkyl, —C(═O)C₁₋₆alkyl-OH, —C(═O)C₁₋₆alkyl—N(H)ₑ(C₁₋₄alkyl)₂₋ₑ, —C(═O)N(H)ₑ(C₁₋₄alkyl)₂₋ₑ, —(CH₂)ᵣ—CO₂C₁₋₆alkyl, —(CH₂)ᵣ—CO₂H, —N(H)ₑ(C₁₋₄alkyl)₂₋ₑ, —C₁₋₆alkyl—N(H)ₑ(C₁₋₄alkyl)₂₋ₑ, heterocyclyl group with 3 to 6 ring members, heterocyclyl group with 3 to 6 ring members substituted by —C(═O)C₁₋₄alkyl, heterocyclyl group with 3 to 6 ring members substituted by —C(═O)OC₁₋₄alkyl, heterocyclyl group with 3 to 6 ring members substituted by —C(═O)N(H)ₑ(C₁₋₄alkyl)₂₋ₑ, —C(═O)heterocyclyl group with 3 to 6 ring members, C₃₋₈cycloalkyl and C₃₋₈cycloalkenyl, wherein if R⁷ is pyridine then R^{Z} is other than —NH2;
a, j, d, e, n, r and p are independently selected from 0, 1 and 2;
k and m are independently selected from 1 and 2;
u is selected from 0, 1, 2 and 3; and
v is independently selected from 0 and 1

The compounds of the formula (I°):have a chiral centre, marked below with a “*”:

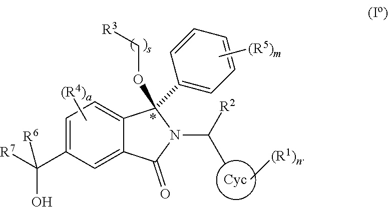

The compounds of formula (I°) include a stereocentre at the position indicated (referred to herein as (3)) and are chiral non-racemic. Compounds of formula (I°) have the stereochemistry shown by the hashed and solid wedged bonds and this stereoisomer predominates.

Typically, at least 55% (e.g. at least 60%, 65%, 70%, 75%, 80%, 85%, 90% or 95%) of the compound of the formula (I°) is present as the shown stereoisomer. In one general embodiment, 97% (e.g. 99%) or more (e.g. substantially all) of the total amount of the compound of the formula (I°) may be present as a single stereoisomer.

The compounds may also include one or more further chiral centres (e.g. in the —CR⁶R⁷OH group and/or in the R³ group and/or in the —CHR² group).

Typically, the compound of formula (I°) has an enantiomeric excess of at least 10% (e.g. at least 20%, 40%, 60%, 80%, 85%, 90% or 95%). In one general embodiment, the compound of formula (I°) has an enantiomeric excess of 97% (e.g. 99%) or more.

For the purposes of this section the isoindolin-1-one ring is numbered as followed:

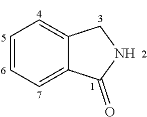

Compounds are named in accordance with protocols utilized by chemical naming software packages.

Compounds of Formula (I°) Wherein Cyc is Phenyl Compounds of formula (I°) wherein cyc is phenyl are disclosed in our earlier international patent application PCT/GB2016/053042 which was published as WO 2017/055860 on 6 Apr. 2017. A cross reference is made to the compounds, subformulae, and substituents disclosed in WO 2017/055860 (e.g. formulae (1), I(e), I(f), I(g), I(g′), I(h), I(i), I(j), I(k), I(L), I(m), I(m′), I(n), I(o), I(o′), I(o″), I(p), I(p′), I(q), I(q′), I(q″), I(q′″), I(q″″), I(r), I(s), I(t), l(u), I(v), I(v′), I(w), I(x), I(x′), I(y), (II), (IIa), (IIb), (IIIa), (IIIb), (IVa), (IVb), (V), (VI), (Via), (VII), (Vila), (VIIb), (VIIc), (Vild), (Vild′), (Vile), (Vile′), (a), (b), (ba), (bb), (bc) or (c)). Accordingly, by virtue of this cross reference, the compounds, subformulae, and substituents of WO 2017/055860 are directly and unambiguously disclosed by the present application.

Particular subformulae, embodiments and compounds of formula (I°) wherein cyc is phenyl include the following: In one embodiment, R¹ is chloro or nitrile, in particular chloro.

When R² is other than hydrogen, the compound of formula (I°) can exist as at least two diastereoisomers:

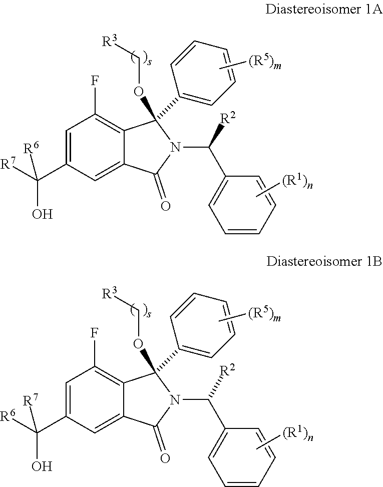

For the avoidance of doubt, the general formula (I°) and all subformulae cover both individual diastereoisomers and mixtures of the diastereoisomers which are related as epimers at the —CHR²— group. In one embodiment the compound of formula (I°) is diastereoisomer 1A or a tautomer or a solvate or a pharmaceutically acceptable salt thereof. In one embodiment the compound of formula (I°) is diastereoisomer 1 B or a tautomer or a solvate or a pharmaceutically acceptable salt thereof.

In one embodiment R² is selected from hydrogen and —(CR^{x}R^{y})ᵤ—CO₂H (e.g. —COOH, —CH₂OOOH, —CH₂CH₂—CO₂H, —(CH(CH₃))—CO₂H and —(C(CH₃)₂)—CO₂H),

In one embodiment, a is 1 and the substituent R⁴ is at the 4-position of the isoindolin-1-one, and the compound of formula (I°) is a compound of formula (Ir) or a tautomer or a solvate or a pharmaceutically acceptable salt thereof:

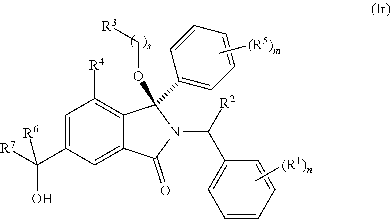

R⁴ is independently selected from halogen, nitrile, C₁₋₄ alkyl, haloC₁₋₄alkyl, C₁₋₄alkoxy and haloC₁₋₄alkoxy.

In one embodiment, R⁴ is halogen. In one embodiment, R⁴ is fluoro or chloro. In another embodiment, R⁴ is fluoro.

In one embodiment, a is 1, the substituent R⁴ is at the 4-position of the isoindolin-1-one, and R⁴ is F and the compound of formula (I°) is a compound of formula (Is) or a tautomer or a solvate or a pharmaceutically acceptable salt thereof:

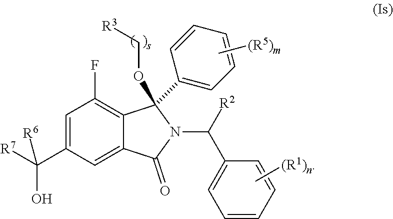

When R⁶ and R⁷ are different, the compound of formula (I°) can exist as at least two diastereoisomers:

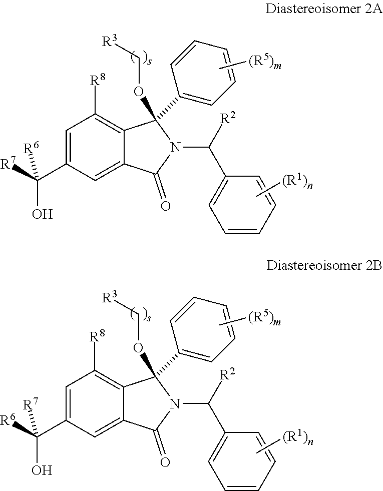

For the avoidance of doubt, the general formula (I°) and all subformulae cover both individual diastereoisomers and mixtures of the diastereoisomers which are related as epimers at the —CR⁶R⁷OH group.

In one embodiment, R⁶ is C₁₋₆alkyl (such as methyl or ethyl e.g. methyl) and R⁷ is oxanyl, and the compound of formula (I°) is a compound of formula (Iw):

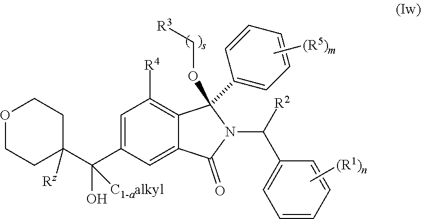

In one embodiment of formula (Iw) R^{z} is hydrogen or fluorine.

### Subformulae

In one embodiment, R⁶ is methyl or ethyl, and the compound of formula (I°) is a compound of formula (IIIb) or a tautomer or a solvate or a pharmaceutically acceptable salt thereof:

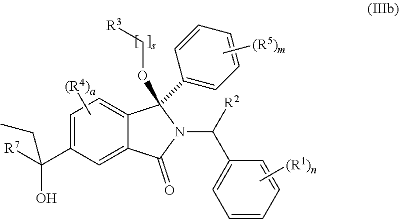

wherein R¹, R², R³, R⁴, R⁵, R⁷, a, m and s are as defined herein.

In one embodiment, s is 0 and the compound of formula (I°) is a compound of formula (IVb) or a tautomer or a solvate or a pharmaceutically acceptable salt thereof:

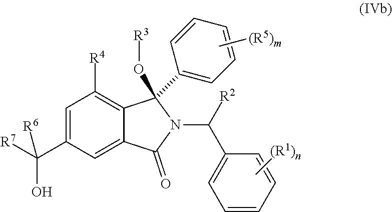

wherein R¹, R², R³, R⁴, R⁵, R⁷, a, m and s are as defined herein.

In one embodiment, m is 1 and the substituent R⁴ is at the 4-position of the phenyl group, and the compound of formula (I°) is a compound of formula (VI) or a tautomer or a solvate or a pharmaceutically acceptable salt thereof:

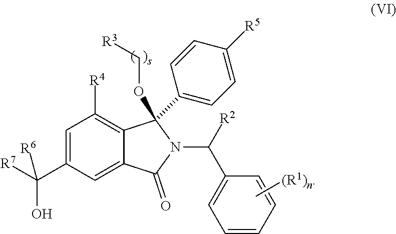

In one embodiment, R⁵ is chloro and the compound of formula (VI) is a compound of formula (VIa) or a tautomer or a solvate or a pharmaceutically acceptable salt thereof:

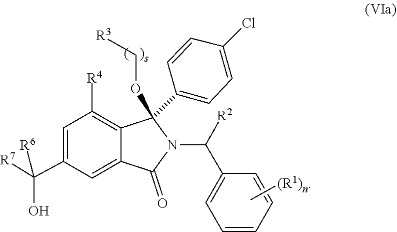

In one embodiment, R³ is methyl, and the compound of formula (VI) is a compound of formula (VIIf) or a tautomer or a solvate or a pharmaceutically acceptable salt thereof:

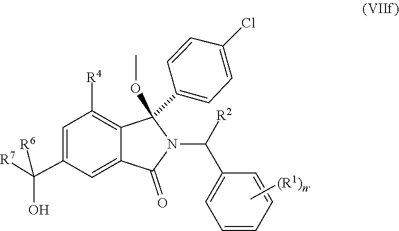

In one embodiment of Formula (VIIf), R⁶ is ethyl.

In one embodiment of the compound of formula (VIIf), R⁷ is selected from methyl, oxanyl, pyrazolyl, imidazolyl, piperidinyl, and cyclohexyl wherein said cycloalkyl and heterocyclic groups are optionally substituted by one or more R^{Z} groups (e.g. methyl, fluorine, or hydroxy).

In one embodiment of the compound of formula (VIIf, R⁷ is selected from oxanyl and methyl.

In one embodiment of the compound of formula (VIIf), R⁷ is selected from piperidinyl optionally substituted by one or more R^{Z} groups (e.g. methyl, fluorine, or hydroxy).

In another embodiment of the subsformulae described hereinabove, R² is selected from —(CH(CH₃))—CO₂H and —(C(CH₃)₂—CO₂H).

In one embodiment, the MDM2 antagonist is a compound of formula (I°) or a tautomer or a solvate or a pharmaceutically acceptable salt thereof, wherein: R¹ is halogen (e.g. Cl), nitrile, O_{0,1}(CR^{x}R^{y})ᵥCOOH (e.g. —COOH, —CH₂OOOH, —OCH₂OOOH or —C(CH₃)₂OOOO;
n is 1 or 2;

R² is selected from hydrogen and —(CR^{x}R^{y})ᵤ—CO₂H (e.g. —COOH, —CH₂OOOH, —CH₂CH₂—CO₂H, —(CH(CH₃))—CO₂H and —(C(CH₃)₂)—CO₂H).

R³ is hydrogen and s is 1;

R⁴ is halogen (e.g. F);

R⁵ is halogen (e.g. Cl);

m is 1;

R⁶ is hydrogen or C₁₋₆alkyl (e.g. —CH₃ or —CH₂CH₃);

R⁷ is C₁₋₄alkyl (e.g. methyl), hydroxylC₁₋₄alkyl (e.g. hydroxylmethyl), methoxyC₁₋₄alkyl (e.g. methoxymethyl), a heterocyclic group with 5 or 6 ring members (e.g. piperidinyl, oxanyl, imidazolyl or pyrazolyl));

wherein said heterocyclic group with 5 or 6 ring members may be optionally substituted with one or two R^{Z} groups independently selected from C₁₋₄alkyl (e.g. methyl).

In one embodiment, the MDM2 antagonist is a compound of formula (I°) which is one of the Examples 1-137 or is selected from the Examples 1-137 or a tautomer, N-oxide, pharmaceutically acceptable salt or solvate thereof described in the first set of examples defined herein i.e. the compounds in which cyc is phenyl, as also described in WO 2017/055860)

In one embodiment, the MDM2 antagonist is a compound of formula (I°) which is one of the Examples 1-97 (examples wherein cyc is phenyl) or is selected from the Examples 1-97 (examples wherein cyc is phenyl) or a tautomer, N-oxide, pharmaceutically acceptable salt or solvate thereof described in the first set of examples defined herein i.e. the compounds in which cyc is phenyl, as also described in WO 2017/055860)

In one embodiment, the MDM2 antagonist is a compound of formula (I°) which is selected from the following compounds, or a tautomer, N-oxide, pharmaceutically acceptable salt or solvate thereof:
4-{[(1R)-1-(4-chlorophenyl)-7-fluoro-5-[1-hydroxy-1-(1-methyl-1 H-imidazol-4-yl)propyl]-1-{[1-(hydroxymethyl)cyclopropyl]methoxy}-3-oxo-2,3-dihydro-1 H-isoindol-2-yl]methyl}benzonitrile

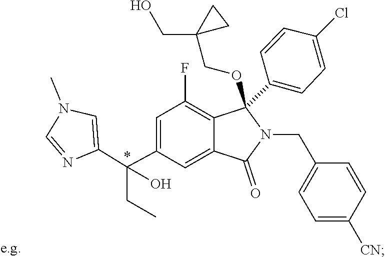 [SMILES: CCC(O)(c1cc(F)c2c(c1)C(=O)N(Cc1ccc(C#N)cc1)[C@@]2(OCC1(CO)CC1)c1ccc(Cl)cc1)c1cn(C)cn1]

(3S)-3-(4-chlorophenyl)-3-[(1R)-1-(4-chlorophenyl)-7-fluoro-5-[1-hydroxy-1-(oxan-4-yl)ethyl]-1-methoxy-3-oxo-2,3-dihydro-1 H-isoindol-2-yl]propanoic acid

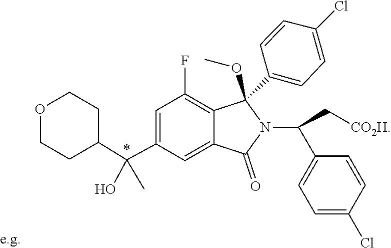 [SMILES: CO[C@]1(c2ccc(Cl)cc2)c2c(F)cc(C(C)(O)C3CCOCC3)cc2C(=O)N1[C@@H](CC(=O)O)c1ccc(Cl)cc1]

In one embodiment, the MDM2 antagonist is a compound of formula (I°) which is selected from the following compounds, or a tautomer, N-oxide, pharmaceutically acceptable salt or solvate thereof:
4-{[(1R)-1-(4-chlorophenyl)-7-fluoro-5-[1-hydroxy-1-(1-methyl-1H-imidazol-4-yl)propyl]-1-{[1-(hydroxymethyl)cyclopropyl]methoxy}-3-oxo-2,3-dihydro-1 H-isoindol-2-yl]methyl}benzonitrile; and
(3S)-3-(4-chlorophenyl)-3-[(1R)-1-(4-chlorophenyl)-7-fluoro-5-[1-hydroxy-1-(oxan-4-yl)ethyl]-1-methoxy-3-oxo-2,3-dihydro-1 H-isoindol-2-yl]propanoic acid.

In one embodiment, the MDM2 antagonist is a compound of formula (I°) which is diastereoisomer 2B and is selected from the following compounds, or a tautomer, N-oxide, pharmaceutically acceptable salt or solvate thereof:
4-{[(1R)-1-(4-chlorophenyl)-7-fluoro-5-[1-hydroxy-1-(1-methyl-1H-imidazol-4-yl)propyl]-1-{[1-(hydroxymethyl)cyclopropyl]methoxy}-3-oxo-2,3-dihydro-1 H-isoindol-2-yl]methyl}benzonitrile; and
(3S)-3-(4-chlorophenyl)-3-[(1R)-1-(4-chlorophenyl)-7-fluoro-5-[1-hydroxy-1-(oxan-4-yl)ethyl]-1-methoxy-3-oxo-2,3-dihydro-1 H-isoindol-2-yl]propanoic acid.

In one embodiment, the compound of formula (I°) is 2-(5-chloro-2-{[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]methyl}phenyl)-2-methylpropanoic acid, or a tautomer, N-oxide, pharmaceutically acceptable salt or solvate thereof.

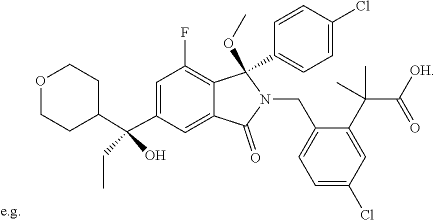 [SMILES: CC[C@@](O)(c1cc(F)c2c(c1)C(=O)N(Cc1ccc(Cl)cc1C(C)(C)C(=O)O)[C@@]2(OC)c1ccc(Cl)cc1)C1CCOCC1]

In one embodiment, the MDM2 antagonist is a compound of formula (I°) which is (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid, (“Compound 1”) or a tautomer, N-oxide, pharmaceutically acceptable salt or solvate thereof

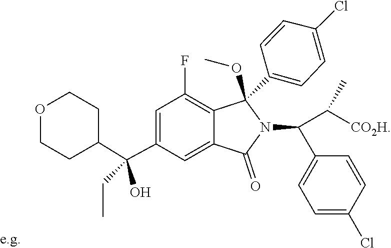 [SMILES: CC[C@@](O)(c1cc(F)c2c(c1)C(=O)N([C@H](c1ccc(Cl)cc1)[C@H](C)C(=O)O)[C@@]2(OC)c1ccc(Cl)cc1)C1CCOCC1]

For the avoidance of doubt, it is to be understood that each general and specific embodiment and example for one substituent may be combined with each general and specific embodiment and example for one or more, in particular all, other substituents as defined herein and that all such embodiments are embraced by this application.

### Compounds of Formula (I°) Wherein Cyc is a Heterocyclic Group

Compounds of formula (I°) wherein cyc is is a heterocyclic group are disclosed in our earlier international patent application PCT/GB2016/053041 which was published as WO 2017/055859 on 6 Apr. 2017.

A cross reference is made to the compounds, subformulae, and substituents disclosed in WO 2017/055859 (e.g. formulae (I), I(a), I(a′), I(b), I(c), I(d), I(e), l(f), I(g), I(g′), I(h), I(i), I(j), I(k), I(L), I(m), I(m′), I(n), I(o), I(o′), I(o″), I(p), I(p′), I(q), I(q′), I(q″), I(q″′), I(q″″), I(r), I(s), I(t), I(u), I(v), I(v′), I(w), I(x), I(x′), I(y), (II), (IIa), (IIb), (IIIa), (IIIIb), (Iva), (IVb), (V), (VI), (Vla), (VII), (VIIa), (VIIb), (VIIc), (VIId), (VIId′), (VIIe), (VIIe′), (a), (b), (ba), (bb), (bc), or (c)) and examples thereof as defined herein, Accordingly, by virtue of this cross reference, the compounds, subformulae, and substituents of WO 2017/055859 are directly and unambiguously disclosed by the present application.

Particular subformulae, embodiments and compounds of formula (I°) wherein cyc is a heterocyclic group include the following: In another embodiment, R² is hydrogen and the compound of formula (I°) is a compound of formula (Ie) or a tautomer or a solvate or a pharmaceutically acceptable salt thereof:

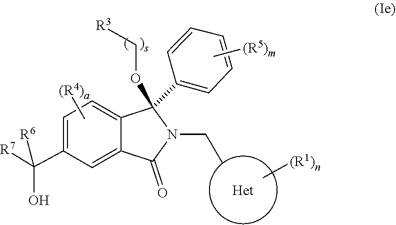

When R² is other than hydrogen, the compound of formula (I°) can exist as at least two diastereoisomers:

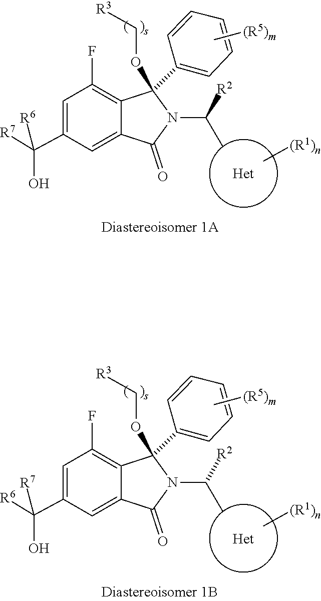

For the avoidance of doubt, the general formula (I°) and all subformulae cover both individual diastereoisomers and mixtures of the diastereoisomers which are related as epimers at the —CHR²— group. In one embodiment the compound of formula I is diastereoisomer 1A or a tautomer or a solvate or a pharmaceutically acceptable salt thereof. In one embodiment the compound of formula I is diastereoisomer 1 B or a tautomer or a solvate or a pharmaceutically acceptable salt thereof.

In one embodiment, A is a C₃₋₆cycloalkyl group (i.e. g is 1, 2 or 3) and t is 1 and s is 0 or 1, and the compound of formula (I°) is a compound of formula (If or a tautomer or a solvate or a pharmaceutically acceptable salt thereof:

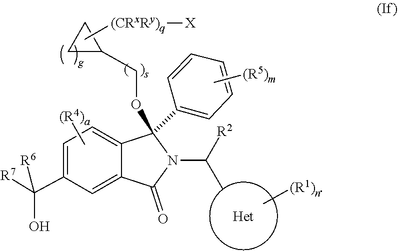

In one embodiment, A is a C₃₋₆cycloalkyl group (i.e. g is 1, 2 or 3) and t is 1 and s is 1, and the compound of formula (I°) is a compound of formula (Ig) or a tautomer or a solvate or a pharmaceutically acceptable salt thereof:

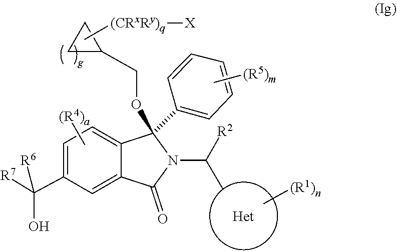

In one embodiment, A is a C₃₋₆cycloalkyl group (i.e. g is 1, 2 or 3) and t is 1 and s is 1, and the cycloalkyl group is geminally disubstituted (i.e. the group —(CR^{x}R^{y})_{q}—X and the —CH₂—O-isoindolinone group are both attached to the same atom of the cycloalkyl group), and the compound of formula (I°) is a compound of formula (Ih) or a tautomer or a solvate or a pharmaceutically acceptable salt thereof:

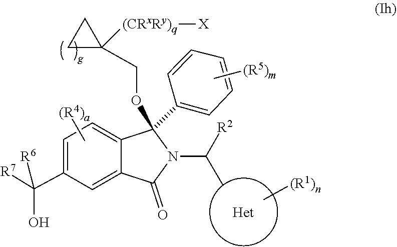

In one embodiment, A is a cyclopropyl group (i.e. g is 1), t is 1 and s is 1. Therefore the cycloalkyl group is a cyclopropyl group and the compound of formula (I°) is a compound of formula (II) or a tautomer or a solvate or a pharmaceutically acceptable salt thereof:

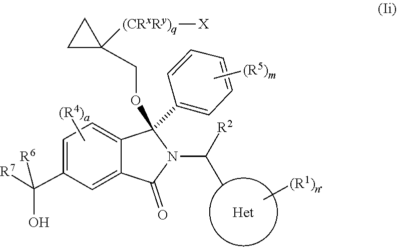

In one embodiment, A is a C₃₋₆cycloalkyl group (i.e. g is 1, 2 or 3), t is 1, s is 1 and X is —CN and the compound of formula (I°) is a compound of the formula (Ik′) or a tautomer or a solvate or a pharmaceutically acceptable salt thereof:

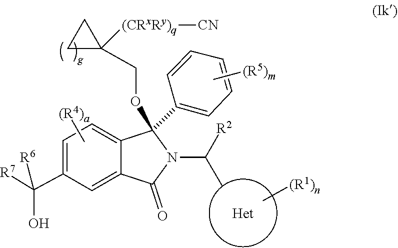

In another embodiment, A is a C₃₋₆cycloalkyl group (i.e. g is 1, 2 or 3), t is 1, s is 1 and R^{x} and R^{y} are hydrogen (including ¹H and ²H) and the compound of formula (I°) is a compound of formula (IL) or a tautomer or a solvate or a pharmaceutically acceptable salt thereof:

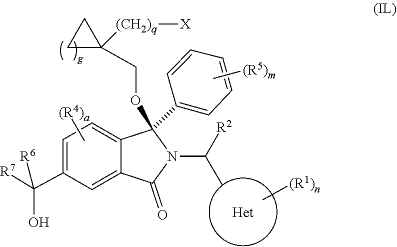

In one embodiment, A is a C₃-cycloalkyl group (i.e. g is 1), t is 1, s is 1 and X is —CN and the compound of formula (I°) is a compound of formula (In′) or a tautomer or a solvate or a pharmaceutically acceptable salt thereof:

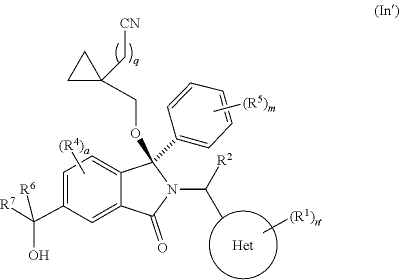

wherein q is 0 or 1. In one embodiment of the compound (In), q is 0.

In one embodiment, R³ is —(CR^{x}R^{y})_{q}—X and s is 1, t is 0 and q is 1 or 2, and the compound of formula (I°) is a compound of the formula (Ip):

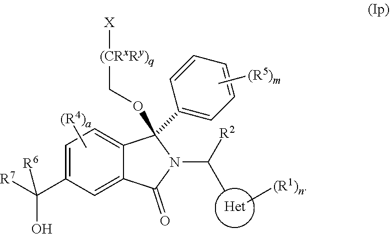

In one embodiment, A is a C₃₋₆cycloalkyl group or saturated heterocyclic group with 3 to 6 ring members, wherein t is 1, and s is 1, Y is independently selected from —CH₂—, 0, or SO₂, i is 0 or 1, g is 1, 2, 3 or 4 and i+g is 1, 2, 3 or 4 and the compound of formula (I°) is a compound of the formula (Iq) or a tautomer or a solvate or a pharmaceutically acceptable salt thereof:

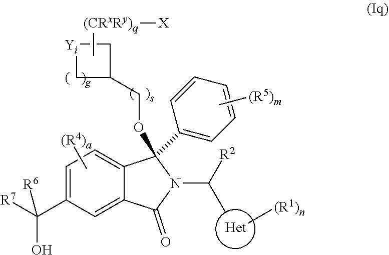

In one embodiment, i is 1 and Y is 0 or SO₂, in particular 0. In one embodiment, the compound of formula (Iq) is a compound of formula (Iq″″) or a tautomer or a solvate or a pharmaceutically acceptable salt thereof:

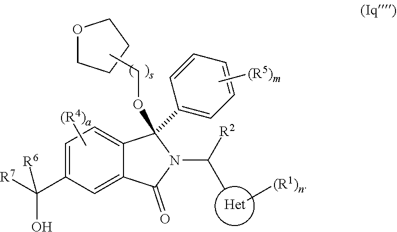

In one embodiment, s is 0, t is 1, A is tetrahydofuranyl, q is 0 and X is hydrogen. In one embodiment, R³ is tetrahydrofuranyl and s is 0.

In one embodiment, a is 1 and the substituent R⁴ is at the 4-position of the isoindolin-1-one, and the compound of formula (I°) is a compound of formula (Ir) or a tautomer or a solvate or a pharmaceutically acceptable salt thereof:

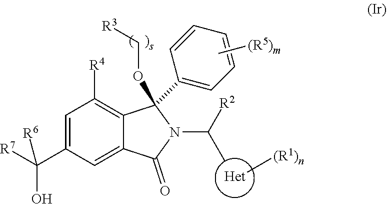

R⁴ is independently selected from halogen, nitrile, C₁₋₄ alkyl, haloC₁₋₄alkyl, C₁₋₄alkoxy and haloC₁₋₄alkoxy.

In one embodiment, R⁴ is halogen. In one embodiment, R⁴ is fluoro or chloro. In another embodiment, R⁴ is fluoro.

In one embodiment, a is 1, the substituent R⁴ is at the 4-position of the isoindolin-1-one, and R⁴ is F and the compound of formula (I°) is a compound of formula (Is) or a tautomer or a solvate or a pharmaceutically acceptable salt thereof:

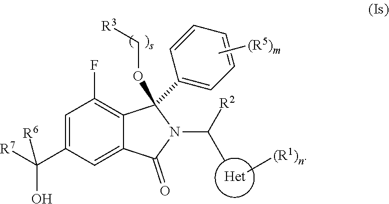

When R⁶ and R⁷ are different, the compound of formula (I°) can exist as at least two diastereoisomers:

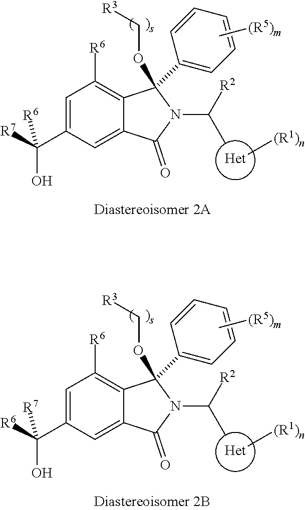

For the avoidance of doubt, the general formula (I°) and all subformulae cover both individual diastereoisomers and mixtures of the diastereoisomers which are related as epimers at the —CR⁶R⁷OH group.

In one embodiment, R⁷ is 4-fluoro-1-methylpiperidin-4-yl and the compound of formula (I°) is a compound of formula (Ix″) or a tautomer or a solvate or a pharmaceutically acceptable salt thereof:

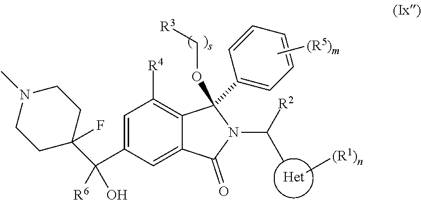

### Subformulae

In one embodiment, the compound of formulae (I°) is a compound of formulae (II) or a tautomer or a solvate or a pharmaceutically acceptable salt thereof:

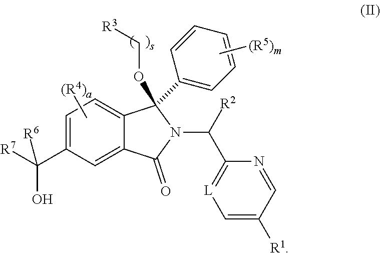

wherein L is CR¹, CH or N and R¹, R², R³, R⁴, R⁵, R⁶, R⁷, a, m and s are as defined herein. In one embodiment L is CH. In one embodiment L is N. In one embodiment L is CR¹ such as C—OH or C-hydroxyC₁₋₄alkyl (e.g. C—OH or C—CH₂OH).

In another embodiment, R¹ is chloro or nitrile and the compound of formula (II) is a compound of formula (IIa) or a tautomer or a solvate or a pharmaceutically acceptable salt thereof:

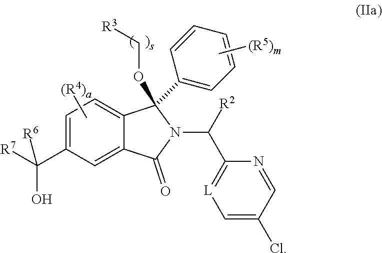

wherein R¹, R², R³, R⁴, R⁵, R⁷, m and s are as defined herein.

In one embodiment, R⁶ is ethyl, and the compound of formula (II) is a compound of formula (IIIb) or a tautomer or a solvate or a pharmaceutically acceptable salt thereof:

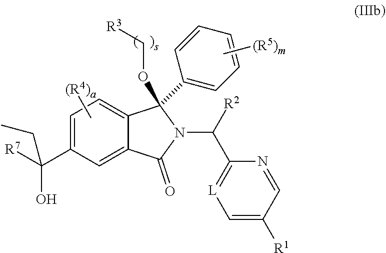

wherein R¹, R², R³, R⁴, R⁵, R⁷, a, m and s are as defined herein.

In one embodiment, s is 0 and the compound of formula (II) is a compound of formula (IVb) or a tautomer or a solvate or a pharmaceutically acceptable salt thereof:

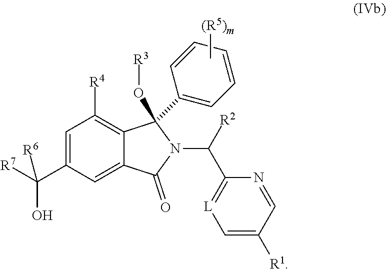

wherein R¹, R², R³, R⁴, R⁵, R⁷, m and s are as defined herein.

In one embodiment, R⁴ is F and the compound of formula (I°) is a compound of formula (V) or a tautomer or a solvate or a pharmaceutically acceptable salt thereof:

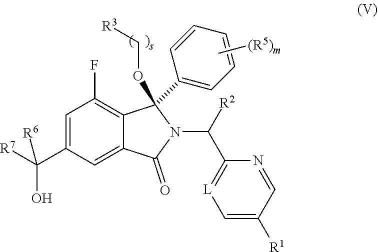

wherein R¹, R², R³, R⁵, R⁷, m and s are as defined herein.

In one embodiment, m is 1 and the substituent R⁴ is at the 4-position of the phenyl group, and the compound of formula (II) is a compound of formula (VI) or a tautomer or a solvate or a pharmaceutically acceptable salt thereof:

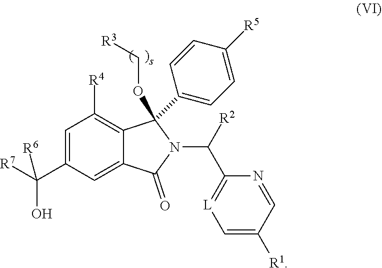

In one embodiment, R⁵ is chloro and the compound of formula (VI) is a compound of formula (VIa) or a tautomer or a solvate or a pharmaceutically acceptable salt thereof:

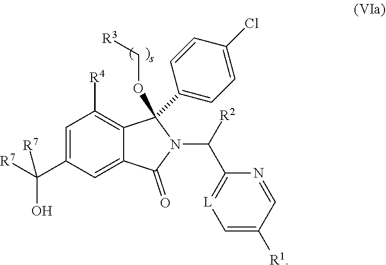

In one embodiment, A is a C₃₋₆cycloalkyl group (g is 1, 2 or 3) and t is 1, and the compound of formula (VI) is a compound of formula (VII) or a tautomer or a solvate or a pharmaceutically acceptable salt thereof:

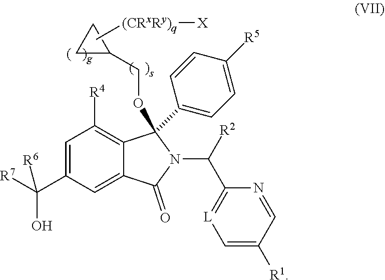

In one embodiment, A is a C₃₋₆cycloalkyl group (g is 1, 2 or 3) and t is 1, and the cycloalkyl group is geminally disubstituted (i.e. the group —(CR^{x}R^{y})—X and the CH₂ group (where s is 1) or the oxygen atom (where s is 0) are both attached to the same atom of the cycloalkyl group, and the compound of formula (VII) is a compound of formula (Vila) or a tautomer or a solvate or a pharmaceutically acceptable salt thereof:

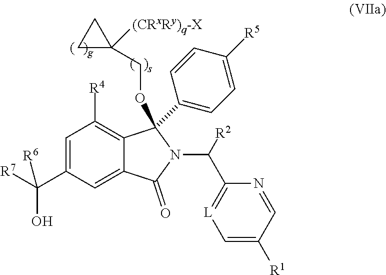

In one embodiment, g is 1, and so the cycloalkyl group is a cyclopropyl group and the compound of formula (VIIa) is a compound of formula (VIIb) or a tautomer or a solvate or a pharmaceutically acceptable salt thereof:

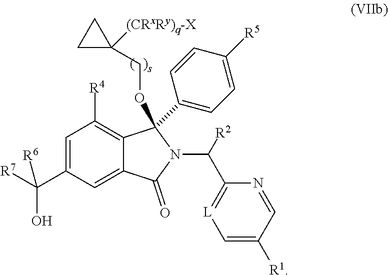

In one embodiment, s is 1, and the compound of formula (VIIb) is a compound of formula (VIIc) or a tautomer or a solvate or a pharmaceutically acceptable salt thereof:

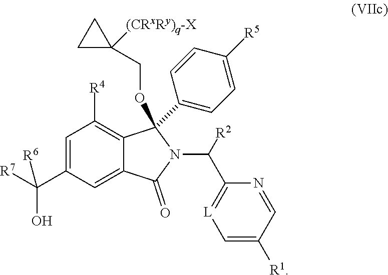

In one embodiment, X is —CN and the compound of formula (Vild) is a compound of the formula (Vile″) or a tautomer or a solvate or a pharmaceutically acceptable salt thereof:

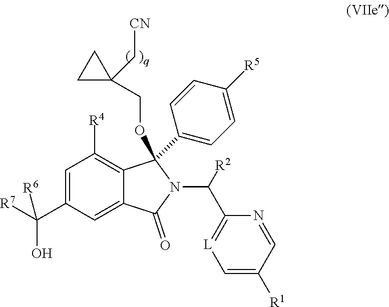

wherein q is 0 or 1, and in particular q is 0.

In one embodiment, R³ is methyl, and the compound of formula (VI) is a compound of formula (VIIf) or a tautomer or a solvate or a pharmaceutically acceptable salt thereof:

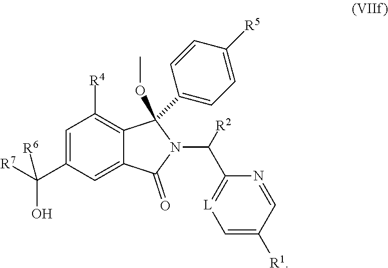

In one embodiment of the compound of formula (a), R⁷ is piperidinyl or piperazinyl, optionally substituted with C₁₋₆ alkyl (e.g. methyl) and/or halo (e.g. flouro).

In one embodiment of the compound of formula (a′), R⁷ is piperidinyl, optionally substituted with C₁₋₆ alkyl (e.g. methyl) and/or halo (e.g. flouro).

In one embodiment, A is a heterocyclyl group with 3 to 6 ring members, wherein the heterocyclic group comprises one or more (e.g. 1, 2, or 3) heteroatoms selected from N, O, S and oxidised forms thereof (t is 1; g is 1, 2, 3 or 4; Z represents N, O, S and oxidised forms thereof; i is 1, 2, or 3; and i+g=2, 3, 4 or 5), and the compound of formula (VI) is a compound of formula (b) or a tautomer or a solvate or a pharmaceutically acceptable salt thereof:

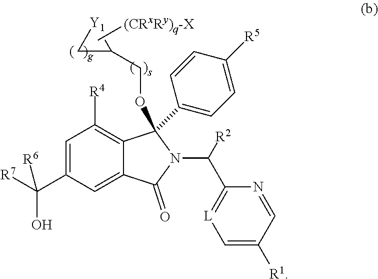

In one embodiment, s is 0, g is 2, q is 0 and X is hydrogen, and the compound of formula (b) is a compound of formula (bb) or a tautomer or a solvate or a pharmaceutically acceptable salt thereof:

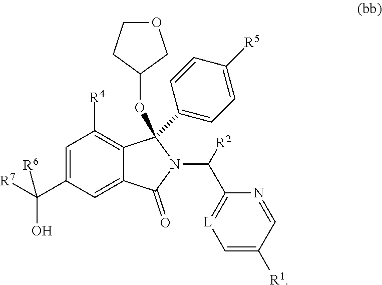

In another embodiment, the compound of formula (I°) is a compound of formula (c) or a tautomer or a solvate or a pharmaceutically acceptable salt thereof:

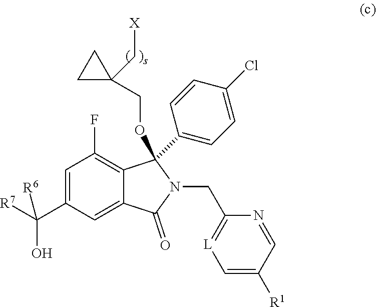

wherein R¹ is chloro or nitrile, s is 1 and X is hydroxyl or s is 0 and X is —C(═O)NH₂.

In another embodiment, the compound of formula (I°) is a compound of formula (c′) or a tautomer or a solvate or a pharmaceutically acceptable salt thereof:

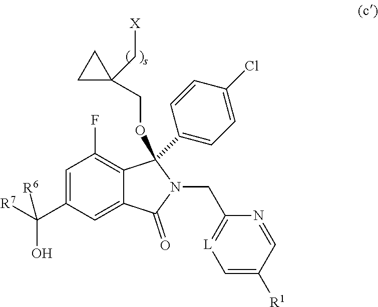

wherein R¹ is chloro or nitrile, s is 1 and X is hydroxyl or s is 0 and X is —CN.

In one embodiment, the MDM2 antagonist is a compound of formula (I°) or a tautomer or a solvate or a pharmaceutically acceptable salt thereof, wherein:

Het is pyridinyl or pyrimidinyl

R¹ is attached to a carbon atom and is independently selected from hydroxy, halogen, nitro, nitrile and C₁₋₄alkyl;

R² is selected from hydrogen, C₁₋₄ alkyl, C₂₋₆alkenyl, hydroxyC₁₋₄alkyl and —CH₂CO₂H;

R³ is hydrogen or —(A)ₜ—(CR^{x}R^{y})_{q}—X;

s and t are independently selected from 0 and 1;

q is selected from 0, 1 and 2;

wherein when R³ is —(A)ₜ—(CR^{x}R^{y})_{q}—X then (i) at least one of s, t and q is other than 0 and (ii) when t is 0

then s is 1 and q is other than 0;

A is a heterocyclic group with 3 to 6 ring members, wherein the heterocyclic group comprises one or more (e.g. 1, 2, or 3) heteroatoms selected from N, O, S and oxidised forms thereof;

X is selected from hydrogen, halogen, —CN and —OR⁹;

R⁴ and R⁵ are independently selected from halogen, nitrile and C₁₋₄ alkyl;

R⁶ is selected from hydrogen and C₁₋₆alkyl;

R⁷ is selected from heterocyclic group with 3 to 7 ring members, —CH₂-heterocyclic group with 3 to 7 ring members, C₃₋₈cycloalkyl, and —CH₂—C₃₋₈cycloalkyl, wherein said cycloalkyl or heterocyclic groups may be optionally substituted by one or more R^{Z} groups, and wherein in each instance the heterocyclic group comprises one or more (e.g. 1, 2, or 3) heteroatoms selected from N, O, S and oxidised forms thereof;

R⁹ is selected from hydrogen and C₁₋₆alkyl;

R^{x} and R^{y} are independently selected from hydrogen and C₁₋₆alkyl;

R^{Z} is independently selected from halogen, nitro, nitrile, C₁₋₆alkyl, haloC₁₋₆alkyl, C₂₋₆alkenyl, hydroxy, hydroxyC₁₋₆alkyl, C₁₋₆alkoxy, —C(═O)C₁₋₆alkyl, and —N(H)ₑ(C₁₋₄alkyl)₂-e;
n and e are independently selected from 0, 1 and 2;
m is selected from 1 and 2; and

a is selected from 0 and 1.

In one embodiment, the MDM2 antagonist is the compound of formula (I°) or a tautomer or a solvate or a pharmaceutically acceptable salt thereof, wherein:

Het is pyridinyl or pyrimidinyl

R¹ is attached to a carbon atom and is independently selected from halogen, hydroxy and nitrile;

R² is selected from hydrogen, C₁₋₄ alkyl and —CH₂CO₂H;

R³ is hydrogen or —(A)ₜ—(CR^{x}R^{y})_{q}—X;

A is a heterocyclic group with 3 to 6 ring members, wherein the heterocyclic group comprises one or more (e.g. 1, 2, or 3) heteroatoms selected from N, O, S and oxidised forms thereof;

s and t are independently selected from 0 and 1;

q is selected from 0, 1 and 2;

wherein when R³ is —(A)ₜ—(CR^{x}R^{y})_{q}—X then (i) at least one of s, t and q is other than 0 and (ii) when t is 0 then s is 1 and q is other than 0;

X is selected from hydrogen, halogen or —OR⁹;

R⁴ and R⁵ are independently selected from halogen;

R⁶ is selected from hydrogen and C₁₋₆alkyl;

R⁷ is selected from heterocyclic group with 3 to 7 ring members, —CH₂-heterocyclic group with 3 to 7 ring members, C₃₋₈cycloalkyl, and —CH₂—C₃₋₈cycloalkyl, wherein said cycloalkyl, cycloalkenyl or heterocyclic groups may be optionally substituted by one or more R^{Z} groups, and wherein in each instance the heterocyclic group comprises one or more (e.g. 1, 2, or 3) heteroatoms selected from N, O, S and oxidised forms thereof;

R⁹ is selected from hydrogen and C₁₋₆alkyl;

R^{x} and R^{y} are independently selected from hydrogen and C₁₋₆alkyl;

R^{Z} is independently selected from halogen, nitro, nitrile, and C₁₋₆alkyl;

n is 1 and m is 1; and

a is selected from 0 and 1.

In one embodiment, the MDM2 antagonist is a compound of formula (I°) or a tautomer or a solvate or a pharmaceutically acceptable salt thereof, wherein:

Het is pyridinyl or pyrimidinyl

R¹ is attached to a carbon atom and is independently selected from halogen, hydroxy and nitrile;

R² is selected from hydrogen, C₁₋₄ alkyl and —CH₂CO₂H;

R³ is —(A)ₜ—(CR^{x}R^{y})_{q}—X;

A is a heterocyclic group with 3 to 6 ring members, wherein the heterocyclic group comprises one or more (e.g. 1, 2, or 3) heteroatoms selected from N, O, S and oxidised forms thereof;

s and t are independently selected from 0 and 1;

q is selected from 0, 1 and 2;

wherein (i) at least one of s, t and q is other than 0 and (ii) when t is 0 then s is 1 and q is other than 0;

X is selected from hydrogen, halogen and —OR⁹;

R⁴ and R⁵ are independently selected from halogen;

R⁶ is selected from hydrogen and C₁₋₆alkyl;

R⁷ is a heterocyclic group with 3 to 7 ring members optionally substituted by one or more R^{Z} groups;

R⁹ is selected from hydrogen and C₁₋₆alkyl;

R^{x} and R^{y} are independently selected from hydrogen and C₁₋₆alkyl;

R^{Z} is independently selected from halogen and C₁₋₆alkyl;

n is, 1 and m is 1 and

a is 1.

In one embodiment, the MDM2 antagonist is a compound of formula (I°) which is one of the Examples 1-580 (examples wherein cyc is a heterocyclic group or is selected from the Examples 1-580 or a tautomer, N-oxide, pharmaceutically acceptable salt or solvate thereof (the compounds of formula I° described in the second set of examples defined herein i.e. the compounds in which cyc is Het, as also described in WO 2017/055859).

In one embodiment, the MDM2 antagonist is a compound of formula (I°) which is one of the Examples 1-460 or is selected from the Examples 1-460 or a tautomer, N-oxide, pharmaceutically acceptable salt or solvate thereof (the compounds of formula I° described in the second set of examples defined herein i.e. the compounds in which cyc is Het, as also described in WO 2017/055859).

In one embodiment, the MDM2 antagonist is a compound of formula (I°) which is one of the Examples 1-459 or is selected from the Examples 1-459 or a tautomer, N-oxide, pharmaceutically acceptable salt or solvate thereof (the compounds of formula I° described in the second set of examples defined herein i.e. the compounds in which cyc is Het, as also described in WO 2017/055859).

In one embodiment, the MDM2 antagonist is a compound of formula (I°) which is selected from the following compounds, or a tautomer, N-oxide, pharmaceutically acceptable salt or solvate thereof:

(3R)-3-(4-chlorophenyl)-2-[(5-chloropyrimidin-2-yl)methyl]-4-fluoro-6-{1-hydroxy-1-[trans-4-hydroxycyclohexyl]ethyl}-3-{[1-(hydroxymethyl)cyclopropyl]methoxy}-2,3-dihydro-1 H-isoindol-1-one;

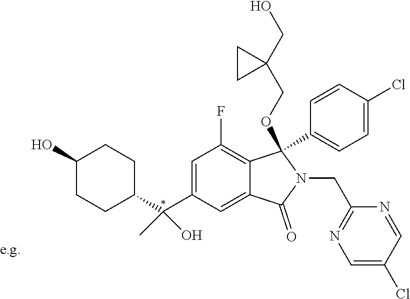 [SMILES: CC(O)(c1cc(F)c2c(c1)C(=O)N(Cc1ncc(Cl)cn1)[C@@]2(OCC1(CO)CC1)c1ccc(Cl)cc1)[C@H]1CC[C@H](O)CC1]

2-{[(1R)-1-(4-chlorophenyl)-7-fluoro-5-[1-hydroxy-1-(1-methyl-1 H-imidazol-4-yl)propyl]-3-oxo-1-[(3S)-oxolan-3-yloxy]-2,3-dihydro-1 H-isoindol-2-yl]methyl}pyrimidine-5-carbonitrile;

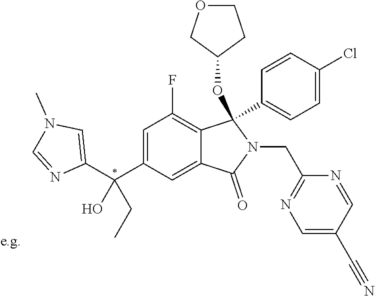 [SMILES: CCC(O)(c1cc(F)c2c(c1)C(=O)N(Cc1ncc(C#N)cn1)[C@@]2(O[C@H]1CCOC1)c1ccc(Cl)cc1)c1cn(C)cn1]

(3R)-2-[(5-chloro-3-hydroxypyridin-2-yl)methyl]-3-(4-chlorophenyl)-4-fluoro-6-[1-hydroxy-1-(1-methyl-1 H-imidazol-4-yl)propyl]-3-(2-hydroxyethoxy)-2,3-dihydro-1 H-isoindol-1-one;

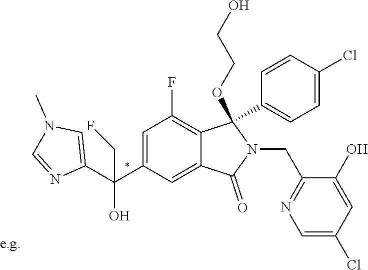 [SMILES: Cn1cnc(C(O)(CF)c2cc(F)c3c(c2)C(=O)N(Cc2ncc(Cl)cc2O)[C@@]3(OCCO)c2ccc(Cl)cc2)c1]

6-{[(1R)-1-(4-chlorophenyl)-7-fluoro-5-[1-(4-fluorooxan-4-yl)-1-hydroxypropyl]-3-oxo-1-[(3S)-oxolan-3-yloxy]-2,3-dihydro-1 H-isoindol-2-yl]methyl}pyridine-3-carbonitrile;

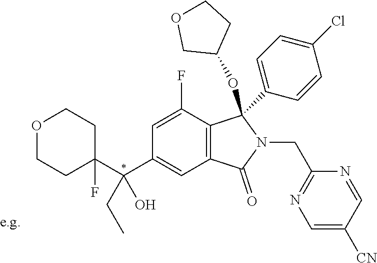 [SMILES: CCC(O)(c1cc(F)c2c(c1)C(=O)N(Cc1ncc(C#N)cn1)[C@@]2(O[C@H]1CCOC1)c1ccc(Cl)cc1)C1(F)CCOCC1]

6-{[(1R)-1-(4-chlorophenyl)-7-fluoro-1-[(3-fluorooxetan-3-yl)methoxy]-5-[1-hydroxy-1-(1-methyl-1 H-imidazol-4-yl)propyl]-3-oxo-2,3-dihydro-1 H-isoindol-2-yl]methyl}pyridine-3-carbonitrile;

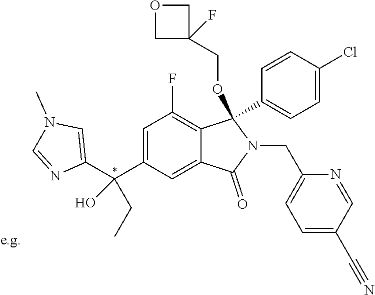 [SMILES: CCC(O)(c1cc(F)c2c(c1)C(=O)N(Cc1ccc(C#N)cn1)[C@@]2(OCC1(F)COC1)c1ccc(Cl)cc1)c1cn(C)cn1]

6-{[(1R)-1-(4-chlorophenyl)-7-fluoro-1-({1-[hydroxy(²H₂)methyl]cyclopropyl}(2H₂)methoxy)-5-[1-hydroxy-1-(1-methyl-1 H-imidazol-4-yl)propyl]-3-oxo-2,3-dihydro-1 H-isoindol-2-yl]methyl}pyridine-3-carbonitrile;

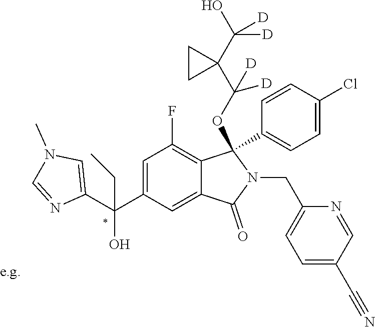 [SMILES: [2H]C([2H])(O)C1(C([2H])([2H])O[C@]2(c3ccc(Cl)cc3)c3c(F)cc(C(O)(CC)c4cn(C)cn4)cc3C(=O)N2Cc2ccc(C#N)cn2)CC1]

and

(3R)-3-(4-chlorophenyl)-2-[(5-chloropyrimidin-2-yl)methyl]-4-fluoro-6-[1-hydroxy-1-(1-methylpiperidin-4-yl)propyl]-3-[(3S)-oxolan-3-yloxy]-2,3-dihydro-1 H-isoindol-1-one

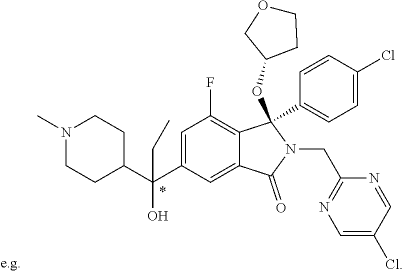 [SMILES: CCC(O)(c1cc(F)c2c(c1)C(=O)N(Cc1ncc(Cl)cn1)[C@@]2(O[C@H]1CCOC1)c1ccc(Cl)cc1)C1CCN(C)CC1]

In one embodiment, the MDM2 antagonist is a compound of formula (I°) which is diastereoisomer 2A and is selected from the following compounds, or a tautomer, N-oxide, pharmaceutically acceptable salt or solvate thereof:
(3R)-3-(4-chlorophenyl)-2-[(5-chloropyrimidin-2-yl)methyl]-4-fluoro-6-{1-hydroxy-1-[trans-4-hydroxycyclohexyl]ethyl}-3-{[1-(hydroxymethyl)cyclopropyl]methoxy}-2,3-dihydro-1 H-isoindol-1-one;
2-{[(1R)-1-(4-chlorophenyl)-7-fluoro-5-[1-hydroxy-1-(1-methyl-1 H-imidazol-4-yl)propyl]-3-oxo-1-[(3S)-oxolan-3-yloxy]-2,3-dihydro-1 H-isoindol-2-yl]methyl}pyrimidine-5-carbonitrile;
(3R)-2-[(5-chloro-3-hydroxypyridin-2-yl)methyl]-3-(4-chlorophenyl)-4-fluoro-6-[1-hydroxy-1-(1-methyl-1 H-imidazol-4-yl)propyl]-3-(2-hydroxyethoxy)-2,3-dihydro-1 H-isoindol-1-one;
6-{[(1R)-1-(4-chlorophenyl)-7-fluoro-5-[1-(4-fluorooxan-4-yl)-1-hydroxypropyl]-3-oxo-1-[(3S)-oxolan-3-yloxy]-2,3-dihydro-1 H-isoindol-2-yl]methyl}pyridine-3-carbonitrile;
6-{[(1R)-1-(4-chlorophenyl)-7-fluoro-1-[(3-fluorooxetan-3-yl)methoxy]-5-[1-hydroxy-1-(1-methyl-1 H-imidazol-4-yl)propyl]-3-oxo-2,3-dihydro-1 H-isoindol-2-yl]methyl}pyridine-3-carbonitrile;
6-{[(1R)-1-(4-chlorophenyl)-7-fluoro-1-({1-[hydroxy(²H₂)methyl]cyclopropyl}(2H₂)methoxy)-5-[1-hydroxy-1-(1-methyl-1 H-imidazol-4-yl)propyl]-3-oxo-2,3-dihydro-1 H-isoindol-2-yl]methyl}pyridine-3-carbonitrile; and
(3R)-3-(4-chlorophenyl)-2-[(5-chloropyrimidin-2-yl)methyl]-4-fluoro-6-[1-hydroxy-1-(1-methylpiperidin-4-yl)propyl]-3-[(3S)-oxolan-3-yloxy]-2,3-dihydro-1 H-isoindol-1-one.
In one embodiment, the MDM2 antagonist is a compound of formula (I°) which is diastereoisomer 2B and is selected from the following compounds, or a tautomer, N-oxide, pharmaceutically acceptable salt or solvate thereof:
(3R)-3-(4-chlorophenyl)-2-[(5-chloropyrimidin-2-yl)methyl]-4-fluoro-6-{1-hydroxy-1-[trans-4-hydroxycyclohexyl]ethyl}-3-{[1-(hydroxymethyl)cyclopropyl]methoxy}-2,3-dihydro-1 H-isoindol-1-one;
2-{[(1R)-1-(4-chlorophenyl)-7-fluoro-5-[1-hydroxy-1-(1-methyl-1 H-imidazol-4-yl)propyl]-3-oxo-1-[(3S)-oxolan-3-yloxy]-2,3-dihydro-1 H-isoindol-2-yl]methyl}pyrimidine-5-carbonitrile;
(3R)-2-[(5-chloro-3-hydroxypyridin-2-yl)methyl]-3-(4-chlorophenyl)-4-fluoro-6-[1-hydroxy-1-(1-methyl-1 H-imidazol-4-yl)propyl]-3-(2-hydroxyethoxy)-2,3-dihydro-1 H-isoindol-1-one;
6-{[(1R)-1-(4-chlorophenyl)-7-fluoro-5-[1-(4-fluorooxan-4-yl)-1-hydroxypropyl]-3-oxo-1-[(3S)-oxolan-3-yloxy]-2,3-dihydro-1 H-isoindol-2-yl]methyl}pyridine-3-carbonitrile;
6-{[(1R)-1-(4-chlorophenyl)-7-fluoro-1-[(3-fluorooxetan-3-yl)methoxy]-5-[1-hydroxy-1-(1-methyl-1 H-imidazol-4-yl)propyl]-3-oxo-2,3-dihydro-1 H-isoindol-2-yl]methyl}pyridine-3-carbonitrile;
6-{[(1R)-1-(4-chlorophenyl)-7-fluoro-1-({1-[hydroxy(²H₂)methyl]cyclopropyl}(2H₂)methoxy)-5-[1-hydroxy-1-(1-methyl-1 H-imidazol-4-yl)propyl]-3-oxo-2,3-dihydro-1 H-isoindol-2-yl]methyl}pyridine-3-carbonitrile; and
(3R)-3-(4-chlorophenyl)-2-[(5-chloropyrimidin-2-yl)methyl]-4-fluoro-6-[1-hydroxy-1-(1-methylpiperidin-4-yl)propyl]-3-[(3S)-oxolan-3-yloxy]-2,3-dihydro-1 H-isoindol-1-one.
In one embodiment, the MDM2 antagonist is a compound of formula (I°) which is selected from the following compounds, or a tautomer, N-oxide, pharmaceutically acceptable salt or solvate thereof:
(3R)-3-(4-chlorophenyl)-2-[(5-chloropyrimidin-2-yl)methyl]-4-fluoro-6-[2-hydroxy-1-(4-methylpiperazin-1-yl)butan-2-yl]-3-[(3S)-oxolan-3-yloxy]-2,3-dihydro-1 H-isoindol-1-one;

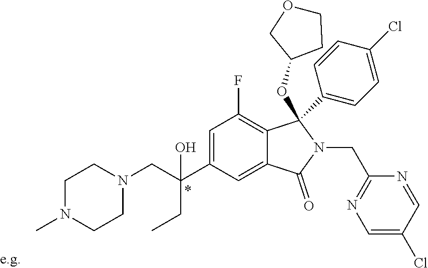 [SMILES: CCC(O)(CN1CCN(C)CC1)c1cc(F)c2c(c1)C(=O)N(Cc1ncc(Cl)cn1)[C@@]2(O[C@H]1CCOC1)c1ccc(Cl)cc1]

(3R)-3-(4-chlorophenyl)-2-[(5-chloropyrimidin-2-yl)methyl]-4-fluoro-6-[1-(4-fluoro-1-methylpiperidin-4-yl)-1-hydroxypropyl]-3-methoxy-2,3-dihydro-1 H-isoindol-1-one;

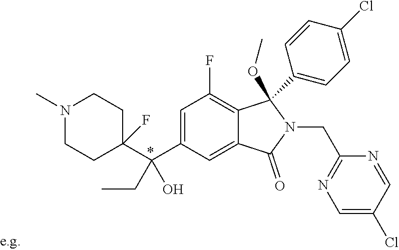 [SMILES: CCC(O)(c1cc(F)c2c(c1)C(=O)N(Cc1ncc(Cl)cn1)[C@@]2(OC)c1ccc(Cl)cc1)C1(F)CCN(C)CC1]

1-({[(1R)-1-(4-chlorophenyl)-2-[(5-chloropyrimidin-2-yl)methyl]-7-fluoro-5-[1-(4-fluoro-1-methylpiperidin-4-yl)-1-hydroxypropyl]-3-oxo-2,3-dihydro-1 H-isoindol-1-yl]oxy}methyl)cyclopropane-1-carbonitrile;

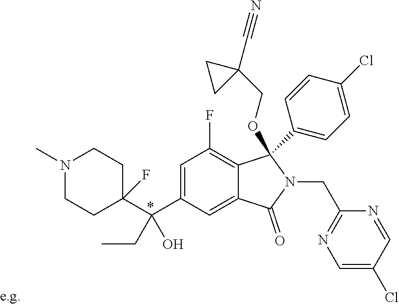 [SMILES: CCC(O)(c1cc(F)c2c(c1)C(=O)N(Cc1ncc(Cl)cn1)[C@@]2(OCC1(C#N)CC1)c1ccc(Cl)cc1)C1(F)CCN(C)CC1]

(3R)-3-(4-chlorophenyl)-2-[(5-chloropyrimidin-2-yl)methyl]-4-fluoro-6-[1-(4-fluoro-1-methylpiperidin-4-yl)-1-hydroxypropyl]-3-[cis-3-hydroxycyclobutoxy]-2,3-dihydro-1 H-isoindol-1-one;

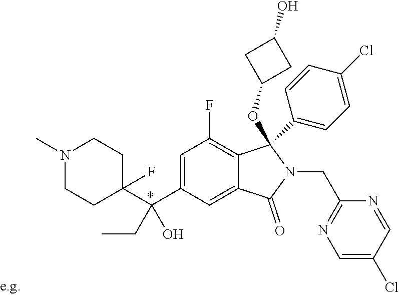 [SMILES: CCC(O)(c1cc(F)c2c(c1)C(=O)N(Cc1ncc(Cl)cn1)[C@@]2(O[C@H]1C[C@@H](O)C1)c1ccc(Cl)cc1)C1(F)CCN(C)CC1]

and
(3R)-3-(4-chlorophenyl)-2-[(5-chloropyrimidin-2-yl)methyl]-4-fluoro-6-[1-(4-fluoro-1-methylpiperidin-4-yl)-1-hydroxypropyl]-3-[(2R)-2-hydroxypropoxy]-2,3-dihydro-1 H-isoindol-1-one

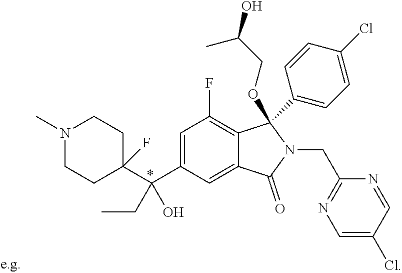 [SMILES: CCC(O)(c1cc(F)c2c(c1)C(=O)N(Cc1ncc(Cl)cn1)[C@@]2(OC[C@@H](C)O)c1ccc(Cl)cc1)C1(F)CCN(C)CC1]

In one embodiment, the MDM2 antagonist is a compound of formula (I°) which is 1-({[(1R)-1-(4-chlorophenyl)-2-[(5-chloropyrimidin-2-yl)methyl]-7-fluoro-5-[1-(4-fluoro-1-methylpiperidin-4-yl)-1-hydroxypropyl]-3-oxo-2,3-dihydro-1H-isoindol-1-yl]oxy}methyl)cyclopropane-1-carbonitrile, or a tautomer, N-oxide, pharmaceutically acceptable salt or solvate thereof.

In one embodiment, the MDM2 antagonist is a compound of formula (I°) which is (3R)-3-(4-chlorophenyl)-2-[(5-chloropyrimidin-2-yl)methyl]-4-fluoro-6-[1-(4-fluoro-1-methylpiperidin-4-yl)-1-hydroxypropyl]-3-methoxy-2,3-dihydro-1H-isoindol-1-one, or a tautomer, N-oxide, pharmaceutically acceptable salt or solvate thereof.

In one embodiment, the MDM2 antagonist is a compound of formula (I°) which is diastereoisomer 2A and is 1-({[(1R)-1-(4-chlorophenyl)-2-[(5-chloropyrimidin-2-yl)methyl]-7-fluoro-5-[1-(4-fluoro-1-methylpiperidin-4-yl)-1-hydroxypropyl]-3-oxo-2,3-dihydro-1 H-isoindol-1-yl]oxy}methyl)cyclopropane-1-carbonitrile, or a tautomer, N-oxide, pharmaceutically acceptable salt or solvate thereof.

In one embodiment, the MDM2 antagonist is a compound of formula (I°) which is diastereoisomer 2A and is (3R)-3-(4-chlorophenyl)-2-[(5-chloropyrimidin-2-yl)methyl]-4-fluoro-6-[1-(4-fluoro-1-methylpiperidin-4-yl)-1-hydroxypropyl]-3-methoxy-2,3-dihydro-1H-isoindol-1-one, or a tautomer, N-oxide, pharmaceutically acceptable salt or solvate thereof.

In one embodiment, the MDM2 antagonist is a compound of formula (I°) which is diastereoisomer 2B and is 1-({[(1R)-1-(4-chlorophenyl)-2-[(5-chloropyrimidin-2-yl)methyl]-7-fluoro-5-[1-(4-fluoro-1-methylpiperidin-4-yl)-1-hydroxypropyl]-3-oxo-2,3-dihydro-1 H-isoindol-1-yl]oxy}methyl)cyclopropane-1-carbonitrile, or a tautomer, N-oxide, pharmaceutically acceptable salt or solvate thereof.

In one embodiment, the MDM2 antagonist is a compound of formula (I°) which is diastereoisomer 2B and is (3R)-3-(4-chlorophenyl)-2-[(5-chloropyrimidin-2-yl)methyl]-4-fluoro-6-[1-(4-fluoro-1-methylpiperidin-4-yl)-1-hydroxypropyl]-3-methoxy-2,3-dihydro-1H-isoindol-1-one, or a tautomer, N-oxide, pharmaceutically acceptable salt or solvate thereof.

In one embodiment the MDM2 antagonist is (3R)-3-(4-chlorophenyl)-2-[(5-chloropyrimidin-2-yl)methyl]-4-fluoro-6-[(1S)-1-(4-fluoro-1-methylpiperidin-4-yl)-1-hydroxypropyl]-3-methoxy-2,3-dihydro-1 H-isoindol-1-one, or a tautomer, N-oxide, pharmaceutically acceptable salt or solvate thereof.

In one embodiment the MDM2 antagonist is (3R)-3-(4-chlorophenyl)-2-[(5-chloropyrimidin-2-yl)methyl]-4-fluoro-6-[(1R)-1-(4-fluoro-1-methylpiperidin-4-yl)-1-hydroxypropyl]-3-methoxy-2,3-dihydro-1 H-isoindol-1-one, or a tautomer, N-oxide, pharmaceutically acceptable salt or solvate thereof.

In one embodiment the MDM2 antagonist is 1-({[(1R)-1-(4-chlorophenyl)-2-[(5-chloropyrimidin-2-yl)methyl]-7-fluoro-5-[(1S)-1-(4-fluoro-1-methylpiperidin-4-yl)-1-hydroxypropyl]-3-oxo-2,3-dihydro-1 H-isoindol-1-yl]oxy}methyl)cyclopropane-1-carbonitrile, or a tautomer, N-oxide, pharmaceutically acceptable salt or solvate thereof.

In one embodiment the MDM2 antagonist is 1-({[(1R)-1-(4-chlorophenyl)-2-[(5-chloropyrimidin-2-yl)methyl]-7-fluoro-5-[(1R)-1-(4-fluoro-1-methylpiperidin-4-yl)-1-hydroxypropyl]-3-oxo-2,3-dihydro-1 H-isoindol-1-yl]oxy}methyl)cyclopropane-1-carbonitrile, or a tautomer, N-oxide, pharmaceutically acceptable salt or solvate thereof.

For the avoidance of doubt, it is to be understood that each general and specific embodiment and example for one substituent may be combined with each general and specific embodiment and example for one or more, in particular all, other substituents as defined herein and that all such embodiments are embraced by this application.

### Particular Compounds

The uses and methods of the invention apply to all compound of formula I° described herein i.e. the MDM2 antagonist may be a compound of formula I°, any subformulae thereof, or any specific compound described herein, or a tautomer, N-oxide, pharmaceutically acceptable salt or solvate thereof.

In one embodiment, the MDM2 antagonist is a compound of formula I° selected from Examples 1 to 134 as described in the first set of examples defined herein (i.e. the compounds in which cyc is phenyl, as also described in WO 2017/055860).

In one embodiment, the MDM2 antagonist is a compound of formula I° selected from Examples 1 to 580 as described in the second set of examples defined herein (i.e. the compounds in which cyc is Het, as also described in WO 2017/055859).

In one particular embodiment of the invention, the MDM2 antagonist is a compound of formula (I°) or a tautomer, N-oxide, pharmaceutically acceptable salt or solvate thereof as defined herein, which is (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid.
(2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid is referred to herein as

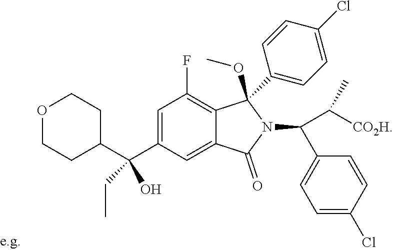 [SMILES: CC[C@@](O)(c1cc(F)c2c(c1)C(=O)N([C@H](c1ccc(Cl)cc1)[C@H](C)C(=O)O)[C@@]2(OC)c1ccc(Cl)cc1)C1CCOCC1]

(2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid is disclosed as Example 124 in international patent application no PCT/GB2016/053042 which was published as WO 2017/055860 on 6 Apr. 2017.

Methods for the preparation of compound 1 can be found in international patent application no PCT/GB2018/050845 which was published as WO 2018/178691 on 4 Oct. 2018.

In one embodiment, the MDM2 antagonist is compound 1 in the form of the free acid. In another embodiment, the MDM2 antagonist is a pharmaceutically acceptable salt of compound 1.

### General

Other MDM2 antagonists may be prepared in conventional manner for example by processes analogous to those described.

The posology of the MDM2 antagonists is known to a person skilled in the art. It will be appreciated that the preferred method of administration and the dosage amounts and regimes for each MDM2 antagonist will depend on the particular tumour being treated and the particular host being treated. The optimum method, administration schedule, the dosage amounts and regime can be readily determined by those skilled in the art using conventional methods and in view of the information set out herein.

Salts, Solvates, Tautomers, Isomers, N-Oxides, Esters, Prodrugs and Isotopes A reference to any compound herein also includes ionic forms, salts, solvates, isomers (including geometric and stereochemical isomers unless specified), tautomers, N-oxides, esters, prodrugs, isotopes and protected forms thereof, for example, as discussed below; in particular, the salts or tautomers or isomers or N-oxides or solvates thereof; and more particularly the salts or tautomers or N-oxides or solvates thereof. In one embodiment reference to a compound also includes the salts or tautomers or solvates thereof.

### Salts

The compounds can exist in the form of salts, for example acid addition salts or, in certain cases salts of organic and inorganic bases such as carboxylate, sulfonate and phosphate salts. All such salts are within the scope of this invention, and references to compounds of the formula (I°) include the salt forms of the compounds.

### N-Oxides

Compounds containing an amine function may also form N-oxides. A reference herein to a compound that contains an amine function also includes the N-oxide.

### Geometric Isomers and Tautomers

The compounds may exist in a number of different geometric isomeric, and tautomeric forms and references to compounds of the formula (I°) include all such forms. For the avoidance of doubt, where a compound can exist in one of several geometric isomeric or tautomeric forms and only one is specifically described or shown, all others are nevertheless embraced for use in the invention.

For example, certain heteroaryl rings can exist in the two tautomeric forms such as A and B shown below. For simplicity, a formula may illustrate one form but the formula is to be taken as embracing both tautomeric forms.

### Stereoisomers

Unless otherwise mentioned or indicated, the chemical designation of compounds denotes the mixture of all possible stereochemically isomeric forms.

### Compounds of Formula (I°)

Stereocentres are illustrated in the usual fashion, using ‘hashed’ or ‘solid’ wedged lines. e.g.

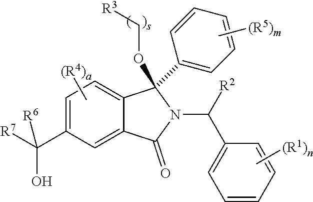

Where a compound is described as a mixture of two diastereoisomers/epimers, the configuration of the stereocentre is not specified and is represented by straight lines.

Where compounds contain one or more chiral centres, and can exist in the form of two or more optical isomers, references to compounds include all optical isomeric forms thereof (e.g. enantiomers, epimers and diastereoisomers), either as individual optical isomers, or mixtures (e.g. racemic or scalemic mixtures) or two or more optical isomers, unless the context requires otherwise.

Of special interest are those compounds which are stereochemically pure. When a compound is for instance specified as R, this means that the compound is substantially free of the S isomer. If a compound is for instance specified as E, this means that the compound is substantially free of the Z isomer. The terms cis, trans, R, S, E and Z are well known to a person skilled in the art.

### Isotopic Variations

The present invention includes use of all pharmaceutically acceptable isotopically-labeled compounds, i.e. compounds, wherein one or more atoms are replaced by atoms having the same atomic number, but an atomic mass or mass number different from the atomic mass or mass number usually found in nature.

### Solvates and Crystalline Forms

Also encompassed by the compounds are any polymorphic forms of the compounds, and solvates such as hydrates, alcoholates and the like.

In one embodiment, the MDM2 antagonist is a crystalline form of the free acid of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid.

In one embodiment, the MDM2 antagonist is a crystalline form of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid having:
(a) an X-ray powder diffraction pattern characterised by peaks at diffraction angles 15.1, 15.5, 15.8 and 22.3 degrees 26 (±0.2 degrees 26); or
(b) interplanar spacings of 3.99, 5.62, 5.71 and 5.87 Å.

In particular, the crystalline form of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid has:
(a) an X-ray powder diffraction pattern characterised by peaks at diffraction angles 11.3, 15.1, 15.5, 15.8, 17.2, 20.8, 22.3 and 28.6 degrees 26 (±0.2 degrees 26); or
(b) interplanar spacings at 3.12, 3.99, 4.27, 5.17, 5.62, 5.71, 5.87 and 7.85 Å.

In particular, the crystalline form of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid has an X-ray powder diffraction pattern characterised by the presence of major peaks at the diffraction angles (26), interplanar spacings (d) and intensities set forth in Table 6 herein.

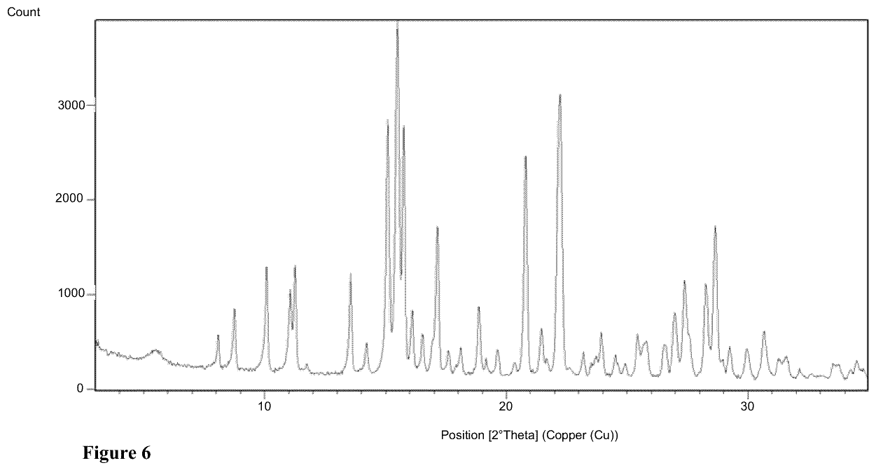

In particular, the crystalline form of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid has an X-ray powder diffraction pattern which exhibits peaks at the same diffraction angles as those of the X-ray powder diffraction pattern shown in FIG. 6, and preferably wherein the peaks have the same relative intensity as the peaks in FIG. 6.

In particular, the crystalline form of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid has an X-ray powder diffraction pattern substantially as shown in FIG. 6.

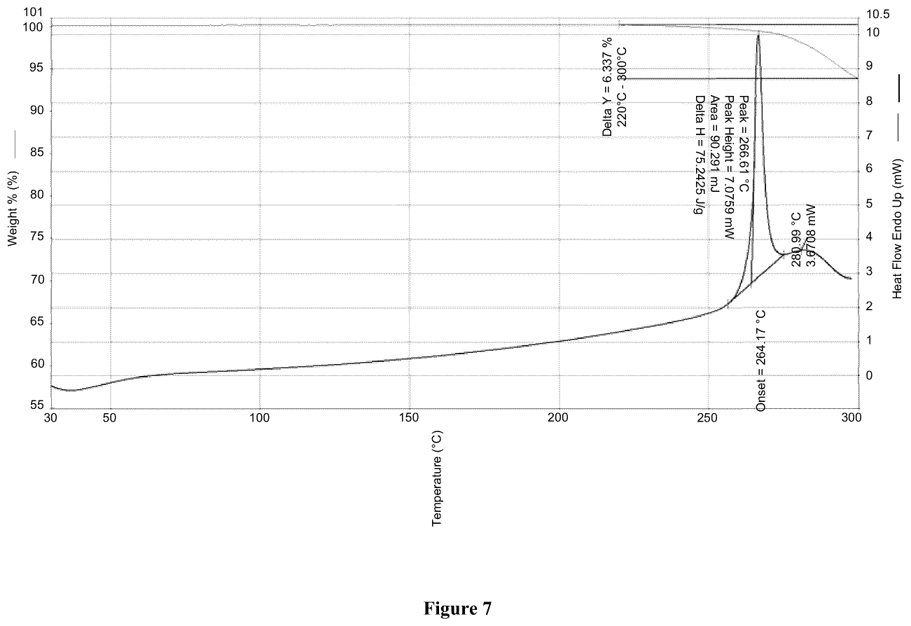

In one embodiment, the crystalline form of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid exhibits an exothermic peak at 266-267° C. (e.g. 266.61° C.) when subjected to DSC (FIG. 7).

The crystalline forms may be substantially crystalline, which means that one single crystalline form may predominate, although other crystalline forms may be present in minor and preferably negligible amounts.

For example, a crystalline form may contain no more than 5% by weight of any other crystalline form.

### Complexes

The compounds also includes within their scope complexes (e.g. inclusion complexes or clathrates with compounds such as cyclodextrins, or complexes with metals) of the compounds. Inclusion complexes, clathrates and metal complexes can be formed by means of methods well known to the skilled person.

### Prodrugs

Also encompassed by the compounds are any pro-drugs of the compounds. By “prodrugs” is meant for example any compound that is converted in vivo into the biologically active compounds.

### Methods for the Preparation of Compounds Used in the Invention

Compounds of the Formula (I°) In this section, as in all other sections of this application unless the context indicates otherwise, references to formula 1° also include all other subformulae and examples thereof as defined herein, unless the context indicates otherwise.

Compounds of the formula (I°) can be prepared in accordance with synthetic methods well known to the skilled person.

The required intermediates are either commercially available, known in the literature, prepared by methods analogous to those in the literature or prepared by methods analogous to those described in the example experimental procedures below. Other compounds may be prepared by functional group interconversion of the groups using methods well known in the art.

General processes for preparing, isolating and purifying the compounds wherein cyc is phenyl can be found in international patent application no PCT/GB2016/053042 which was published as WO 2017/055860 on 06.04.2017: General processes for preparing, isolating and purifying the compounds wherein cyc is Het can be found in international patent application no PCT/GB2016/053041 which was published as WO 2017/055859 on 06.04.2017.

### Biomarker Detection

In some embodiments, a sample of patient tissue is tested. The tissue may comprise one or more cancer cells, or may comprise nucleic acid, typically DNA, from cancer cells such as circulating tumour DNA (ctDNA) obtainable from blood.

In some embodiments, the sample is entered into an in vitro diagnostic device, which measures the relevant expression or activity of the biomarker or biomarkers of interest.

The patient may typically be known or suspected to have cancer when the invention is carried out to confirm whether treatment is likely to be effective. In certain embodiments therefore, the method is for assessing whether a human patient, known or suspected to have cancer, can be treated using an MDM2 antagonist.

A method of the invention typically comprises detecting one or more of the identified biomarkers, and optionally further biomarkers, by using one or more detection reagents and/or detection techniques. The detection is typically carried out ex vivo on a sample from the patient, for example in vitro. In one embodiment, the biomarker is measured directly. In another embodiment, a biomarker substrate may be measured to measure biomarker levels indirectly.

By “detecting” is meant measuring, quantifying, scoring, or assaying the expression or activity level of the biomarkers. Methods of evaluating biological compounds, including biomarker proteins, genes or mRNA transcripts, are known in the art. It is recognized that methods of detecting a biomarker include direct measurements and indirect measurements. One skilled in the art will be able to select an appropriate method of assaying a particular biomarker.

A “detection reagent” is an agent or compound that specifically (or selectively) binds to, interacts with or detects the biomarker of interest. Such detection reagents may include, but are not limited to, an antibody, polyclonal antibody, or monoclonal antibody that preferentially binds a protein biomarker, or an oligonucleotide that is complementary to and binds selectively to an mRNA or DNA biomarker, typically under stringent hybridising conditions.

The phrase “specifically (or selectively) binds” or “specifically (or selectively) immunoreactive with,” when referring to a detection reagent, refers to a binding reaction that is determinative of the presence of the biomarker in a heterogeneous population of biological molecules. For example under designated immunoassay conditions, the specified detection reagent (e.g. antibody) binds to a particular protein at least two times the background and does not substantially bind in a significant amount to other proteins present in the sample. Specific binding under such conditions may require an antibody that is selected for its specificity for a particular protein. A variety of immunoassay formats may be used to select antibodies specifically immunoreactive with a particular protein. For example, solid-phase ELISA immunoassays (enzyme linked immunosorbent assay) are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice the background signal or noise and more typically more than 10 to 100 times the background.

Technologies such as in situ hybridization (ISH), quantitative real-time polymerase chain reaction (qRT PCR) and immuno-histochemistry (IHC) have been traditionally used for diagnosing or detecting disease biomarkers. However, the emergence of high throughput, sensitive approaches such as next-generation sequencing, single molecule real-time sequencing, digital pathology and quantitative histopathology have created a shift in the enabling technology platform for a companion diagnostic or CDx. Quantitative histopathology and digital pathology are both medical imaging-based diagnostics approaches; they provide localization and measurement of protein biomarkers in a tissue sample. Tissue markers are identified and quantified using an automated, fluorescence-based imaging platform.

When the biomarker to be detected is a protein, methods for detection include antibody-based assays, protein array assays, mass spectrometry (MS) based assays, and (near) infrared spectroscopy based assays. For example, immunoassays, include but are not limited to competitive and non-competitive assay systems using techniques such as Western blots, radioimmunoassays, ELISA, “sandwich” immunoassays, immunoprecipitation assays, precipitin reactions, gel diffusion precipitin reactions, immunodiffusion assays, fluorescent immunoassays and the like. Such assays are routine and well known in the art.

To “analyze” includes determining a set of values associated with a sample by measurement of a marker (such as, e.g., presence or absence of a marker or constituent expression or activity levels) in the sample and comparing the measurement against measurement in a sample or set of samples from the same subject or other control subject(s). The markers of the present teachings can be analyzed by any of various conventional methods known in the art. To “analyze” can include performing a statistical analysis to, e.g., determine whether a subject is a responder or a non-responder to a therapy (e.g., an MDM2 antagonist treatment as described herein).

A “sample” in the context of the present teachings refers to any biological sample that is isolated from a subject, e.g., a blood sample or a biopsy. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leukocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art.

Analysis Techniques

Prior to administration of a MDM2 antagonist, a patient may be screened to determine whether a disease or condition from which the patient is or may be suffering is one which would be susceptible to treatment with a compound which inhibits MDM2/p53. The term ‘patient’ includes human and veterinary subjects such as primates, in particular human patients.

For example, a biological sample taken from a patient may be analysed to determine whether a condition or disease, such as cancer, that the patient is or may be suffering from is one which is characterised by a genetic abnormality or abnormal protein expression which leads to up-regulation of the levels of MDM2 or to upregulation of a biochemical pathway downstream of MDM2/p53. Furthermore the biological sample taken from a patient may be analysed to determine whether a condition or disease, such as cancer, that the patient is or may be suffering from is one which is characterised by the biomarkers of the invention.

Examples of such abnormalities that result in activation or sensitisation of MDM2, loss of, or inhibition of regulatory pathways impacting on MDM2 expression, up-regulation of receptors or their ligands, cytogenetic aberrations or presence of mutant variants of the receptors or ligands. Tumours with up-regulation of MDM2/p53, in particular over-expression of MDM2 or exhibit wild-type p53, may be particularly sensitive to inhibitors of MDM2/p53. For example, amplification of MDM2 and/or deletion of its negative regulator such as p14ARF has been identified in a range of cancers as discussed herein.

The terms “elevated” and “increased” includes up-regulated expression or over-expression, including gene amplification (i.e. multiple gene copies), cytogenetic aberration and increased expression by a transcriptional effect or post-translational effect. Thus, the patient may be subjected to a diagnostic test to detect a suitable protein or marker characteristic of up-regulation of the biomarkers of the invention.

The term diagnosis includes screening.

The term “marker” or “biomarker” includes genetic markers including, for example, the measurement of DNA composition to identify presence of mutations in p53 or amplification MDM2 or deletion (loss) of p14ARF, or typically the biomarkers of the invention discussed extensively herein. The term marker also includes markers which are characteristic of up regulation of MDM2/p53 or upregulation or down regulation of the biomarkers outlined herein, including protein levels, protein state and mRNA levels of the aforementioned proteins. Gene amplification includes greater than 7 copies, as well as gains of between 2 and 7 copies.

The terms “reduced”, “depleted” or “decreased” includes lowered expression or reduced-expression, including down regulation (i.e. reduced gene copies), cytogenetic aberration and decreased expression by a transcriptional effect. Thus, the patient may be subjected to a diagnostic test to detect lower levels of a biomarker of the invention.

The diagnostic tests and screens are typically conducted on a biological sample (i.e. body tissue or body fluids) selected from tumour biopsy samples, blood samples (isolation and enrichment of shed tumour cells or isolation of circulating tumour DNA), cerebrospinal fluid, plasma, serum, saliva, stool biopsies, sputum, chromosome analysis, pleural fluid, peritoneal fluid, buccal spears, skin biopsy or urine.

Furthermore liquid biopsies such as blood-based (systematic) circulating tumour DNA (ctDNA) tests or NGS-based liquid biopsy tests can also be used, in particular to detect cancer or identify mutations.

Liquid-based biopsies involving next-generation sequencing (NGS) supplement traditional detection methods of PCR and tumour biopsies for example by whole genome sequencing on circulating tumour cells (CTCs) or massively parallel sequencing of circulating tumour DNA (ctDNA).

In one embodiment, the sample obtained is a blood sample e.g. a plasma or serum sample, in particular a serum sample. In one embodiment, the sample obtained is a tumour biopsy sample.

In one embodiment, blood, usually collected in a serum-separating tube, is analysed in a medical laboratory or at the point of care. In a second embodiment the tumour is analysed by biopsy and analysed in a medical laboratory.

Screening methods could include, but are not limited to, standard methods such as reverse-transcriptase polymerase chain reaction (RT-PCR), protein analysis or in-situ hybridization such as fluorescence in situ hybridization (FISH).

Methods of identification and analysis of cytogenetic aberration, genetic amplification, deletions, down regulation, mutations and up-regulation of proteins are known to a person skilled in the art. Screening methods could include, but are not limited to, standard methods such as DNA sequence analysis by conventional Sanger or next-generation sequencing methods, reverse-transcriptase polymerase chain reaction (RT-PCR), RNA sequencing (RNAseq), Nanostring hybridisation proximity RNA nCounter assays, or in-situ hybridization such as fluorescence in situ hybridization (FISH) or allele-specific polymerase chain reaction (PCR). In addition, methods for assessing protein levels include immunohistochemistry or other immunoassays. Therefore, in one embodiment protein expression is analysed in the patient sample. In another embodiment gene expression is analysed in the patient sample for example gene aberration, using techniques such as FISH. Methods for assessing gene copy changes include techniques commonly used in cytogenetic laboratories such as MLPA (Multiplex Ligation-dependent Probe Amplification) a multiplex PCR method detecting abnormal copy numbers, or other PCR techniques which can detect gene amplification, gain and deletion.

In screening by RT-PCR, the level of mRNA in the tumour is assessed by creating a cDNA copy of the mRNA followed by amplification of the cDNA by PCR. Methods of PCR amplification, the selection of primers, and conditions for amplification, are known to a person skilled in the art. Nucleic acid manipulations and PCR are carried out by standard methods, as described for example in Ausubel, F. M. et al., eds. (2004) Current Protocols in Molecular Biology, John Wiley & Sons Inc., or Innis, M. A. et al., eds. (1990) PCR Protocols: a guide to methods and applications, Academic Press, San Diego. Reactions and manipulations involving nucleic acid techniques are also described in Sambrook et al., (2001), 3rd Ed, Molecular Cloning: A Laboratory Manual, Cold Spring Harbor Laboratory Press. Alternatively a commercially available kit for RT-PCR (for example Roche Molecular Biochemicals) may be used, or methodology as set forth in U.S. Pat. Nos. 4,666,828; 4,683,202; 4,801,531; 5,192,659, 5,272,057, 5,882,864, and 6,218,529 and incorporated herein by reference. Mutations, for example in the genes outlined herein, can be determined by PCR. In one embodiment the specific primer pairs are commercially available or as described in the literature.

An example of an in-situ hybridisation technique for assessing mRNA expression would be fluorescence in-situ hybridisation (FISH) (see Angerer (1987) Meth. Enzymol., 152: 649).

Next generation sequencing (NGS), DNA sequencing or Nanostring can be performed.

Generally, in situ hybridization comprises the following major steps: (1) fixation of tissue to be analyzed; (2) prehybridization treatment of the sample to increase accessibility of target nucleic acid, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments. The probes used in such applications are typically labelled, for example, with radioisotopes or fluorescent reporters. Certain probes are sufficiently long, for example, from about 50, 100, or 200 nucleotides to about 1000 or more nucleotides, to enable specific hybridization with the target nucleic acid(s) under stringent conditions. Standard methods for carrying out FISH are described in Ausubel, F. M. et al., eds. (2004) Current Protocols in Molecular Biology, John Wiley & Sons Inc and Fluorescence In Situ Hybridization: Technical Overview by John M. S. Bartlett in Molecular Diagnosis of Cancer, Methods and Protocols, 2nd ed.; ISBN: 1-59259-760-2; March 2004, pps. 077-088; Series: Methods in Molecular Medicine.

Methods for gene expression profiling are described by (DePrimo et al. (2003), BMC Cancer, 3:3). Briefly, the protocol is as follows: double-stranded cDNA is synthesized from total RNA using a (dT)24 oligomer for priming first-strand cDNA synthesis, followed by second strand cDNA synthesis with random hexamer primers. The double-stranded cDNA is used as a template for in vitro transcription of cRNA using biotinylated ribonucleotides. cRNA is chemically fragmented according to protocols described by Affymetrix (Santa Clara, CA, USA), and then hybridized overnight on Human Genome Arrays. Alternatively, single nucleotide polymorphism (SNP) arrays, a type of DNA microarray, can be used to detect polymorphisms within a population.

In addition, test kits may use Nanostring technology or ddPCR.

Alternatively, the protein products expressed from the mRNAs may be assayed by immunohistochemistry of tumour samples (or other immunoassays), solid phase immunoassay with microtitre plates, Western blotting, 2-dimensional SDS-polyacrylamide gel electrophoresis, ELISA, flow cytometry and other methods known in the art for detection of specific proteins e.g. capillary electrophoresis. Detection methods would include the use of site specific antibodies. The skilled person will recognise that all such well-known techniques for detection of upregulation of MDM2 and p53, detection of MDM2 or p53 variants or mutants, or loss of negative regulators of MDM2 (e.g. p14ARF), or the genes described herein are applicable in the present case. In particular levels of the genes described herein can be measured using immunohistochemistry. Expression in the cytoplasm can be assessed by staining of tumour cells. In some embodiments, one or both of the protein biomarkers of the invention are assayed using these techniques. In some embodiments, one or more biomarker substrates are assayed using these techniques.

Levels of proteins, in particular increased, decreased or abnormal levels of proteins can be measured using standard protein assays. Elevated or lowered levels, or under- or over-expression could also be detected in a tissue sample, for example, a tumour tissue by measuring the protein levels with an assay such as that from Chemicon International. The protein of interest would be immunoprecipitated from the sample lysate and its levels measured.

In the embodiment where the gene is a DDR biomarker, it will be appreciated that there are various analytical methods are available for determination, such as ELISA, immunoturbidimetry, rapid immunodiffusion, and visual agglutination.

In the embodiment where gene expression is tested, for example for the IFN signature biomarkers, it will be appreciated that there are various analytical methods available for determination.

In one embodiment which comprises detection of loss of one or more DDR biomarkers, such detection may typically be conducted at the DNA (i.e. DNA sequencing), RNA (i.e. qPCR, gene array, exome sequencing and the like) or protein (i.e. immunohistochemistry) level using clinical validated assays on biopsies. In an alternative embodiment, the detection of loss of one or more DDR biomarkers comprises one or more of: reverse phase protein array, western blotting, semi-quantitative or quantitative IHC.

Immunohistochemistry (IHC) is an important technique for biomarker detection. First, it allows direct visualization of biomarker expression in histologically relevant regions of the examined cancer tissue.

Second, IHC is run on FFPE tissue sections processed by standard methods, ensuring the biomarker assay can be run on clinically available of specimens. Third, validated IHC assays can be implemented readily into clinical practice. For example, there are multiple validated IHC assays used clinically, such as assays to detect PD-L1, HER2 and ALK (https://www.fda.gov/medical-devices/vitro-diagnostics/list-cleared-or-approved-companion-diagnostic-devices-vitro-and-imaging-tools). Traditionally, pathologists have visually scored IHC data. For example, in the calculation of an HSCORE, a summation of the percentage of area stained at each intensity level multiplied by the weighted intensity (e.g., 1, 2, or 3; where 0 is no staining, 1 is weak staining, 2 is moderate staining and 3 is strong staining) of staining is generated [McCarty et al: Cancer Res 1986, 46:4244s-4248s]. For assay validation purposes these analyses are frequently performed on specimens arrayed on stained TMA sections allowing representation of a sufficiently large number of specimens to for statistically rigorous testing. Tissue specimens are adequately represented by tissue cores on very few slides minimizing IHC cost and tissue usage, and facilitating intra-observer, inter-observer and inter-laboratory studies. Computer aided methods to classify image areas of interest (e.g., carcinomatous areas of tissue specimens) and quantify IHC staining intensity within those areas can also be utilised to generate data.

Such techniques will find equal applicability in the detection of other genes described herein. In some embodiments, detection of the increased levels of the genes described herein comprises a polymerase chain reaction (PCR) assay, or direct nucleic acid sequencing or hybridization with a nucleic acid probe specific for the genes.

Therefore all of these techniques could also be used to identify tumours particularly suitable for treatment with the MDM2 antagonists.

Ex-vivo functional assays could also be utilised where appropriate, for example measurement of circulating leukemia cells in a cancer patient, to assess the response to challenge with an MDM2/p53 inhibitor.

Therefore in a further aspect of the invention includes use of MDM2 antagonist for the manufacture of a medicament for the treatment or prophylaxis of a disease state or condition in a patient who has been screened and has been determined as suffering from, or being at risk of suffering from, a disease or condition which would be susceptible to treatment with an MDM2/p53 inhibitor.

Another aspect of the invention includes a MDM2 antagonist for use in the prophylaxis or treatment of cancer in a patient selected from a sub-population possessing loss of one or more DDR biomarkers.

Another aspect of the invention includes a MDM2 antagonist for use in the prophylaxis or treatment of cancer in a patient selected from a sub-population possessing p53 wild-type and loss of one or more DDR biomarkers.

Another aspect of the invention includes a MDM2 antagonist for use in the prophylaxis or treatment of cancer in a patient possessing loss of a MDM2 negative regulator such as p14ARF and loss of one or more DDR biomarkers.

MRI determination of vessel normalization (e.g. using MRI gradient echo, spin echo, and contrast enhancement to measure blood volume, relative vessel size, and vascular permeability) in combination with circulating biomarkers may also be used to identify patients suitable for treatment with a compound of formula (I°).

Thus a further aspect of the invention is a method for the diagnosis and treatment of a disease state or condition mediated by MDM2/p53, which method comprises (i) screening a patient to determine whether a disease or condition from which the patient is or may be suffering is one which would be susceptible to treatment with MDM2/p53 inhibitor; and (ii) where it is indicated that the disease or condition from which the patient is thus susceptible, thereafter administering to the patient a MDM2 antagonists and sub-groups or examples thereof as defined herein.

In one embodiment, the method of the invention additionally comprises the step of screening a patient possessing overexpression of one or more of the MDM family members (e.g. MDM2 and/or MDMx).

In one embodiment, the method of the invention additionally comprises the step of screening a patient possessing a cytogenetic aberration that results in overexpression of MDM2, for example, a patient selected as possessing the loss of negative regulator p14ARF.

In one embodiment the samples obtained from the patient are contacted with a primer, antibody, substrate or probe to determine the levels of genes described herein.

In one embodiment the method comprises: (i) contacting the patient sample with a primer, antibody, substrate or probe, and (ii) determining the levels of genes described herein.

Basal levels can be analysed by performing intracellular staining of untreated cells with an antibody for example an antibody conjugated to fluorescent probe. Antibodies against the biomarkers described herein are commercially available from a range of suppliers. In particular the antibody to be used may be part of an FDA approved in vitro diagnostic kit (IVD).

In one embodiment the method comprises: (i) contacting the patient sample with an antibody, and (ii) determining the levels of one or more biomarkers described herein. In an alternative embodiment, step (i) of the method comprises contacting the patient sample with one or more PCR primers for one or more biomarker substrates.

In one embodiment the method comprises: (i) contacting the patient sample with an antibody, and (ii) determining the level of nuclear localisation to assess the level of one or more biomarkers described herein. In an alternative embodiment, step (i) of the method comprises contacting the patient sample with a biomarker substrate antibody.

Where appropriate, the level of nuclear localisation can be determined using immunohistochemistry or immunofluorescence using an antibody.

Mutations which result in loss of DDR biomarkers can be detected using reverse phase protein array, western blotting, semi-quantitative or quantitative IHC, or DNA sequencing. In one embodiment the method comprises: (i) contacting the patient sample with an anti-mutant antibody, and (ii) determining that the patients tumour is DDR biomarker loss thereof. In one embodiment the method comprises: (i) contacting the patient sample with an anti-mutant antibody, and (ii) determining the levels of one or more DDR biomarker (or loss thereof).

Detection of DDR biomarker deletions and mutations can be performed by extraction of DNA from a patient sample, for example a tumour biopsy, amplification by PCR and DNA sequencing using an appropriate primer. PCR primers can be designed or are commercially available. Mutation array kits are also commercially available.

In one embodiment the method comprises: (i) contacting the patient sample with one or more DDR biomarker PCR primers, and (ii) determining the presence or absence of a DDR biomarker mutation or deletion. In an alternative embodiment, step (i) of the method comprises contacting the patient sample with one or more PCR primers for one or more biomarker substrates.

In one embodiment the method comprises: (i) contacting the patient sample with a DDR biomarker antibody, and (ii) determining the presence or absence of a DDR biomarker mutation or deletion. In an alternative embodiment, step (i) of the method comprises contacting the patient sample with a biomarker substrate antibody.

Protein levels can be determined using an ELISA Kit. ELISA kits for use on patient samples may be used in a clinical setting to assess blood chemistry. These utilise an antibody specific for the protein for example an anti-biomarker antibody such as anti-ATM or anti-ATRX, or a conjugated antibody. In particular the antibody to be used is part of an FDA approved in vitro diagnostic kit. In one embodiment, the level is determined using a test that complies with the standard as defined by the Association for Clinical Biochemistry (ACB).

In one embodiment the method comprises: (i) contacting the patient sample with an antibody, and (ii) determining the levels of proteins from the genes described herein.

In particular, the sample is contacted under conditions to quantify the levels.

For example, in the contacting step above the sample is contacted with primer, probe, substrate or antibody typically in the presence of a buffer. The substrate may be e.g. a fluorescent probe.

### Patient Selection

It will be appreciated that the patient selected for treatment with an MDM2 antagonist according to the invention will be tested for or will be measured for one or more DDR biomarkers in accordance with the methodology described in the previous section.

For example, such a selected patient will have:
decreased or low expression or activity of one or more DDR biomarkers.

In one embodiment, the selected patient exhibits or presents with at least one symptom of cancer in particular, a TP53 wild-type tumour.

In one embodiment, the selected cancer patient has not previously been treated with an MDM2 antagonist. In one embodiment, the selected patient has not previously responded to therapy with an MDM2 antagonist.

In some embodiments, a nucleic acid expression profile (e.g. the IFN gene signature) is determined by PCR, HTG EdgeSeq or a quantitative gene expression assay such as NanoString nCounter. In some embodiments, a protein expression profile (e.g. DDR pathway gene product, or e.g. BAP1 and/or CDKN2 Å) is determined by an immunoassay.

### Optional Interferon Gene Signature (IFN)

Increased DNA damage can induce an interferon response. This can be identified by the “interferon signature”. The expression of these proteins is typically determined by measuring mRNA transcripts.

In one embodiment, the patient or sample will have:
decreased or low expression or activity of one or more DDR biomarkers; and
increased expression of one, two, three, four, five or more the interferon signature genes.

In one embodiment, the increased expression of one, two, three, four, five or more the interferon signature genes is increased or high expression of one, two three, four, five or more of CXCL10, CXCL11, RSAD2, MX1, BATF2, IFI44L, IFITM1, ISG15, CMPK2, IF127, CD74, IFIH1, CCRL2, IFI44, HERC6, ISG20, IFIT3, HLA-C, OAS1, IFI35, IRF9, EPSTI1, USP18, BST2, CSF1, C1S, DHX58, TRIM14, OASL, IRF7, LGALS3BP, DDX60, LAP3, LAMP3, PARP12, PARP9, SP110, PLSCR1, WARS, STAT1, IRF3, IRF5, MSC, JUN, SP11, IRF1, COMMD3-BM11, STAT2, RUNX3, SREBF1, and/or FLI1.

In one embodiment, the RNA level of one or more of CXCL10, CXCL11, RSAD2, MX1, BATF2, IFI44L, IFITM1, ISG15, CMPK2, IF127, CD74, IFIH1, CCRL2, IFI44, HERC6, ISG20, IFIT3, HLA-C, OAS1, IFI35, IRF9, EPSTI1, USP18, BST2, CSF1, C1S, DHX58, TRIM14, OASL, IRF7, LGALS3BP, DDX60, LAP3, LAMP3, PARP12, PARP9, SP110, PLSCR1, WARS, STAT1, IRF3, IRF5, MSC, JUN, SP11, IRF1, COMMD3-BM11, STAT2, RUNX3, SREBF1, and/or FLI1 is elevated relative to the amount of said RNA in a control sample obtained from a normal subject not suffering from cancer.

In an alternative embodiment, the RNA level of CXCL10, CXCL11, RSAD2, MX1, BATF2, IFI44L, IFITM1, ISG15, CMPK2, IF127, CD74, IFIH1, CCRL2, IFI44, HERC6, ISG20, IFIT3, HLA-C, OAS1, IFI35, IRF9, EPSTI1, USP18, BST2, CSF1, C1S, DHX58, TRIM14, OASL, IRF7, LGALS3BP, DDX60, LAP3, LAMP3, PARP12, PARP9, SP110, PLSCR1, WARS, STAT1, IRF3, IRF5, MSC, JUN, SP11, IRF1, COMMD3-BM11, STAT2, RUNX3, SREBF1, and/or FLI1 is elevated in tumour relative to the amount of said RNA in a non-tumour sample obtained from the same patient.

In one embodiment the cancer shows increased expression of CXCL10 or CXCL11.

In another embodiment the cancer shows increased expression of IRF7, IFITM1, IRF9, MX1, or IFI35.

In another embodiment the cancer shows increased expression of one or more, e.g. two or more of IRF7, IFITM1, IRF9, MX1, IFI35, CXCL10 or CXCL11.

In one embodiment the cancer shows increased expression of one, two, three, four, five or more of: CXCL10, CXCL11, RSAD2, MX1, BATF2, IFI44L, IFITM1, ISG15, CMPK2, IF127, CD74, IFIH1, CCRL2, IFI44, HERC6, ISG20, IFIT3, HLA-C, OAS1, IFI35, IRF9, EPSTI1, USP18, BST2, CSF1, C1S, DHX58, TRIM14, OASL, IRF7, LGALS3BP, DDX60, LAP3, LAMP3, PARP12, PARP9, SP110, PLSCR1 and WARS.

In one embodiment the cancer shows increased expression of one, two, three, four, five or more of IRF7, STAT1, IRF3, IRF5, MSC, JUN, SP11, IRF1, COMMD3-BM11, STAT2, RUNX3, SREBF1, IRF9, and FLI1.

In another embodiment the cancer shows increased expression of IRF7, IFITM1, IRF9, MX1, or IFI35.

In another embodiment the cancer shows increased expression of one or more, e.g. two or more of IRF7, IFITM1, IRF9, MX1, IF135, CXCL10 or CXCL11.

In some embodiments, the elevated level is relative to the amount of RNA determined in samples from MDM2 inhibitor non-responsive subjects.

In one embodiment it is elevated or increased relative to normal levels.

Upper limit of normal (ULN) refers to those levels that are at 95% of the whole range. It is a set of values within which 95 percent of the normal population falls (that is, 95% prediction interval).

In one embodiment, the elevated level is a>1 fold difference relative to the control sample, upper limit of normal (ULN) or sample taken from said patient, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15, 20, 25, 30, 35, 40, 45, 50, 55, 60, 65, 70, 75, 80, 85, 90, 95, 100 or any ranges therebetween. In one embodiment, the elevated level is between 1 and 50 fold difference relative to the control sample or ULN. In one embodiment, the elevated level is very high for example a>10 fold difference relative to the control sample, ULN or sample taken from said patient, such as a fold difference of 10, 10.5, 11, 11.5, 12, 12.5, 15, 20, 25, 30, 35, 40, 45, 50, 55, 60, 65, 70, 75, 80, 85, 90, 95, 100, 1000 or any ranges therebetween.

In one embodiment, the elevated level is between 10 and 1000 fold difference relative to the control sample or ULN. In one embodiment, the elevated level is between 2 and 10 fold difference relative to the control sample (e.g. 5 fold).

The fold difference can be determined between disease individual and normal individual (reference value or control sample). This reference value can be calculated from normal individuals or based on a pool of samples excluding the sample type to be tested (eg. TP53 wild and DDR biomarker loss). In one embodiment the difference in expression of interferon genes in normal tissues (source: GTEx; Nat Biotechnol. 2017 Apr. 11; 35(4):314-316) to the patient mesothelioma samples (source: TCGA) is from more than 5-fold to 0.05 fold (log 2 scale) in particular there is an average of 1.5 fold (log 2 scale) increase across a set of genes.

In one embodiment, the concentration of the RNA is determined by RT-PCR and/or microarray and/or nanostring. It is typical for each assay to have an “upper limit of normal” (ULN) value associated with the specific assay method. Such ULN is typically determined from a sufficient sample size of normal, healthy subjects using the particular assay method to measure the RNA concentration. The ULN is then typically determined to be the highest RNA concentration that is still considered within the normal range (e.g. within two standard deviations of the mean). Since such ULN values will vary depending on the particular assay method employed to measure concentration, each specific assay will have a unique ULN value that is associated with that assay method.

As shown herein, concentrations can be used to predict whether a cancer patient will be likely to benefit from MDM2 antagonist treatment.

### DDR Biomarker Assays

In one embodiment, the protein level of one or more of the DDR biomarkers is decreased relative to the amount of said protein in a control sample obtained from a normal subject not suffering from cancer.

In an alternative embodiment, the protein level of one or more DDR biomarkers is decreased relative to the amount of said protein in an earlier sample obtained from the same patient.

In one embodiment it is reduced or decreased relative to normal levels.

Upper limit of normal (ULN) refers to those levels that are at 95% of the whole range. It is a set of values within which 95 percent of the normal population falls (that is, 95% prediction interval).

In one embodiment, the reduced level is a<1 fold difference relative to the control sample, upper limit of normal (ULN) or sample taken from said patient, such as a fold difference of 0.75, 0.5, 0.4, 0.3, 0.2, 0.15, 0.1, 0.09, 0.08, 0.07, 0.06, 0.05, 0.04, 0.03, 0.02 or 0.01 or any ranges therebetween. In one embodiment, the reduced level is between 1 and 0.01 fold difference relative to the control sample or ULN. In one embodiment, the reduced level is very low for example a>0.01 fold difference relative to the control sample, ULN or sample taken from said patient, such as a fold difference of 0.001 or any ranges therebetween. In one embodiment, the reduced level is 0 i.e. completely absent.

In another embodiment the level of the DDR biomarker or biomarkers is determined by immunohistochemistry.

Proteins, protein complexes or proteomic markers may be specifically identified and/or quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, some chromatographic techniques (i.e. immunoaffinity chromatography), flow cytometry, immunoprecipitation and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, sequencing, protease digests, and the like. Such methods are based on the mass, charge, hydrophobicity or hydrophilicity, which is derived from the amino acid complement of the protein or protein complex, and the specific sequence of the amino acids.

In one embodiment there is no expression of the one or more DDR biomarkers. Samples having low levels of one or more DDR biomarkers can be identified as DDR biomarker negative, for example DDR biomarker loss.

In one embodiment the loss of one or more DDR biomarkers is assessed by mutational analysis for example DNA sequencing.

Levels of cytoplasmic as well as nuclear expression of one or more DDR biomarkers can also be determined. Nuclear localisation of a protein is a marker in cells. Levels of nuclear expression can be scored using histology by a score (range, 0-100) expressing the percentage of positive cells was obtained following treatment with an antibody (e.g. monoclonal antihuman antibody against the biomarker). The immunostaining expression scores can be made.

Levels of the one or more DDR biomarkers in the cytoplasm can also be measured using immunohistochemistry or immunofluorescence.

In one embodiment, the level of one or more of the DDR biomarkers is reduced relative to the amount of said protein in a control sample obtained from a normal subject not suffering from cancer.

In one embodiment, the level of one or more of the DDR biomarkers is reduced in tumour relative to the amount of said protein in a non-tumour sample obtained from the same patient.

In one embodiment, the expression level of one or more of the DDR biomarkers is reduced by 50%, 60%, 70%, 80%, 90%, 95%, 96, 97%, 98%, 99%, 99.5%, 99.9% or 100%. 100% reduction in expression is completely reduced i.e. total loss. In some embodiments, at least 50% reduction is provided. In some embodiments, at least 75% reduction is provided.

In some embodiments, at least 80% reduction is provided.

In some embodiments, at least 95% reduction is provided, for example at least 99%.

Methods of quantifying The invention relates to identifying a patient for treatment with an MDM2 antagonist. In some embodiments, the methods comprise at least the steps of:
(a) contacting a sample from the patient with an antibody against one or more DDR biomarker, (or one or more DDR biomarker substrates);
(b) performing an ELISA or immunohistochemical assay on said sample;
(c) determining the level of one or more DDR biomarker; and
(d) identifying the patient as a candidate for treatment with an MDM2 antagonist when (i) the level of one or more DDR biomarkers is reduced relative to the upper limit of normal (ULN); or (ii) one or more DDR biomarkers are absent; or (iii) the level of one or more DDR biomarkers is low relative to the upper limit of normal (ULN).

In other embodiments, the method for identifying a patient for treatment with an MDM2 antagonist comprises:
(a) contacting a sample from the patient with an antibody against one or more DDR biomarkers (and/or one or more DDR biomarker substrates) to determine the level of protein expression; and/or
(b) contacting a sample from the patient with an antibody against one or more different DDR biomarkers to (a) (and/or one or more DDR biomarker substrates) to determine the level of protein expression;
(c) treating the patient with an MDM2 antagonist when the level of the one or more DDR biomarkers is reduced relative to the upper limit of normal (ULN)

Also described is a method for identifying or selecting a patient for treatment with an MDM2 antagonist, the method comprising:
(a) contacting a sample from the patient with an antibody one or more DDR biomarkers to determine the level of protein expression; and/or
(b) contacting a sample from the patient with an antibody against one or more DDR biomarkers to determine the level of protein expression; and/or
(c) contacting a sample from the patient with a plurality of oligonucleotide primers, said plurality of primers comprising at least one pair of oligonucleotide primers for any one or more DDR biomarkers;
(d) treating the patient with an MDM2 antagonist when the level of one or more DDR biomarkers is reduced relative to the upper limit of normal (ULN).

The selected patient is typically a cancer patient. A patient is typically selected when the patient has a level of one or more DDR biomarkers in the biological sample from the patient that is lower than a predetermined value (or is absent).

A method for predicting efficacy of an MDM2 antagonist for a cancer in a patient, comprises determining the level of one or more DDR biomarkers in the biological sample from the patient, where a biological sample level of one or more DDR biomarkers is less than a predetermined value is predictive of efficacy in the patient.

### Systems for Carrying Out the Methods

The methods described herein can make use of a system to assist in the assessment or prognosis of the patient. The system can be a single apparatus having various device components (units) integrated therein. The system can also have its various components, or some of these components, as separate apparatuses. The components can comprise a measurement device, a graphical user interface and a computer-processing unit.

The system typically comprises a data connection to an interface, whereby the interface itself can be a part of the system or can be a remote interface. The latter refers to the possibility to use a different apparatus, preferably a handheld apparatus such as a smartphone or a tablet computer, for providing the actual interface. The data connection in such cases will preferably involve wireless data transfer such as by Wi-Fi or Bluetooth, or by other techniques or standards.

In certain embodiments, the measurement device is configured to receive a tissue sample, for example by putting one or more cancer cells or a drop of blood on a cartridge, which can be inserted into the device. The device can be an existing device that is capable to determine, from the same sample, the levels of the biomarker or biomarkers. A processing unit can receive numerical values for the protein concentrations from the measurement device. The processing unit is typically provided with software (typically embedded software) allowing it to calculate a score based on the input data.

In another embodiment, a system for assessing whether a human cancer patient is suitable for treatment with an MDM2 antagonist comprises:
(a) detection means able and adapted to detect in a sample from the human patient the biomarker or biomarkers of the invention. Such means are known, and easily accessible to the skilled person. Typically, there is provided a container for receiving a sample of a subject therein, the container provided with the detection means;
(b) a processor able and adapted to determine from the determined concentrations of said proteins an indication of the patient's likelihood of being treated with an MDM2 antagonist.

Optionally, the system comprises a user interface (or a data connection to remote interface), particularly a graphical user interface (GUI), capable of presenting information; a GUI is a type of user interface that allows users to interact with electronic devices through graphical icons and visual indicators such as secondary notation, instead of text-based user interfaces, typed command labels or text navigation (none of such interface types being excluded in the present invention); GUIs are generally known, and are used typically in handheld mobile devices such as MP3 players, portable media players, gaming devices, smartphones and smaller household, office and industrial controls; as said, the interface optionally can also be chosen so as to be capable of putting in information, such as, information on the patient.

In one embodiment, a system for determining the suitability of a human cancer patient for treatment with an MDM2 antagonist comprises a storage memory for storing data associated with a sample from the patient comprising data associated with a panel of biomarkers indicating biomarker expression levels in the sample from the subject, the panel of biomarkers comprising one or more biomarkers of the invention; and

a processor communicatively coupled to the storage memory for classifying the patient.

### Kits

The invention also provides, either separately or as part of the aforementioned system, a kit for detecting one or more of the biomarkers of the invention, to assess a patient's likelihood of responding to MDM2 inhibition for cancer therapy. The kit typically comprises one or more detection reagents for detecting one or more of the biomarkers of the invention. These reagents may be for direct detection or indirect detection of the biomarker, for example detection of a correlated substrate.

Typically, the kit comprises two or more, or three or more, detection reagents, each directed to a different biomarker of the invention.

As discussed above with reference to the methods of the invention, the kit may comprise more detection reagents, such as for other proteins. In a preferred embodiment the detection reagents made available in the kit consist of the detection reagents for the detection of two, three or four proteins making up a biomarker panel of the invention, as mentioned.

The kit may comprise a solid support, such as a chip, a microtiter plate or a bead or resin comprising said detection reagents. In some embodiments, the kits comprise mass spectrometry probes.

The kit may also provide washing solutions and/or detection reagents specific for either unbound detection reagent or for said biomarkers (sandwich type assay).

Such kits will suitably comprise a biosensor for detection and/or quantification of one or more of the biomarkers of the invention, optionally together with instructions for use of the kit in accordance with the methodology as described herein.

There are well established genetic and biochemical means of characterising the state of one or more of the biomarkers of the invention. There are also well established biochemical means of characterising the amount of proteins in blood e.g. serum samples.

In one embodiment, the invention includes a packaged cancer treatment. The packaged treatment includes a composition packaged with instructions for using an effective amount of the composition of the invention for an intended use in a patient selected using the present invention. In other embodiments, the present invention provides a use of any of the compositions of the invention for manufacture of a medicament to treat cancer in a subject.

In one embodiment the invention provides a kit or panel or array for determining the level of one or more of the biomarkers of the invention from a single patient sample.

### Biological Effects

The compounds described herein, subgroups and examples thereof, have been shown to inhibit the interaction of p53 with MDM2. Such inhibition leads to cell proliferative arrest and cell death (typically apoptosis), which may be useful in preventing or treating disease states or conditions described herein, for example the diseases and conditions discussed below and the diseases and conditions described above in which p53 and MDM2 play a role. Thus, for example, it is envisaged that the compounds for use in the invention may be useful in alleviating or reducing the incidence of cancer.

The compounds described herein may be useful for the treatment of the adult population. The compounds of the present invention may be useful for the treatment of the pediatric population.

The compounds described herein have been shown to be good antagonists of the formation of MDM2-p53 complex. The compounds described herein are capable of binding to MDM2 and exhibiting potency for MDM2. The efficacies of the compounds of the present invention have been determined against MDM2/p53 using the assay protocol described herein and other methods known in the art. More particularly, the compounds of the formula (I°) and sub-groups thereof have affinity for MDM2/p53.

Certain compounds for use in the invention are those having IC₅₀ values of less than 0.1 μM in particular less than 0.01 or 0.001 μM.

MDM2/p53 function has been implicated in many diseases due to its role in a variety of process for example vascular remodelling and antiangiogenic processes and regulation of metabolic pathways, as well as in oncogenesis. As a consequence of their affinity for MDM2 it is anticipated that the compounds may prove useful in treating or preventing a range of diseases or conditions including autoimmune conditions; diabetes mellitus; chronic inflammatory diseases, for example lupus nephritis, systemic lupus erythematosus (SLE), autoimmune mediated glomerulonephritis, rheumatoid arthritis, psoriasis, inflammatory bowel disease, autoimmune diabetes mellitus, Eczema hypersensitivity reactions, asthma, COPD, rhinitis, and upper respiratory tract disease; hyperkeratotic diseases such as autosomal recessive congenital ichthyosis (ARCI); kidney diseases including glomerular disorders, chronic kidney disease (CKD) renal inflammation, podocyte loss, glomerulosclerosis, proteinuria, and progressive kidney disease; cardiovascular diseases for example cardiac hypertrophy, restenosis, arrhythmia, atherosclerosis; ischemic injury associated myocardial infarctions, vascular injury, stroke and reperfusion injury; vascular proliferative diseases; ocular diseases such as age-related macular degeneration in particular wet form of age-related macular degeneration, ischemic proliferative retinopathies such as retinopathy of prematurity (ROP) and diabetic retinopathy, and hemangioma.

As a consequence of their affinity for MDM2 it is anticipated that the compounds may prove useful in treating or preventing proliferative disorders such as cancers.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, bowel, colorectal, rectum and anus), liver (hepatocellular carcinoma), gall bladder and biliary system, exocrine pancreas, kidney (for example renal cell carcinoma), lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, testes, cervix, myometrium, endometrium, thyroid (for example thyroid follicular carcinoma), brain, adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukemia [AML], chronic myelogenous leukemia [CML], chronic myelomonocytic leukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcoma protuberans; tumours of the central or peripheral nervous system (for example astrocytomas (e.g. gliomas), neuromas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wilms tumour, and primitive neuroectodermal tumours); or syndromes, congenital or otherwise, which leave the patient susceptible to malignancy (for example Xeroderma Pigmentosum).

Growth of cells is a closely controlled function. Cancer, a condition of abnormal cell growth, results when cells replicate in an uncontrolled manner (increasing in number), uncontrollably grow (getting larger) and/or experience reduced cell death by apoptosis (programmed cell death), necrosis, or annoikis. In one embodiment abnormal cell growth is selected from uncontrolled cell proliferation, excessive cell growth or reduced programmed cell death. In particular, the condition or disease of abnormal cell growth is a cancer.

Thus, in the pharmaceutical compositions, uses or methods of this invention for treating a disease or condition comprising abnormal cell growth (i.e. uncontrolled and/or rapid cell growth), the disease or condition comprising abnormal cell growth in one embodiment is a cancer.

Many diseases are characterized by persistent and unregulated angiogenesis. Chronic proliferative diseases are often accompanied by profound angiogenesis, which can contribute to or maintain an inflammatory and/or proliferative state, or which leads to tissue destruction through the invasive proliferation of blood vessels. Tumour growth and metastasis have been found to be angiogenesis-dependent. Compounds for use in the invention may therefore be useful in preventing and disrupting initiation of tumour angiogenesis.

Angiogenesis is generally used to describe the development of new or replacement blood vessels, or neovascularisation. It is a necessary and physiological normal process by which vasculature is established in the embryo. Angiogenesis does not occur, in general, in most normal adult tissues, exceptions being sites of ovulation, menses and wound healing. Many diseases, however, are characterized by persistent and unregulated angiogenesis. For instance, in arthritis, new capillary blood vessels invade the joint and destroy cartilage. In diabetes (and in many different eye diseases), new vessels invade the macula or retina or other ocular structures, and may cause blindness. The process of atherosclerosis has been linked to angiogenesis. Tumour growth and metastasis have been found to be angiogenesis-dependent. The compounds may be beneficial in the treatment of diseases such as cancer and metastasis, ocular diseases, arthritis and hemangioma.

Therefore, the compounds for use in the invention may be useful in the treatment of metastasis and metastatic cancers. Metastasis or metastatic disease is the spread of a disease from one organ or part to another non-adjacent organ or part. The cancers which can be treated by the compounds of the invention include primary tumours (i.e. cancer cells at the originating site), local invasion (cancer cells which penetrate and infiltrate surrounding normal tissues in the local area), and metastatic (or secondary) tumours i.e. tumours that have formed from malignant cells which have circulated through the bloodstream (haematogenous spread) or via lymphatics or across body cavities (trans-coelomic) to other sites and tissues in the body. In particular, the compounds for use in the invention may be useful in the treatment of metastasis and metastatic cancers.

In one embodiment the haematological malignancies is a leukaemia. In another embodiment the haematological malignancies is a lymphoma. In one embodiment the cancer is AML. In another embodiment the cancer is CLL.

In one embodiment the compound used in the invention is for use in the prophylaxis or treatment of leukemia, such as acute or chronic leukaemia, in particular acute myeloid leukaemia (AML), acute lymphocytic leukaemia (ALL), chronic lymphocytic leukaemia (CLL), or chronic myeloid leukemia (CML).

In one embodiment the compound used in the invention is for use in the prophylaxis or treatment of lymphoma, such as acute or chronic lymphoma, in particular Burkitt lymphoma, Hodgkin lymphoma, non-Hodgkin lymphoma or diffuse large B-cell lymphoma.

In one embodiment the compound used in the invention is for use in the prophylaxis or treatment of acute myeloid leukaemia (AML) or acute lymphocytic leukaemia (ALL).

In one embodiment the compound used in the invention is for use in the prophylaxis or treatment of haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukemia [AML], chronic myelogenous leukemia [CML], chronic myelomonocytic leukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukemia.

One embodiment includes a compound used in the invention for use in the prophylaxis or treatment of cancer in a patient selected from a sub-population possessing cancers which are p53 wild-type or have an MDM2 amplification The cancers may be cancers which are sensitive to treatment with MDM2 antagonists. The cancers may be cancers which overexpress MDM2. The cancer may be cancers which are p53 wild-type.

Particular cancers include those with an MDM2 amplification and/or MDM2 overexpression, for example, hepatocellular carcinoma, lung, sarcomas, osteosarcomas, and Hodgkin disease.

Particular cancers include those with wild-type p53. Particulars cancers include those cancer cells with wild-type p53, particularly but not exclusively, if MDM2 is highly expressed.

In one embodiment the cancer is a p53 functional tumours. In one embodiment this disease to be treated is p53 functional solid and haematological malignancies. In another embodiment the patientto be treated has p53 mutant tumour for example AML patients with p53 mutant tumour.

In one embodiment the cancer is a tumour of the brain, for example glioma, or neuroblastoma.

In one embodiment the cancer is a cancer of the skin, for example melanoma.

In one embodiment the cancer is a cancer of the lung, for example NSCLC or mesothelioma. In one embodiment the cancer is a cancer of the lung, for example mesothelioma. In one embodiment the mesothelioma is malignant peritoneal mesothelioma or malignant pleural mesothelioma.

In one embodiment the cancer is a cancer of the gastrointestinal tract, for example GIST, gastric, colorectal or bowel.

In one embodiment the cancer is osteosarcoma.

In one embodiment the cancer is liposarcoma.

In one embodiment the cancer is Ewing's sarcoma.

In one embodiment, the cancer is liposarcoma, soft tissue sarcoma, osteosarcoma, oesophageal cancer, and certain paediatric malignancies including B-cell malignancies.

In one embodiment, the cancer is colorectal, breast, lung and brain In one embodiment, the cancer is a paediatric cancer.

In one embodiment, the cancer is a p53 wild-type.

In one embodiment the cancer is a cancer of the lung, for example NSCLC or mesothelioma, Renal e.g. KIRC or cancer of the brain such as glioblastoma.

Whether a particular cancer is one which is sensitive to MDM2 antagonists, may be determined by a method as set out in the section headed “Methods of Diagnosis”.

A further aspect provides the use of a compound for the manufacture of a medicament for the treatment of a disease or condition as described herein, in particular cancer.

Individuals with fanconi anemia have an increased risk of developing acute myeloid leukemia (AML), squamous cell carcinoma or tumors of the head, neck, skin, gastrointestinal system, or genital tract.

Homologous Recombination Deficiency (HRD) is enhanced in prostate, ovarian, breast and gynaecological cancers. Therefore in one embodiment, the cancer is prostate, ovarian, breast or gynaecological cancer. In one embodiment the HRR pathway deficient (HRD) cancer is prostate, ovarian, breast or a gynaecological cancer.

MSI-H is enriched in colorectal, gastric and gynaecological cancers. Therefore in one embodiment, the cancer is colorectal, gastric and gynaecological cancers Certain cancers are resistant to treatment with particular drugs. This can be due to the type of the tumour (most common epithelial malignancies are inherently chemoresistant and prostate is relatively resistant to currently available regimens of chemotherapy or radiation therapy) or resistance can arise spontaneously as the disease progresses or as a result of treatment. In this regard, references to prostate includes prostate with resistance towards anti-androgen therapy, in particular abiraterone or enzalutamide, or castrate-resistant prostate. Similarly references to multiple myeloma includes bortezomib-insensitive multiple myeloma or refractory multiple myeloma and references to chronic myelogenous leukemia includes imitanib-insensitive chronic myelogenous leukemia and refractory chronic myelogenous leukemia. In this regard, references to mesothelioma includes mesothelioma with resistance towards topoisomerase poisons, alkylating agents, antitubulines, antifolates, platinum compounds and radiation therapy, in particular cisplatin-resistant mesothelioma.

The compounds may also be useful in the treatment of tumour growth, pathogenesis, resistance to chemo- and radio-therapy by sensitising cells to chemotherapy and as an anti-metastatic agent.

Therapeutic anticancer interventions of all types necessarily increase the stresses imposed on the target tumour cells. Antagonists of MDM2/p53 represent a class of chemotherapeutics with the potential for: (i) sensitizing malignant cells to anticancer drugs and/or treatments; (ii) alleviating or reducing the incidence of resistance to anticancer drugs and/or treatments; (iii) reversing resistance to anticancer drugs and/or treatments; (iv) potentiating the activity of anticancer drugs and/or treatments; (v) delaying or preventing the onset of resistance to anticancer drugs and/or treatments.

In one embodiment the invention provides a compound for use in the treatment of a disease or condition which is mediated by MDM2. In a further embodiment the disease or condition which is mediated by MDM2 is a cancer which is characterised by overexpression and/or increased activity of MDM2, or high copy number MDM2 and/or wildtype p53.

A further aspect provides the use of a compound for the manufacture of a medicament for the treatment of a disease or condition as described herein, in particular cancer.

In one embodiment there is provided a compound for use in the prophylaxis or treatment of a disease or condition mediated by MDM2/p53. In one embodiment there is provided a compound for inhibiting the interaction between of MDM2 protein with p53.

In one embodiment there is provided a pharmaceutical composition comprising an effective amount of at least one compound as defined.

In one embodiment there is provided a method for the prophylaxis or treatment of cancer comprising the steps of administering to a mammal a medicament comprising at least one compound as defined.

### Pharmaceutical Formulations

While it is possible for the active compound to be administered alone, it is generally presented as a pharmaceutical composition (e.g. formulation).

Thus, the present invention further provides pharmaceutical compositions, as defined above, and methods of making a pharmaceutical composition comprising (e.g admixing) at least one MDM2 antagonist, including one compound of formula (I°) (and sub-groups thereof as defined herein), together with one or more pharmaceutically acceptable excipients and optionally other therapeutic or prophylactic agents as described herein.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any other excipients conventionally used in pharmaceutical compositions. Examples of excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. a human subject) without excessive toxicity, irritation, allergic response, or other problem or complication, commensurate with a reasonable benefit/risk ratio. Each excipient must also be “acceptable” in the sense of being compatible with the other ingredients of the formulation.

Pharmaceutical compositions containing an MDM2 antagonist, including compounds of the formula (I°) can be formulated in accordance with known techniques, see for example, Remington's Pharmaceutical Sciences, Mack Publishing Company, Easton, PA, USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short-term infusion or longer term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use. In one embodiment, the formulation is provided as an active pharmaceutical ingredient in a bottle for subsequent reconstitution using an appropriate diluent.

The pharmaceutical formulation can be prepared by lyophilsing an MDM2 antagonist, including a compound of formula (I°), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

In one typical embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another typical embodiment, the pharmaceutical composition is in a form suitable for sub-cutaneous (s.c.) administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The MDM2 antagonist, including a compound of formula (I°) may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published Mar. 13, 2006. Nanoparticles for drug delivery are also described in J. Control. Release, 2003, 91 (1-2), 167-172, and in Sinha et aL., Mol. Cancer Ther. August 1, (2006) 5, 1909.

The pharmaceutical compositions typically comprise from approximately 1% (w/w) to approximately 95% active ingredient and from 99% (w/w) to 5% (w/w) of a pharmaceutically acceptable excipient or combination of excipients. Typically, the compositions comprise from approximately 20% (w/w) to approximately 90%,% (w/w) active ingredient and from 80% (w/w) to 10% of a pharmaceutically acceptable excipient or combination of excipients. The pharmaceutical compositions comprise from approximately 1% to approximately 95%, typically from approximately 20% to approximately 90%, active ingredient. Pharmaceutical compositions according to the invention may be, for example, in unit dose form, such as in the form of ampoules, vials, suppositories, pre-filled syringes, dragées, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example tablets and capsules typically contain 0-20% disintegrants, 0-5% lubricants, 0-5% flow aids and/or 0-99% (w/w) fillers/or bulking agents (depending on drug dose). They may also contain 0-10% (w/w) polymer binders, 0-5% (w/w) antioxidants, 0-5% (w/w) pigments. Slow release tablets would in addition contain 0-99% (w/w) polymers (depending on dose). The film coats of the tablet or capsule typically contain 0-10% (w/w) release-controlling (e.g. delaying) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds used in the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described herein. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound. Solutions of the active compound may also be used for rectal administration.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

The MDM2 antagonist, including a compound of formula (I°) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

Combinations with Other Anticancer Agents

The MDM2 antagonist as defined herein may be useful in the prophylaxis or treatment of a range of disease states or conditions mediated by MDM2/p53. Examples of such disease states and conditions are set out above.

The compounds are generally administered to a subject in need of such administration, for example a human or animal patient, typically a human.

The compounds will typically be administered in amounts that are therapeutically or prophylactically useful and which generally are non-toxic. However, in certain situations (for example in the case of life threatening diseases), the benefits of administering a compound used in the invention (e.g. a compound of formula (I°)) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a continuous manner or in a manner that provides intermittent dosing (e.g. a pulsatile manner).

A typical daily dose of the MDM2 antagonists can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of formula (I°) can be administered on a daily basis or on a repeat basis every 2, or 3, or 4, or 5, or 6, or 7, or 10 or 14, or 21, or 28 days for example.

Dosages may also be expressed as the amount of drug administered relative to the body surface area of the patient (mg/m²). A typical daily dose of the MDM2 antagonists can be in the range from 3700 pg/m² to 3700 mg/m², more typically 185 ng/m² to 925 mg/m², and more usually 370 ng/m² to 555 mg/m² (e.g. 370 ng/m² to 370 mg/m², and more typically 37 mg/m² to 740 mg/m², for example 37 mg/m² to 370 mg/m²) although higher or lower doses may be administered where required. The compound of the formula (I°) can be administered on a daily basis or on a repeat basis every 2, or 3, or 4, or 5, or 6, or 7, or 10 or 14, or 21, or 28 days for example.

The compounds of the invention may be administered orally in a range of doses, for example 0.1 to 5000 mg, or 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day. The compound can be administered continuously (i.e. taken every day without a break for the duration of the treatment regimen). Alternatively, the compound can be administered intermittently (i.e. taken continuously for a given period such as a week, then discontinued for a period such as a week and then taken continuously for another period such as a week and so on throughout the duration of the treatment regimen). Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles. This discontinuous treatment can also be based upon numbers of days rather than a full week. For example, the treatment can comprise daily dosing for 1 to 6 days, no dosing for 1 to 6 days with this pattern repeating during the treatment protocol. The number of days (or weeks) wherein the compounds used in the invention are not dosed do not necessarily have to equal the number of days (or weeks) wherein the compounds used in the invention are dosed.

In one embodiment, the compounds used in the invention can be administered in amounts from 3 mg/m² to 125 mg/m² daily. Treatment can be by continuous daily dosing or more usually consist of multiple cycles of treatment separated by treatment breaks. One example of a single treatment cycle is 5 consecutive daily doses followed by 3 weeks without treatment.

One particular dosing regimen is once a day (e.g. orally) for a week (e.g. 5 days of treatment), followed by a treatment break of 1, 2, or 3 weeks. An alternative dosing regimen is once a week (e.g. orally), for 1, 2, 3 or 4 weeks.

In one particular dosing schedule, a patient will be given an infusion of a compound of formula (I°) for periods of one hour daily for up to ten days in particular up to five days for one week, and the treatment repeated at a desired interval such as two to four weeks, in particular every three weeks.

More particularly, a patient may be given an infusion of a compound of formula (I°) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

The compounds used in the invention can also be administered by bolus or continuous infusion. The compound used in the invention can be given daily to once every week, or once every two weeks, or once every three weeks, or once every four weeks during the treatment cycle. If administered daily during a treatment cycle, this daily dosing can be discontinuous over the number of weeks of the treatment cycle: for example, dosed for a week (or a number of days), no dosing for a week (or a number of days, with the pattern repeating during the treatment cycle.

In a further particular dosing schedule, a patient is given a continuous infusion for a period of 12 hours to 5 days, and in particular a continuous infusion of 24 hours to 72 hours.

Ultimately, however, the quantity of compound administered and the type of composition used will be commensurate with the nature of the disease or physiological condition being treated and will be at the discretion of the physician.

It may be beneficial to use a compound used in the invention as a single agent or to combine the compound used in the invention with another agent which acts via a different mechanism to regulate cell growth thus treating two of the characteristic features of cancer development. Combination experiments can be performed, for example, as described in Chou TC, Talalay P. Quantitative analysis of dose-effect relationships: the combined effects of multiple drugs or enzyme inhibitors. Adv Enzyme Regulat 1984;22: 27-55.

The compounds as defined herein can be administered as the sole therapeutic agent or they can be administered in combination therapy with one of more other compounds (or therapies) for treatment of a particular disease state, for example a neoplastic disease such as a cancer as hereinbefore defined.

For the treatment of the above conditions, the compounds used in the invention may be advantageously employed in combination with one or more other medicinal agents, more particularly, with other anti-cancer agents or adjuvants (supporting agents in the therapy) in cancer therapy. Examples of other therapeutic agents or treatments that may be administered together (whether concurrently or at different time intervals) with the MDM2 antagonists include but are not limited to:
Topoisomerase I inhibitors
Antimetabolites
Tubulin targeting agents
DNA binder and topoisomerase II inhibitors
Alkylating Agents
Monoclonal Antibodies.
Anti-Hormones
Signal Transduction Inhibitors
Proteasome Inhibitors
DNA methyl transferase inhibitors
Cytokines and retinoids
Chromatin targeted therapies
Radiotherapy, and,
Other therapeutic or prophylactic agents.

Particular examples of anti-cancer agents or adjuvants (or salts thereof), include but are not limited to any of the agents selected from groups (i)-(xlviii), and optionally group (xlix), below:
(i) Platinum compounds, for example cisplatin (optionally combined with amifostine), carboplatin or oxaliplatin;
(ii) Taxane compounds, for example paclitaxel, paclitaxel protein bound particles (Abraxane™) docetaxel, cabazitaxel or larotaxel;
(iii) Topoisomerase I inhibitors, for example camptothecin compounds, for example camptothecin, irinotecan(CPT11), SN-38, or topotecan;
(iv) Topoisomerase II inhibitors, for example anti-tumour epipodophyllotoxins or podophyllotoxin derivatives for example etoposide, or teniposide;
(v) Vinca alkaloids, for example vinblastine, vincristine, liposomal vincristine (Onco-TCS), vinorelbine, vindesine, vinflunine or vinvesir;
(vi) Nucleoside derivatives, for example 5-fluorouracil (5-FU, optionally in combination with leucovorin), gemcitabine, capecitabine, tegafur, UFT, S1, cladribine, cytarabine (Ara-C, cytosine arabinoside), fludarabine, clofarabine, or nelarabine;
(vii) Antimetabolites, for example clofarabine, aminopterin, or methotrexate, azacitidine, cytarabine, floxuridine, pentostatin, thioguanine, thiopurine, 6-mercaptopurine, or hydroxyurea (hydroxycarbamide);
(viii) Alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU);
(ix) Anthracyclines, anthracenediones and related drugs, for example daunorubicin, doxorubicin (optionally in combination with dexrazoxane), liposomal formulations of doxorubicin (eg. Caelyx™, Myocet™, Doxil™), idarubicin, mitoxantrone, epirubicin, amsacrine, or valrubicin;
(x) Epothilones, for example ixabepilone, patupilone, BMS-310705, KOS-862 and ZK-EPO, epothilone A, epothilone B, desoxyepothilone B (also known as epothilone D or KOS-862), aza-epothilone B (also known as BMS-247550), aulimalide, isolaulimalide, or luetherobin;
(xi) DNA methyl transferase inhibitors, for example temozolomide, azacytidine, or decitabine;
(xii) Antifolates, for example methotrexate, pemetrexed disodium, or raltitrexed;
(xiii) Cytotoxic antibiotics, for example antinomycin D, bleomycin, mitomycin C, dactinomycin, carminomycin, daunomycin, levamisole, plicamycin, or mithramycin;
(xiv) Tubulin-binding agents, for example combrestatin, colchicines or nocodazole;
(xv) Signal Transduction inhibitors such as Kinase inhibitors for example receptor tyrosine kinase inhibitors (e.g. EGFR (epithelial growth factor receptor) inhibitors, VEGFR (vascular endothelial growth factor receptor) inhibitors, PDGFR (platelet-derived growth factor receptor) inhibitors, Axl inhibitors, MTKI (multi target kinase inhibitors), Raf inhibitors, ROCK inhibitors, mTOR inhibitors, MEK inhibitors or PI3K Inhibitors) for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), vemurafenib (PLX4032 or RG7204), dabrafenib, encorafenib, selumetinib (AZD6244), trametinib (GSK121120212), dactolisib (BEZ235), buparlisib (BKM-120; NVP-BKM-120), BYL719, copanlisib (BAY-80-6946), ZSTK-474, CUDC-907, apitolisib (GDC-0980; RG-7422), pictilisib (pictrelisib, GDC-0941, RG-7321), GDC-0032, GDC-0068, GSK-2636771, idelalisib (formerly CAL-101, GS 1101, GS-1101), MLN1117 (INK1117), MLN0128 (INK128), IPI-145 (INK1197), LY-3023414, ipatasertib, afuresertib, MK-2206, MK-8156, LY-3023414, LY294002, SF1126 or PI-103, sonolisib (PX-866), or AT13148.
(xvi) Aurora kinase inhibitors for example AT9283, barasertib (AZD1152), TAK-901, MK0457 (VX680), cenisertib (R-763), danusertib (PHA-739358), alisertib (MLN-8237), or MP-470;
(xvii) CDK inhibitors for example AT7519, roscovitine, seliciclib, alvocidib (flavopiridol), dinaciclib (SCH-727965), 7-hydroxy-staurosporine (UCN-01), JNJ-7706621, BMS-387032 (a.k.a. SNS-032), PHA533533, ZK-304709, orAZD-5438 and including CDK4 inhibitors such as palbociclib (PD332991) and ribociclib (LEE-011);
(xviii) PKA/B inhibitors and PKB (akt) pathway inhibitors for example AT13148, AZ-5363, Semaphore, SF1126 and MTOR inhibitors such as rapamycin analogues, AP23841 and AP23573, calmodulin inhibitors (forkhead translocation inhibitors), API-2/TCN (triciribine), RX-0201, enzastaurin HCl (LY317615), NL-71-101, SR-13668, PX-316, or KRX-0401 (perifosine/NSC 639966);
(xix) Hsp90 inhibitors for example onalespib (AT13387), herbimycin, geldanamycin (GA), 17-allylamino-17-desmethoxygeldanamycin (17-AAG) e.g. NSC-330507, Kos-953 and CNF-1010, 17-dimethylaminoethylamino-17-demethoxygeldanamycin hydrochloride (17-DMAG) e.g. NSC-707545 and Kos-1022, NVP-AUY922 (VER-52296), NVP-BEP800, CNF-2024 (BIIB-021 an oral purine), ganetespib (STA-9090), SNX-5422 (SC-102112) or IPI-504;
(xx) Monoclonal Antibodies (unconjugated or conjugated to radioisotopes, toxins or other agents), antibody derivatives and related agents, such as anti-CD, anti-VEGFR, anti-HER2 or anti-EGFR antibodies, for example rituximab (CD20), ofatumumab (CD20), ibritumomab tiuxetan (CD20), GA101 (CD20), tositumomab (CD20), epratuzumab (CD22), lintuzumab (CD33), gemtuzumab ozogamicin (CD33), alemtuzumab (CD52), galiximab (CD80), trastuzumab (HER2 antibody), pertuzumab (HER2), trastuzumab-DM1 (HER2), ertumaxomab (HER2 and CD3), cetuximab (EGFR), panitumumab (EGFR), necitumumab (EGFR), nimotuzumab (EGFR), bevacizumab (VEGF), catumaxumab (EpCAM and CD3), abagovomab (CA125), farletuzumab (folate receptor), elotuzumab (CS1), denosumab (RANK ligand), figitumumab (IGF1R), CP751,871 (IGF1R), mapatumumab (TRAIL receptor), metMAB (met), mitumomab (GD3 ganglioside), naptumomab estafenatox (5T4), or siltuximab (1L6) or immunomodulating agents such as CTLA-4 blocking antibodies and/or antibodies against PD-1 and PD-L1 and/or PD-L2 for example ipilimumab (CTLA4), MK-3475 (pembrolizumab, formerly lambrolizumab, anti-PD-1), nivolumab (a anti-PD-1), BMS-936559 (anti-PD-L1), MPDL320 Å, AMP-514 or MED14736 (anti-PD-L1), or tremelimumab (formerly ticilimumab, CP-675,206, anti-CTLA-4); (xxi) Estrogen receptor antagonists or selective estrogen receptor modulators (SERMs) or inhibitors of estrogen synthesis, for example tamoxifen, fulvestrant, toremifene, droloxifene, faslodex, or raloxifene;
(xxii) Aromatase inhibitors and related drugs, such as exemestane, anastrozole, letrazole, testolactone aminoglutethimide, mitotane or vorozole;
(xxiii) Antiandrogens (i.e. androgen receptor antagonists) and related agents for example bicalutamide, nilutamide, flutamide, cyproterone, or ketoconazole;
(xxiv) Hormones and analogues thereof such as medroxyprogesterone, diethylstilbestrol (a.k.a. diethylstilboestrol) or octreotide;
(xxv) Steroids for example dromostanolone propionate, megestrol acetate, nandrolone (decanoate, phenpropionate), fluoxymestrone or gossypol,
(xxvi) Steroidal cytochrome P450 17alpha-hydroxylase-17,20-lyase inhibitor (CYP17), e.g. abiraterone;
(xxvii) Gonadotropin releasing hormone agonists or antagonists (GnRAs) for example abarelix, goserelin acetate, histrelin acetate, leuprolide acetate, triptorelin, buserelin, or deslorelin; (xxviii) Glucocorticoids, for example prednisone, prednisolone, dexamethasone;
(xxix) Differentiating agents, such as retinoids, rexinoids, vitamin D or retinoic acid and retinoic acid metabolism blocking agents (RAMBA) for example accutane, alitretinoin, bexarotene, or tretinoin;
(xxx) Farnesyltransferase inhibitors for example tipifarnib;
(xxxi) Chromatin targeted therapies such as histone deacetylase (HDAC) inhibitors for example sodium butyrate, suberoylanilide hydroxamide acid (SAHA), depsipeptide (FR 901228), dacinostat (NVP-LAQ824), R306465/JNJ-16241199, JNJ-26481585, trichostatin A, vorinostat, chlamydocin, A-173, JNJ-MGCD-0103, PXD-101, or apicidin;
(xxxii) Drugs targeting the ubiquitin-proteasome pathway including proteasome Inhibitors for example bortezomib, carfilzomib, CEP-18770, MLN-9708, or ONX-0912; NEDD8 inhibitors; HDM2 antagonist and deubiquitinases (DUBs);
(xxxiii) Photodynamic drugs for example porfimer sodium or temoporfin;
(xxxiv) Marine organism-derived anticancer agents such as trabectidin;
(xxxv) Radiolabelled drugs for radioimmunotherapy for example with a beta particle-emitting isotope (e.g., Iodine-131, Yittrium-90) or an alpha particle-emitting isotope (e.g., Bismuth-213 or Actinium-225) for example ibritumomab or Iodine tositumomab or alpha radium 223;
(xxxvi) Telomerase inhibitors for example telomestatin;
(xxxvii) Matrix metalloproteinase inhibitors for example batimastat, marimastat, prinostat or metastat; (xxxviii) Recombinant interferons (such as interferon-y and interferon a) and interleukins (e.g. interleukin 2), for example aldesleukin, denileukin diftitox, interferon alfa 2a, interferon alfa 2b, or peginterferon alfa 2b;
(xxxix) Selective immunoresponse modulators for example thalidomide, or lenalidomide;
(xl) Therapeutic Vaccines such as sipuleucel-T (Provenge) or OncoVex;
(xli) Cytokine-activating agents include Picibanil, Romurtide, Sizofiran, Virulizin, or Thymosin;
(xlii) Arsenic trioxide;
(xliii) Inhibitors of G-protein coupled receptors (GPCR) for example atrasentan;
(xliv) Enzymes such as L-asparaginase, pegaspargase, rasburicase, or pegademase;
(xlv) DNA repair inhibitors such as PARP inhibitors for example, olaparib, velaparib, iniparib, INO-1001, AG-014699, or ONO-2231;
(xlvi) Agonists of Death receptor (e.g. TNF-related apoptosis inducing ligand (TRAIL) receptor), such as mapatumumab (formerly HGS-ETR1), conatumumab (formerly AMG 655), PR095780, lexatumumab, dulanermin, CS-1008, apomab or recombinant TRAIL ligands such as recombinant Human TRAIL/Apo2 Ligand;
(xlvii) Immunotherapies such as immune checkpoint inhibitors; cancer vaccines and CAR-T cell therapy;
(xlviii) Regulators of Cell death (apoptosis) including Bcl-2 (B-cell lymphoma 2) antagonists such as venetoclax (ABT-199 or GDC-0199), ABT-737, ABT-263, TW-37, sabutoclax, obatoclax, and MIM1 and IAP antagonists including LCL-161 (Novartis), Debio-1143 (Debiopharma/Ascenta), AZD5582, Birinapant/TL-32711 (TetraLogic), CUDC-427/GDC-0917/RG-7459 (Genentech), JP1201 (Joyant), T-3256336 (Takeda), GDC-0152 (Genentech) or HGS-1029/AEG-40826 (HGS/Aegera);
(xlix) Prophylactic agents (adjuncts); i.e. agents that reduce or alleviate some of the side effects associated with chemotherapy agents, for example
   anti-emetic agents,
   agents that prevent or decrease the duration of chemotherapy-associated neutropenia and prevent complications that arise from reduced levels of platelets, red blood cells or white blood cells, for example interleukin-11 (e.g. oprelvekin), erythropoietin (EPO) and analogues thereof (e.g. darbepoetin alfa), colony-stimulating factor analogs such as granulocyte macrophage-colony stimulating factor (GM-CSF) (e.g. sargramostim), and granulocyte-colony stimulating factor (G-CSF) and analogues thereof (e.g. filgrastim, pegfilgrastim),
   agents that inhibit bone resorption such as denosumab or bisphosphonates e.g. zoledronate, zoledronic acid, pamidronate and ibandronate,
   agents that suppress inflammatory responses such as dexamethasone, prednisone, and prednisolone,
   agents used to reduce blood levels of growth hormone and IGF-I (and other hormones) in patients with acromegaly or other rare hormone-producing tumours, such as synthetic forms of the hormone somatostatin e.g. octreotide acetate,
   antidote to drugs that decrease levels of folic acid such as leucovorin, or folinic acid,
   agents for pain e.g. opiates such as morphine, diamorphine and fentanyl,
   non-steroidal anti-inflammatory drugs (NSAID) such as COX-2 inhibitors for example celecoxib, etoricoxib and lumiracoxib,
   agents for mucositis e.g. palifermin,
   agents for the treatment of side-effects including anorexia, cachexia, oedema or thromoembolic episodes, such as megestrol acetate.

In one embodiment the biomarkers of the invention can be used to select a patient for treatment with an MDM2 antagonist in combination with (i)-(xlix) above. In one embodiment the biomarker(s) of the invention can be used to select a patient to treat with an MDM2 antagonist in combination with recombinant interferons; DNA repair inhibitors such as PARP inhibitors; IAP antagonists; platinum compounds; alkylating agents; and/or radiation therapy.

In one embodiment the patient's tumour is determined not to be suitable for treatment with single agent MDM2 inhibitor due to the presence of normal or high levels of DDR pathway genes or gene products, and hence the patient could be treated with MDM2 inhibitor in combination with an additional agent that can be used to cause tumour sensitivity to an MDM2 antagonist. In one embodiment the patient's tumour is determined to be ATM, ATRX, BRCA1 and/or BRCA2 normal or high and/or MSI normal or low, and is treated with an MDM2 antagonist in combination with an additional anti-cancer agent. In one embodiment the patient's tumour is determined to have wildtype ATM, ATRX, BRCA1 and/or BRCA2 or and/or normal level or high levels of ATM, ATRX, BRCA1 and/or BRCA2 gene expression, and is treated with an MDM2 antagonist in combination with one or more of the agents listed_in (i)-(xlix) above.

In one embodiment the biomarker(s) of the invention can be used to select a patient for treatment with an MDM2 antagonist in combination with one or more of the agents listed in (i)-(xlix) above.

In one embodiment the biomarkers of the invention, can be used to select a patient for treatment with an MDM2 antagonist in combination with DNA-damaging agents such as chemotherapy and radiotherapy.

In one embodiment the biomarkers of the invention, can be used to select a patient for treatment with an MDM2 antagonist in combination with immune checkpoint inhibitors for the treatment of MSI-H tumours.

In one embodiment the biomarkers of the invention, can be used to select a patient for treatment with an MDM2 antagonist in combination with recombinant interferons (such as interferon-y and interferon a) and interleukins (e.g. interleukin 2), for example aldesleukin, denileukin diftitox, interferon alfa 2a, interferon alfa 2b, or peginterferon alfa 2b. In one embodiment the patient's tumour is determined to be have normal or high levels of DDR pathway genes or gene products and is treated with an MDM2 antagonist in combination with one or more recombinant interferons.

In one embodiment the biomarkers of the invention can be used to select a patient for treatment with an MDM2 antagonist in combination with DNA repair inhibitors such as PARP inhibitors for example, olaparib, velaparib, iniparib, INO-1001, AG-014699, or ONO-2231. In one embodiment the PARP inhibitor is selected from PARP inhibitors for example, olaparib, rucaparib, veliparib, iniparib, INO-1001, AG-014699, ONO-2231; or or talazoparib. In one embodiment the patient's tumour is determined to be have normal or high levels of DDR pathway genes or gene products and is treated with an MDM2 antagonist in combination with a PARP inhibitor. In one embodiment PARPi is niraparib, olaparib, rucaparib, veliparib, iniparib, INO-1001, AG-014699, ONO-2231; or talazoparib. In one embodiment PARPi is niraparib, olaparib, rucaparib, or talazoparib. In one embodiment, the PARPi is olaparib. In one embodiment, the PARPi is talazoparib. In one embodiment PARPi is stenoparib or pamiparib.

In one embodiment the biomarkers of the invention can be used to select a patient for treatment with an MDM2 antagonist in combination with IAP antagonists including LCL-161 (Novartis), Debio-1143 (xevinapant) (Debiopharma/Ascenta), AZD5582, Birinapant/TL-32711 (TetraLogic), CUDC-427/GDC-0917/RG-7459 (Genentech), JP1201 (Joyant), T-3256336 (Takeda), GDC-0152 (Genentech) or HGS-1029/AEG-40826 (HGS/Aegera). In one embodiment the IAP antagonist is, for example, selected from LCL-161 (Novartis), Debio-1143 (Debiopharma/Ascenta) (xevinapant), AZD5582, Birinapant/TL-32711 (TetraLogic), CUDC-427/GDC-0917/RG-7459 (Genentech), JP1201 (Joyant), T-3256336 (Takeda), GDC-0152 (Genentech), ASTX660 (tolinapant) and HGS-1029/AEG-40826 (HGS/Aegera), Debio-4028 and Ascentage IAP inhibitor, APG-1387. In one embodiment the patient's tumour is determined to be have normal or high levels of DDR pathway genes or gene products and is treated with an MDM2 antagonist in combination with an IAP antagonist.

In one embodiment the biomarkers of the invention, can be used to select a patient for treatment with an MDM2 antagonist in combination with platinum compounds, for example cisplatin (optionally combined with amifostine), carboplatin or oxaliplatin; alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU), and/or radiation therapy. In one embodiment the platinum compound is selected from, for example, cisplatin (optionally combined with amifostine), carboplatin, oxaliplatin, dicycloplatin, heptaplatin, lobaplatin, nedaplatin, satraplatin or triplatin tetranitrate, in particular cisplatin, carboplatin, and oxaliplatin. In one embodiment the alkylating agents, such as nitrogen mustards or nitrosourea, is selected from, for example, cyclophosphamide, chlorambucil, carmustine (BCNU), ambamustine, bendamustine, thiotepa, melphalan, treosulfan, lomustine (CCNU), busulfan, dacarbazine, estramustine, fotemustine, ifosfamide (optionally in combination with mesna), pipobroman, procarbazine, streptozocin, temozolomide, uracil, mechlorethamine, mechlorethamine oxide hydrochloride, methylcyclohexylchloroethylnitrosurea, nimustine (ACNU), prednimustine, meclorethamine, etoglucid; streptozotocin, irofulven, mitolactol, glufosfamide, evofosfamide, ethylenimines or methylamelamines including altretamine, triethylenemelamine, trimethylolomelamine, triethylenephosphoramide, triethylenethiophosphoramide, or trimemylolomelamine. In one embodiment the patient's tumour is determined to normal or high levels of DDR pathway genes or gene products and is treated with an MDM2 antagonist in combination with radiation therapy.

In one embodiment the patient's tumour is determined to be have normal or high levels of DDR pathway genes or gene products and is treated with an MDM2 antagonist in combination with DNA-damaging agents such as chemotherapy and/or radiotherapy.

In one embodiment the patient's tumour is determined to have normal or high levels of DDR pathway genes or gene products and is treated with an MDM2 antagonist in combination with immune checkpoint inhibitors, such as CTLA-4 blocking antibodies and/or antibodies against PD-1 and PD-L1 and/or PD-L2 for example ipilimumab (CTLA4), MK-3475 (pembrolizumab, formerly lambrolizumab, anti-PD-1), nivolumab (an anti-PD-1), BMS-936559 (anti-PD-L1), MPDL320 Å, AMP-514 or MEDI4736 (anti-PD-L1), or tremelimumab (formerly ticilimumab, CP-675,206, anti-CTLA-4); optionally for the treatment of MSI-H tumours, which can identified by microsatellite instability (MSI) testing.

In another embodiment there is provided a method of treating cancer in a patient wherein said method comprises the steps of selecting a patient:
(a) having normal or high levels of a DDR biomarker within a biological sample obtained from said patient; and
(b) administering, to said patient selected in step (a), a therapeutically effective amount of an MDM2 antagonist and an agent to induce sensitivity to an MDM2 antagonist for example by lowering the levels of a DDR biomarker.

In one embodiment the agent or treatment to lower the levels of a DDR biomarker is an anticancer agent or treatment. In one embodiment the agent or treatment to lower the levels of a DDR biomarker is recombinant interferons (such as interferon-y and interferon a) and interleukins (e.g. interleukin 2), for example aldesleukin, denileukin diftitox, interferon alfa 2a, interferon alfa 2b, or peginterferon alfa 2b, or DNA repair inhibitors such as PARP inhibitors, or IAP antagonists or platinum compounds, for example cisplatin (optionally combined with amifostine), carboplatin or oxaliplatin; alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU), and/or radiation therapy.

In one embodiment the agent or treatment to induce sensitivity, is recombinant interferons and interleukins, DNA repair inhibitors, IAP antagonists or platinum compounds. In one embodiment the agent or treatment to induce sensitivity is IAP antagonist.

In one embodiment the agent or treatment to trigger apoptosis is an IAP antagonist. In one embodiment the IAP antagonist is LCL-161 (Novartis), Debio-1143 (Debiopharma/Ascenta), AZD5582, Birinapant/TL-32711 (TetraLogic), CUDC-427/GDC-0917/RG-7459 (Genentech), JP1201 (Joyant), T-3256336 (Takeda), GDC-0152 (Genentech) or HGS-1029/AEG-40826 (HGS/Aegera).

In one embodiment the IAP antagonist is ASTX660, LCL-161 (Novartis), Debio-1143 (Debiopharma/Ascenta), AZD5582, Birinapant/TL-32711 (TetraLogic), CUDC-427/GDC-0917/RG-7459 (Genentech), JP1201 (Joyant), T-3256336 (Takeda), GDC-0152 (Genentech) or HGS-1029/AEG-40826 (HGS/Aegera), Debio-4028 and Ascentage IAP inhibitor, APG-1387. In one embodiment the IAP antagonist is ASTX660 (tolinapant). In one embodiment the invention relates to a combination of an MDM2 antagonist e.g. (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid and ASTX660.

In one aspect, the invention provides a combination of
(i) (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid (“the isoindolin-1-one compound”) or a tautomer or a solvate or a pharmaceutically acceptable salt thereof; and
(ii) 1-{6-[(4-fluorophenyl)methyl]-5-(hydroxymethyl)-3,3-dimethyl-1H,2H,3H-pyrrolo[3,2-b]pyridin-1-yl}-2-[(2R,5R)-5-methyl-2-{[(3R)-3-methylmorpholin-4-yl]methyl}piperazin-1-yl]ethan-1-one (“ASTX660”) or a tautomer or a solvate or a pharmaceutically acceptable salt thereof.

In particular, this aspect of the invention provides: A combination comprising a combination as disclosed herein (e.g. a combination of the isoindolin-1-one compound or a tautomer or a solvate or a pharmaceutically acceptable salt thereof and ASTX660 or a tautomer or a solvate or a pharmaceutically acceptable salt thereof) and optionally one or more (e.g. 1 or 2) other therapeutic agents (e.g. anticancer agents).

A combination as disclosed herein comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof, wherein the isoindolin-1-one compound or a tautomer or a solvate or a pharmaceutically acceptable salt thereof and the additional therapeutic agent e.g. ASTX660 or a tautomer or a solvate or a pharmaceutically acceptable salt thereof are physically associated.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof as disclosed herein wherein the isoindolin-1-one compound or a tautomer or a solvate or a pharmaceutically acceptable salt thereof and the additional therapeutic agent e.g. ASTX660 or a tautomer or a solvate or a pharmaceutically acceptable salt thereof are: (a) in admixture; (b) chemically/physicochemically linked; (c) chemically/physicochemically co-packaged; or (d) unmixed but co-packaged or co-presented.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof as disclosed herein wherein the isoindolin-1-one compound or a tautomer or a solvate or a pharmaceutically acceptable salt thereof and the therapeutic agent e.g. ASTX660 or a tautomer or a solvate or a pharmaceutically acceptable salt thereof are non-physically associated.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof as disclosed herein wherein the combination comprises: (a) at least one of the two or more compounds together with instructions for the extemporaneous association of the at least one compound to form a physical association of the two or more compounds; or (b) at least one of the two or more compounds together with instructions for combination therapy with the two or more compounds; or (c) at least one of the two or more compounds together with instructions for administration to a patient population in which the other(s) of the two or more compounds have been (or are being) administered; or (d) at least one of the two or more compounds in an amount or in a form which is specifically adapted for use in combination with the other(s) of the two or more compounds.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof as disclosed herein in the form of a pharmaceutical kit or patient pack.

A pharmaceutical composition comprising a combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof as disclosed herein.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination as disclosed herein for use in therapy.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination as disclosed herein for use in the prophylaxis or treatment of a disease state or condition as described herein.

A use of a combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination as disclosed herein for the manufacture of a medicament for use in the prophylaxis or treatment of a disease state or condition as described herein.

A method for the prophylaxis or treatment of a disease or condition as described herein comprising administering to a patient a combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination as disclosed herein.

A method for the prophylaxis or treatment of a disease or condition as described herein, comprising administering to patient in need thereof (i) the additional therapeutic agent e.g. ASTX660, or a tautomer, N-oxide, pharmaceutically acceptable salt or solvate thereof and (ii) the isoindolin-1-one compound as defined herein, or a tautomer, N-oxide, pharmaceutically acceptable salt or solvate thereof.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination for use as disclosed herein, in particular for use in a method for the prophylaxis or treatment as disclosed herein, wherein the disease state or condition is mediated by MDM2-p53.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination for use as disclosed herein, or a method for the prophylaxis or treatment using the combination as disclosed herein, wherein patient is selected according the biomarkers described herein.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination for use as disclosed herein, or a method for the prophylaxis or treatment using the combination as disclosed herein, wherein patient is selected as having a tumour which is DDR normal or high.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination for use as disclosed herein, or a method for the prophylaxis or treatment using the combination as disclosed herein, wherein the disease state or condition is cancer.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof or a pharmaceutical composition comprising the combination for use as disclosed herein, or a method for the prophylaxis or treatment using the combination as disclosed herein, wherein the disease state or condition is a cancer which is acute myeloid leukaemia.

A combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof as disclosed herein for use as disclosed herein for the prophylaxis or treatment of acute myeloid leukaemia.

The isoindolin-1-one compound, or a tautomer, N-oxide, pharmaceutically acceptable salt or solvate thereof, for use in the prophylaxis or treatment of a disease state or condition as described herein, wherein the isoindolin-1-one compound is used in combination with an additional therapeutic agent e.g.

ASTX660, or a tautomer, N-oxide, pharmaceutically acceptable salt or solvate thereof.

The isoindolin-1-one compound or a tautomer, N-oxide, pharmaceutically acceptable salt or solvate thereof, for use in the prophylaxis or treatment of a cancer as described herein, wherein the isoindolin-1-one compound is used in combination with an additional therapeutic agent e.g. ASTX660, or a tautomer, N-oxide, pharmaceutically acceptable salt or solvate thereof.

ASTX660, or a tautomer, N-oxide, pharmaceutically acceptable salt or solvate thereof, for use in the prophylaxis or treatment of a disease state or condition as described herein, wherein the therapeutic agent is used in combination with the isoindolin-1-one compound, or a tautomer, N-oxide, pharmaceutically acceptable salt or solvate thereof.

The isoindolin-1-one compound, or a tautomer, N-oxide, pharmaceutically acceptable salt or solvate thereof, for use in preventing, treating or managing cancer in a patient in need thereof in combination therapy with an additional therapeutic agent e.g. ASTX660 or a tautomer or a solvate or a pharmaceutically acceptable salt thereof, and optionally with one or more other therapeutic agents.

The use of the isoindolin-1-one compound, or a tautomer, N-oxide, pharmaceutically acceptable salt or solvate thereof, for the manufacture of a medicament for the treatment of a cancer where the patient is being treated with another therapeutic agent e.g. ASTX660, or a tautomer, N-oxide, pharmaceutically acceptable salt or solvate thereof.

The use of a therapeutic agent e.g. ASTX660, or a tautomer, N-oxide, pharmaceutically acceptable salt or solvate thereof, for the manufacture of a medicament for the treatment of a cancer where the patient is being treated with the isoindolin-1-one compound, or a tautomer, N-oxide, pharmaceutically acceptable salt or solvate thereof, as disclosed herein.

The use of the isoindolin-1-one compound, or a tautomer, N-oxide, pharmaceutically acceptable salt or solvate thereof, for the manufacture of a medicament for use in enhancing or potentiating the response rate in a patient suffering from a cancer where the patient is being treated with another therapeutic agent e.g. ASTX660, or a tautomer, N-oxide, pharmaceutically acceptable salt or solvate thereof.

The isoindolin-1-one compound, or a tautomer, N-oxide, pharmaceutically acceptable salt or solvate thereof, for use in treating a disease or condition comprising or arising from abnormal cell growth in a mammal, wherein the mammal is undergoing treatment with another therapeutic agent e.g. ASTX660 or a tautomer, N-oxide, pharmaceutically acceptable salt or solvate thereof.

The isoindolin-1-one compound, or a tautomer, N-oxide, pharmaceutically acceptable salt or solvate thereof, for use in alleviating or reducing the incidence of a disease or condition comprising or arising from abnormal cell growth in a mammal, wherein the mammal is undergoing treatment with another therapeutic agent e.g. ASTX660, or a tautomer, N-oxide, pharmaceutically acceptable salt or solvate thereof, The use of a combination as disclosed herein (e.g a combination comprising the isoindolin-1-one compound or a tautomer or a solvate or a pharmaceutically acceptable salt thereof and an additional therapeutic agent e.g. ASTX660 or a tautomer or a solvate or a pharmaceutically acceptable salt thereof) in the manufacture of a pharmaceutical composition for inhibiting the growth of tumour cells.

A product containing as a first active ingredient the isoindolin-1-one compound, or a tautomer, N-oxide, pharmaceutically acceptable salt or solvate thereof, and as a further active ingredient an additional therapeutic agent e.g. ASTX660, or a tautomer, N-oxide, pharmaceutically acceptable salt or solvate thereof, as a combined preparation for simultaneous, separate or sequential use in the treatment of cancer.

In one embodiment the additional therapeutic agent used in combination is an agent or treatment to lower the levels of one or more DDR pathway gene products. In one embodiment the agent or treatment to lower the levels of one or more DDR pathway gene products is Recombinant interferons (such as interferon-y and interferon a) and interleukins (e.g. interleukin 2), for example aldesleukin, denileukin diftitox, interferon alfa 2a, interferon alfa 2b, or peginterferon alfa 2b, or DNA repair inhibitors such as PARP inhibitors, or IAP antagonists or platinum compounds, for example cisplatin (optionally combined with amifostine), carboplatin or oxaliplatin; alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU), and/or radiation therapy.

In one embodiment the agent to lower the levels of one or more DDR pathway gene products is an inhibitor of BRCA1, BRCA2, ATM and/or ATRX.

In one embodiment the MDM2 antagonist is used in combination with an ATM inhibitor, for example an ATM inhibitor selected from AZD-1390, M-4076 (from Merck KGaA) or IMP-08 (from IMPACT Therapeutics).

In one embodiment the MDM2 antagonist is used in combination with an ATRX modulator.

In one embodiment the MDM2 antagonist is used in combination with imatinib, nilotinib, dasatinib, flumatinib, asciminib, bosutinib, ponatinib, or radotinib.

A specific process for preparing, isolating and purifying 1-{6-[(4-fluorophenyl)methyl]-5-(hydroxymethyl)-3,3-dimethyl-1 H,2H,3H-pyrrolo[3,2-b]pyridin-1-yl}-2-[(2R,5R)-5-methyl-2-{[(3R)-3-methylmorpholin-4-yl]methyl}piperazin-1-yl]ethan-1-one (ASTX660) and pharmaceutically acceptable salts thereof including the lactate salt can be found at Example 2 in international patent application no PCT/GB2014/053778 which was published as WO 2015/092420 on 25.06.2015. in one embodiment it is the lactate salt of 1-{6-[(4-fluorophenyl)methyl]-5-(hydroxymethyl)-3,3-dimethyl-1 H,2H,3H-pyrrolo[3,2-b]pyridin-1-yl}-2-[(2R,5R)-5-methyl-2-{[(3R)-3-methylmorpholin-4-yl]methyl}piperazin-1-yl]ethan-1-one.

Each of the compounds present in the combinations of the invention may be given in individually varying dose schedules and via different routes. As such, the posology of each of the two or more agents may differ: each may be administered at the same time or at different times. A person skilled in the art would know through his or her common general knowledge the dosing regimes and combination therapies to use. For example, the compound of formula (I°) may be used in combination with one or more other agents which are administered according to their existing combination regimen. Examples of standard combination regimens are provided below.

The taxane compound is advantageously administered in a dosage of 50 to 400 mg per square meter (mg/m²) of body surface area, for example 75 to 250 mg/m², particularly for paclitaxel in a dosage of about 175 to 250 mg/m² and for docetaxel in about 75 to 150 mg/m² per course of treatment.

The camptothecin compound is advantageously administered in a dosage of 0.1 to 400 mg per square meter (mg/m²) of body surface area, for example 1 to 300 mg/m², particularly for irinotecan in a dosage of about 100 to 350 mg/m² and for topotecan in about 1 to 2 mg/m² per course of treatment.

The anti-tumour podophyllotoxin derivative is advantageously administered in a dosage of 30 to 300 mg per square meter (mg/m²) of body surface area, for example 50 to 250 mg/m², particularly for etoposide in a dosage of about 35 to 100 mg/m² and for teniposide in about 50 to 250 mg/m² per course of treatment.

The anti-tumour vinca alkaloid is advantageously administered in a dosage of 2 to 30 mg per square meter (mg/m²) of body surface area, particularly for vinblastine in a dosage of about 3 to 12 mg/m², for vincristine in a dosage of about 1 to 2 mg/m², and for vinorelbine in dosage of about 10 to 30 mg/m² per course of treatment.

The anti-tumour nucleoside derivative is advantageously administered in a dosage of 200 to 2500 mg per square meter (mg/m²) of body surface area, for example 700 to 1500 mg/m², particularly for 5-FU in a dosage of 200 to 500 mg/m², for gemcitabine in a dosage of about 800 to 1200 mg/m² and for capecitabine in about 1000 to 2500 mg/m² per course of treatment.

The alkylating agents such as nitrogen mustard or nitrosourea is advantageously administered in a dosage of 100 to 500 mg per square meter (mg/m²) of body surface area, for example 120 to 200 mg/m², particularly for cyclophosphamide in a dosage of about 100 to 500 mg/m², for chlorambucil in a dosage of about 0.1 to 0.2 mg/kg, for carmustine in a dosage of about 150 to 200 mg/m², and for Iomustine in a dosage of about 100 to 150 mg/m² per course of treatment.

The anti-tumour anthracycline derivative is advantageously administered in a dosage of 10 to 75 mg per square meter (mg/m²) of body surface area, for example 15 to 60 mg/m², particularly for doxorubicin in a dosage of about 40 to 75 mg/m², for daunorubicin in a dosage of about 25 to 45 mg/m², and for idarubicin in a dosage of about 10 to 15 mg/m² per course of treatment.

The antiestrogen agent is advantageously administered in a dosage of about 1 to 100 mg daily depending on the particular agent and the condition being treated. Tamoxifen is advantageously administered orally in a dosage of 5 to 50 mg, typically 10 to 20 mg twice a day, continuing the therapy for sufficient time to achieve and maintain a therapeutic effect. Toremifene is advantageously administered orally in a dosage of about 60 mg once a day, continuing the therapy for sufficient time to achieve and maintain a therapeutic effect. Anastrozole is advantageously administered orally in a dosage of about 1 mg once a day. Droloxifene is advantageously administered orally in a dosage of about 20-100 mg once a day. Raloxifene is advantageously administered orally in a dosage of about 60 mg once a day. Exemestane is advantageously administered orally in a dosage of about 25 mg once a day.

Antibodies are advantageously administered in a dosage of about 1 to 5 mg per square meter (mg/m²) of body surface area, or as known in the art, if different. Trastuzumab is advantageously administered in a dosage of 1 to 5 mg per square meter (mg/m²) of body surface area, particularly 2 to 4 mg/m² per course of treatment.

Where the compound of the formula (I°) is administered in combination therapy with one, two, three, four or more other therapeutic agents (typically one or two, more typically one), the compounds can be administered simultaneously or sequentially. In the latter case, the two or more compounds will be administered within a period and in an amount and manner that is sufficient to ensure that an advantageous or synergistic effect is achieved. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s). These dosages may be administered for example once, twice or more per course of treatment, which may be repeated for example every 7, 14, 21 or 28 days.

It will be appreciated that the typical method and order of administration and the respective dosage amounts and regimes for each component of the combination will depend on the particular other medicinal agent and compound of the present invention being administered, their route of administration, the particular tumour being treated and the particular host being treated. The optimum method and order of administration and the dosage amounts and regime can be readily determined by those skilled in the art using conventional methods and in view of the information set out herein.

The weight ratio of the compound according to the present invention and the one or more other anticancer agent(s) when given as a combination may be determined by the person skilled in the art.

Said ratio and the exact dosage and frequency of administration depends on the particular compound according to the invention and the other anticancer agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present MDM2 antagonists and another anticancer agent may range from 1/10 to 10/1, more in particular from 1/5 to 5/1, even more in particular from 1/3 to 3/1.

The compounds of the invention may also be administered in conjunction with non-chemotherapeutic treatments such as radiotherapy, photodynamic therapy, gene therapy; surgery and controlled diets.

Radiotherapy may be for radical, palliative, adjuvant, neoadjuvant or prophylactic purposes.

The compounds for use in the present invention also have therapeutic applications in sensitising tumour cells for radiotherapy and chemotherapy. Hence the compounds of the present invention can be used as “radiosensitizer” and/or “chemosensitizer” or can be given in combination with another “radiosensitizer” and/or “chemosensitizer”. In one embodiment the compound of formula (I°) is for use as chemosensitiser.

The term “radiosensitizer” is defined as a molecule administered to patients in therapeutically effective amounts to increase the sensitivity of the cells to ionizing radiation and/or to promote the treatment of diseases which are treatable with ionizing radiation.

The term “chemosensitizer” is defined as a molecule administered to patients in therapeutically effective amounts to increase the sensitivity of cells to chemotherapy and/or promote the treatment of diseases which are treatable with chemotherapeutics.

Many cancer treatment protocols currently employ radiosensitizers in conjunction with radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin, benzoporphyrin derivatives, tin etioporphyrin, pheoborbide-a, bacteriochlorophyll-a, naphthalocyanines, phthalocyanines, zinc phthalocyanine, and therapeutically effective analogs and derivatives of the same.

Radiosensitizers may be administered in conjunction with a therapeutically effective amount of one or more other compounds, including but not limited to: compounds which promote the incorporation of radiosensitizers to the target cells; compounds which control the flow of therapeutics, nutrients, and/or oxygen to the target cells; chemotherapeutic agents which act on the tumour with or without additional radiation; or other therapeutically effective compounds for treating cancer or other diseases.

Chemosensitizers may be administered in conjunction with a therapeutically effective amount of one or more other compounds, including but not limited to: compounds which promote the incorporation of chemosensitizers to the target cells; compounds which control the flow of therapeutics, nutrients, and/or oxygen to the target cells; chemotherapeutic agents which act on the tumour or other therapeutically effective compounds for treating cancer or other disease. Calcium antagonists, for example verapamil, are found useful in combination with antineoplastic agents to establish chemosensitivity in tumour cells resistant to accepted chemotherapeutic agents and to potentiate the efficacy of such compounds in drug-sensitive malignancies.

For use in combination therapy with another chemotherapeutic agent, the compound of the formula (I°) and one, two, three, four or more other therapeutic agents can be, for example, formulated together in a dosage form containing two, three, four or more therapeutic agents i.e. in a unitary pharmaceutical composition containing all components. In an alternative, the individual therapeutic agents may be formulated separately and presented together in the form of a kit, optionally with instructions for their use.

In one embodiment the pharmaceutical composition comprises a compound of formula (I°) together with a pharmaceutically acceptable carrier and optionally one or more therapeutic agent(s) In another embodiment the invention relates to the use of a combination according to the invention in the manufacture of a pharmaceutical composition for inhibiting the growth of tumour cells.

In a further embodiment the invention relates to a product containing a compound of formula (I°) and one or more anticancer agent, as a combined preparation for simultaneous, separate or sequential use in the treatment of patients suffering from cancer.

### Numbered Embodiments

The invention includes at least the following numbered embodiments:
1. An MDM2 antagonist for use in a method of treating a cancer, wherein the cancer is depleted of one or more genes or gene products in one or more DNA damage repair (DDR) pathways, or wherein the cancer has at least one loss of function mutation in at least one DDR pathway gene.
2. An MDM2 antagonist for use in a method according to embodiment 1, wherein the DDR pathway is:
   a. the homologous recombination repair (HRR) pathway;
   b. the non-homologous end joining (NHEJ) pathway;
   c. the mismatch repair (MMR) pathway;
   d. the Fanconi Anemia (FA) pathway; and/or e. the base excision repair (BER) pathway.
3. An MDM2 antagonist for use in a method according to embodiment 1 or embodiment 2, wherein: the one or more genes or gene products comprise or consist of HRR pathway genes or gene products other than ATM; or the one or more genes or gene products comprise or consist of BRCA1, BRCA2 and/or ATM.
4. An MDM2 antagonist for use in a method according to any of embodiments 1 to 3, wherein the one or more genes or gene products comprise or consist of ATRX.
5. An MDM2 antagonist for use in a method according to any of embodiments 1 to 4, wherein: the one or more genes or gene products comprise or consist of MSH2, MSH3, MSH6, MLH1, MLH3, PMS2, POLE and/or POLD1; or the cancer comprises mutational signature SBS6 or SBS26 associated with defects in DNA mismatch repair, and/or the POLD1 mutational signature SBS20.
6. An MDM2 antagonist for use in a method according to any of embodiments 1 to 5, wherein the one or more genes or gene products comprise or consist of FANCA, FANCB, FANCC, FANCD1, FANCD2, FANCE, FANCF, FANCG, FANCI, FANCJ, FANCL, FANCM, FANCN, FANCO, FANCP, FANCQ, FANCR, FANCS, FANCT, FANCU, FANCV and/or FANCW.
7. An MDM2 antagonist for use in a method according to any preceding embodiment, wherein the depletion or mutation in a DDR gene is detected by assessing the microsatellite instability status and/or tumour mutational burden of the cancer, optionally wherein the cancer is MSI-high.
8. An MDM2 antagonist for use according to any of embodiments 1 to 7, wherein a sample of patient tissue is tested to determine the cancer expression profile prior to treatment.
9. An MDM2 antagonist for use according to embodiment 8, wherein the sample comprises cancer DNA, ctDNA, or cancer cells.
10. An MDM2 antagonist for use according to embodiment 8 or embodiment 9, wherein the testing comprises an assay to detect protein, mRNA and/or ctDNA.
11. An MDM2 antagonist for use according to embodiment 10, wherein (i) protein is detected using an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, a radioimmunoassay, immunofluorescence, immunochemiluminescence, immunoelectrochemiluminescence, immunoelectrophoretic, a competitive immunoassay, or immunoprecipitation; and/or (ii) wherein mRNA is detected using RT-PCR or a quantitative gene expression assay.
12. An MDM2 antagonist for use according to any of embodiments 8 to 11, wherein the patient is selected for treatment based on the determined expression profile.
13. An MDM2 antagonist for use according to any preceding embodiment, wherein the cancer is: non-small-cell lung carcinoma, mesothelioma, glioblastoma or Kidney Renal Clear Cell Carcinoma; or Uterine, endometrial, bladder, gastric, colorectal, prostate cancer, or DLBCL; or brain, clear cell renal cell carcinoma (ccRCC), oesophageal cancer or melanoma.
14. An MDM2 antagonist for use according to any preceding embodiment, wherein the cancer is P53 wild-type.
15. An MDM2 antagonist for use according to any preceding embodiment, wherein the cancer cells undergo apoptosis following the treatment step.
16. An MDM2 antagonist for use according to any preceding embodiment, wherein activated caspase-3 is induced by the MDM2 antagonist in at least a proportion of the cancer cells.
17. An MDM2 antagonist for use according to embodiment 16, wherein activated caspase-3 is induced by the MDM2 antagonist in at least 40% of the cancer cells or at least 60% of the cancer cells.
18. An MDM2 antagonist for use according to any preceding embodiment, wherein the cancer shows:
   decreased expression, relative to a control, of one, two or three of CDKN2 Å, BAP1 and SKP2; and/or
   increased expression, relative to a control, of one, two, three, four, five or more of the interferon signature genes.
19. An MDM2 antagonist for use according to embodiment 18, wherein:
   the interferon signature genes are CXCL10, CXCL11, RSAD2, MX1, BATF2, IF144L, IFITM1, ISG15, CMPK2, IF127, CD74, IFIH1, CCRL2, IF144, HERC6, ISG20, IFIT3, HLA-C, OAS1, IF135, IRF9, EPST11, USP18, BST2, CSF1, C1S, DHX58, TRIM14, OASL, IRF7, LGALS3BP, DDX60, LAP3, LAMP3, PARP12, PARP9, SP110, PLSCR1, WARS, STAT1, IRF3, IRF5, MSC, JUN, SP11, IRF1, COMMD3-BM11, STAT2, RUNX3, SREBF1 and FLI1; or the cancer shows increased expression of CXCL10 or CXCL11.
20. An MDM2 antagonist for use according to any preceding embodiment, wherein the cancer shows increased expression of one, two, three, four, five or more of IRF7, STAT1, IRF3, IRF5, MSC, JUN, SP11, IRF1, COMMD3-BM11, STAT2, RUNX3, SREBF1, IRF9 and FLI1.
21. An MDM2 antagonist for use according to any preceding embodiment, wherein the MDM2 antagonist is a compound of formula (I°) or a tautomer, N-oxide, pharmaceutically acceptable salt or solvate thereof as defined herein, for example (2S,3S)-3-(4-chlorophenyl)-3-[(1R)-1-(4-chlorophenyl)-7-fluoro-5-[(1 S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof.
22. An MDM2 antagonist for use according to any preceding embodiment, wherein the MDM2 antagonist is selected from the group consisting of idasanutlin (RG-7388), HDM-201, KRT-232 (AMG-232), ALRN-6924, MI-773 (SAR405838), CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

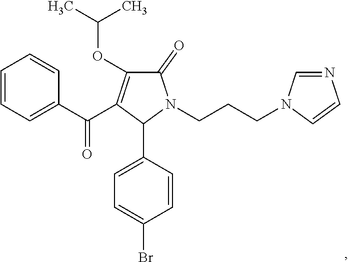 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.
23. Use of the expression or activity level of one or more in one or more DDR pathway genes or gene products in a cancer cell sample of a human patient, as a biomarker or biomarkers for assessing whether the cancer is susceptible to treatment with an MDM2 antagonist, for example wherein the MDM2 antagonist is a compound of formula (I°) or a tautomer, N-oxide, pharmaceutically acceptable salt or solvate thereof as defined herein, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof.
24. A method for prognosing or assessing the responsiveness of a human cancer patient to treatment with an MDM2 antagonist, comprising assessing the expression or activity level in a sample from a cancer patient of one or more DDR pathway genes, and determining whether the tested expression or activity level indicates that the cancer should be treated with an MDM2 antagonist.
25. A method according to embodiment 24, wherein the assessment step comprises comparing the expression or activity level with the expression or activity level (i) associated with responsiveness or non-responsiveness to treatment with an MDM2 antagonist or (ii) from a healthy non-cancer cell of the same type.
26. A method according to embodiment 24 or embodiment 25, wherein the patient is classified into a group based on the biomarker profile, optionally wherein the groups comprise or consist of: (i) responders and non-responders; or
(ii) strong responders.
27. A method according to any of embodiments 24 to 26, wherein a patient is identified as particularly suitable for treatment when 1, 2, 3, 4, 5, 6, 7, 8, 9, 10 or more DDR pathway genes are expressed at a lower level than in a patient identified as not suitable for treatment.
28. A method according to any of embodiments 24 to 27, wherein the patient is identified for treatment with the MDM2 antagonist when decreased expression of one or DDR pathway genes is detected, relative to the expression level (i) associated with non-responsiveness to treatment with an MDM2 antagonist or (ii) from a healthy non-cancer cell of the same type.
29. A method according to any of embodiments 24 to 28, comprising the step of detecting the expression or activity level of the biomarkers in a sample of cancer cells from said human patient.
30. A method according to embodiment 29, wherein the detection is carried out using an in vitro detection assay.
31. A method of determining the susceptibility of a human cancer patient to treatment with an MDM2 antagonist, comprising detecting in a sample of cancer cells from the patient the expression or activity of one or more DDR pathway genes, and assessing whether the cancer in the patient is likely to respond to treatment with a MDM2 antagonist on the basis of the expression or activity level of the biomarkers in the sample.
32. A method of detecting the expression or activity level of one or more DDR pathway genes in a human patient suffering from cancer.
33. A method according to embodiment 32, comprising the steps of:
   (a) obtaining a sample of cancer cells from a human patient; and
   (b) detecting whether said biomarker or biomarkers are expressed in the sampled cancer cells by contacting the sample with one or more reagents for detecting expression of the biomarker or biomarkers.
34. A method according to any of embodiments 24 to 33, wherein the MDM2 antagonist is a compound of formula (I°) or a tautomer, N-oxide, pharmaceutically acceptable salt or solvate thereof as defined herein, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof.
35. A method according to any of embodiments 24 to 33, wherein the MDM2 antagonist is selected from the group consisting of idasanutlin, HDM-201, KRT-232, ALRN-6924, ALRN-6924, CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

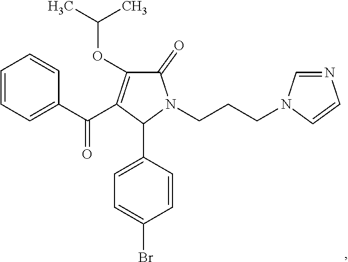 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.
36. A method according to any of embodiments 24 to 35, further comprising the step of treating the cancer in the patient by administering an MDM2 antagonist.
37. A method according to embodiment 36, wherein the MDM2 antagonist is a compound of formula (I°) or a tautomer, N-oxide, pharmaceutically acceptable salt or solvate thereof as defined herein, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof.
38. A method according to embodiment 36, wherein the MDM2 antagonist is selected from the group consisting of idasanutlin, HDM-201, KRT-232, ALRN-6924, ALRN-6924, CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

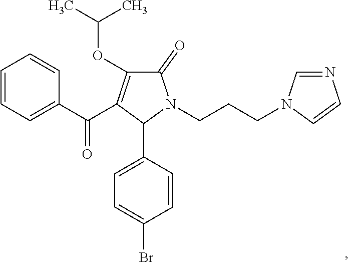 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.
39. A method according to any one of embodiments 36 to 38, wherein the treatment is provided to the patient based on the outcome of the method.
40. A kit or device for detecting the expression or activity level of at least one biomarkers for sensitivity to MDM2 inhibition in a sample from a human patient, comprising detection reagents for detecting one or more DDR pathway genes or gene products.
41. A system for determining the suitability of a human cancer patient for treatment with an MDM2 antagonist, comprising a storage memory for storing data associated with a sample from the patient comprising data associated with a panel of biomarkers indicating biomarker expression or activity levels in the sample from the subject, the panel of biomarkers comprising one or more DDR pathway genes or gene products; and a processor communicatively coupled to the storage memory for classifying the patient.
42. An MDM2 antagonist for use, use, method, kit or system according to any preceding embodiment, wherein the cancer shows loss of one or more DDR pathway genes, gene products or activities.
43. An MDM2 antagonist for use, use, or method according to any of embodiments 1 to 39 or 42, wherein the MDM2 antagonist is part of a combination therapy with a second therapeutic agent.
44. An MDM2 antagonist for use in a method of treating a cancer,
   wherein the cancer has normal or high levels of one or more genes or gene products in one or more DNA damage repair (DDR) pathways, or wherein the cancer has no detectable loss of function mutation in any DDR pathway gene,
      in combination with an agent to induce sensitivity to an MDM2 antagonist for example to lower the levels of one or more genes or gene products in a DNA damage repair (DDR) pathway.
45. A method of treating cancer in a patient wherein said method comprises the steps of selecting a patient:
   (a) having normal or high levels of DDR pathway genes or gene products within a biological sample obtained from said patient; and
   (b) administering a therapeutically effective amount of an MDM2 antagonist and an agent to induce sensitivity to an MDM2 antagonist for example by lowering the levels of one or more genes or gene products in a DNA damage repair (DDR) pathway, to said patient selected in step (a).
46. An MDM2 antagonist according to embodiment 44 or a method according to embodiment 45, wherein the agent to induce sensitivity to an MDM2 antagonist is a DNA-damaging agent or a DNA repair inhibitor.
47. A pharmaceutical composition comprising an MDM2 inhibitor, wherein the MDM2 inhibitor is a compound of formula (I°) or a tautomer, N-oxide, pharmaceutically acceptable salt or solvate thereof, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof, for use in the treatment of cancer in a patient, wherein the cancer is as defined in any of embodiments 1 to 7.
48. An MDM2 antagonist for use in a method of treating a patient with cancer, wherein the method comprises:
   (i) determining that a sample from the patient is depleted of one or more genes or gene products in one or more DNA damage repair (DDR) pathways, or wherein the cancer has at least one loss of function mutation in a DDR pathway gene:
   (ii) administering an effective amount of the MDM2 antagonist to the patient.
49. An MDM2 antagonist for use in a method of treating a cancer, wherein the cancer has low levels of one or more genes or gene products in one or more DNA damage repair (DDR) pathways, or wherein the cancer has detectable loss of function mutation in any DDR pathway gene, in combination with an anticancer agent e.g. a DNA-damaging agent or a DNA repair inhibitor.
50. A method of treating cancer in a patient wherein said method comprises the steps of selecting a patient:
   (a) having low levels of DDR pathway genes or gene products within a biological sample obtained from said patient; and
   (b) administering a therapeutically effective amount of an MDM2 antagonist and an anticancer agent e.g a DNA-damaging agent or a DNA repair inhibitor, to said patient selected in step (a).
51. A method of treating a cancer in a subject, comprising administering an MDM2 antagonist to the subject, wherein the cancer is depleted of one or more genes or gene products in one or more DNA damage repair (DDR) pathways, or wherein the cancer has at least one loss of function mutation in at least one DDR pathway gene, optionally wherein the one or more DDR pathway genes or gene products comprise BRCA1 and/or BRCA2.
52. The method of embodiment 51, wherein the DDR pathway is:
   a. the homologous recombination repair (HRR) pathway;
   b. the non-homologous end joining (NHEJ) pathway;
   c. the mismatch repair (MMR) pathway;
   d. the Fanconi Anemia (FA) pathway; and/or
   e. the base excision repair (BER) pathway.
53. The method of embodiment 51 or embodiment 52, wherein:
   one or more of the one or more genes or gene products is selected from HRR pathway genes and gene products other than ATM; or one or more of the one or more genes or gene products is selected from BRCA1, BRCA2 and ATM, or are selected from BRCA1 and/or BRCA2, and ATM.
54. The method of any one of embodiments 51 to 53, wherein one or more of the one or more genes or gene products comprises ATRX.
55. The method of any one of embodiments 51 to 54, wherein:
   one or more of the one or more genes or gene products is selected from MSH2, MSH3, MSH6, MLH1, MLH3, PMS2, POLE and POLD1; or
   the cancer comprises one or more of mutational signature SBS6 or SBS26 associated with defects in DNA mismatch repair, and the POLD1 mutational signature SBS20.
56. The method of any one of embodiments 51 to 55, wherein one or more of the one or more genes or gene products is selected from FANCA, FANCB, FANCC, FANCD1, FANCD2, FANCE, FANCF, FANCG, FANCI, FANCJ, FANCL, FANCM, FANCN, FANCO, FANCP, FANCQ, FANCR, FANCS, FANCT, FANCU, FANCV and FANCW.
57. The method of any one of embodiments 51 to 56, further comprising assessing or having assessed one or more of a microsatellite instability status of the cancer and a tumor mutational burden of the cancer, and the depletion or mutation in a DDR pathway gene is determined by one or more of the microsatellite instability status and the tumour mutational burden.
58. The method of embodiment 57, wherein the cancer is MSI-high.
59. The method of any one of embodiments 51 to 58, further comprising testing or having tested a sample of patient tissue to determine a cancer expression profile prior to the administering.
60. The method of embodiment 59, wherein the sample comprises cancer DNA, ctDNA, or cancer cells.
61. The method of embodiment 59 or embodiment 60, wherein the testing comprises an assay to detect one or more of protein, mRNA and ctDNA.
62. The method of embodiment 61, wherein one or both of (i) protein is detected using an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, a radioimmunoassay, immunofluorescence, immunochemiluminescence, immunoelectrochemiluminescence, immunoelectrophoretic, a competitive immunoassay, or immunoprecipitation; and (ii) mRNA is detected using RT-PCR or a quantitative gene expression assay; and/or (iii) wherein DNA or RNA is detected by Next Generation Sequencing; and/or (iv) wherein protein is detected by immunohistochemistry.
63. The method of any one of embodiments 59 to 62, wherein the patient is selected for treatment based on the determined cancer expression profile.
64. The method of any preceding embodiment, wherein the cancer is:
   non-small-cell lung carcinoma, mesothelioma, glioblastoma or Kidney Renal Clear Cell carcinoma; or
   Uterine, endometrial, bladder, gastric, colorectal, prostate cancer, or DLBCL; or brain, clear cell renal cell carcinoma (ccRCC), oesophageal cancer or melanoma, or
   acute myeloid leukemia (AML), squamous cell carcinoma or tumors of the head, neck, skin, gastrointestinal system or genital tract; or
   prostate, ovarian, breast or gynaecological cancers; or colorectal, gastric or gynaecological cancers.
65. The method of any preceding claim, wherein the cancer is P53 wild-type.
66. The method of any preceding claim, wherein cancer cells undergo apoptosis following the administering.
67. The method of any preceding embodiment, wherein activated caspase-3 is induced by the MDM2 antagonist in at least a proportion of cancer cells.
68. The method of embodiment 67, wherein activated caspase-3 is induced by the MDM2 antagonist in at least 40% of the cancer cells or at least 60% of the cancer cells.
69. The method of any preceding embodiment, wherein the cancer shows one or more of:
   decreased expression, relative to a control, of one, two or three of CDKN2 Å, BAP1 and SKP2; and
   increased expression, relative to a control, of one, two, three, four, five or more interferon signature gene.
70. The method of embodiment 69, wherein:
   one or more interferon signature gene is selected from CXCL10, CXCL11, RSAD2, MX1, BATF2, IF144L, IFITM1, ISG15, CMPK2, IF127, CD74, IFIH1, CCRL2, IF144, HERC6, ISG20, IFIT3, HLA-C, OAS1, IF135, IRF9, EPST11, USP18, BST2, CSF1, C1S, DHX58, TRIM14, OASL, IRF7, LGALS3BP, DDX60, LAP3, LAMP3, PARP12, PARP9, SP110, PLSCR1, WARS, STAT1, IRF3, IRF5, MSC, JUN, SP11, IRF1, COMMD3-BM11, STAT2, RUNX3, SREBF1 and FLI1; or
   the cancer shows increased expression of CXCL10 or CXCL11.
71. The method of any preceding embodiment, wherein the cancer shows increased expression of one, two, three, four, five or more of IRF7, STAT1, IRF3, IRF5, MSC, JUN, SP11, IRF1, COMMD3-BM11, STAT2, RUNX3, SREBF1, IRF9 and FLI1.
72. The method of any preceding embodiment, wherein the MDM2 antagonist is a compound of formula (I°) or a tautomer, N-oxide, pharmaceutically acceptable salt or solvate thereof.
73. The method of any preceding embodiment, wherein the MDM2 antagonist is (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof.
74. The method of any preceding embodiment, wherein the MDM2 antagonist is selected from Compound 1, idasanutlin (RG-7388), HDM-201, KRT-232 (AMG-232), ALRN-6924, MI-773 (SAR405838), CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64,

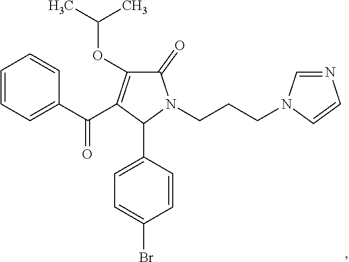 [SMILES: CC(C)OC1=C(C(=O)c2ccccc2)C(c2ccc(Br)cc2)N(CCCn2ccnc2)C1=O]

and one or more of a tautomer, a solvate, and a pharmaceutically acceptable salt of any of the foregoing.
75. A method for treating an MDM2 antagonist susceptible cancer in a patient, comprising detecting or having detected a level of expression or activity in a cancer cell sample from said patient of one or more DDR pathway genes, and administering the MDM2 antagonist to the patient if the level of expression or activity of the one or more DDR pathway genes in the cancer cell sample from the patient is lower than a level of expression or activity, respectively, of one or more of the one or more DDR pathway genes in a non-cancer cell sample or a cell sample from a second patient wherein the second patient has a cancer not susceptible to MDM2 antagonist treatment, optionally wherein the one or more DDR pathway genes comprise BRCA1 and/or BRCA2.
76. The method of embodiment 75, further comprising classifying the patient into a group based level of expression or activity in a cancer cell sample from the patient of one or more DDR pathway genes
77. The method of embodiment 76, wherein the group is selected from:
   (i) responders and non-responders; and
   (ii) strong responders.
78. The method of any one of embodiments 75 to 77, comprising administering the MDM2 antagonist to the patient if the level of expression or activity of 1, 2, 3, 4, 5, 6, 7, 8, 9, 10 or more DDR pathway genes is lower than a level of expression or activity, respectively, in a non-cancer cell sample or a cell sample from a patient having a cancer not susceptible to MDM2 antagonist treatment.
79. The method of any one of embodiments 75 to 78, wherein the detection is carried out using an in vitro detection assay.
80. The method of any one of embodiments 75 to 79, wherein the MDM2 antagonist is a compound of formula (I°) or a tautomer, N-oxide, pharmaceutically acceptable salt or solvate thereof as defined herein.
81. The method of any one of embodiments 75 to 80, wherein the MDM2 antagonist is (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoic acid or a tautomer, N-oxide, pharmaceutically acceptable salt or solvate thereof. 82. The method of any one of embodiments 75 to 81, wherein the MDM2 antagonist is selected from Compound 1, idasanutlin, HDM-201, KRT-232, ALRN-6924, ALRN-6924, CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and

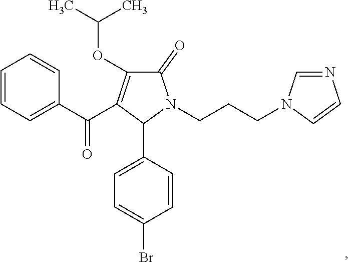 [SMILES: CC(C)OC1=C(C(=O)c2ccccc2)C(c2ccc(Br)cc2)N(CCCn2ccnc2)C1=O]

or a tautomer or a solvate or a pharmaceutically acceptable salt thereof.
83. A method of treating cancer in a patient, wherein the cancer shows loss of one or more DDR pathway genes, gene products or activities, comprising administering an MDM2 antagonist to the patient, optionally wherein the one or more DDR pathway genes comprise BRCA1 and/or BRCA2.
84. The method of any one of the preceding embodiments, further comprising administering a second therapeutic agent (e.g. a PARP inhibitor) to the patient as part of a combination therapy.
85. A method of treating a cancer in a patient, wherein the cancer has normal or high levels of one or more genes or gene products in one or more DNA damage repair (DDR) pathways, or wherein the cancer has no detectable loss of function mutation in any DDR pathway gene, comprising administering to the patient an MDM2 antagonist in combination with an agent to induce sensitivity to the MDM2 antagonist.
86. The method of embodiment 85, wherein the agent to induce sensitivity to the MDM2 antagonist lowers a level of one or more genes or gene products in a DNA damage repair (DDR) pathway.
87. The method of embodiment 85 or embodiment 86, wherein the agent to induce sensitivity to an MDM2 antagonist is a DNA-damaging agent or a DNA repair inhibitor.
88. A method of treating a patient with cancer susceptible to MDM2 antagonist treatment, wherein the method comprises:
   (i) determining that a sample from the patient is depleted of one or more genes or gene products in one or more DNA damage repair (DDR) pathways, or wherein the cancer has at least one loss of function mutation in a DDR pathway gene:
   (ii) administering an effective amount of the MDM2 antagonist to the patient.
89. A method of treating cancer in a patient, comprising administering an MDM2 antagonist in combination with an anticancer agent e.g. a DNA-damaging agent or a DNA repair inhibitor to the patient, wherein
   the cancer has low levels of one or more genes or gene products in one or more DNA damage repair (DDR) pathways, or wherein the cancer has detectable loss of function mutation in any DDR pathway gene.
90. The method embodiments above wherein the one or more DDR pathway genes comprise BRCA1 and/or BRCA2.
91. The method embodiments above wherein the two or more DDR pathway genes do not comprise ATM and/or ATR.
92. The method embodiments above wherein the two or more DDR pathway genes comprise ATR and/or ATM.
93. The method or use embodiments above wherein the two or more DDR pathway genes comprise BRCA1 and/or BRCA2 and ATM.
94. The method or use embodiment above wherein the second agent is a PARP inhibitor.

The invention is now described further with reference to the following non-limiting examples.

### Examples

MDM2 antagonists for use in the invention will now be illustrated, but not limited, by reference to the specific embodiments described in the following examples. Compounds are named using an automated naming package such as AutoNom (MDL) or ChemAxon Structure to Name or are as named by the chemical supplier.

The following first set of examples of MDM2 antagonists, in which cyc is phenyl, can be prepared as described in international patent application no PCT/GB2016/053042 which was published as WO 2017/055860 on 06.04.2017:

| Ex. | Name |
|---|---|
| 1 | (3R)-3-(4-chlorophenyl)-2-[(4-chlorophenyl)methyl]- |
| | 3-{[1-(hydroxymethyl)cyclopropyl]methoxy}-6- |
| | (2-hydroxypropan-2-yl)-2,3-dihydro-1H-isoindol-1-one |
| 2 | (3R)-3-(4-chlorophenyl)-2-[(4-chlorophenyl)methyl]-4-fluoro- |
| | 3-{[1-(hydroxymethyl)cyclopropyl]methoxy}-6-(2- |
| | hydroxypropan-2-yl)-2,3-dihydro-1H-isoindol-1-one |
| 3 | (3R)-3-(4-chlorophenyl)-2-[(4-chlorophenyl)methyl]- |
| | 3-(2-hydroxyethoxy)-6-(2-hydroxypropan-2-yl)- |
| | 2,3-dihydro-1H-isoindol-1-one |
| 4 | (3R)-3-(4-chlorophenyl)-2-[(4-chlorophenyl)methyl]-3- |
| | {[3-(hydroxymethyl)oxetan-3-yl]methoxy}-6- |
| | (2-hydroxypropan-2-yl)-2,3-dihydro-1H-isoindol-1-one |
| 5 | 1-({[(1R)-1-(4-chlorophenyl)-2-[(4- |
| | chlorophenyl)methyl]-5-(2-hydroxypropan-2-yl)-3-oxo-2,3- |
| | dihydro-1H-isoindol-1-yl]oxy}methyl)cyclopropane- |
| | 1-carboxylic acid |
| 6 | (3R)-3-(4-chlorophenyl)-2-[(1S)-1-(4- |
| | chlorophenyl)ethyl]-3-(2,3-dihydroxy-2-methylpropoxy)-6-(2- |
| | hydroxypropan-2-yl)-2,3-dihydro-1H-isoindol-1-one |
| 7 | (3R)-3-(4-chlorophenyl)-2-[(1S)-1-(4-chlorophenyl)ethyl]-3-{[1- |
| | (hydroxymethyl)cyclopropyl]methoxy}-6-(2- |
| | hydroxypropan-2-yl)-2,3-dihydro-1H-isoindol-1-one |
| 8 | (3R)-3-(4-chlorophenyl)-2-[(4-chlorophenyl)methyl]- |
| and | 6-(1,2-dihydroxypropan-2-yl)-3-{[1- |
| 9 | (hydroxymethyl)cyclopropyl]methoxy}- |
| | 2,3-dihydro-1H-isoindol-1-one |
| 10 | (3R)-3-(4-chlorophenyl)-2-[(1S)-1-(4- |
| and | chlorophenyl)ethyl]-6-(2-hydroxy-1-methoxypropan-2-yl)-3- |
| 11 | {[1-(hydroxymethyl)cyclopropyl]methoxy}- |
| | 2,3-dihydro-1H-isoindol-1-one |
| 12 | (3R)-3-(4-chlorophenyl)-2-[(4-chlorophenyl)methyl]- |
| and | 6-[1-(dimethylamino)-2-hydroxypropan-2-yl]-3- |
| 13 | {[1-(hydroxymethyl)cyclopropyl]methoxy}- |
| | 2,3-dihydro-1H-isoindol-1-one |
| 14 | (3S)-3-(4-chlorophenyl)-3-[(1R)-1-(4-chlorophenyl)-1- |
| | {[1-(hydroxymethyl)cyclopropyl]methoxy}-5- |
| | (2-hydroxypropan-2-yl)-3-oxo-2,3-dihydro-1H-isoindol- |
| | 2-yl]propanoic acid |
| 15 | (3R)-3-(4-chlorophenyl)-2-[(1S)-1-(4- |
| | chlorophenyl)ethyl]-6-(1,2-dihydroxypropan-2-yl)-3-{[1- |
| | (hydroxymethyl)cyclopropyl]methoxy}- |
| | 2,3-dihydro-1H-isoindol-1-one |
| 16 | (3R)-3-(4-chlorophenyl)-2-[(4-chlorophenyl)methyl]- |
| | 3-(3-hydroxy-3-methylbutoxy)-6-(2- |
| | hydroxypropan-2-yl)-2,3-dihydro-1H-isoindol-1-one |
| 17 | (3R)-3-(4-chlorophenyl)-2-[(4-chlorophenyl)methyl]- |
| | 6-(2-hydroxypropan-2-yl)-3-[(1H-pyrazol-4- |
| | yl)methoxy]-2,3-dihydro-1H-isoindol-1-one |
| 18 | 1-({[(1R)-1-(4-chlorophenyl)-2-[(4- |
| | chlorophenyl)methyl]-5-(2-hydroxypropan-2-yl)-3-oxo-2,3- |
| | dihydro-1H-isoindol-1-yl]oxy}methyl)cyclopropane- |
| | 1-carbonitrile |
| 19 | N-{[1-({[(1R)-1-(4-chlorophenyl)-2-[(4- |
| | chlorophenyl)methyl]-5-(2-hydroxypropan-2-yl)-3-oxo-2,3- |
| | dihydro-1H-isoindol-1-yl]oxy}methyl)cyclo- |
| | propyl]methyl}methanesulfonamide |
| 20 | (3R)-3-(4-chlorophenyl)-2-[(4-ethynylphenyl)methyl]-3- |
| | {[1-(hydroxymethyl)cyclopropyl]methoxy}-6- |
| | (2-hydroxypropan-2-yl)-2,3-dihydro-1H-isoindol-1-one |
| 21 | (3R)-3-(4-chlorophenyl)-2-[(4-ethynylphenyl)methyl]- |
| | 4-fluoro-3-{[1- |
| | (hydroxymethyl)cyclopropyl]methoxy}-6-(2- |
| | hydroxypropan-2-yl)-2,3-dihydro-1H-isoindol-1-one |
| 22 | (3R)-3-(4-chlorophenyl)-6-(1,2-dihydroxypropan-2-yl)-2- |
| and | [(4-ethynylphenyl)methyl]-4-fluoro-3-{[1- |
| 23 | (hydroxymethyl)cyclopropyl]methoxy}-2,3-dihydro- |
| | 1H-isoindol-1-one |
| 24 | 4-{[(1R)-1-(4-chlorophenyl)-7-fluoro-1-({1- |
| | [hydroxy(²H₂)methyl]cyclopropyl}(²H₂)methoxy)-5-(2- |
| | hydroxypropan-2-yl)-3-oxo-2,3-dihydro-1H-isoindol- |
| | 2-yl]methyl}benzonitrile |
| 25 | 4-{[(1R)-1-(4-chlorophenyl)-1-{[1- |
| | (hydroxymethyl)cyclopropyl]methoxy}-5-(2-hydroxypropan- |
| | 2-yl)-3-oxo-2,3-dihydro-1H-isoindol-2-yl]methyl}benzonitrile |
| 26 | (3R)-3-(4-chlorophenyl)-2-[(4-chlorophenyl)methyl]- |
| | 6-(2-hydroxypropan-2-yl)-3-[(3-methyloxetan- |
| | 3-yl)methoxy]-2,3-dihydro-1H-isoindol-1-one |
| 27 | 4-{[(1R)-1-(4-chlorophenyl)-5-(1,2- |
| and | dihydroxypropan-2-yl)-1-{[1- |
| 28 | (hydroxymethyl)cyclopropyl]methoxy}-3-oxo- |
| | 2,3-dihydro-1H-isoindol-2-yl]methyl}benzonitrile |
| 29 | (3R)-3-(4-chlorophenyl)-2-[(4-chlorophenyl)methyl]-3- |
| | [(1-hydroxycyclopropyl)methoxy]-6-(2- |
| | hydroxypropan-2-yl)-2,3-dihydro-1H-isoindol-1-one |
| 30 | 2-{[(1R)-1-(4-Chlorophenyl)-2-[(4- |
| | chlorophenyl)methyl]-5-(2-hydroxypropan-2-yl)-3-oxo-2,3- |
| | dihydro-1H-isoindol-1-yl]oxy}-N,N-dimethylacetamide |
| 31 | (3R)-3-(4-Chlorophenyl)-2-[(4-chlorophenyl)methyl]- |
| | 6-(2-hydroxypropan-2-yl)-3-{[1- |
| | (methoxymethyl)cyclopropyl]methoxy}- |
| | 2,3-dihydro-1H-isoindol-1-one |
| 32 | (3R)-3-(4-Chlorophenyl)-2-[(4-chlorophenyl)methyl]-3- |
| | {[1-(hydroxymethyl)cyclobutyl]methoxy}-6- |
| | (2-hydroxypropan-2-yl)-2,3-dihydro-1H-isoindol-1-one |
| 33 | 5-chloro-2-{[(1R)-1-(4-chlorophenyl)-1-{[1- |
| | (hydroxymethyl)cyclopropyl]methoxy}-5-(2- |
| | hydroxypropan-2-yl)-3-oxo-2,3-dihydro-1H-isoindol- |
| | 2-yl]methyl}benzoic acid |
| 34 | (3R)-2-{[4-chloro-2-(morpholine-4- |
| | sulfonyl)phenyl]methyl}-3-(4-chlorophenyl)-3-{[1- |
| | (hydroxymethyl)cyclopropyl]methoxy}-6-(2- |
| | hydroxypropan-2-yl)-2,3-dihydro-1H-isoindol-1-one |
| 35 | 1-({[(1R)-2-[(4-chloro-2-methanesulfonylphenyl)methyl]- |
| | 1-(4-chlorophenyl)-7-fluoro-5-(2- |
| | hydroxypropan-2-yl)-3-oxo-2,3-dihydro-1H-isoindol-1- |
| | yl]oxy}methyl)cyclopropane-1-carboxamide |
| 36 | (3R)-2-[(4-chloro-2-methanesulfonylphenyl)methyl]- |
| | 3-(4-chlorophenyl)-3-({1- |
| | [hydroxy(²H₂)methyl]cyclopropyl}(²H₂)methoxy)-6- |
| | (2-hydroxypropan-2-yl)-2,3-dihydro-1H-isoindol- |
| | 1-one |
| 37 | (3R)-3-(4-Chlorophenyl)-2-[(4-chlorophenyl)methyl]- |
| and | 6-(2-hydroxypropan-2-yl)-3-(oxolan-3-yloxy)- |
| 38 | 2,3-dihydro-1H-isoindol-1-one |
| 39 | (3R)-3-(4-chlorophenyl)-2-[(4-chlorophenyl)methyl]- |
| and | 6-(2-hydroxypropan-2-yl)-3-[(oxolan-3- |
| 40 | yl)methoxy]-2,3-dihydro-1H-isoindol-1-one |
| 41 | (3R)-2-[(4-chloro-2-methanesulfonylphenyl)methyl]- |
| and | 3-(4-chlorophenyl)-4-fluoro-6-[1-hydroxy-1- |
| 42 | (oxan-4-yl)ethyl]-3-{[1- |
| | (hydroxymethyl)cyclopropyl]methoxy}-2,3-dihydro- |
| | 1H-isoindol-1-one |
| 43 | (3R)-2-[(4-chloro-2-methanesulfonylphenyl)methyl]- |
| and | 3-(4-chlorophenyl)-4-fluoro-6-[2-hydroxy-1- |
| 44 | (piperazin-1-yl)propan-2-yl]-3-{[1- |
| | (hydroxymethyl)cyclopropyl]methoxy}-2,3-dihydro- |
| | 1H-isoindol-1-one |
| 45 | (3R)-3-(4-Chlorophenyl)-2-[(1S)-1-(4-chlorophenyl)ethyl]- |
| | 3-{[(3S,4R)-4-hydroxyoxolan-3-yl]oxy}-6- |
| | (2-hydroxypropan-2-yl)-2,3-dihydro-1H-isoindol-1-one |
| 46 | (3R)-3-(4-chlorophenyl)-2-[(1S)-1-(4-chlorophenyl)ethyl]- |
| | 3-{[(3R,4S)-4-hydroxyoxolan-3-yl]oxy}-6- |
| | (2-hydroxypropan-2-yl)-2,3-dihydro-1H-isoindol-1-one |
| 47 | (3R)-3-(4-Chlorophenyl)-2-[(4-chlorophenyl)methyl]- |
| | 6-(2-hydroxypropan-2-yl)-3-methoxy-2,3- |
| | dihydro-1H-isoindol-1-one |
| 48 | (3R)-3-(4-Chlorophenyl)-2-[(4-chlorophenyl)methyl]-3-({1- |
| | [hydroxy(²H₂)methyl]cyclopropyl}(²H₂)methoxy)- |
| | 6-(2-hydroxypropan-2-yl)-2,3-dihydro-1H- |
| | isoindol-1-one |
| 49 | (3R)-3-(4-Chlorophenyl)-2-[(4-chlorophenyl)methyl]- |
| | 6-(2-hydroxypropan-2-yl)-3-(3- |
| | hydroxypropoxy)-2,3-dihydro-1H-isoindol-1-one |
| 50 | (3R)-2-[(4-chloro-2-methanesulfonylphenyl)methyl]- |
| | 3-(4-chlorophenyl)-4-fluoro-3-{[1- |
| | (hydroxymethyl)cyclopropyl]methoxy}-6-(2- |
| | hydroxypropan-2-yl)-2,3-dihydro-1H-isoindol-1-one |
| 51 | (3R)-3-(4-Chlorophenyl)-2-[(4-chlorophenyl)methyl]- |
| | 3-(2,2-difluoro-3-hydroxypropoxy)-6-(2- |
| | hydroxypropan-2-yl)-2,3-dihydro-1H-isoindol-1-one |
| 52 | (3R)-3-(4-Chlorophenyl)-2-[(4-chlorophenyl)methyl]- |
| and | 3-{[2-(hydroxymethyl)cyclobutyl]methoxy}-6- |
| 53 | (2-hydroxypropan-2-yl)-2,3-dihydro-1H-isoindol-1-one |
| 54 | (3R)-3-(4-chlorophenyl)-2-[(4-chlorophenyl)methyl]- |
| and | 6-[2-hydroxy-1-oxo-1-(pyrrolidin-1-yl)propan- |
| 55 | 2-yl]-3-{[1-(hydroxymethyl)cyclopropyl]methoxy}- |
| | 2,3-dihydro-1H-isoindol-1-one |
| 56 | 2-[(1R)-1-(4-chlorophenyl)-2-[(4-chlorophenyl)methyl]- |
| and | 1-{[1-(hydroxymethyl)cyclopropyl]methoxy}- |
| 57 | 3-oxo-2,3-dihydro-1H-isoindol-5-yl]-2-hydroxy- |
| | N,N-dimethylpropanamide |
| 58 | 2-[(1R)-1-(4-Chlorophenyl)-2-[(4-chlorophenyl)methyl]-1-{[1- |
| and | (hydroxymethyl)cyclopropyl]methoxy}-3- |
| 59 | oxo-2,3-dihydro-1H-isoindol-5-yl]-2-hydroxy-N- |
| | methylpropanamide |
| 60 | (3R)-2-{[4-chloro-2-(methylsulfanyl)phenyl]methyl}- |
| | 3-(4-chlorophenyl)-4-fluoro-3-{[1- |
| | (hydroxymethyl)cyclopropyl]methoxy}-6-(2- |
| | hydroxypropan-2-yl)-2,3-dihydro-1H-isoindol-1-one |
| 61 | (3R)-2-[(4-Chloro-2-methanesulfinylphenyl)methyl]- |
| and | 3-(4-chlorophenyl)-4-fluoro-3-{[1- |
| 62 | (hydroxymethyl)cyclopropyl]methoxy}-6-(2- |
| | hydroxypropan-2-yl)-2,3-dihydro-1H-isoindol-1-one |
| 63 | (3R)-2-[(4-Chloro-2-methanesulfonylphenyl)methyl]- |
| and | 3-(4-chlorophenyl)-4-fluoro-6-(2-hydroxy-1- |
| 64 | methoxypropan-2-yl)-3-{[1-(hydroxymethyl)cyclo |
| | propyl]methoxy}-2,3-dihydro-1H-isoindol-1-one |
| 65 | (3R)-2-[(4-Chloro-2-methanesulfonylphenyl)methyl]- |
| and | 3-(4-chlorophenyl)-6-(1,2-dihydroxypropan-2- |
| 66 | yl)-4-fluoro-3-{[1-(hydroxymethyl)cyclopropyl]methoxy}- |
| | 2,3-dihydro-1H-isoindol-1-one |
| 67 | (3R)-2-[(4-Chloro-2-methanesulfonylphenyl)methyl]- |
| and | 3-(4-chlorophenyl)-4-fluoro-6-[2-hydroxy-1-(4- |
| 68 | methylpiperazin-1-yl)propan-2-yl]-3-[(3R)-oxolan- |
| | 3-yloxy]-2,3-dihydro-1H-isoindol-1-one |
| 69 | (3R)-2-[(4-Chloro-2-methanesulfonylphenyl)methyl]- |
| and | 3-(4-chlorophenyl)-4-fluoro-6-[2-hydroxy-1-(4- |
| 70 | methylpiperazin-1-yl)propan-2-yl]-3-[(3S)-oxolan- |
| | 3-yloxy]-2,3-dihydro-1H-isoindol-1-one |
| 71 | (3S)-3-(4-Chlorophenyl)-3-[(1R)-1-(4-chlorophenyl)- |
| | 7-fluoro-5-(2-hydroxypropan-2-yl)-3-oxo-1- |
| | [(3S)-oxolan-3-yloxy]-2,3-dihydro-1H-isoindol-2- |
| | yl]propanoic acid |
| 72 | 1-({[(1R)-2-{[4-Chloro-2-(hydroxymethyl)phenyl]methyl}- |
| | 1-(4-chlorophenyl)-7-fluoro-5-(2- |
| | hydroxypropan-2-yl)-3-oxo-2,3-dihydro-1H-isoindol-1- |
| | yl]oxy}methyl)cyclopropane-1-carbonitrile |
| 73 | 1-({[(1R)-2-[(4-chloro-2-methanesulfonylphenyl)methyl]- |
| and | 1-(4-chlorophenyl)-7-fluoro-5-[1-hydroxy- |
| 74 | 1-(1-methyl-1H-pyrazol-4-yl)ethyl]-3-oxo-2,3-dihydro- |
| | 1H-isoindol-1-yl]oxy}methyl)cyclopropane-1- |
| | carboxamide |
| 75 | (3R)-2-[(4-chloro-2-methanesulfonylphenyl)methyl]- |
| and | 3-(4-chlorophenyl)-4-fluoro-6-[1-hydroxy-1-(1- |
| 76 | methyl-1H-pyrazol-4-yl)ethyl]-3-[(1- |
| | hydroxycyclopropyl)methoxy]-2,3-dihydro-1H-isoindol-1-one |
| 77 | (3R)-2-[(4-chloro-2-methanesulfonylphenyl)methyl]- |
| and | 3-(4-chlorophenyl)-4-fluoro-6-[2-hydroxy-1-(4- |
| 78 | methylpiperazin-1-yl)propan-2-yl]-3-{[1- |
| | (hydroxymethyl)cyclopropyl]methoxy}-2,3-dihydro-1H- |
| | isoindol-1-one |
| 79 | 5-chloro-2-{[(1R)-1-(4-chlorophenyl)-1-[(1- |
| | cyanocyclopropyl)methoxy]-7-fluoro-5-(2- |
| | hydroxypropan-2-yl)-3-oxo-2,3-dihydro-1H-isoindol- |
| | 2-yl]methyl}benzoic acid |
| 80 | (3R)-2-[(4-chloro-2-methanesulfonylphenyl)methyl]- |
| and | 3-(4-chlorophenyl)-4-fluoro-6-[1-hydroxy-1-(1- |
| 81 | methylpiperidin-4-yl)ethyl]-3-{[1- |
| | (hydroxymethyl)cyclopropyl]methoxy}-2,3-dihydro- |
| | 1H-isoindol-1-one |
| 82 | (3R)-2-{[4-chloro-2-(dimethylphosphoryl)phenyl]methyl}- |
| | 3-(4-chlorophenyl)-4-fluoro-3-{[1- |
| | (hydroxymethyl)cyclopropyl]methoxy}-6- |
| | (2-hydroxypropan-2-yl)-2,3-dihydro-1H-isoindol-1-one |
| 83 | (3R)-2-[(4-chloro-2-methanesulfonylphenyl)methyl]- |
| and | 3-(4-chlorophenyl)-4-fluoro-6-[hydroxy(oxan- |
| 84 | 4-yl)methyl]-3-{[1-(hydroxymethyl)cyclopropyl]methoxy}- |
| | 2,3-dihydro-1H-isoindol-1-one |
| 85 | 1-({[(1R)-2-[(4-chloro-2-methanesulfonylphenyl)methyl]- |
| and | 1-(4-chlorophenyl)-7-fluoro-5-[1-hydroxy- |
| 86 | 1-(oxan-4-yl)ethyl]-3-oxo-2,3-dihydro-1H-isoindol- |
| | 1-yl]oxy}methyl)cyclopropane-1-carboxamide |
| 87 | 5-chloro-2-{[(1R)-1-(4-chlorophenyl)-7-fluoro-5- |
| | [1-hydroxy-1-(1-methylpiperidin-4-yl)ethyl]-1- |
| | methoxy-3-oxo-2,3-dihydro-1H-isoindol-2- |
| | yl]methyl}benzoic acid |
| 88 | (3S)-3-(4-chlorophenyl)-3-[(1R)-1-(4-chlorophenyl)- |
| and | 7-fluoro-5-[1-hydroxy-1-(oxan-4-yl)ethyl]-1- |
| 89 | methoxy-3-oxo-2,3-dihydro-1H-isoindol-2-yl]propanoic acid |
| 90 | 4-[(1R)-1-[(1R)-1-(4-Chlorophenyl)-7-fluoro-5-[1- |
| and | hydroxy-1-(1-methyl-1H-imidazol-4-yl)propyl]-3- |
| 91 | oxo-1-[(3S)-oxolan-3-yloxy]-2,3-dihydro-1H- |
| | isoindol-2-yl]-2-hydroxyethyl]benzonitrile |
| 92 | 4-{[(1R)-1-(4-chlorophenyl)-7-fluoro-5-[1- |
| and | hydroxy-1-(1-methyl-1H-imidazol-4-yl)propyl]-3-oxo-1- |
| 93 | [(3S)-oxolan-3-yloxy]-2,3-dihydro-1H-isoindol- |
| | 2-yl]methyl}-3-(hydroxymethyl)benzonitrile |
| 94 | 4-{[(1R)-1-(4-chlorophenyl)-7-fluoro-5-[1-hydroxy- |
| and | 1-(1-methyl-1H-imidazol-4-yl)propyl]-1-{[1- |
| 95 | (hydroxymethyl)cyclopropyl]methoxy}-3-oxo- |
| | 2,3-dihydro-1H-isoindol-2-yl]methyl}benzonitrile |
| 96 | 4-{[(1R)-1-(4-chlorophenyl)-7-fluoro-5-[1-hydroxy- |
| and | 1-(1-methyl-1H-imidazol-4-yl)propyl]-3-oxo-1- |
| 97 | [(3S)-oxolan-3-yloxy]-2,3-dihydro-1H-isoindol- |
| | 2-yl]methyl}benzonitrile |
| 98 | (3S)-3-(4-chlorophenyl)-3-[(1R)-1-(4-chlorophenyl)- |
| and | 7-fluoro-5-[1-(4-fluorooxan-4-yl)-1- |
| 99 | hydroxyethyl]-1-methoxy-3-oxo-2,3-dihydro- |
| | 1H-isoindol-2-yl]propanoic acid |
| 100 | (4S)-4-(4-chlorophenyl)-4-[(1R)-1-(4-chlorophenyl)- |
| | 7-fluoro-5-[1-hydroxy-1-(1-methyl-1H-pyrazol- |
| | 3-yl)propyl]-1-methoxy-3-oxo-2,3-dihydro- |
| | 1H-isoindol-2-yl]butanoic acid |
| 101 | (3S)-3-(4-chlorophenyl)-3-[(1R)-1-(4-chlorophenyl)- |
| and | 7-fluoro-5-[1-(4-fluorooxan-4-yl)-1- |
| 102 | hydroxypropyl]-1-methoxy-3-oxo-2,3-dihydro- |
| | 1H-isoindol-2-yl]propanoic acid |
| 103 | (3S)-3-(4-chlorophenyl)-3-[(1R)-1-(4-chlorophenyl)- |
| and | 5-(1-cyclobutyl-1-hydroxyethyl)-7-fluoro-1- |
| 104 | methoxy-3-oxo-2,3-dihydro-1H-isoindol-2-yl]propanoic acid |
| 105 | (3S)-3-(4-chlorophenyl)-3-[(1R)-1-(4-chlorophenyl)- |
| | 7-fluoro-5-[(1S)-1-hydroxy-1-(oxan-4- |
| | yl)propyl]-1-methoxy-3-oxo-2,3-dihydro-1H- |
| | isoindol-2-yl]propanoic acid |
| 106 | (3S)-3-(4-chlorophenyl)-3-[(1R)-1-(4-chlorophenyl)- |
| | 7-fluoro-5-[(1R)-1-hydroxy-1-(oxan-4- |
| | yl)propyl]-1-methoxy-3-oxo-2,3-dihydro-1H- |
| | isoindol-2-yl]propanoic acid |
| 107 | (4S)-4-(4-chlorophenyl)-4-[(1R)-1-(4-chlorophenyl)- |
| | 7-fluoro-5-[(1S)-1-hydroxy-1-(oxan-4- |
| | yl)propyl]-1-methoxy-3-oxo-2,3-dihydro- |
| | 1H-isoindol-2-yl]butanoic acid |
| 108 | (4S)-4-(4-chlorophenyl)-4-[(1R)-1-(4-chlorophenyl)- |
| | 7-fluoro-5-[(1R)-1-hydroxy-1-(oxan-4- |
| | yl)propyl]-1-methoxy-3-oxo-2,3-dihydro- |
| | 1H-isoindol-2-yl] butanoic acid |
| 109 | (4S)-4-(4-Chlorophenyl)-4-[(1R)-1-(4-chlorophenyl)- |
| | 7-fluoro-5-[(1R)-1-(4-fluorooxan-4-yl)-1- |
| | hydroxypropyl]-1-methoxy-3-oxo-2,3-dihydro- |
| | 1H-isoindol-2-yl]butanoic acid |
| | (tris(hydroxymethyl)aminomethane salt) |
| 110 | (3S)-3-(4-chlorophenyl)-3-[(1R)-1-(4-chlorophenyl)- |
| | 7-fluoro-5-[(1R)-1-(4-fluorooxan-4-yl)-1- |
| | hydroxypropyl]-1-trideuteromethoxy-3-oxo-2,3- |
| | dihydro-1H-isoindol-2-yl]propanoic acid |
| 111 | (3S)-3-(4-chlorophenyl)-3-[(1R)-1-(4-chlorophenyl)- |
| | 1-ethoxy-7-fluoro-5-[(1R)-1-(4-fluorooxan-4- |
| | yl)-1-hydroxypropyl]-3-oxo-2,3-dihydro-1H-isoindol- |
| | 2-yl]propanoic acid |
| 113 | 4S)-4-[(1R)-1-(4-chlorophenyl)-7-fluoro-5-[(1R)- |
| | 1-(4-fluorooxan-4-yl)-1-hydroxypropyl]-1-methoxy- |
| | 3-oxo-2,3-dihydro-1H-isoindol-2-yl]-4-(4- |
| | methoxyphenyl)butanoic acid |
| 114 | (4S)-4-(4-chlorophenyl)-4-[(1R)-1-(4-chlorophenyl)- |
| | 7-fluoro-5-{1-hydroxy-1-[trans-4- |
| | hydroxycyclohexyl]propyl}-1-methoxy-3-oxo-2,3- |
| | dihydro-1H-isoindol-2-yl] butanoic acid |
| 115 | 2-(5-chloro-2-{[1-(4-chlorophenyl)-7-fluoro-5- |
| | [(1R)-1-(4-fluorooxan-4-yl)-1-hydroxypropyl]-1- |
| | methoxy-3-oxo-2,3-dihydro-1H-isoindol-2- |
| | yl]methyl}phenoxy)acetic acid |
| | (tris(hydroxymethyl)aminomethane salt) |
| 116 | 5-chloro-2-{[(1R)-1-(4-chlorophenyl)-7-fluoro-5- |
| | [(1S)-1-hydroxy-1-(oxan-4-yl)propyl]-1-methoxy-3- |
| | oxo-2,3-dihydro-1H-isoindol-2-yl]methyl}benzoic acid |
| 117 | 5-chloro-2-{[(1R)-1-(4-chlorophenyl)-7-fluoro-5-[(1R)- |
| | 1-hydroxy-1-(oxan-4-yl)propyl]-1-methoxy-3- |
| | oxo-2,3-dihydro-1H-isoindol-2-yl]methyl}benzoic acid |
| 118 | 5-chloro-2-{[(1R)-1-(4-chlorophenyl)-1-ethoxy-7-fluoro- |
| | 5-[(1S)-1-hydroxy-1-(oxan-4-yl)propyl]-3- |
| | oxo-2,3-dihydro-1H-isoindol-2-yl]methyl}benzoic |
| | acid-(tris(hydroxymethyl)aminomethane salt) |
| 119 | 2-{[(1R)-1-(4-chlorophenyl)-7-fluoro-5-[(1R)- |
| | 1-(4-fluorooxan-4-yl)-1-hydroxypropyl]-1-methoxy-3- |
| | oxo-2,3-dihydro-1H-isoindol-2-yl]methyl}-5- |
| | methylbenzoic acid |
| 120 | 2-{[(1R)-1-(4-chlorophenyl)-7-fluoro-5-[(1R)-1- |
| | (4-fluorooxan-4-yl)-1-hydroxypropyl]-1-methoxy-3- |
| | oxo-2,3-dihydro-1H-isoindol-2-yl]methyl}-5-methoxybenzoic |
| | acid-tris(hydroxymethyl)aminomethane salt |
| 121 | 2-(5-chloro-2-{[(1R)-1-(4-chlorophenyl)-7-fluoro-5- |
| | [(1S)-1-hydroxy-1-(oxan-4-yl)propyl]-1-methoxy- |
| | 3-oxo-2,3-dihydro-1H-isoindol-2-yl]methyl}phenyl)- |
| | 2-methylpropanoic acid |
| | (tris(hydroxymethyl)aminomethane salt) |
| 122 | 2-(5-chloro-2-{[(1R)-1-(4-chlorophenyl)-7-fluoro-5-[(1S)-1- |
| | hydroxy-1-(oxan-4-yl)propyl]-1-methoxy- |
| | 3-oxo-2,3-dihydro-1H-isoindol-2-yl]methyl}phenyl)acetic |
| | acid (tris(hydroxymethyl)aminomethane |
| | salt) |
| 123 | 2-(5-chloro-2-{[(1R)-1-(4-chlorophenyl)-7-fluoro- |
| | 5-[(1R)-1-hydroxy-1-(oxan-4-yl)propyl]-1- |
| | methoxy-3-oxo-2,3-dihydro-1H-isoindol-2- |
| | yl]methyl}phenyl)acetic acid |
| 124 | (2S,3S)-3-(4-chlorophenyl)-3-[(1R)-1-(4-chlorophenyl)- |
| | 7-fluoro-5-[(1S)-1-hydroxy-1-(oxan-4- |
| | yl)propyl]-1-methoxy-3-oxo-2,3-dihydro-1H-isoindol-2- |
| | yl]-2-methylpropanoic acid (“Compound 1”) |
| 124 | (2S,3S)-3-(4-chlorophenyl)-3-[(1R)-1-(4-chlorophenyl)- |
| a | 7-fluoro-5-[(1S)-1-hydroxy-1-(oxan-4- |
| | yl)propyl]-1-methoxy-3-oxo-2,3-dihydro-1H-isoindol-2- |
| | yl]-2-methylpropanoic acid (“Compound 1”) |
| | “(tris(hydroxymethyl)aminomethane salt) |
| 125 | (3S)-3-(4-chlorophenyl)-3-[(1R)-1-(4-chlorophenyl)-7- |
| and | fluoro-1-[(3-fluorooxetan-3-yl)methoxy]-5-(2- |
| 126 | hydroxybutan-2-yl)-3-oxo-2,3-dihydro-1H-isoindol- |
| | 2-yl]propanoic acid |
| 127 | (3S)-3-(4-chlorophenyl)-3-[(1R)-1-(4-chlorophenyl)-7- |
| and | fluoro-5-[1-hydroxy-1-(pyridin-2-yl)propyl]-1- |
| 128 | methoxy-3-oxo-2,3-dihydro-1H-isoindol-2-yl] propanoic acid |
| 129 | (3R)-2-[(4-chloro-2-methanesulfonylphenyl)methyl]- |
| | 3-(4-chlorophenyl)-4-fluoro-6-[1-(4- |
| | fluoropiperidin-4-yl)-1-hydroxypropyl]-3-methoxy- |
| | 2,3-dihydro-1H-isoindol-1-one |
| 130 | 4-{[(1R)-1-(4-chlorophenyl)-7-fluoro-5-[(1S)-1- |
| | hydroxy-1-(1-methylpiperidin-4-yl)propyl]-3-oxo-1- |
| | [cis-3-hydroxycyclobutoxy]-2,3-dihydro-1H-isoindol- |
| | 2-yl]methyl}benzonitrile |
| 131 | (3S)-3-(4-chlorophenyl)-3-[(1R)-1-(4-chlorophenyl)- |
| | 7-fluoro-5-[1-(4-fluoro-1-methylpiperidin-4-yl)- |
| | 1-hydroxypropyl]-1-methoxy-3-oxo-2,3-dihydro- |
| | 1H-isoindol-2-yl]propanoic acid |
| 132 | tert-butyl 2-{4-[(1S)-1-[(1R)-1-(4-chlorophenyl)-2- |
| and | [(4-chlorophenyl)methyl]-7-fluoro-1-methoxy-3- |
| 133 | oxo-2,3-dihydro-1H-isoindol-5-yl]-1-hydroxypropyl]piperidin- |
| | 1-yl}acetate and tert-butyl 2-{4-[(1R)- |
| | 1-[(1R)-1-(4-chlorophenyl)-2-[(4-chlorophenyl)methyl]- |
| | 7-fluoro-1-methoxy-3-oxo-2,3-dihydro-1H- |
| | isoindol-5-yl]-1-hydroxypropyl]piperidin-1-yl}acetate |
| 134 | 2-{4-[(1S)-1-[(1R)-1-(4-chlorophenyl)-2-[(4- |
| | chlorophenyl)methyl]-7-fluoro-1-methoxy-3-oxo-2,3- |
| | dihydro-1H-isoindol-5-yl]-1-hydroxypropyl]piperidin- |
| | 1-yl}acetic acid |
| 135 | 2-{4-[(1R)-1-[(1R)-1-(4-chlorophenyl)-2-[(4- |
| | chlorophenyl)methyl]-7-fluoro-1-methoxy-3-oxo-2,3- |
| | dihydro-1H-isoindol-5-yl]-1-hydroxypropyl]piperidin- |
| | 1-yl}acetic acid |
| 136 | Methyl 3-{4-[(1S)-1-[(1R)-1-(4-chlorophenyl)-2- |
| | [(4-chlorophenyl)methyl]-7-fluoro-1-methoxy-3-oxo- |
| | 2,3-dihydro-1H-isoindol-5-yl]-1-hydroxypropyl]piperidin- |
| | 1-yl}propanoate |
| 137 | 3-{4-[(1S)-1-[(1R)-1-(4-chlorophenyl)-2-[(4- |
| | chlorophenyl)methyl]-7-fluoro-1-methoxy-3-oxo-2,3- |
| | dihydro-1H-isoindol-5-yl]-1-hydroxypropyl]piperidin- |
| | 1-yl}propanoic acid |

| Ex. | Name |
|---|---|
| 1 | (3R)-3-(4-chlorophenyl)-2-[(5-chloropyridin- |
| | 2-yl)methyl]-4-fluoro-3-({1- |
| | [hydroxy(²H₂)methyl]cyclopropyl}(²H₂)methoxy)- |
| | 6-(2-hydroxypropan-2-yl)-2,3-dihydro-1H-isoindol- |
| | 1-one |
| 2 | (3R)-3-(4-Chlorophenyl)-2-[(5-chloropyridin-2- |
| | yl)methyl]-4-fluoro-6-(2-hydroxypropan-2-yl)-3- |
| | methoxy-2,3-dihydro-1H-isoindol-1-one |
| 3 | 6-{[(1R)-1-(4-chlorophenyl)-7-fluoro-1-[(1-hydroxycyclo- |
| | propyl)methoxy]-5-(2-hydroxypropan-2-yl)- |
| | 3-oxo-2,3-dihydro-1H-isoindol-2-yl]methyl}pyridine- |
| | 3-carbonitrile |
| 4 | 1-({[(1R)-1-(4-chlorophenyl)-2-[(5-chloropyridin-2- |
| | yl)methyl]-7-fluoro-5-(2-hydroxypropan-2-yl)-3- |
| | oxo-2,3-dihydro-1H-isoindol-1- |
| | yl]oxy}methyl)cyclopropane-1-carboxamide |
| 5 | 6-{[(1R)-1-(4-Chlorophenyl)-7-fluoro-1-({1- |
| | [hydroxy(²H₂)methyl]cyclopropyl}(²H₂)methoxy)-5-(2- |
| | hydroxypropan-2-yl)-3-oxo-2,3-dihydro-1H-isoindol- |
| | 2-yl]methyl}pyridine-3-carbonitrile |
| 6 | (3R)-3-(4-chlorophenyl)-2-[(5-chloropyridin-2- |
| | yl)methyl]-3-(2-hydroxyethoxy)-6-(2-hydroxypropan- |
| | 2-yl)-2,3-dihydro-1H-isoindol-1-one |
| 7 | (3R)-3-(4-Chlorophenyl)-2-[(5-chloropyridin-2-yl)methyl]- |
| | 3-{[1-(hydroxymethyl)cyclopropyl]methoxy}-6-(2- |
| | hydroxypropan-2-yl)-2,3-dihydro-1H-isoindol-1-one |
| 8 | (3R)-3-(4-Chlorophenyl)-2-[(5-chloropyridin-2- |
| | yl)methyl]-6-(2-hydroxypropan-2-yl)-3-methoxy-2,3- |
| | dihydro-1H-isoindol-1-one |
| 9 | (3R)-3-(4-Chlorophenyl)-2-[(5-chloropyridin- |
| | 2-yl)methyl]-6-(2-hydroxypropan-2-yl)-3-(3- |
| | hydroxypropoxy)-2,3-dihydro-1H-isoindol-1-one |
| 10 | (3R)-2-[(5-Chloro-1-oxo-1λ⁵-pyridin-2- |
| | yl)methyl]-3-(4-chlorophenyl)-3-{[1- |
| | (hydroxymethyl)cyclopropyl]methoxy}-6-(2- |
| | hydroxypropan-2-yl)-2,3-dihydro-1H-isoindol-1-one |
| 11 | (3R)-3-(4-Chlorophenyl)-2-[(5-chloropyridin-2- |
| | yl)methyl]-4-fluoro-3-[(1-hydroxycyclopropyl)methoxy]-6- |
| | (2-hydroxypropan-2-yl)-2,3-dihydro-1H-isoindol-1-one |
| 12 | (3R)-3-(4-Chlorophenyl)-4-fluoro-3-({1- |
| | [hydroxy(²H₂)methyl]cyclopropyl}(²H₂)methoxy)-6-(2- |
| | hydroxypropan-2-yl)-2-[(6-methylpyridazin- |
| | 3-yl)methyl]-2,3-dihydro-1H-isoindol-1-one |
| 13 | (3R)-3-(4-Chlorophenyl)-2-[(5-chloropyridin- |
| | 2-yl)methyl]-4-fluoro-6-(2-hydroxypropan-2-yl)-3-[(1- |
| 14 | (3R)-3-(4-Chlorophenyl)-2-[(5-chloropyridin- |
| and | 2-yl)methyl]-6-(1,2-dihydroxypropan-2-yl)-3-{[1- |
| 15 | (hydroxymethyl)cyclopropyl]methoxy}-2,3-dihydro- |
| | 1H-isoindol-1-one |
| 16 | (3R)-3-(4-Chlorophenyl)-2-[(5-chloropyridin-2- |
| and | yl)methyl]-6-(1,2-dihydroxypropan-2-yl)-4-fluoro-3- |
| 17 | ({1-[hydroxy(²H₂)methyl]cyclopropyl}(²H₂)methoxy)- |
| | 2,3-dihydro-1H-isoindol-1-one |
| 18 | (3R)-3-(4-Chlorophenyl)-2-[(5-chloropyridin-2- |
| and | yl)methyl]-6-(2,4-dihydroxybutan-2-yl)-3-{[1- |
| 19 | (hydroxymethyl)cyclopropyl]methoxy}-2,3- |
| | dihydro-1H-isoindol-1-one |
| 20 | 6-{[(1R)-1-(4-Chlorophenyl)-5-(2,4-dihydroxybutan- |
| and | 2-yl)-7-fluoro-1-({1- |
| 21 | [hydroxy(²H₂)methyl]cyclopropyl}(²H₂)methoxy)- |
| | 3-oxo-2,3-dihydro-1H-isoindol-2- |
| | yl]methyl}pyridine-3-carbonitrile |
| 22 | (3R)-3-(4-Chlorophenyl)-2-[(5-chloropyridin-2- |
| and | yl)methyl]-6-[1-(dimethylamino)-2-hydroxypropan-2- |
| 23 | yl]-4-fluoro-3-({1-[hydroxy(²H₂)methyl]cyclo- |
| | propyl}(²H₂)methoxy)-2,3-dihydro-1H-isoindol-1-one |
| 24 | (3R)-3-(4-Chlorophenyl)-2-[(5- |
| and | chloropyridin-2-yl)methyl]-4-fluoro-3-({1- |
| 25 | [hydroxy(²H₂)methyl]cyclopropyl}(²H₂)methoxy)- |
| | 6-(2-hydroxy-1-methoxypropan-2-yl)-2,3-dihydro- |
| | 1H-isoindol-1-one |
| 26 | (3R)-3-(4-Chlorophenyl)-2-[(5-chloropyridin-2- |
| | yl)methyl]-4-fluoro-3-[3-hydroxy-2-(hydroxymethyl)- |
| | 2-methylpropoxy]-6-(2-hydroxypropan-2-yl)- |
| | 2,3-dihydro-1H-isoindol-1-one |
| 27 | 1-({[(1R)-1-(4-chlorophenyl)-2-[(5-chloropyridin-2- |
| | yl)methyl]-7-fluoro-5-(2-hydroxypropan-2-yl)-3- |
| | oxo-2,3-dihydro-1H-isoindol-1- |
| | yl]oxy}methyl)cyclopropane-1-carbonitrile |
| 28 | (3R)-3-(4-Chlorophenyl)-4-fluoro-3-{[1- |
| | (hydroxymethyl)cyclopropyl]methoxy}-6-(2-hydroxypropan- |
| | 2-yl)-2-[(5-methylpyridin-2-yl)methyl]- |
| | 2,3-dihydro-1H-isoindol-1-one |
| 29 | (3R)-3-(4-Chlorophenyl)-4-fluoro-3-{[1- |
| | (hydroxymethyl)cyclopropyl]methoxy}-6-(2- |
| | hydroxypropan-2-yl)-2-[(5-methoxypyridin-2- |
| | yl)methyl]-2,3-dihydro-1H-isoindol-1-one |
| 30 | 3-(4-Chlorophenyl)-2-[(5-chloropyrimidin- |
| | 2-yl)methyl]-4-fluoro-3-{[1- |
| | (hydroxymethyl)cyclopropyl]methoxy}-6-(2- |
| | hydroxypropan-2-yl)-2,3-dihydro-1H-isoindol-1-one |
| 31 | (3R)-3-(4-chlorophenyl)-2-[(5-chloropyridin-2- |
| | yl)methyl]-3-[(1-hydroxycyclopropyl)methoxy]-6-(2- |
| | hydroxypropan-2-yl)-2,3-dihydro-1H-isoindol-1-one |
| 32 | (3R)-3-(4-Chlorophenyl)-2-[(5-chloropyridin-2-yl)methyl]- |
| | 3-({1-[hydroxy(²H₂)methyl]cyclopropyl}(²H₂)methoxy)- |
| | 6-(2-hydroxypropan-2-yl)-2,3-dihydro-1H-isoindol- |
| | 1-one |
| 33 | (3R)-3-(4-chlorophenyl)-2-[(5-chloropyridin-2- |
| | yl)methyl]-4-fluoro-6-(2-hydroxypropan-2-yl)-3-[(1- |
| | methanesulfonylcyclopropyl)methoxy]-2,3- |
| | dihydro-1H-isoindol-1-one |
| 34 | N-[1-({[(1R)-1-(4-chlorophenyl)-2-[(5-chloropyridin- |
| | 2-yl)methyl]-7-fluoro-5-(2-hydroxypropan-2-yl)- |
| | 3-oxo-2,3-dihydro-1H-isoindol-1- |
| | yl]oxy}methyl)cyclopropyl]acetamide |
| 35 | 6-{[(1R)-1-(4-Chlorophenyl)-1-({1- |
| | [hydroxy(²H₂)methyl]cyclopropyl}(²H₂)methoxy)-5-(2- |
| | hydroxypropan-2-yl)-3-oxo-2,3-dihydro-1H- |
| | isoindol-2-yl]methyl}pyridine-3-carbonitrile |
| 36 | 6-{[(1R)-1-(4-chlorophenyl)-7-fluoro-1- |
| | {[1-(hydroxymethyl)cyclopropyl]methoxy}-5-(2- |
| | hydroxypropan-2-yl)-3-oxo-2,3-dihydro-1H- |
| | isoindol-2-yl]methyl}pyridine-3-carbonitrile |
| 37 | (3R)-3-(4-Chlorophenyl)-4-fluoro-3-({1- |
| | [hydroxy(²H₂)methyl]cyclopropyl}(²H₂)methoxy)-6-(2- |
| | hydroxypropan-2-yl)-2-[(6-methoxypyridin-3- |
| | yl)methyl]-2,3-dihydro-1H-isoindol-1-one |
| 38 | (3R)-3-(4-chlorophenyl)-2-[(5-chloropyridin-2- |
| and | yl)methyl]-3-{[(1S,3R)-3-hydroxycyclopentyl]oxy}-6- |
| 39 | (2-hydroxypropan-2-yl)-2,3-dihydro-1H-isoindol- |
| | 1-one and (3R)-3-(4-chlorophenyl)-2-[(5- |
| | chloropyridin-2-yl)methyl]-3-{[(1R,3S)-3- |
| | hydroxycyclopentyl]oxy}-6-(2-hydroxypropan-2-yl)-2,3- |
| | dihydro-1H-isoindol-1-one |
| 40 | 6-{[(1R)-1-(4-chlorophenyl)-7-fluoro-1-{[(1S,3R)- |
| and | 3-hydroxycyclopentyl]oxy}-5-(2-hydroxypropan-2- |
| 41 | yl)-3-oxo-2,3-dihydro-1H-isoindol-2- |
| | yl]methyl}pyridine-3-carbonitrile and 6-{[(1R)-1-(4- |
| | chlorophenyl)-7-fluoro-1-{[(1R,3S)-3- |
| | hydroxycyclopentyl]oxy}-5-(2-hydroxypropan-2-yl)-3-oxo-2,3- |
| | dihydro-1H-isoindol-2-yl]methyl}pyridine-3-carbonitrile |
| 42 | 6-{[(1R)-1-(4-chlorophenyl)-7-fluoro-1-[(3- |
| and | hydroxycyclopentyl)oxy]-5-(2-hydroxypropan-2-yl)-3- |
| 43 | oxo-2,3-dihydro-1H-isoindol-2-yl]methyl}pyridine- |
| | 3-carbonitrile |
| 44 | (3R)-3-(4-chlorophenyl)-2-[(5-chloropyridin-2- |
| and | yl)methyl]-3-{[(1R,3R)-3-hydroxycyclopentyl]oxy}-6- |
| 45 | (2-hydroxypropan-2-yl)-2,3-dihydro-1H-isoindol- |
| | 1-one and (3R)-3-(4-chlorophenyl)-2-[(5- |
| | chloropyridin-2-yl)methyl]-3-{[(1S,3S)-3- |
| | hydroxycyclopentyl]oxy}-6-(2-hydroxypropan-2-yl)-2,3- |
| | dihydro-1H-isoindol-1-one |
| 46 | (3S)-3-(4-Chloro-2-fluorophenyl)-2- |
| | [(5-chloropyridin-2-yl)methyl]-3-{[1- |
| | (hydroxymethyl)cyclopropyl]methoxy}-6-(2- |
| | hydroxypropan-2-yl)-2,3-dihydro-1H-isoindol-1-one |
| 47 | ((3R)-2-[(5-chloropyridin-2-yl)methyl]-3-(4-ethylphenyl)- |
| | 3-{[1-(hydroxymethyl)cyclopropyl]methoxy}-6-(2- |
| | hydroxypropan-2-yl)-2,3-dihydro-1H-isoindol-1-one |
| 48 | 4-[(1R)-2-[(5-Chloropyridin-2-yl)methyl]-1-({1- |
| | [hydroxy(²H₂) methyl]cyclopropyl}(²H₂)methoxy)-5-(2- |
| | hydroxypropan-2-yl)-3-oxo-2,3-dihydro- |
| | 1H-isoindol-1-yl]benzonitrile |
| 49 | (3R)-2-[(5-Chloropyridin-2-yl)methyl]-3-(4-fluorophenyl)- |
| | 3-({1-[hydroxy(²H₂)methyl]cyclopropyl}(²H₂)methoxy)- |
| | 6-(2-hydroxypropan-2-yl)-2,3-dihydro-1H-isoindol- |
| | 1-one |
| 50 | (3R)-2-[(5-Chloropyridin-2-yl)methyl]-3-({1- |
| | [hydroxy(²H₂)methyl]cyclopropyl}(²H₂)methoxy)-6-(2- |
| | hydroxypropan-2-yl)-3-[4-(trifluoromethyl)phenyl]- |
| | 2,3-dihydro-1H-isoindol-1-one |
| 51 | (3R)-2-[(5-chloropyridin-2-yl)methyl]-3- |
| | [4-(1,1-difluoroethyl)phenyl]-3-{[1- |
| | (hydroxymethyl)cyclopropyl]methoxy}-6-(2- |
| | hydroxypropan-2-yl)-2,3-dihydro-1H-isoindol-1-one |
| 52 | (3R)-2-[(5-chloropyridin-2-yl)methyl]-3-(3,4-difluorophenyl)- |
| | 3-({1-[hydroxy(²H₂)methyl]cyclopropyl}(²H₂)methoxy)- |
| | 6-(2-hydroxypropan-2-yl)-2,3-dihydro-1H-isoindol- |
| | 1-one |
| 53 | (3R)-2-[(5-chloropyridin-2-yl)methyl]-3-({1- |
| | [hydroxy(²H₂)methyl]cyclopropyl}(²H₂)methoxy)-6-(2- |
| | hydroxypropan-2-yl)-3-[4-(trifluoromethoxy)phenyl]- |
| | 2,3-dihydro-1H-isoindol-1-one |
| 54 | (3R)-4-Chloro-3-(4-chlorophenyl)-2-[(5-chloropyridin- |
| | 2-yl)methyl]-6-(2-hydroxypropan-2-yl)-3- |
| | methoxy-2,3-dihydro-1H-isoindol-1-one |
| 55 | (3R)-3-(4-Chlorophenyl)-2-[(5-chloropyridin-2- |
| and | yl)methyl]-6-[1-hydroxy-1-(1H-pyrazol-4-yl)ethyl]-3- |
| 56 | methoxy-2,3-dihydro-1H-isoindol-1-one |
| 57 | (3R)-3-(4-chlorophenyl)-2-[(5-chloropyridin-2- |
| and | yl)methyl]-6-[1-hydroxy-1-(1-methyl-1H-pyrazol-4- |
| 58 | yl)ethyl]-3-methoxy-2,3-dihydro-1H-isoindol-1-one |
| 59 | (3R)-3-(4-Chlorophenyl)-2-[(5-chloropyridin-2- |
| | yl)methyl]-3-[(2S)-3-hydroxy-2-methyl(3,3- |
| | ²H₂)propoxy]-6-(2-hydroxypropan-2-yl)- |
| | 2,3-dihydro-1H-isoindol-1-one |
| 60 | (3R)-3-(4-Chlorophenyl)-2-[(5-chloropyridin-2- |
| | yl)methyl]-3-[(2R)-3-hydroxy-2-methyl(3,3- |
| | ²H₂)propoxy]-6-(2-hydroxypropan-2-yl)-2,3- |
| | dihydro-1H-isoindol-1-one |
| 61 | 3-{[(1R)-1-(4-Chlorophenyl)-2-[(5-chloropyridin- |
| | 2-yl)methyl]-5-(2-hydroxypropan-2-yl)-3-oxo-2,3- |
| | dihydro-1H-isoindol-1-yl]oxy}-1λ6-thiolane- |
| | 1,1-dione-Isomer 1 |
| 62 | 3-{[(1R)-1-(4-Chlorophenyl)-2-[(5-chloropyridin-2- |
| | yl)methyl]-5-(2-hydroxypropan-2-yl)-3-oxo-2,3- |
| | dihydro-1H-isoindol-1-yl]oxy}-1λ6-thiolane-1,1-dione-Isomer 2 |
| 63 | 2-[1-({[(1R)-1-(4-chlorophenyl)-2-[(5-chloropyridin- |
| | 2-yl)methyl]-5-(2-hydroxypropan-2-yl)-3-oxo- |
| | 2,3-dihydro-1H-isoindol-1- |
| | yl]oxy}methyl)cyclopropyl]acetonitrile |
| 64 | (3R)-3-[(1-acetylazetidin-3-yl)methoxy]-3-(4- |
| | chlorophenyl)-2-[(5-chloropyridin-2-yl)methyl]-6-(2- |
| | hydroxypropan-2-yl)-2,3-dihydro-1H-isoindol-1-one |
| 65 | (3R)-3-(4-chlorophenyl)-2-[(5-chloropyridin-2- |
| | yl)methyl]-3-[3-(hydroxymethyl)cyclobutoxy]-6-(2- |
| | hydroxypropan-2-yl)-2,3-dihydro-1H-isoindol-1-one |
| 66 | (3R)-3-[(1-Aminocyclopropyl)methoxy]-3-(4- |
| | chlorophenyl)-2-[(5-chloropyridin-2-yl)methyl]-4- |
| | fluoro-6-(2-hydroxypropan-2-yl)-2,3-dihydro-1H-isoindol-1-one |
| 67 | 1-({[(1R)-1-(4-Chlorophenyl)-2-[(5-chloropyridin-2- |
| | yl)methyl]-7-fluoro-5-(2-hydroxypropan-2-yl)-3- |
| | oxo-2,3-dihydro-1H-isoindol-1-yl]oxy}methyl)- |
| | N-methylcyclopropane-1-carboxamide |
| 68 | 1-({[(1R)-1-(4-Chlorophenyl)-2-[(5-chloropyridin- |
| and | 2-yl)methyl]-7-fluoro-5-[2-hydroxy-1-(piperazin-1- |
| 69 | yl)propan-2-yl]-3-oxo-2,3-dihydro-1H-isoindol-1- |
| | yl]oxy}methyl)cyclopropane-1-carboxamide |
| 70 | 1-({[(1R)-1-(4-chlorophenyl)-2-[(1S)-1-(5- |
| and | chloropyridin-2-yl)ethyl]-7-fluoro-5-(2-hydroxypropan-2- |
| 71 | yl)-3-oxo-2,3-dihydro-1H-isoindol-1-yl]oxy}methyl)cyclo- |
| | propane-1-carboxamide and 1-({[(1R)-1- |
| | (4-chlorophenyl)-2-[(1R)-1-(5-chloropyridin-2- |
| | yl)ethyl]-7-fluoro-5-(2-hydroxypropan-2-yl)-3-oxo- |
| | 2,3-dihydro-1H-isoindol-1-yl]oxy}methyl)cyclopropane- |
| | 1-carboxamide |
| 72 | (3R)-3-(4-Chlorophenyl)-2-[(1S)-1-(5-chloropyridin- |
| | 2-yl)ethyl]-3-[(1-hydroxycyclopropyl)methoxy]- |
| | 6-(2-hydroxypropan-2-yl)-2,3-dihydro-1H-isoindol-1-one |
| 73 | 6-{[(1R)-1-(4-chlorophenyl)-7-fluoro-1-{[2- |
| | (hydroxymethyl)cyclopentyl]oxy}-5-(2-hydroxypropan-2- |
| | yl)-3-oxo-2,3-dihydro-1H-isoindol-2-yl]methyl}pyridine- |
| | 3-carbonitrile |
| 74 | (3R)-3-(4-Chlorophenyl)-2-[(5-chloropyridin- |
| | 2-yl)methyl]-6-(2-hydroxypropan-2-yl)-3-[(3- |
| | methyloxetan-3-yl)methoxy]-2,3-dihydro-1H-isoindol-1-one |
| 75 | (3R)-3-(4-chlorophenyl)-2-[(5-chloropyridin-2- |
| | yl)methyl]-4-fluoro-6-(2-hydroxypropan-2-yl)-3-[(3S)- |
| | oxolan-3-yloxy]-2,3-dihydro-1H-isoindol-1-one |
| 76 | (3R)-3-(4-chlorophenyl)-2-[(5-chloropyridin-2- |
| | yl)methyl]-4-fluoro-6-(2-hydroxypropan-2-yl)-3-[(3R)- |
| | oxolan-3-yloxy]-2,3-dihydro-1H-isoindol-1-one |
| 77 | (3R)-3-(4-chlorophenyl)-2-[(5-chloropyridin-2- |
| and | yl)methyl]-6-[1-hydroxy-1-(pyridin-3-yl)ethyl]-3- |
| 78 | methoxy-2,3-dihydro-1H-isoindol-1-one |
| 79 | (3R)-3-(4-chlorophenyl)-2-[(5-chloropyridin-2- |
| and | yl)methyl]-6-[1-hydroxy-1-(oxan-4-yl)ethyl]-3- |
| 80 | methoxy-2,3-dihydro-1H-isoindol-1-one |
| 81 | 6-{[(1R)-1-(4-chlorophenyl)-7-fluoro-1-[(cis-3- |
| | hydroxycyclobutyl)methoxy]-5-(2-hydroxypropan-2- |
| | yl)-3-oxo-2,3-dihydro-1H-isoindol-2-yl]methyl}pyridine- |
| | 3-carbonitrile |
| 82 | 1-({[(1R)-1-(4-chlorophenyl)-2-[(5-chloropyridin- |
| and | 2-yl)methyl]-7-fluoro-5-[1-hydroxy-1-(oxan-4- |
| 83 | yl)ethyl]-3-oxo-2,3-dihydro-1H-isoindol-1- |
| | yl]oxy}methyl)cyclopropane-1-carboxamide |
| 84 | 6-{[(1R)-1-(4-chlorophenyl)-7-fluoro-1-(3- |
| | hydroxycyclobutoxy)-5-(2-hydroxypropan-2-yl)-3-oxo- |
| | 2,3-dihydro-1H-isoindol-2-yl]methyl}pyridine-3-carbonitrile |
| 85 | (3R)-6-[1-(1-acetylpiperidin-4-yl)-1-hydroxyethyl]- |
| and | 3-(4-chlorophenyl)-2-[(5-chloropyridin-2- |
| 86 | yl)methyl]-3-methoxy-2,3-dihydro-1H-isoindol-1-one |
| 87 | 6-{[(1R)-1-(4-chlorophenyl)-1-(cyclopropylmethoxy)- |
| | 5-(2-hydroxypropan-2-yl)-3-oxo-2,3-dihydro- |
| | 1H-isoindol-2-yl]methyl}pyridine-3-carbonitrile |
| 88 | (3R)-3-(4-Chlorophenyl)-2-[(5-chloropyridin-2- |
| | yl)methyl]-6-[1-hydroxy-1-(1-oxo-1λ⁵-pyridin-3- |
| | yl)ethyl]-3-methoxy-2,3-dihydro-1H-isoindol-1-one |
| 89 | 6-{[(1R)-1-(4-chlorophenyl)-7-fluoro-5- |
| and | [1-hydroxy-1-(oxan-4-yl)ethyl]-1-{[1- |
| 90 | (hydroxymethyl)cyclopropyl]methoxy}-3-oxo-2,3- |
| | dihydro-1H-isoindol-2-yl]methyl}pyridine-3- |
| | carbonitrile |
| 91 | (3R)-3-(4-chlorophenyl)-2-[(5-chloropyridin-2- |
| and | yl)methyl]-6-[2-hydroxy-1-(oxan-4-yl)propan-2-yl]-3- |
| 92 | methoxy-2,3-dihydro-1H-isoindol-1-one |
| 93 | (3R)-3-(4-Chlorophenyl)-2-[(5-chloropyridin-2- |
| | yl)methyl]-3-(3-hydroxy-3-methylbutoxy)-6-(2- |
| | hydroxypropan-2-yl)-2,3-dihydro-1H-isoindol-1-one |
| 94 | (3R)-3-(4-Chlorophenyl)-2-[(5-chloropyridin- |
| | 2-yl)methyl]-6-(2-hydroxypropan-2-yl)-3-(2- |
| | methanesulfonylethoxy)-2,3-dihydro-1H-isoindol-1-one |
| 95 | (3R)-3-(4-chlorophenyl)-2-[(5-chloropyridin- |
| | 2-yl)methyl]-3-(cyclobutylmethoxy)-6-(2- |
| | hydroxypropan-2-yl)-2,3-dihydro-1H-isoindol-1-one |
| 96 | (3R)-3-(4-chlorophenyl)-2-[(5-chloropyridin-2- |
| | yl)methyl]-3-(2-hydroxy-2-methylpropoxy)-6-(2- |
| | hydroxypropan-2-yl)-2,3-dihydro-1H-isoindol-1-one |
| 97 | (3R)-3-(4-chlorophenyl)-2-[(5-chloropyridin-2- |
| and | yl)methyl]-4-fluoro-3-(2-hydroxybutoxy)-6-(2- |
| 98 | hydroxypropan-2-yl)-2,3-dihydro-1H-isoindol-1-one |
| 99 | 2-{2-[(1R)-1-(4-chlorophenyl)-2-[(5-chloropyridin- |
| and | 2-yl)methyl]-1-methoxy-3-oxo-2,3-dihydro-1H- |
| 100 | isoindol-5-yl]-2-hydroxypropoxy}-N,N-dimethylacetamide |
| 101 | (3R)-3-(4-chlorophenyl)-2-[(5-chloropyridin- |
| | 2-yl)methyl]-4-fluoro-3-{[1-(2- |
| | hydroxyethoxy)cyclopropyl]methoxy}-6-(2- |
| | hydroxypropan-2-yl)-2,3-dihydro-1H-isoindol-1-one |
| 102 | (3R)-3-(4-chlorophenyl)-2-[(5-chloropyridin-2- |
| and | yl)methyl]-6-[2-hydroxy-1-(2-hydroxyethoxy)propan- |
| 103 | 2-yl]-3-methoxy-2,3-dihydro-1H-isoindol-1-one |
| 104 | (3R)-3-(4-chlorophenyl)-2-[(5-chloropyridin-2- |
| and | yl)methyl]-6-[2-hydroxy-1-(piperazin-1-yl)propan-2- |
| 105 | yl]-3-methoxy-2,3-dihydro-1H-isoindol-1-one |
| 106 | (3R)-3-(4-chlorophenyl)-2-[(5-chloropyridin-2- |
| and | yl)methyl]-6-[2-hydroxy-1-(morpholin-4-yl)propan-2- |
| 107 | yl]-3-methoxy-2,3-dihydro-1H-isoindol-1-one |
| 108 | (3R)-3-(4-chlorophenyl)-2-[(5-chloropyridin-2- |
| and | yl)methyl]-6-[2-hydroxy-1-(methylamino)propan-2- |
| 109 | yl]-3-methoxy-2,3-dihydro-1H-isoindol-1-one |
| 110 | (3R)-3-(4-chlorophenyl)-2-[(5-chloropyridin-2- |
| and | yl)methyl]-6-[1-(cyclopropylamino)-2-hydroxypropan- |
| 111 | 2-yl]-3-methoxy-2,3-dihydro-1H-isoindol-1-one |
| 112 | (3R)-3-(4-chlorophenyl)-2-[(5-chloropyridin-2- |
| and | yl)methyl]-6-[2-hydroxy-1-(4-methyl-3-oxopiperazin- |
| 113 | 1-yl)propan-2-yl]-3-methoxy-2,3-dihydro-1H-isoindol-1-one |
| 114 | N-{2-[(1R)-1-(4-chlorophenyl)-2-[(5-chloropyridin- |
| and | 2-yl)methyl]-1-methoxy-3-oxo-2,3-dihydro-1H- |
| 115 | isoindol-5-yl]-2-hydroxypropyl}acetamide |
| 116 | (3R)-6-[1-(4-acetylpiperazin-1-yl)-2-hydroxypropan- |
| and | 2-yl]-3-(4-chlorophenyl)-2-[(5-chloropyridin-2- |
| 117 | yl)methyl]-3-methoxy-2,3-dihydro-1H-isoindol-1-one |
| 118 | 6-{[(1R)-1-(4-chlorophenyl)-7-fluoro-1-[(2- |
| and | hydroxycyclopentyl)oxy]-5-(2-hydroxypropan-2-yl)-3- |
| 119 | oxo-2,3-dihydro-1H-isoindol-2-yl]methyl}pyridine- |
| | 3-carbonitrile |
| 120 | (3R)-3-(4-chlorophenyl)-2-[(5-chloropyridin-2- |
| and | yl)methyl]-6-[1-hydroxy-1-(pyrimidin-5-yl)ethyl]-3- |
| 121 | methoxy-2,3-dihydro-1H-isoindol-1-one |
| 122 | (3R)-3-(4-chlorophenyl)-2-[(5-chloropyridin-2- |
| and | yl)methyl]-6-[1-hydroxy-1-(pyridin-4-yl)ethyl]-3- |
| 123 | methoxy-2,3-dihydro-1H-isoindol-1-one |
| 124 | 6-{[(1R)-1-(4-chlorophenyl)-7-fluoro-1-[(2- |
| | hydroxycyclopentyl)oxy]-5-(2-hydroxypropan-2-yl)-3- |
| | oxo-2,3-dihydro-1H-isoindol-2-yl]methyl}pyridine- |
| | 3-carbonitrile |
| 125 | (3R)-3-(4-chlorophenyl)-2-[(5-chloropyridin-2- |
| and | yl)methyl]-6-[1-hydroxy-1-(2-methoxypyridin-4- |
| 126 | yl)ethyl]-3-methoxy-2,3-dihydro-1H-isoindol-1-one |
| 127 | 1-({[(1R)-5-[1-(4-Acetylpiperazin-1-yl)-2- |
| | hydroxypropan-2-yl]-1-(4-chlorophenyl)-2-[(5- |
| | chloropyridin-2-yl)methyl]-7-fluoro-3-oxo-2,3-dihydro- |
| | 1H-isoindol-1-yl]oxy}methyl)cyclopropane-1- |
| | carboxamide |
| 128 | 1-({[(1R)-1-(4-Chlorophenyl)-2-[(5-chloropyridin- |
| and | 2-yl)methyl]-7-fluoro-5-[2-hydroxy-1-(4- |
| 129 | methylpiperazin-1-yl)propan-2-yl]-3-oxo-2,3-dihydro- |
| | 1H-isoindol-1-yl]oxy}methyl)cyclopropane-1- |
| | carboxamide |
| 130 | 1-({[(1R)-1-(4-Chlorophenyl)-2-[(5-chloropyridin- |
| and | 2-yl)methyl]-7-fluoro-5-(2-hydroxy-1- |
| 131 | methoxypropan-2-yl)-3-oxo-2,3-dihydro-1H-isoindol- |
| | 1-yl]oxy}methyl)cyclopropane-1-carboxamide |
| 132 | (3R)-3-(4-Chlorophenyl)-2-[(5-chloropyridin-2- |
| and | yl)methyl]-6-[1-hydroxy-1-(1-methyl-1H-imidazol-5- |
| 133 | yl)ethyl]-3-methoxy-2,3-dihydro-1H-isoindol-1-one |
| 134 | (3R)-3-(4-Chlorophenyl)-2-[(5-chloropyridin-2- |
| and | yl)methyl]-6-[1-hydroxy-1-(1H-pyrazol-5-yl)ethyl]-3- |
| 135 | methoxy-2,3-dihydro-1H-isoindol-1-one |
| 136 | (3R)-3-(4-Chlorophenyl)-2-[(5-chloropyridin- |
| and | 2-yl)methyl]-4-fluoro-6-[2-hydroxy-1-(4- |
| 137 | methylpiperazin-1-yl)propan-2-yl]-3-[(1- |
| | hydroxycyclopropyl)methoxy]-2,3-dihydro-1H-isoindol-1- |
| | one |
| 138 | (3R)-3-(4-Chlorophenyl)-2-[(5-chloropyridin-2- |
| and | yl)methyl]-6-[1-(dimethylamino)-2-hydroxypropan-2- |
| 139 | yl]-4-fluoro-3-[(3S)-oxolan-3-yloxy]-2,3-dihydro- |
| | 1H-isoindol-1-one |
| 140 | (3R)-3-(4-Chlorophenyl)-2-[(5-chloropyridin-2- |
| and | yl)methyl]-6-(1-ethoxy-2-hydroxypropan-2-yl)-4- |
| 141 | fluoro-3-[(3S)-oxolan-3-yloxy]-2,3-dihydro-1H-isoindol-1-one |
| 142 | (3R)-3-(4-Chlorophenyl)-2-[(5-chloropyridin- |
| and | 2-yl)(²H₂)methyl]-4-fluoro-6-[2-hydroxy-1- |
| 143 | (2H3)methoxypropan-2-yl]-3-[(3S)-oxolan- |
| | 3-yloxy]-2,3-dihydro-1H-isoindol-1-one |
| 144 | 2-{[1-{{[(1R)-1-(4-Chlorophenyl)-2-[(5-chloropyridin- |
| | 2-yl)methyl]-7-fluoro-5-(2-hydroxypropan-2-yl)- |
| | 3-oxo-2,3-dihydro-1H-isoindol-1- |
| | yl]oxy}methyl)cyclopropyl]methoxy}acetic acid |
| 145 | 2-[(1R)-1-(4-Chlorophenyl)-2-[(5-chloropyridin- |
| and | 2-yl)methyl]-1-methoxy-3-oxo-2,3-dihydro-1H- |
| 146 | isoindol-5-yl]-2-hydroxy-N-methylpropanamide |
| 147 | 2-[(1R)-1-(4-Chlorophenyl)-2-[(5-chloropyridin- |
| and | 2-yl)methyl]-1-methoxy-3-oxo-2,3-dihydro-1H- |
| 148 | isoindol-5-yl]-N-ethyl-2-hydroxypropanamide |
| 149 | 2-[(1R)-1-(4-Chlorophenyl)-2-[(5-chloropyridin- |
| and | 2-yl)methyl]-1-methoxy-3-oxo-2,3-dihydro-1H- |
| 150 | isoindol-5-yl]-N-[2-(dimethylamino)ethyl]- |
| | 2-hydroxypropanamide |
| 151 | 2-[(1R)-1-(4-Chlorophenyl)-2-[(5-chloropyridin-2- |
| and | yl)methyl]-1-methoxy-3-oxo-2,3-dihydro-1H- |
| 152 | isoindol-5-yl]-2-hydroxy-N-(propan-2-yl)propanamide |
| 153 | 6-{[(1R)-1-(4-Chlorophenyl)-7-fluoro-1- |
| and | {[1-(1-hydroxyethyl)cyclopropyl]methoxy}-5-(2- |
| 154 | hydroxypropan-2-yl)-3-oxo-2,3-dihydro-1H- |
| | isoindol-2-yl]methyl}pyridine-3-carbonitrile |
| 155 | 2-({[1-{{[(1R)-1-(4-chlorophenyl)-2-[(5- |
| | chloropyridin-2-yl)methyl]-7-fluoro-5-(2-hydroxypropan-2- |
| | yl)-3-oxo-2,3-dihydro-1H-isoindol-1-yl]oxy}methyl)cyclo- |
| | propyl]methyl}amino)-N-methylacetamide |
| 156 | N-{[1-({[(1R)-1-(4-Chlorophenyl)-2-[(5- |
| | chloropyridin-2-yl)methyl]-7-fluoro-5-(2-hydroxypropan-2- |
| | yl)-3-oxo-2,3-dihydro-1H-isoindol-1- |
| | yl]oxy}methyl)cyclopropyl]methyl}acetamide |
| 157 | (3R)-3-(4-Chlorophenyl)-2-[(5-chloropyridin- |
| and | 2-yl)methyl]-6-[2-hydroxy-1-(2-oxoimidazolidin-1- |
| 158 | yl)propan-2-yl]-3-methoxy-2,3-dihydro-1H-isoindol-1-one |
| 159 | (3R)-3-(4-Chlorophenyl)-2-[(5-chloropyridin-2- |
| and | yl)methyl]-6-[2-hydroxy-1-(1H-imidazol-1-yl)propan- |
| 160 | 2-yl]-3-methoxy-2,3-dihydro-1H-isoindol-1-one |
| 161 | (3R)-3-(4-Chlorophenyl)-2-[(5-chloropyridin- |
| and | 2-yl)methyl]-6-[1-(1,2-dimethyl-1H-imidazol-4-yl)-1- |
| 162 | hydroxyethyl]-4-fluoro-3-methoxy-2,3-dihydro- |
| | 1H-isoindol-1-one |
| 163 | (3R)-3-(4-chlorophenyl)-2-[(5-chloropyridin-2- |
| and | yl)methyl]-4-fluoro-6-[1-hydroxy-1-(1H-imidazol-2- |
| 164 | yl)ethyl]-3-methoxy-2,3-dihydro-1H-isoindol-1-one |
| 165 | (3R)-3-(4-Chlorophenyl)-2-[(5-chloropyridin- |
| and | 2-yl)methyl]-4-fluoro-6-[1-hydroxy-1-(1,3-thiazol-2- |
| 166 | yl)ethyl]-3-methoxy-2,3-dihydro-1H-isoindol-1-one |
| 167 | (2S)-3-{[(1R)-1-(4-Chlorophenyl)-2-[(5- |
| | chloropyridin-2-yl)methyl]-7-fluoro-5-(2-hydroxypropan-2- |
| | yl)-3-oxo-2,3-dihydro-1H-isoindol-1- |
| | yl]oxy}-2-methylpropanamide |
| 168 | (2R)-3-{[(1R)-1-(4-Chlorophenyl)-2-[(5-chloropyridin- |
| | 2-yl)methyl]-7-fluoro-5-(2-hydroxypropan-2- |
| | yl)-3-oxo-2,3-dihydro-1H-isoindol-1-yl]oxy}-2- |
| | methylpropanamide |
| 169 | 6-[(1S)-1-[(1R)-1-(4-Chlorophenyl)-7-fluoro-1- |
| | {[1-(hydroxymethyl)cyclopropyl]methoxy}-5-(2- |
| | hydroxypropan-2-yl)-3-oxo-2,3-dihydro-1H- |
| | isoindol-2-yl]ethyl]pyridine-3-carbonitrile |
| 170 | 6-[(1R)-1-[(1R)-1-(4-Chlorophenyl)-7-fluoro-1- |
| | {[1-(hydroxymethyl)cyclopropyl]methoxy}-5-(2- |
| | hydroxypropan-2-yl)-3-oxo-2,3-dihydro-1H- |
| | isoindol-2-yl]ethyl]pyridine-3-carbonitrile |
| 171 | (3R)-3-(4-Chlorophenyl)-2-[(5-chloropyridin-2- |
| and | yl)methyl]-4-fluoro-6-(2-hydroxypropan-2-yl)-3-[(1- |
| 172 | methanesulfinylcyclopropyl)methoxy]-2,3- |
| | dihydro-1H-isoindol-1-one |
| 173 | 6-{[(1R)-1-(4-Chlorophenyl)-7-fluoro-5-(2- |
| | hydroxypropan-2-yl)-1-methoxy-3-oxo-2,3-dihydro-1H- |
| | isoindol-2-yl]methyl}pyridine-3-carbonitrile. |
| 174 | (3R)-3-(4-Chlorophenyl)-2-[(1S)-1-(5- |
| | chloropyridin-2-yl)prop-2-en-1-yl]-4-fluoro-3-[(1- |
| | hydroxycyclopropyl)methoxy]-6-(2-hydroxypropan- |
| | 2-yl)-2,3-dihydro-1H-isoindol-1-one |
| 175 | 1-({[(1R)-1-(4-Chlorophenyl)-2-[(5-chloropyridin- |
| and | 2-yl)methyl]-7-fluoro-5-[hydroxy(1-methyl-1H- |
| 176 | pyrazol-4-yl)methyl]-3-oxo-2,3-dihydro-1H-isoindol- |
| | 1-yl]oxy}methyl)cyclopropane-1-carboxamide |
| 177 | 1-({[(1R)-1-(4-Chlorophenyl)-2-[(5-chloropyridin- |
| and | 2-yl)methyl]-7-fluoro-5-[1-hydroxy-1-(1-methyl- |
| 178 | 1H-pyrazol-4-yl)ethyl]-3-oxo-2,3-dihydro-1H- |
| | isoindol-1-yl]oxy}methyl)cyclopropane-1- |
| | carboxamide |
| 179 | 1-({[(1R)-1-(4-Chlorophenyl)-2-[(5-chloropyridin- |
| and | 2-yl)methyl]-5-[1-(1-ethyl-1H-pyrazol-4-yl)-1- |
| 180 | hydroxyethyl]-7-fluoro-3-oxo-2,3-dihydro- |
| | 1H-isoindol-1-yl]oxy}methyl)cyclopropane-1- |
| | carboxamide |
| 18 | (3R)-6-{1-[1-(1-Acetylazetidin-3-yl)-1H-pyrazol- |
| and | 4-yl]-1-hydroxyethyl}-3-(4-chlorophenyl)-2-[(5- |
| 182 | chloropyridin-2-yl)methyl]-4-fluoro-3- |
| | methoxy-2,3-dihydro-1H-isoindol-1-one |
| 183 | (3R)-3-(4-Chlorophenyl)-2-[(5-chloropyridin-2- |
| and | yl)methyl]-6-[1-(1-ethyl-1H-pyrazol-4-yl)-1- |
| 184 | hydroxyethyl]-4-fluoro-3-[(1-hydroxycyclopropyl)methoxy]- |
| | 2,3-dihydro-1H-isoindol-1-one |
| 185 | (3R)-3-(4-Chlorophenyl)-2-[(5-chloropyridin-2- |
| and | yl)methyl]-4-fluoro-6-[1-hydroxy-1-(5-methyl-1,3,4- |
| 186 | oxadiazol-2-yl)ethyl]-3-[(1-hydroxycyclopropyl)methoxy]- |
| | 2,3-dihydro-1H-isoindol-1-one |
| 187 | (3R)-3-(4-Chlorophenyl)-2-[(5-chloropyridin-2- |
| and | yl)methyl]-4-fluoro-6-[1-hydroxy-1-(1-methyl-1H- |
| 188 | 1,2,3-triazol-4-yl)ethyl]-3-methoxy-2,3-dihydro- |
| | 1H-isoindol-1-one |
| 189 | (3R)-3-(4-Chlorophenyl)-2-[(5-chloropyridin-2- |
| and | yl)methyl]-4-fluoro-6-[1-hydroxy-1-(1-methyl-1H- |
| 190 | pyrazol-3-yl)ethyl]-3-methoxy-2,3-dihydro-1H-isoindol-1-one |
| 191 | (3R)-3-(4-Chlorophenyl)-2-[(5-chloropyridin-2- |
| and | yl)methyl]-6-(1-{1-[2-(dimethylamino)ethyl]-1H- |
| 192 | pyrazol-4-yl}-1-hydroxyethyl)-4-fluoro-3-methoxy- |
| | 2,3-dihydro-1H-isoindol-1-one |
| 193 | 1-({[(1R)-1-(4-chlorophenyl)-2-[(5-chloropyridin- |
| and | 2-yl)methyl]-7-fluoro-5-[1-hydroxy-1-(1,3-thiazol-4- |
| 194 | yl)ethyl]-3-oxo-2,3-dihydro-1H-isoindol-1- |
| | yl]oxy}methyl)cyclopropane-1-carboxamide |
| 195 | (3R)-3-(4-chlorophenyl)-2-[(5-chloropyridin-2- |
| and | yl)methyl]-4-fluoro-6-[1-hydroxy-1-(1-methyl-1H- |
| 196 | imidazol-4-yl)ethyl]-3-[(1-hydroxycyclopropyl)methoxy]- |
| | 2,3-dihydro-1H-isoindol-1-one |
| 197 | 1-({[(1R)-1-(4-chlorophenyl)-2-[(5-chloropyridin-2- |
| and | yl)methyl]-5-[1-(1,2-dimethyl-1H-imidazol-4-yl)- |
| 198 | 1-hydroxyethyl]-7-fluoro-3-oxo-2,3-dihydro- |
| | 1H-isoindol-1-yl]oxy}methyl)cyclopropane-1- |
| | carboxamide |
| 199 | (3R)-3-(4-chlorophenyl)-2-[(5-chloropyridin-2- |
| and | yl)methyl]-4-fluoro-6-[1-hydroxy-1-(1-methyl-1H- |
| 200 | pyrazol-4-yl)ethyl]-3-[(1-hydroxycyclopropyl)methoxy]- |
| | 2,3-dihydro-1H-isoindol-1-one |
| 201 | 1-({[(1R)-1-(4-chlorophenyl)-2-[(5-chloropyridin-2- |
| and | yl)methyl]-7-fluoro-5-[1-hydroxy-1-(1-methyl-1H- |
| 202 | pyrazol-4-yl)ethyl]-3-oxo-2,3-dihydro-1H-isoindol- |
| | 1-yl]oxy}methyl)cyclopropane-1-carbonitrile |
| 203 | (3R)-3-(4-chlorophenyl)-2-[(5-chloropyrimidin- |
| and | 2-yl)methyl]-4-fluoro-6-[1-hydroxy-1-(1-methyl-1H- |
| 204 | pyrazol-4-yl)ethyl]-3-[(1-hydroxycyclopropyl)methoxy]- |
| | 2,3-dihydro-1H-isoindol-1-one |
| 205 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[cis-3-hydroxycyclobutoxy]- |
| | 2,3-dihydro-1H-isoindol-1-one |
| 206 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[trans-3-hydroxycyclobutoxy]- |
| | 2,3-dihydro-1H-isoindol-1-one |
| 207 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[trans-3-hydroxycyclobutoxy]- |
| | 2,3-dihydro-1H-isoindol-1-one |
| 208 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[cis-3-hydroxycyclobutoxy]- |
| | 2,3-dihydro-1H-isoindol-1-one |
| 209 | 1-({[(1R)-1-(4-chlorophenyl)-7-fluoro-2-[(5- |
| | fluoropyridin-2-yl)methyl]-5-[1-hydroxy-1-(1-methyl-1H- |
| | pyrazol-4-yl)ethyl]-3-oxo-2,3-dihydro-1H-isoindol- |
| | 1-yl]oxy}methyl)cyclopropane-1-carboxamide |
| 210 | 1-({[(1R)-1-(4-chlorophenyl)-7-fluoro-2-[(5- |
| | fluoropyridin-2-yl)methyl]-5-[1-hydroxy-1-(1-methyl-1H- |
| | pyrazol-4-yl)ethyl]-3-oxo-2,3-dihydro-1H-isoindol- |
| | 1-yl]oxy}methyl)cyclopropane-1-carboxamide |
| 211 | 6-{[(1R)-1-(4-chlorophenyl)-1-[(1-cyanocyclo- |
| | propyl)methoxy]-7-fluoro-5-[1-hydroxy-1-(1-methyl- |
| | 1H-pyrazol-4-yl)ethyl]-3-oxo-2,3-dihydro-1H- |
| | isoindol-2-yl]methyl}pyridine-3-carbonitrile |
| 212 | 6-{[(1R)-1-(4-chlorophenyl)-1-[(1-cyanocyclopro- |
| | pyl)methoxy]-7-fluoro-5-[1-hydroxy-1-(1-methyl- |
| | 1H-pyrazol-4-yl)ethyl]-3-oxo-2,3-dihydro-1H-isoindol- |
| | 2-yl]methyl}pyridine-3-carbonitrile |
| 213 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-(2-hydroxyethoxy)- |
| | 2,3-dihydro-1H-isoindol-1-one |
| 214 | 1-({[(1R)-1-(4-chlorophenyl)-2-[(5-chloropyrimidin- |
| | 2-yl)methyl]-7-fluoro-5-[1-hydroxy-1-(1-methyl- |
| | 1H-pyrazol-4-yl)ethyl]-3-oxo-2,3-dihydro- |
| | 1H-isoindol-1-yl]oxy}methyl)cyclopropane-1- |
| | carboxamide |
| 215 | 1-({[(1R)-1-(4-chlorophenyl)-2-[(5-chloropyrimidin- |
| | 2-yl)methyl]-7-fluoro-5-[1-hydroxy-1-(1-methyl- |
| | 1H-pyrazol-4-yl)ethyl]-3-oxo-2,3-dihydro-1H- |
| | isoindol-1-yl]oxy}methyl)cyclopropane-1- |
| | carboxamide |
| 216 | 1-({[(1R)-1-(4-chlorophenyl)-7-fluoro-5-[1- |
| | hydroxy-1-(1-methyl-1H-pyrazol-4-yl)ethyl]-2-[(6- |
| | methoxypyridin-3-yl)methyl]-3-oxo-2,3-dihydro- |
| | 1H-isoindol-1-yl]oxy}methyl)cyclopropane-1- |
| | carboxamide |
| 217 | 1-({[(1R)-1-(4-chlorophenyl)-7-fluoro-5-[1- |
| | hydroxy-1-(1-methyl-1H-pyrazol-4-yl)ethyl]-2-[(6- |
| | methoxypyridin-3-yl)methyl]-3-oxo-2,3-dihydro- |
| | 1H-isoindol-1-yl]oxy}methyl)cyclopropane-1- |
| | carboxamide |
| 218 | 6-{[(1R)-1-(4-chlorophenyl)-7-fluoro-5-[1- |
| | hydroxy-1-(1-methyl-1H-pyrazol-4-yl)ethyl]-3-oxo-1- |
| | [(3S)-oxolan-3-yloxy]-2,3-dihydro-1H-isoindol- |
| | 2-yl]methyl}pyridine-3-carbonitrile |
| 219 | (3R)-3-(4-chlorophenyl)-4-fluoro-6-[1-hydroxy- |
| | 1-(1-methyl-1H-pyrazol-4-yl)ethyl]-3-{[1- |
| | (hydroxymethyl)cyclopropyl]methoxy}-2-[(6- |
| | methoxypyridin-3-yl)methyl]-2,3-dihydro-1H-isoindol- |
| | 1-one |
| 220 | (3R)-3-(4-chlorophenyl)-4-fluoro-6-[1-hydroxy- |
| | 1-(1-methyl-1H-pyrazol-4-yl)ethyl]-3-{[1- |
| | (hydroxymethyl)cyclopropyl]methoxy}-2-[(6- |
| | methoxypyridin-3-yl)methyl]-2,3-dihydro-1H-isoindol- |
| | 1-one |
| 221 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[(2R)-2-hydroxypropoxy]- |
| | 2,3-dihydro-1H-isoindol-1-one |
| 222 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[(3S)-oxolan-3-yloxy]-2,3- |
| | dihydro-1H-isoindol-1-one |
| 223 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[(3S)-oxolan-3-yloxy]-2,3- |
| | dihydro-1H-isoindol-1-one |
| 224 | (3R)-3-(4-chlorophenyl)-4-fluoro-6-[1-hydroxy- |
| | 1-(1-methyl-1H-pyrazol-4-yl)ethyl]-3-{[1- |
| | (hydroxymethyl)cyclopropyl]methoxy}-2-[(5- |
| | methoxypyridin-2-yl)methyl]-2,3-dihydro-1H-isoindol- |
| | 1-one |
| 225 | (3R)-3-(4-chlorophenyl)-4-fluoro-6-[1-hydroxy- |
| | 1-(1-methyl-1H-pyrazol-4-yl)ethyl]-3-[(1- |
| | hydroxycyclopropyl) methoxy]-2-[(5-methylpyridin- |
| | 2-yl)methyl]-2,3-dihydro-1H-isoindol-1-one |
| 226 | (3R)-3-(4-chlorophenyl)-4-fluoro-6-[1-hydroxy- |
| | 1-(1-methyl-1H-pyrazol-4-yl)ethyl]-3-{[1- |
| | (hydroxymethyl)cyclopropyl]methoxy}-2-[(5- |
| | methoxypyridin-2-yl)methyl]-2,3-dihydro-1H-isoindol- |
| | 1-one |
| 227 | (3R)-3-(4-chlorophenyl)-4-fluoro-6-[1-hydroxy- |
| | 1-(1-methyl-1H-pyrazol-4-yl)ethyl]-3-[(1- |
| | hydroxycyclopropyl) methoxy]-2-[(5-methylpyridin- |
| | 2-yl)methyl]-2,3-dihydro-1H-isoindol-1-one |
| 228 | (3R)-3-(4-chlorophenyl)-4-fluoro-6-[1-hydroxy- |
| | 1-(1-methyl-1H-pyrazol-4-yl)ethyl]-3-[(1- |
| | hydroxycyclopropyl)methoxy]-2-[(5-methoxypyridin- |
| | 2-yl)methyl]-2,3-dihydro-1H-isoindol-1-one |
| 229 | (3R)-3-(4-chlorophenyl)-4-fluoro-6-[1-hydroxy- |
| | 1-(1-methyl-1H-pyrazol-4-yl)ethyl]-3-[(1- |
| | hydroxycyclopropyl)methoxy]-2-[(6-methoxypyridin- |
| | 3-yl)methyl]-2,3-dihydro-1H-isoindol-1-one |
| 230 | (3R)-3-(4-chlorophenyl)-4-fluoro-6-[1-hydroxy- |
| | 1-(1-methyl-1H-pyrazol-4-yl)ethyl]-3-[(1- |
| | hydroxycyclopropyl)methoxy]-2-[(6-methoxypyridin- |
| | 3-yl)methyl]-2,3-dihydro-1H-isoindol-1-one |
| 231 | (3R)-3-(4-chlorophenyl)-4-fluoro-2-[(5-fluoropyridin- |
| | 2-yl)methyl]-6-[1-hydroxy-1-(1-methyl-1H- |
| | pyrazol-4-yl)ethyl]-3-[(1-hydroxycyclopro- |
| | pyl)methoxy]-2,3-dihydro-1H-isoindol-1-one |
| 232 | (3R)-3-(4-chlorophenyl)-4-fluoro-2-[(5- |
| | fluoropyridin-2-yl)methyl]-6-[1-hydroxy-1-(1-methyl-1H- |
| | pyrazol-4-yl)ethyl]-3-[(1-hydroxycyclo- |
| | propyl)methoxy]-2,3-dihydro-1H-isoindol-1-one |
| 233 | (3R)-3-(4-chlorophenyl)-2-[(5-chloropyridin-2- |
| and | yl)methyl]-6-[2-hydroxy-1-(pyridin-3-yloxy)propan-2- |
| 234 | yl]-3-methoxy-2,3-dihydro-1H-isoindol-1-one |
| 235 | (3R)-3-(4-chlorophenyl)-2-[(5-chloropyridin-2- |
| | yl)methyl]-6-(1-ethoxy-2-hydroxypropan-2-yl)-3- |
| | methoxy-2,3-dihydro-1H-isoindol-1-one |
| 236 | (3R)-3-(4-chlorophenyl)-2-[(5-chloropyridin-2- |
| | yl)methyl]-6-(1-ethoxy-2-hydroxypropan-2-yl)-3- |
| | methoxy-2,3-dihydro-1H-isoindol-1-one |
| 237 | (3R)-3-(4-chlorophenyl)-2-[(5-chloropyridin- |
| | 2-yl)methyl]-6-{2-hydroxy-1-[4-(2- |
| | hydroxyethyl)piperazin-1-yl]propan-2-yl}-3- |
| | methoxy-2,3-dihydro-1H-isoindol-1-one |
| 238 | (3R)-3-(4-chlorophenyl)-2-[(5-chloropyridin-2- |
| | yl)methyl]-6-[2-hydroxy-1-(4-hydroxypiperidin-1- |
| | yl)propan-2-yl]-3-methoxy-2,3-dihydro-1H-isoindol-1-one |
| 239 | (3R)-3-(4-chlorophenyl)-2-[(5-chloropyridin-2- |
| | yl)methyl]-6-{2-hydroxy-1-[methyl(1-methylpiperidin- |
| | 4-yl)amino]propan-2-yl}-3-methoxy-2,3-dihydro- |
| | 1H-isoindol-1-one |
| 240 | (3R)-3-(4-chlorophenyl)-2-[(5-chloropyridin-2- |
| | yl)methyl]-6-{2-hydroxy-1-[(oxan-4-yl)amino]propan- |
| | 2-yl}-3-methoxy-2,3-dihydro-1H-isoindol-1-one |
| 241 | (3R)-3-(4-chlorophenyl)-2-[(5-chloropyridin- |
| | 2-yl)methyl]-6-[2-hydroxy-1-(3-oxopiperazin-1- |
| | yl)propan-2-yl]-3-methoxy-2,3-dihydro-1H-isoindol-1-one |
| 242 | (3R)-3-(4-chlorophenyl)-2-[(5-chloropyridin-2- |
| | yl)methyl]-6-[1-(1,4-diazepan-1-yl)-2-hydroxypropan- |
| | 2-yl]-3-methoxy-2,3-dihydro-1H-isoindol-1-one |
| 243 | (3R)-3-(4-chlorophenyl)-2-[(5-chloropyridin-2- |
| | yl)methyl]-6-[1-(1,4-diazepan-1-yl)-2-hydroxypropan- |
| | 2-yl]-3-methoxy-2,3-dihydro-1H-isoindol-1-one |
| 244 | (3R)-3-(4-chlorophenyl)-2-[(5-chloropyridin-2- |
| | yl)methyl]-6-{2-hydroxy-1-[(oxan-4-yl)amino]propan- |
| | 2-yl}-3-methoxy-2,3-dihydro-1H-isoindol-1-one |
| 245 | (3R)-3-(4-chlorophenyl)-2-[(5-chloropyridin-2- |
| | yl)methyl]-6-[2-hydroxy-1-(3-oxopiperazin-1- |
| | yl)propan-2-yl]-3-methoxy-2,3-dihydro-1H-isoindol-1-one |
| 246 | (3R)-3-(4-chlorophenyl)-2-[(5-chloropyridin- |
| | 2-yl)methyl]-6-{2-hydroxy-1-[4-(2- |
| | hydroxyethyl)piperazin-1-yl]propan-2-yl}-3- |
| | methoxy-2,3-dihydro-1H-isoindol-1-one |
| 247 | (3R)-3-(4-chlorophenyl)-2-[(5-chloropyridin-2- |
| | yl)methyl]-6-[2-hydroxy-1-(4-hydroxypiperidin-1- |
| | yl)propan-2-yl]-3-methoxy-2,3-dihydro-1H-isoindol-1-one |
| 248 | (3R)-3-(4-chlorophenyl)-2-[(5-chloropyridin-2- |
| | yl)methyl]-6-{2-hydroxy-1-[methyl(1-methylpiperidin- |
| | 4-yl)amino]propan-2-yl}-3-methoxy-2,3-dihydro- |
| | 1H-isoindol-1-one |
| 249 | 4-{2-[(1R)-1-(4-chlorophenyl)-2-[(5-chloropyridin- |
| | 2-yl)methyl]-1-methoxy-3-oxo-2,3-dihydro-1H- |
| | isoindol-5-yl]-2-hydroxypropyl}-1λ6-thiomorpholine-1,1-dione |
| 250 | (3R)-3-(4-chlorophenyl)-2-[(5-chloropyridin-2- |
| | yl)methyl]-6-{2-hydroxy-1-[(2S)-2- |
| | (hydroxymethyl)pyrrolidin-1-yl]propan-2-yl}-3- |
| | methoxy-2,3-dihydro-1H-isoindol-1-one |
| 251 | (3R)-3-(4-chlorophenyl)-2-[(5-chloropyridin- |
| | 2-yl)methyl]-6-{2-hydroxy-1-[(2S)-2- |
| | (hydroxymethyl)pyrrolidin-1-yl]propan-2-yl}- |
| | 3-methoxy-2,3-dihydro-1H-isoindol-1-one |
| 252 | 4-{2-[(1R)-1-(4-chlorophenyl)-2-[(5-chloropyridin- |
| | 2-yl)methyl]-1-methoxy-3-oxo-2,3-dihydro-1H- |
| | isoindol-5-yl]-2-hydroxypropyl}-1λ6-thiomorpholine-1,1-dione |
| 253 | (3R)-6-{1-[(1-acetylpiperidin-4-yl)(methyl)amino]- |
| | 2-hydroxypropan-2-yl}-3-(4-chlorophenyl)-2-[(5- |
| | chloropyridin-2-yl)methyl]-3-methoxy-2,3-dihydro- |
| | 1H-isoindol-1-one |
| 253 | (3R)-6-{1-[(1-acetylpiperidin-4-yl)(methyl)amino]- |
| a | 2-hydroxypropan-2-yl}-3-(4-chlorophenyl)-2-[(5- |
| | chloropyridin-2-yl)methyl]-3-methoxy-2,3-dihydro- |
| | 1H-isoindol-1-one |
| 254 | (3R)-6-[1-(4-aminopiperidin-1-yl)-2-hydroxypropan- |
| | 2-yl]-3-(4-chlorophenyl)-2-[(5-chloropyridin-2- |
| | yl)methyl]-3-methoxy-2,3-dihydro-1H-isoindol-1-one |
| 255 | (3R)-3-(4-chlorophenyl)-2-[(5-chloropyridin- |
| | 2-yl)methyl]-6-[2-hydroxy-1-(4-methylpiperazin-1- |
| | yl)propan-2-yl]-3-methoxy-2,3-dihydro-1H-isoindol-1-one |
| 256 | (3R)-3-(4-chlorophenyl)-2-[(5-chloropyridin-2- |
| | yl)methyl]-6-[2-hydroxy-1-(4-methylpiperazin-1- |
| | yl)propan-2-yl]-3-methoxy-2,3-dihydro-1H-isoindol-1-one |
| 257 | (3R)-3-(4-chlorophenyl)-2-[(5-chloropyridin-2- |
| | yl)methyl]-6-[2-hydroxy-1-(2-oxopyrrolidin-1- |
| | yl)propan-2-yl]-3-methoxy-2,3-dihydro-1H-isoindol-1-one |
| 258 | (3R)-3-(4-chlorophenyl)-2-[(5-chloropyridin-2- |
| | yl)methyl]-6-[2-hydroxy-1-(2-oxopyrrolidin-1- |
| | yl)propan-2-yl]-3-methoxy-2,3-dihydro-1H-isoindol-1-one |
| 259 | (3R)-6-[1-(4-aminopiperidin-1-yl)-2-hydroxypropan- |
| | 2-yl]-3-(4-chlorophenyl)-2-[(5-chloropyridin-2- |
| | yl)methyl]-3-methoxy-2,3-dihydro-1H-isoindol-1-one |
| 260 | (3R)-3-(4-chlorophenyl)-2-[(5-chloropyridin-2- |
| | yl)methyl]-6-[2-hydroxy-1-(5-oxo-1,4-diazepan-1- |
| | yl)propan-2-yl]-3-methoxy-2,3-dihydro-1H-isoindol-1-one |
| 261 | (3R)-3-(4-chlorophenyl)-2-[(5-chloropyridin-2- |
| | yl)methyl]-6-[2-hydroxy-1-(5-oxo-1,4-diazepan-1- |
| | yl)propan-2-yl]-3-methoxy-2,3-dihydro-1H-isoindol-1-one |
| 262 | (3R)-3-(4-chlorophenyl)-2-[(5-chloropyridin- |
| | 2-yl)methyl]-6-(2-hydroxy-1-{4H,5H,6H,7H- |
| | [1,2,3]triazolo[1,5-a]pyrazin-5-yl}propan-2-yl)- |
| | 3-methoxy-2,3-dihydro-1H-isoindol-1-one |
| 263 | (3R)-3-(4-chlorophenyl)-2-[(5-chloropyridin-2- |
| | yl)methyl]-6-{2-hydroxy-1-[(1S,4S)-5-methyl-2,5- |
| | diazabicyclo[2.2.1]heptan-2-yl]propan-2-yl}- |
| | 3-methoxy-2,3-dihydro-1H-isoindol-1-one |
| 264 | (3R)-3-(4-chlorophenyl)-2-[(5-chloropyridin-2- |
| | yl)methyl]-6-{2-hydroxy-1-[(1S,4S)-5-methyl-2,5- |
| | diazabicyclo[2.2.1]heptan-2-yl]propan-2-yl}- |
| | 3-methoxy-2,3-dihydro-1H-isoindol-1-one |
| 265 | (3R)-3-(4-chlorophenyl)-2-[(5-chloropyridin- |
| | 2-yl)methyl]-6-{2-hydroxy-1-[(2R)-2- |
| | (hydroxymethyl)pyrrolidin-1-yl]propan-2-yl}- |
| | 3-methoxy-2,3-dihydro-1H-isoindol-1-one |
| 266 | (3R)-3-(4-chlorophenyl)-2-[(5-chloropyridin-2- |
| | yl)methyl]-6-[2-hydroxy-1-(4-methyl-1,4-diazepan-1- |
| | yl)propan-2-yl]-3-methoxy-2,3-dihydro-1H-isoindol-1-one |
| 267 | (3R)-3-(4-chlorophenyl)-2-[(5-chloropyridin- |
| | 2-yl)methyl]-6-{2-hydroxy-1-[(2R)-2- |
| | (hydroxymethyl)pyrrolidin-1-yl]propan-2-yl}-3- |
| | methoxy-2,3-dihydro-1H-isoindol-1-one |
| 268 | (3R)-3-(4-chlorophenyl)-2-[(5-chloropyridin-2- |
| | yl)methyl]-6-[2-hydroxy-1-(4-methyl-1,4-diazepan-1- |
| | yl)propan-2-yl]-3-methoxy-2,3-dihydro-1H-isoindol-1-one |
| 269 | (3R)-6-[1-(azetidin-1-yl)-2-hydroxypropan-2-yl]- |
| | 3-(4-chlorophenyl)-2-[(5-chloropyridin-2-yl)methyl]- |
| | 3-methoxy-2,3-dihydro-1H-isoindol-1-one |
| 270 | (3R)-6-[1-(azetidin-1-yl)-2-hydroxypropan-2-yl]- |
| | 3-(4-chlorophenyl)-2-[(5-chloropyridin-2-yl)methyl]- |
| | 3-methoxy-2,3-dihydro-1H-isoindol-1-one |
| 271 | (3R)-3-(4-chlorophenyl)-2-[(5-chloropyridin-2- |
| | yl)methyl]-6-{1-[(3S)-3,4-dimethylpiperazin-1-yl]-2- |
| | hydroxypropan-2-yl}-3-methoxy-2,3-dihydro- |
| | 1H-isoindol-1-one |
| 272 | (3R)-3-(4-chlorophenyl)-2-[(5-chloropyridin-2- |
| | yl)methyl]-6-{1-[(3R)-3,4-dimethylpiperazin-1-yl]-2- |
| | hydroxypropan-2-yl}-3-methoxy-2,3-dihydro- |
| | 1H-isoindol-1-one |
| 273 | (3R)-3-(4-chlorophenyl)-2-[(5-chloropyridin-2- |
| | yl)methyl]-6-{1-[(3R)-3,4-dimethylpiperazin-1-yl]-2- |
| | hydroxypropan-2-yl}-3-methoxy-2,3-dihydro- |
| | 1H-isoindol-1-one |
| 274 | (3R)-3-(4-chlorophenyl)-2-[(5-chloropyridin-2- |
| | yl)methyl]-6-{1-[(2S)-2,4-dimethylpiperazin-1-yl]-2- |
| | hydroxypropan-2-yl}-3-methoxy-2,3-dihydro- |
| | 1H-isoindol-1-one |
| 275 | (3R)-3-(4-chlorophenyl)-2-[(5-chloropyridin-2- |
| | yl)methyl]-6-(2-hydroxy-1-{4H,5H,6H,7H- |
| | [1,2,3]triazolo[1,5-a]pyrazin-5-yl}propan-2-yl)- |
| | 3-methoxy-2,3-dihydro-1H-isoindol-1-one |
| 276 | 6-{[(1R)-1-(4-chlorophenyl)-1-[(1-cyanocyclo- |
| and | propyl)methoxy]-7-fluoro-5-[2-hydroxy-1-(pyrrolidin-1- |
| 277 | yl)propan-2-yl]-3-oxo-2,3-dihydro-1H-isoindol-2- |
| | yl]methyl}pyridine-3-carbonitrile |
| 278 | 6-{[(1R)-1-(4-chlorophenyl)-1-[(1-cyanocyclo- |
| and | propyl)methoxy]-7-fluoro-5-[2-hydroxy-1-(4- |
| 279 | methylpiperazin-1-yl)propan-2-yl]-3-oxo-2,3-dihydro- |
| | 1H-isoindol-2-yl]methyl}pyridine-3-carbonitrile |
| 280 | 1-({[(1R)-1-(4-chlorophenyl)-2-[(5-cyanopyridin- |
| | 2-yl)methyl]-7-fluoro-5-[1-hydroxy-1-(oxan-4- |
| | yl)ethyl]-3-oxo-2,3-dihydro-1H-isoindol-1- |
| | yl]oxy}methyl)cyclopropane-1-carboxamide |
| 281 | 1-{{[(1R)-1-(4-chlorophenyl)-2-[(5-cyanopyridin- |
| | 2-yl)methyl]-7-fluoro-5-[1-hydroxy-1-(oxan-4- |
| | yl)ethyl]-3-oxo-2,3-dihydro-1H-isoindol-1- |
| | yl]oxy}methyl)cyclopropane-1-carboxamide |
| 282 | 6-{[(1R)-1-(4-chlorophenyl)-1-[(1-cyanocyclo- |
| and | propyl)methoxy]-7-fluoro-5-[1-hydroxy-1-(oxan-4- |
| 283 | yl)ethyl]-3-oxo-2,3-dihydro-1H-isoindol-2- |
| | yl]methyl}pyridine-3-carbonitrile |
| 284 | 1-({[(1R)-1-(4-chlorophenyl)-2-[(5-chloropyrimidin- |
| and | 2-yl)methyl]-7-fluoro-5-[1-hydroxy-1-(oxan-4- |
| 285 | yl)ethyl]-3-oxo-2,3-dihydro-1H-isoindol-1- |
| | yl]oxy}methyl)cyclopropane-1-carboxamide |
| 286 | (3R)-3-(4-chlorophenyl)-2-[(5-chloropyridin-2- |
| | yl)methyl]-4-fluoro-6-{1-hydroxy-1-[1-(pyrimidin-2- |
| | yl)piperidin-4-yl]ethyl}-3-methoxy-2,3- |
| | dihydro-1H-isoindol-1-one |
| 287 | 6-{[(1R)-1-(4-chlorophenyl)-7-fluoro-5-[1-hydroxy- |
| | 1-(1-methylpiperidin-4-yl)ethyl]-1-{[1- |
| | (hydroxymethyl)cyclopropyl]methoxy}-3-oxo-2,3- |
| | dihydro-1H-isoindol-2-yl]methyl}pyridine-3- |
| | carbonitrile |
| 288 | 6-{[(1R)-1-(4-chlorophenyl)-7-fluoro-5-[1- |
| | hydroxy-1-(1-methylpiperidin-4-yl)ethyl]-1-{[1- |
| | (hydroxymethyl)cyclopropyl]methoxy}-3-oxo- |
| | 2,3-dihydro-1H-isoindol-2-yl]methyl}pyridine-3- |
| | carbonitrile |
| 289 | 6-{[(1R)-1-(4-chlorophenyl)-7-fluoro-5-[1- |
| and | hydroxy-1-(piperidin-4-yl)ethyl]-1-{[1- |
| 290 | (hydroxymethyl)cyclopropyl]methoxy}-3-oxo- |
| | 2,3-dihydro-1H-isoindol-2-yl]methyl}pyridine-3- |
| | carbonitrile |
| 291 | 6-{[(1R)-5-[1-(1-acetylpiperidin-4-yl)-1- |
| and | hydroxyethyl]-1-(4-chlorophenyl)-7-fluoro-1-{[1- |
| 292 | (hydroxymethyl)cyclopropyl]methoxy}-3-oxo- |
| | 2,3-dihydro-1H-isoindol-2-yl]methyl}pyridine-3- |
| | carbonitrile |
| 293 | (3R)-6-[1-(1-acetylpiperidin-4-yl)-1-hydroxyethyl]- |
| | 3-(4-chlorophenyl)-2-[(5-chloropyridin-2- |
| | yl)methyl]-4-fluoro-3-methoxy-2,3-dihydro- |
| | 1H-isoindol-1-oner |
| 294 | (3R)-3-(4-chlorophenyl)-2-[(5-chloropyridin- |
| | 2-yl)methyl]-4-fluoro-6-[1-hydroxy-1-(1- |
| | methanesulfonylpiperidin-4-yl)ethyl]-3-methoxy- |
| | 2,3-dihydro-1H-isoindol-1-one |
| 295 | (3R)-3-(4-chlorophenyl)-2-[(5-chloropyridin-2-yl)methyl]- |
| | 4-fluoro-6-{1-hydroxy-1-[1-(1,3-oxazole-2- |
| | carbonyl)piperidin-4-yl]ethyl}-3-methoxy- |
| | 2,3-dihydro-1H-isoindol-1-one |
| 296 | (3R)-3-(4-chlorophenyl)-2-[(5-chloropyridin-2- |
| | yl)methyl]-4-fluoro-6-{1-hydroxy-1-[1-(2- |
| | hydroxyacetyl)piperidin-4-yl]ethyl}-3- |
| | methoxy-2,3-dihydro-1H-isoindol-1-one |
| 297 | 6-{[(1R)-5-[1-(1-acetylpiperidin-4-yl)-1-hydroxyethyl]- |
| | 1-(4-chlorophenyl)-7-fluoro-1-methoxy-3-oxo- |
| | 2,3-dihydro-1H-isoindol-2-yl]methyl}pyridine-3-carbonitrile |
| 298 | (3R)-3-(4-chlorophenyl)-2-[(5-chloropyridin-2- |
| and | yl)methyl]-6-(1-{1-[2-(dimethylamino)acetyl]piperidin- |
| 299 | 4-yl}-1-hydroxyethyl)-4-fluoro-3-methoxy- |
| | 2,3-dihydro-1H-isoindol-1-one |
| 300 | (3R)-6-[1-(1-acetylpiperidin-4-yl)-1-hydroxyethyl]- |
| | 3-(4-chlorophenyl)-2-[(5-chloropyridin-2- |
| | yl)methyl]-4-fluoro-3-[(1-hydroxycyclopropyl)methoxy]- |
| | 2,3-dihydro-1H-isoindol-1-one |
| 301 | 1-({[(1R)-5-[1-(1-acetylpiperidin-4-yl)-1-hydroxyethyl]- |
| | 1-(4-chlorophenyl)-2-[(5-chloropyridin-2- |
| | yl)methyl]-7-fluoro-3-oxo-2,3-dihydro-1H-isoindol- |
| | 1-yl]oxy}methyl)cyclopropane-1-carbonitrile |
| 302 | 1-({[(1R)-5-[1-(1-acetylpiperidin-4-yl)-1-hydroxyethyl]- |
| and | 1-(4-chlorophenyl)-2-[(5-chloropyridin-2- |
| 303 | yl)methyl]-7-fluoro-3-oxo-2,3-dihydro-1H-isoindol- |
| | 1-yl]oxy}methyl)cyclopropane-1-carboxamide |
| 304 | (3R)-3-(4-chlorophenyl)-2-[(5-chloropyridin- |
| | 2-yl)methyl]-4-fluoro-6-[1-hydroxy-1-(1- |
| | methylpiperidin-4-yl)ethyl]-3-methoxy- |
| | 2,3-dihydro-1H-isoindol-1-one |
| 305 | 1-({[(1R)-1-(4-chlorophenyl)-2-[(5-chloropyridin- |
| | 2-yl)methyl]-7-fluoro-5-[1-hydroxy-1-(1- |
| | methylpiperidin-4-yl)ethyl]-3-oxo-2,3-dihydro- |
| | 1H-isoindol-1-yl]oxy}methyl)cyclopropane-1- |
| | carboxamide |
| 306 | 1-({[(1R)-1-(4-chlorophenyl)-2-[(5-chloropyridin- |
| | 2-yl)methyl]-7-fluoro-5-[1-hydroxy-1-(1- |
| | methylpiperidin-4-yl)ethyl]-3-oxo-2,3-dihydro- |
| | 1H-isoindol-1-yl]oxy}methyl)cyclopropane-1- |
| | carboxamide |
| 307 | (3R)-3-(4-chlorophenyl)-2-[(5-chloropyridin-2- |
| and | yl)methyl]-4-fluoro-6-[1-hydroxy-1-(1-methylazetidin- |
| 308 | 3-yl)ethyl]-3-methoxy-2,3-dihydro-1H-isoindol-1-one |
| 309 | 6-{[(1R)-1-(4-chlorophenyl)-7-fluoro-5- |
| and | [1-hydroxy-1-(pyridin-2-yl)ethyl]-1-{[1- |
| 310 | (hydroxymethyl)cyclopropyl]methoxy}-3-oxo- |
| | 2,3-dihydro-1H-isoindol-2-yl]methyl}pyridine-3- |
| | carbonitrile |
| 311 | 4-{1-[(1R)-1-(4-chlorophenyl)-2-[(5-chloropyridin- |
| and | 2-yl)methyl]-7-fluoro-1-methoxy-3-oxo-2,3- |
| 312 | dihydro-1H-isoindol-5-yl]-1-hydroxyethyl}- |
| | 1λ6-thiane-1,1-dione |
| 313 | 4-{1-[(1R)-1-(4-chlorophenyl)-2-[(5-chloropyridin-2- |
| and | yl)methyl]-7-fluoro-1-(2-hydroxyethoxy)-3-oxo- |
| 314 | 2,3-dihydro-1H-isoindol-5-yl]-1-hydroxyethyl}- |
| | 1λ6-thiane-1,1-dione |
| 315 | (3R)-3-(4-chlorophenyl)-4-fluoro-6-(2- |
| | hydroxypropan-2-yl)-3-methoxy-2-[(2-methoxy-6- |
| | methylpyridin-3-yl)methyl]-2,3-dihydro-1H-isoindol-1-one |
| 316 | 6-{[(1R)-1-(4-chlorophenyl)-7-fluoro-5-[1-hydroxy- |
| and | 1-(1-methyl-1H-pyrazol-4-yl)ethyl]-1-{[1- |
| 317 | (hydroxymethyl)cyclopropyl]methoxy}-3-oxo-2,3- |
| | dihydro-1H-isoindol-2-yl]methyl}pyridine-3- |
| | carbonitrile |
| 318 | (3R)-3-(4-chlorophenyl)-2-[(5-chloropyridin-2- |
| and | yl)methyl]-6-[1-hydroxy-1-(1-methylpiperidin-4- |
| 319 | yl)ethyl]-3-methoxy-2,3-dihydro-1H-isoindol-1-one |
| 320 | (3R)-3-(4-chlorophenyl)-2-[(5-chloropyridin-2- |
| and | yl)methyl]-6-[1-hydroxy-1-(pyridin-2-yl)ethyl]-3- |
| 321 | methoxy-2,3-dihydro-1H-isoindol-1-one |
| 322 | (3R)-3-(4-chlorophenyl)-2-[(5-chloropyridin-2-yl)methyl]- |
| and | 6-[2-hydroxy-1-(pyridin-4-yl)propan-2-yl]- |
| 323 | 3-methoxy-2,3-dihydro-1H-isoindol-1-one |
| 324 | (3R)-3-(4-chlorophenyl)-2-[(5-chloropyridin-2- |
| and | yl)methyl]-6-[1-(1,2-dimethyl-1H-imidazol-4-yl)-1- |
| 325 | hydroxyethyl]-4-fluoro-3-[(1-hydroxycyclopropyl)methoxy]- |
| | 2,3-dihydro-1H-isoindol-1-one |
| 326 | (3R)-3-(4-chlorophenyl)-2-[(5-chloropyridin- |
| | 2-yl)methyl]-4-fluoro-3-[(1-{[(2- |
| | hydroxyethyl)amino]methyl}cyclopropyl)methoxy]- |
| | 6-(2-hydroxypropan-2-yl)-2,3-dihydro-1H- |
| | isoindol-1-one |
| 327 | (3R)-3-(4-Chlorophenyl)-2-[(5-chloropyridin-2- |
| and | yl)methyl]-6-[2-hydroxy-1-(3-oxomorpholin-4- |
| 328 | yl)propan-2-yl]-3-methoxy-2,3-dihydro-1H-isoindol-1-one |
| 329 | 1-{2-[(1R)-1-(4-Chlorophenyl)-2-[(5-chloropyridin- |
| and | 2-yl)methyl]-1-methoxy-3-oxo-2,3-dihydro-1H- |
| 330 | isoindol-5-yl]-2-hydroxypropyl}imidazolidine-2,4-dione |
| 331 | (3R)-3-(4-Chlorophenyl)-2-[(1R)-1-(5-chloropyridin- |
| | 2-yl)-2,3-dihydroxypropyl]-4-fluoro-3-[(1- |
| | hydroxycyclopropyl)methoxy]-6-(2-hydroxypropan- |
| | 2-yl)-2,3-dihydro-1H-isoindol-1-one |
| 332 | (3R)-3-(4-chlorophenyl)-2-[(5-chloropyridin-2- |
| | yl)methyl]-4-fluoro-6-[1-hydroxy-1-(4-methyl-1H- |
| | imidazol-2-yl)ethyl]-3-[(1-hydroxycyclopro- |
| | pyl)methoxy]-2,3-dihydro-1H-isoindol-1-one |
| 333 | 6-{[(1R)-1-(4-Chlorophenyl)-7-fluoro-5-[1-hydroxy- |
| and | 1-(1,3-thiazol-4-yl)propyl]-1-(2-hydroxyethoxy)- |
| 334 | 3-oxo-2,3-dihydro-1H-isoindol-2-yl]methyl}pyridine- |
| | 3-carbonitrile |
| 335 | 6-{[1-(4-chlorophenyl)-7-fluoro-5-[1-hydroxy-1- |
| and | (1-methyl-1H-pyrazol-3-yl)propyl]-3-oxo-1-[(3S)- |
| 336 | oxolan-3-yloxy]-2,3-dihydro-1H-isoindol-2- |
| | yl]methyl}pyridine-3-carbonitrile |
| 337 | 6-{[(1R)-1-(4-chlorophenyl)-7-fluoro-5-[1-hydroxy- |
| and | 1-(1-methyl-1H-imidazol-4-yl)propyl]-1-{[1- |
| 338 | (hydroxymethyl)cyclopropyl]methoxy}-3-oxo-2,3- |
| | dihydro-1H-isoindol-2-yl]methyl}pyridine-3- |
| | carbonitrile |
| 339 | 6-{[(1R)-1-(4-chlorophenyl)-7-fluoro-5-[1-hydroxy-1- |
| | (1-methyl-1H-pyrazol-4-yl)propyl]-3-oxo-1-[cis- |
| | 3-hydroxycyclobutoxy]-2,3-dihydro-1H-isoindol-2- |
| | yl]methyl}pyridine-3-carbonitrile |
| 340 | 6-{[(1R)-1-(4-chlorophenyl)-7-fluoro-5-[1-hydroxy- |
| | 1-(1-methyl-1H-pyrazol-4-yl)ethyl]-1-[(1- |
| | hydroxycyclopropyl)methoxy]-3-oxo-2,3-dihydro- |
| | 1H-isoindol-2-yl]methyl}pyridine-3-carbonitrile |
| 34 | 6-{[(1R)-1-(4-chlorophenyl)-7-fluoro-5-[1-hydroxy- |
| | 1-(1-methyl-1H-pyrazol-4-yl)ethyl]-1-[(1- |
| | hydroxycyclopropyl)methoxy]-3-oxo-2,3-dihydro- |
| | 1H-isoindol-2-yl]methyl}pyridine-3-carbonitrile |
| 342 | 6-{[(1R)-1-(4-chlorophenyl)-7-fluoro-5-[1-hydroxy-1- |
| | (1-methyl-1H-pyrazol-4-yl)propyl]-3-oxo-1-[cis- |
| | 3-hydroxycyclobutoxy]-2,3-dihydro-1H-isoindol- |
| | 2-yl]methyl}pyridine-3-carbonitrile |
| 343 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-(2-hydroxyethoxy)- |
| | 2,3-dihydro-1H-isoindol-1-one |
| 344 | 6-{[(1R)-1-(4-chlorophenyl)-7-fluoro-5-[1-hydroxy- |
| | 1-(1-methyl-1H-pyrazol-4-yl)ethyl]-3-oxo-1- |
| | [(3S)-oxolan-3-yloxy]-2,3-dihydro-1H-isoindol- |
| | 2-yl]methyl}pyridine-3-carbonitrile |
| 345 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-{[1-(hydroxymethyl)cyclo- |
| | propyl]methoxy}-2,3-dihydro-1H-isoindol-1-one |
| 346 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-{[1-(hydroxymethyl)cyclo- |
| | propyl]methoxy}-2,3-dihydro-1H-isoindol-1-one |
| 347 | (3R)-3-(4-chlorophenyl)-4-fluoro-6-[1-hydroxy-1- |
| | (1-methyl-1H-pyrazol-4-yl)ethyl]-3-[(1- |
| | hydroxycyclopropyl)methoxy]-2-[(5-methoxypyridin- |
| | 2-yl)methyl]-2,3-dihydro-1H-isoindol-1-one |
| 348 | (3R)-3-(4-chlorophenyl)-2-[(5-chloropyrimidin-2- |
| | yl)methyl]-4-fluoro-6-[1-hydroxy-1-(1-methyl-1H- |
| | pyrazol-4-yl)ethyl]-3-{[1-(hydroxymethyl)cyclopro- |
| | pyl]methoxy}-2,3-dihydro-1H-isoindol-1-one |
| 349 | (3R)-3-(4-chlorophenyl)-2-[(5-chloropyrimidin-2- |
| | yl)methyl]-4-fluoro-6-[1-hydroxy-1-(1-methyl-1H- |
| | pyrazol-4-yl)ethyl]-3-{[1-(hydroxymethyl)cyclo- |
| | propyl]methoxy}-2,3-dihydro-1H-isoindol-1-one |
| 350 | (3R)-3-(4-chlorophenyl)-2-[(3,5-difluoropyridin-2- |
| | yl)methyl]-4-fluoro-6-[1-hydroxy-1-(1-methyl-1H- |
| | pyrazol-4-yl)ethyl]-3-[(1-hydroxycyclopropyl)methoxy]- |
| | 2,3-dihydro-1H-isoindol-1-one |
| 351 | (3R)-3-(4-chlorophenyl)-2-[(3,5-difluoropyridin-2- |
| | yl)methyl]-4-fluoro-6-[1-hydroxy-1-(1-methyl-1H- |
| | pyrazol-4-yl)ethyl]-3-[(1-hydroxycyclopropyl)methoxy]- |
| | 2,3-dihydro-1H-isoindol-1-one |
| 352 | 6-{[(1R)-1-(4-chlorophenyl)-7-fluoro-1-({1- |
| | [hydroxy(²H₂)methyl]cyclopropyl}(²H₂)methoxy)-5-[1- |
| | hydroxy-1-(1-methyl-1H-pyrazol-4-yl)ethyl]-3- |
| | oxo-2,3-dihydro-1H-isoindol-2-yl]methyl}pyridine-3- |
| | carbonitrile |
| 353 | 6-{[(1R)-1-(4-chlorophenyl)-7-fluoro-1-({1- |
| | [hydroxy(²H₂)methyl]cyclopropyl}(²H₂)methoxy)-5-[1- |
| | hydroxy-1-(1-methyl-1H-pyrazol-4-yl)ethyl]- |
| | 3-oxo-2,3-dihydro-1H-isoindol-2-yl]methyl}pyridine-3- |
| | carbonitrile |
| 354 | (3R)-3-(4-chlorophenyl)-2-[(5-chloropyridin-2- |
| | yl)methyl]-4-fluoro-6-[1-hydroxy-1-(1-methyl-1H- |
| | pyrazol-4-yl)ethyl]-3-[(2R)-2-hydroxypropoxy]- |
| | 2,3-dihydro-1H-isoindol-1-one |
| 355 | 6-{[1-(4-chlorophenyl)-7-fluoro-5-[1-hydroxy- |
| | 1-(1-methyl-1H-pyrazol-4-yl)ethyl]-3-oxo-1-[cis-3- |
| | hydroxycyclobutoxy]-2,3-dihydro-1H-isoindol- |
| | 2-yl]methyl}pyridine-3-carbonitrile |
| 356 | 6-{[1-(4-chlorophenyl)-7-fluoro-5-[1-hydroxy-1- |
| | (1-methyl-1H-pyrazol-4-yl)ethyl]-3-oxo-1-[cis-3- |
| | hydroxycyclobutoxy]-2,3-dihydro-1H-isoindol- |
| | 2-yl]methyl}pyridine-3-carbonitrile |
| 357 | 6-{[(1R)-1-(4-chlorophenyl)-7-fluoro-5-[hydroxy(1- |
| | methyl-1H-pyrazol-4-yl)methyl]-3-oxo-1-[(3S)- |
| | oxolan-3-yloxy]-2,3-dihydro-1H-isoindol-2- |
| | yl]methyl}pyridine-3-carbonitrile |
| 358 | 6-{[(1R)-1-(4-chlorophenyl)-7-fluoro-5-[1-hydroxy- |
| | 1-(1-methyl-1H-pyrazol-4-yl)propyl]-3-oxo-1- |
| | [(3S)-oxolan-3-yloxy]-2,3-dihydro-1H-isoindol- |
| | 2-yl]methyl}pyridine-3-carbonitrile |
| 359 | 6-{[(1R)-1-(4-chlorophenyl)-7-fluoro-5-[1-hydroxy- |
| | 1-(1-methyl-1H-pyrazol-4-yl)propyl]-3-oxo-1- |
| | [(3S)-oxolan-3-yloxy]-2,3-dihydro-1H-isoindol- |
| | 2-yl]methyl}pyridine-3-carbonitrile |
| 360 | 6-{[(1R)-1-(4-chlorophenyl)-7-fluoro-5-[hydroxy(1- |
| | methyl-1H-pyrazol-4-yl)methyl]-3-oxo-1-[(3S)- |
| | oxolan-3-yloxy]-2,3-dihydro-1H-isoindol-2- |
| | yl]methyl}pyridine-3-carbonitrile |
| 361 | 6-{[(1R)-1-(4-chlorophenyl)-7-fluoro-5-[1- |
| | hydroxy-1-(1-methyl-1H-pyrazol-4-yl)prop-2-en-1-yl]-3- |
| | oxo-1-[cis-3-hydroxycyclobutoxy]-2,3-dihydro- |
| | 1H-isoindol-2-yl]methyl}pyridine-3-carbonitrile |
| 362 | 6-{[(1R)-1-(4-chlorophenyl)-7-fluoro-5-[1-hydroxy- |
| | 1-(1-methyl-1H-pyrazol-4-yl)prop-2-en-1-yl]-3- |
| | oxo-1-[cis-3-hydroxycyclobutoxy]-2,3-dihydro- |
| | 1H-isoindol-2-yl]methyl}pyridine-3-carbonitrile |
| 363 | 6-{[(1R)-1-(4-chlorophenyl)-7-fluoro-5-[1-hydroxy- |
| | 1-(1-methyl-1H-imidazol-4-yl)propyl]-1-(2- |
| | hydroxyethoxy)-3-oxo-2,3-dihydro-1H-isoindol- |
| | 2-yl]methyl}pyridine-3-carbonitrile |
| 364 | 6-{[(1R)-1-(4-chlorophenyl)-7-fluoro-5-[1-hydroxy- |
| | 1-(1-methyl-1H-imidazol-4-yl)propyl]-1-(2- |
| | hydroxyethoxy)-3-oxo-2,3-dihydro-1H-isoindol- |
| | 2-yl]methyl}pyridine-3-carbonitrile |
| 365 | 6-{[(1R)-1-(4-chlorophenyl)-7-fluoro-5-[1-hydroxy- |
| | 1-(1-methyl-1H-imidazol-4-yl)propyl]-3-oxo-1- |
| | [(3S)-oxolan-3-yloxy]-2,3-dihydro-1H-isoindol- |
| | 2-yl]methyl}pyridine-3-carbonitrile |
| 366 | 6-{[(1R)-1-(4-chlorophenyl)-7-fluoro-5-[1-hydroxy- |
| | 1-(1-methyl-1H-imidazol-4-yl)propyl]-3-oxo-1- |
| | [(3S)-oxolan-3-yloxy]-2,3-dihydro-1H-isoindol- |
| | 2-yl]methyl}pyridine-3-carbonitrile |
| 367 | (3R)-3-(4-chlorophenyl)-2-[(6-chloropyridin-3- |
| | yl)methyl]-4-fluoro-6-[1-hydroxy-1-(1-methyl-1H- |
| | imidazol-4-yl)propyl]-3-(2-hydroxyethoxy)-2,3- |
| | dihydro-1H-isoindol-1-one |
| 368 | (3R)-3-(4-chlorophenyl)-2-[(6-chloropyridin-3- |
| | yl)methyl]-4-fluoro-6-[1-hydroxy-1-(1-methyl-1H- |
| | imidazol-4-yl)propyl]-3-(2-hydroxyethoxy)- |
| | 2,3-dihydro-1H-isoindol-1-one |
| 369 | 2-{[(1R)-1-(4-chlorophenyl)-7-fluoro-5-[1-hydroxy- |
| | 1-(1-methyl-1H-imidazol-4-yl)propyl]-3-oxo-1- |
| | [(3S)-oxolan-3-yloxy]-2,3-dihydro-1H-isoindol- |
| | 2-yl]methyl}pyrimidine-5-carbonitrile |
| 370 | 2-{[(1R)-1-(4-chlorophenyl)-7-fluoro-5-[1-hydroxy- |
| | 1-(1-methyl-1H-imidazol-4-yl)propyl]-3-oxo-1- |
| | [(3S)-oxolan-3-yloxy]-2,3-dihydro-1H-isoindol- |
| | 2-yl]methyl}pyrimidine-5-carbonitrile |
| 371 | 6-{[(1R)-1-(4-chlorophenyl)-7-fluoro-3-oxo-1- |
| | [(3S)-oxolan-3-yloxy]-5-[2,2,2-trifluoro-1-hydroxy-1- |
| | (1-methyl-1H-pyrazol-4-yl)ethyl]-2,3-dihydro- |
| | 1H-isoindol-2-yl]methyl}pyridine-3-carbonitrile |
| 372 | (3R)-2-[(5-chloro-3-methanesulfonylpyridin-2- |
| | yl)methyl]-3-(4-chlorophenyl)-4-fluoro-6-[1-hydroxy- |
| | 1-(1-methyl-1H-pyrazol-4-yl)ethyl]-3-methoxy- |
| | 2,3-dihydro-1H-isoindol-1-one |
| 373 | (3R)-2-[(5-chloro-3-methanesulfonylpyridin-2- |
| | yl)methyl]-3-(4-chlorophenyl)-4-fluoro-6-[1-hydroxy- |
| | 1-(1-methyl-1H-pyrazol-4-yl)ethyl]-3-methoxy- |
| | 2,3-dihydro-1H-isoindol-1-one |
| 374 | 6-{[(1R)-1-(4-chlorophenyl)-7-fluoro-5-[1- |
| and | hydroxy-1-(1-methyl-2-oxo-1,2-dihydropyridin-4- |
| 375 | yl)propyl]-3-oxo-1-[(3S)-oxolan-3-yloxy]-2,3-dihydro- |
| | 1H-isoindol-2-yl]methyl}pyridine-3-carbonitrile |
| 376 | 6-[1-(1-Acetylpiperidin-4-yl)-1-hydroxyethyl]-3-(4- |
| | chlorophenyl)-2-[(5-chloropyridin-2-yl)methyl]-4- |
| | fluoro-3-hydroxy-2,3-dihydro-1H-isoindol-1-one |
| 377 | (3R)-3-(4-chlorophenyl)-2-[(5-chloropyridin-2- |
| and | yl)methyl]-6-[2-hydroxy-1-(piperidin-4-yloxy)propan- |
| 378 | 2-yl]-3-methoxy-2,3-dihydro-1H-isoindol-1-one |
| 379 | (3R)-3-(4-chlorophenyl)-2-[(5-chloropyridin-2- |
| | yl)methyl]-6-{1-[(3S)-3,4-dimethylpiperazin-1-yl]-2- |
| | hydroxypropan-2-yl}-3-methoxy-2,3-dihydro- |
| | 1H-isoindol-1-one |
| 380 | (3R)-3-(4-chlorophenyl)-2-[(5-chloropyridin-2- |
| | yl)methyl]-6-{1-[(2S)-2,4-dimethylpiperazin-1-yl]-2- |
| | hydroxypropan-2-yl}-3-methoxy-2,3-dihydro- |
| | 1H-isoindol-1-one |
| 381 | (3R)-3-(4-chlorophenyl)-2-[(5-chloropyridin-2- |
| | yl)methyl]-6-{2-hydroxy-1-[(3S)-3-hydroxypyrrolidin- |
| | 1-yl]propan-2-yl}-3-methoxy-2,3-dihydro-1H-isoindol-1-one |
| 382 | (3R)-3-(4-chlorophenyl)-2-[(5-chloropyridin-2- |
| | yl)methyl]-6-{2-hydroxy-1-[(3S)-3-hydroxypyrrolidin- |
| | 1-yl]propan-2-yl}-3-methoxy-2,3-dihydro-1H-isoindol-1-one |
| 383 | (3R)-3-(4-chlorophenyl)-2-[(5-chloropyridin-2- |
| | yl)methyl]-6-{2-hydroxy-1-[(3R)-3-hydroxypyrrolidin- |
| | 1-yl]propan-2-yl}-3-methoxy-2,3-dihydro-1H-isoindol-1-one |
| 384 | (3R)-3-(4-chlorophenyl)-2-[(5-chloropyridin-2- |
| | yl)methyl]-6-{2-hydroxy-1-[(3R)-3-hydroxypyrrolidin- |
| | 1-yl]propan-2-yl}-3-methoxy-2,3-dihydro-1H-isoindol-1-one |
| 385 | (3R)-3-(4-chlorophenyl)-2-[(5-chloropyrimidin- |
| | 2-yl)methyl]-4-fluoro-6-[1-(4-fluorooxan-4-yl)-1- |
| | hydroxyethyl]-3-[(1-hydroxycyclopropyl)methoxy]- |
| | 2,3-dihydro-1H-isoindol-1-one |
| 386 | (3R)-3-(4-chlorophenyl)-2-[(5-chloropyrimidin- |
| | 2-yl)methyl]-4-fluoro-6-[1-(4-fluorooxan-4-yl)-1- |
| | hydroxyethyl]-3-[(1-hydroxycyclopropyl)methoxy]- |
| | 2,3-dihydro-1H-isoindol-1-one |
| 387 | 6-{[(1R)-1-(4-chlorophenyl)-7-fluoro-5-[1-(4- |
| | fluorooxan-4-yl)-1-hydroxyethyl]-3-oxo-1-[(3S)-oxolan- |
| | 3-yloxy]-2,3-dihydro-1H-isoindol-2-yl]methyl}pyridine- |
| | 3-carbonitrile |
| 388 | 6-{[(1R)-1-(4-chlorophenyl)-7-fluoro-5-[1-(4-fluorooxan- |
| | 4-yl)-1-hydroxyethyl]-3-oxo-1-[(3S)-oxolan- |
| | 3-yloxy]-2,3-dihydro-1H-isoindol-2-yl]methyl}pyridine- |
| | 3-carbonitrile |
| 389 | (3R)-6-[1-(1-acetylazetidin-3-yl)-1-hydroxyethyl]- |
| and | 3-(4-chlorophenyl)-2-[(5-chloropyridin-2- |
| 390 | yl)methyl]-4-fluoro-3-methoxy-2,3-dihydro- |
| | 1H-isoindol-1-one |
| 391 | (3R)-3-(4-chlorophenyl)-2-[(5-chloropyridin- |
| and | 2-yl)methyl]-4-fluoro-6-{1-hydroxy-1-[1-(2- |
| 392 | hydroxyacetyl)azetidin-3-yl]ethyl}-3-methoxy- |
| | 2,3-dihydro-1H-isoindol-1-one |
| 393 | 3-{1-[(1R)-1-(4-chlorophenyl)-2-[(5-cyanopyridin- |
| and | 2-yl)methyl]-7-fluoro-1-methoxy-3-oxo-2,3- |
| 394 | dihydro-1H-isoindol-5-yl]-1-hydroxyethyl}- |
| | N,N-dimethylazetidine-1-carboxamide |
| 395 | (3R)-3-(4-chlorophenyl)-2-[(5-chloropyridin-2- |
| and | yl)methyl]-4-fluoro-6-[1-hydroxy-1-(pyrimidin-2- |
| 396 | yl)ethyl]-3-[(1-hydroxycyclopropyl)methoxy]- |
| | 2,3-dihydro-1H-isoindol-1-one |
| 397 | 4-{1-[(1R)-1-(4-chlorophenyl)-2-[(5- |
| and | chloropyrimidin-2-yl)methyl]-7-fluoro-1-[(1- |
| 398 | hydroxycyclopropyl)methoxy]-3-oxo-2,3-dihydro- |
| | 1H-isoindol-5-yl]-1-hydroxyethyl}-1λ6-thiane-1,1-dione |
| 399 | 4-{[(1R)-1-(4-chlorophenyl)-2-[(5- |
| and | chloropyrimidin-2-yl)methyl]-7-fluoro-1-[(1- |
| 400 | hydroxycyclopropyl) methoxy]-3-oxo-2,3-dihydro- |
| | 1H-isoindol-5-yl](hydroxy)methyl}-16-thiane-1,1- |
| | dione |
| 401 | (3R)-3-(4-chlorophenyl)-2-[(5-chloropyrimidin- |
| and | 2-yl)methyl]-4-fluoro-6-{1-hydroxy-1-[trans-4- |
| 402 | hydroxycyclohexyl]ethyl}-3-{[1-(hydroxymethyl)cyclo- |
| | propyl]methoxy}-2,3-dihydro-1H-isoindol-1- |
| | one |
| 403 | 1-({[(1R)-1-(4-chlorophenyl)-2-[(5-chloropyrimidin- |
| and | 2-yl)methyl]-5-(1-cyclobutyl-1-hydroxyethyl)-7- |
| 404 | fluoro-3-oxo-2,3-dihydro-1H-isoindol-1- |
| | yl]oxy}methyl)cyclopropane-1-carboxamide |
| 405 | 6-{[(1R)-1-(4-chlorophenyl)-5-(1-cyclobutyl-1- |
| and | hydroxyethyl)-7-fluoro-1-(2-hydroxyethoxy)-3-oxo- |
| 406 | 2,3-dihydro-1H-isoindol-2-yl]methyl}pyridine-3-carbonitrile |
| 407 | (3R)-2-[(5-chloro-1-oxo-1)5-pyridin-2-yl)methyl]- |
| | 3-(4-chlorophenyl)-4-fluoro-6-[1-hydroxy-1-(1- |
| | methyl-1H-pyrazol-4-yl)ethyl]-3-{[1-(hydroxymethyl)cyclo- |
| | propyl]methoxy}-2,3-dihydro-1H-isoindol- |
| | 1-one |
| 408 | 1-({[(1R)-2-[(5-chloro-1-oxo-1λ⁵-pyridin-2-yl)methyl]- |
| | 1-(4-chlorophenyl)-7-fluoro-5-[1-hydroxy-1-(1- |
| | methyl-1H-pyrazol-4-yl)ethyl]-3-oxo-2,3-dihydro- |
| | 1H-isoindol-1-yl]oxy}methyl)cyclopropane-1- |
| | carbonitrile |
| 409 | (3R)-2-[(5-chloro-1-oxo-1λ⁵-pyridin-2-yl)methyl]- |
| | 3-(4-chlorophenyl)-4-fluoro-6-[1-hydroxy-1-(1- |
| | methyl-1H-pyrazol-4-yl)ethyl]-3-[(1-hydroxycyclo- |
| | propyl)methoxy]-2,3-dihydro-1H-isoindol-1-one |
| 410 | 6-{[(1R)-1-(4-chlorophenyl)-7-fluoro- |
| | 5-(2-hydroxybutan-2-yl)-1-{[1- |
| | (hydroxymethyl)cyclopropyl]methoxy}-3-oxo- |
| | 2,3-dihydro-1H-isoindol-2-yl]methyl}pyridine-3- |
| | carbonitrile |
| 411 | 6-{[(1R)-1-(4-chlorophenyl)-7-fluoro- |
| | 5-(2-hydroxybutan-2-yl)-1-{[1- |
| | (hydroxymethyl)cyclopropyl]methoxy}- |
| | 3-oxo-2,3-dihydro-1H-isoindol-2-yl]methyl}pyridine-3- |
| | carbonitrile |
| 412 | 6-{[(1R)-1-(4-Chlorophenyl)-7-fluoro- |
| | 5-(2-hydroxybut-3-en-2-yl)-1-{[1- |
| | (hydroxymethyl)cyclopropyl]methoxy}-3-oxo- |
| | 2,3-dihydro-1H-isoindol-2-yl]methyl}pyridine-3- |
| | carbonitrile |
| 413 | 6-{[(1R)-1-(4-Chlorophenyl)-7-fluoro- |
| | 5-(2-hydroxybut-3-en-2-yl)-1-{[1- |
| | (hydroxymethyl)cyclopropyl]methoxy}-3-oxo- |
| | 2,3-dihydro-1H-isoindol-2-yl]methyl}pyridine-3- |
| | carbonitrile |
| 414 | 6-{[(1R)-1-(4-Chlorophenyl)-5-(1-cyclopropyl- |
| | 1-hydroxyethyl)-7-fluoro-1-{[1- |
| | (hydroxymethyl)cyclopropyl]methoxy}-3-oxo- |
| | 2,3-dihydro-1H-isoindol-2-yl]methyl}pyridine-3- |
| | carbonitrile |
| 415 | 6-{[(1R)-1-(4-Chlorophenyl)-5-(1-cyclopropyl- |
| | 1-hydroxyethyl)-7-fluoro-1-{[1- |
| | (hydroxymethyl)cyclopropyl]methoxy}-3-oxo-2,3- |
| | dihydro-1H-isoindol-2-yl]methyl}pyridine-3- |
| | carbonitrile |
| 416 | 6-{[(1R)-1-(4-chlorophenyl)-7-fluoro-1-{[1- |
| | (hydroxymethyl)cyclopropyl]methoxy}-3-oxo-5-(1,1,1- |
| | trifluoro-2-hydroxypropan-2-yl)-2,3-dihydro- |
| | 1H-isoindol-2-yl]methyl}pyridine-3-carbonitrile |
| 417 | 6-{[(1R)-1-(4-chlorophenyl)-7-fluoro-1-{[1- |
| | (hydroxymethyl)cyclopropyl]methoxy}-3-oxo-5-(1,1,1- |
| | trifluoro-2-hydroxypropan-2-yl)-2,3-dihydro- |
| | 1H-isoindol-2-yl]methyl}pyridine-3-carbonitrile |
| 418 | (3R)-3-(4-Chlorophenyl)-2-[(1R)-1-(5-chloropyridin- |
| | 2-yl)-2-hydroxyethyl]-4-fluoro-3-{[1- |
| | (hydroxymethyl)cyclopropyl]methoxy}-6-(2- |
| | hydroxypropan-2-yl)-2,3-dihydro-1H-isoindol-1-one |
| 419 | 6-{[(1R)-1-(4-chlorophenyl)-7-fluoro-5-(2- |
| | hydroxypropan-2-yl)-3-oxo-1-{[(trans-3- |
| | hydroxycyclobutyl]methoxy}-2,3-dihydro-1H- |
| | isoindol-2-yl]methyl}pyridine-3-carbonitrile |
| 420 | 1-({[(1R)-1-(4-chlorophenyl)-2-[(5-chloropyridin- |
| | 2-yl)methyl]-7-fluoro-5-{1-hydroxy-1-[1-(2- |
| | hydroxyethyl)piperidin-4-yl]ethyl}-3-oxo-2,3- |
| | dihydro-1H-isoindol-1-yl]oxy}methyl)cyclopropane-1- |
| | carbonitrile |
| 421 | (3R)-6-[1-(1-acetylpiperidin-4-yl)-1-hydroxyethyl]- |
| | 3-(4-chlorophenyl)-2-[(5-chloropyridin-2- |
| | yl)methyl]-4-fluoro-3-(2-hydroxyethoxy)- |
| | 2,3-dihydro-1H-isoindol-1-one |
| 422 | (3R)-6-[1-(1-acetylpiperidin-4-yl)-1-hydroxyethyl]- |
| | 3-(4-chlorophenyl)-2-[(5-chloropyrimidin-2- |
| | yl)methyl]-4-fluoro-3-(2-hydroxyethoxy)- |
| | 2,3-dihydro-1H-isoindol-1-one |
| 423 | (3R)-3-(4-chlorophenyl)-2-[(5-chloropyrimidin- |
| | 2-yl)methyl]-4-fluoro-6-[1-hydroxy-1-(1- |
| | methylpiperidin-4-yl)ethyl]-3-[(3S)-oxolan- |
| | 3-yloxy]-2,3-dihydro-1H-isoindol-1-one |
| 424 | (3R)-6-[1-(1-acetylpiperidin-4-yl)-1-hydroxyethyl]- |
| | 2-{[5-chloro-3-(hydroxymethyl)pyridin-2- |
| | yl]methyl}-3-(4-chlorophenyl)-4-fluoro-3- |
| | methoxy-2,3-dihydro-1H-isoindol-1-one |
| 425 | (3R)-6-[1-(1-acetylpiperidin-4-yl)-1-hydroxyethyl]- |
| | 3-(4-chlorophenyl)-2-[(5-chloropyrimidin-2- |
| | yl)methyl]-4-fluoro-3-[cis-3-hydroxycyclobutoxy]- |
| | 2,3-dihydro-1H-isoindol-1-one |
| 426 | 6-{[(1R)-1-(4-chlorophenyl)-7-fluoro-5-[1-hydroxy- |
| | 1-(1-methyl-1H-imidazol-4-yl)propyl]-3-oxo-1- |
| | [(3R)-oxolan-3-yloxy]-2,3-dihydro-1H-isoindol- |
| | 2-yl]methyl}pyridine-3-carbonitrile |
| 427 | 6-{[(1R)-1-(4-chlorophenyl)-7-fluoro-5-[1-hydroxy- |
| | 1-(1-methyl-1H-imidazol-4-yl)propyl]-3-oxo-1- |
| | [(3R)-oxolan-3-yloxy]-2,3-dihydro-1H-isoindol- |
| | 2-yl]methyl}pyridine-3-carbonitrile |
| 428 | 6-{[(1R)-1-(4-chlorophenyl)-7-fluoro-3-oxo-1- |
| | [(3S)-oxolan-3-yloxy]-5-[2,2,2-trifluoro-1-hydroxy-1- |
| | (1-methyl-1H-pyrazol-4-yl)ethyl]-2,3-dihydro- |
| | 1H-isoindol-2-yl]methyl}pyridine-3-carbonitrile |
| 429 | 6-{[(1R)-1-(4-chlorophenyl)-7-fluoro-5-[1-hydroxy- |
| | 1-(1-methyl-1H-imidazol-4-yl)propyl]-1-(2- |
| | methoxyethoxy)-3-oxo-2,3-dihydro-1H-isoindol- |
| | 2-yl]methyl}pyridine-3-carbonitrile |
| 430 | 6-{[(1R)-1-(4-chlorophenyl)-7-fluoro-5-[1-hydroxy- |
| | 1-(1-methyl-1H-imidazol-4-yl)propyl]-1-(2- |
| | methoxyethoxy)-3-oxo-2,3-dihydro-1H-isoindol- |
| | 2-yl]methyl}pyridine-3-carbonitrile |
| 431 | 5-{[(1R)-1-(4-chlorophenyl)-7-fluoro-5-[1-hydroxy- |
| | 1-(1-methyl-1H-imidazol-4-yl)propyl]-3-oxo-1- |
| | [(3S)-oxolan-3-yloxy]-2,3-dihydro-1H-isoindol- |
| | 2-yl]methyl}pyridine-2-carbonitrile |
| 432 | 5-{[(1R)-1-(4-chlorophenyl)-7-fluoro-5-[1-hydroxy- |
| | 1-(1-methyl-1H-imidazol-4-yl)propyl]-3-oxo-1- |
| | [(3S)-oxolan-3-yloxy]-2,3-dihydro-1H-isoindol- |
| | 2-yl]methyl}pyridine-2-carbonitrile |
| 433 | 6-{[(1R)-1-(4-chlorophenyl)-5-[cyclopropyl(hydroxy)(1- |
| | methyl-1H-imidazol-4-yl)methyl]-7-fluoro-3- |
| | oxo-1-[(3S)-oxolan-3-yloxy]-2,3-dihydro-1H- |
| | isoindol-2-yl]methyl}pyridine-3-carbonitrile |
| 434 | 6-{[(1R)-1-(4-chlorophenyl)-5-[cyclopropyl(hydroxy)(1- |
| | methyl-1H-imidazol-4-yl)methyl]-7-fluoro-3- |
| | oxo-1-[(3S)-oxolan-3-yloxy]-2,3-dihydro-1H- |
| | isoindol-2-yl]methyl}pyridine-3-carbonitrile |
| 435 | 6-{[(1R)-1-(4-chlorophenyl)-7-fluoro-1-[(3- |
| | fluorooxetan-3-yl)methoxy]-5-[1-hydroxy-1-(1-methyl- |
| | 1H-imidazol-4-yl)propyl]-3-oxo-2,3-dihydro- |
| | 1H-isoindol-2-yl]methyl}pyridine-3-carbonitrile |
| 436 | 6-{[(1R)-1-(4-chlorophenyl)-7-fluoro-1-[(3- |
| | fluorooxetan-3-yl)methoxy]-5-[1-hydroxy-1-(1-methyl- |
| | 1H-imidazol-4-yl)propyl]-3-oxo-2,3-dihydro-1H- |
| | isoindol-2-yl]methyl}pyridine-3-carbonitrile |
| 437 | 6-{[(1R)-1-(4-chlorophenyl)-7-fluoro-5-[1-hydroxy- |
| | 1-(1-methyl-1H-imidazol-4-yl)propyl]-1-[(2R)-2- |
| | hydroxypropoxy]-3-oxo-2,3-dihydro-1H-isoindol- |
| | 2-yl]methyl}pyridine-3-carbonitrile |
| 438 | 6-{[(1R)-1-(4-chlorophenyl)-7-fluoro-5-[1-hydroxy- |
| | 1-(1-methyl-1H-imidazol-4-yl)propyl]-1-[(2R)-2- |
| | hydroxypropoxy]-3-oxo-2,3-dihydro-1H-isoindol- |
| | 2-yl]methyl}pyridine-3-carbonitrile |
| 439 | 6-{[(1R)-1-(4-chlorophenyl)-7-fluoro-1-({1- |
| | [hydroxy(²H₂)methyl]cyclopropyl}(²H₂)methoxy)-5-[1- |
| | hydroxy-1-(1-methyl-1H-imidazol-4-yl)propyl]-3- |
| | oxo-2,3-dihydro-1H-isoindol-2-yl]methyl}pyridine- |
| | 3-carbonitrile |
| 440 | 6-{[(1R)-1-(4-chlorophenyl)-7-fluoro-1-({1- |
| | [hydroxy(²H₂)methyl]cyclopropyl}(²H₂)methoxy)-5-[1- |
| | hydroxy-1-(1-methyl-1H-imidazol-4-yl)propyl]- |
| | 3-oxo-2,3-dihydro-1H-isoindol-2-yl]methyl}pyridine- |
| | 3-carbonitrile |
| 441 | 6-{[(1R)-1-(4-Chlorophenyl)-7-fluoro-5-[2-fluoro- |
| and | 1-hydroxy-1-(1-methyl-1H-pyrazol-4-yl)ethyl]-3- |
| 442 | oxo-1-[(3S)-oxolan-3-yloxy]-2,3-dihydro-1H- |
| | isoindol-2-yl]methyl}pyridine-3-carbonitrile |
| 443 | (3R)-2-[(5-chloro-3-hydroxypyridin-2-yl)methyl]- |
| and | 3-(4-chlorophenyl)-4-fluoro-6-[1-hydroxy-1-(1- |
| 444 | methyl-1H-imidazol-4-yl)propyl]-3-(2- |
| | hydroxyethoxy)-2,3-dihydro-1H-isoindol-1-one |
| 445 | 6-{[(1R)-1-(4-chlorophenyl)-7-fluoro-5-[1-(4- |
| | fluorooxan-4-yl)-1-hydroxypropyl]-3-oxo-1-[(3S)- |
| | oxolan-3-yloxy]-2,3-dihydro-1H-isoindol-2- |
| | yl]methyl}pyridine-3-carbonitrile |
| 446 | 6-{[(1R)-1-(4-chlorophenyl)-7-fluoro-5-[1-(4- |
| | fluorooxan-4-yl)-1-hydroxypropyl]-3-oxo-1-[(3S)- |
| | oxolan-3-yloxy]-2,3-dihydro-1H-isoindol- |
| | 2-yl]methyl}pyridine-3-carbonitrile |
| 447 | (3R)-3-(4-Chlorophenyl)-2-[(5-chloropyrimidin- |
| | 2-yl)methyl]-4-fluoro-6-[1-(4-fluorooxan-4-yl)-1- |
| | hydroxyethyl]-3-({1-[hydroxy(²H₂) methyl]cyclo- |
| | propyl}(²H₂)methoxy)-2,3-dihydro-1H-isoindol-1-one |
| 448 | (3R)-3-(4-Chlorophenyl)-2-[(5-chloropyrimidin- |
| | 2-yl)methyl]-4-fluoro-6-[1-hydroxy-1-(piperidin-4- |
| | yl)propyl]-3-[(3S)-oxolan-3-yloxy]-2,3-dihydro- |
| | 1H-isoindol-1-one |
| 449 | (3R)-3-(4-chlorophenyl)-2-[(5-chloropyrimidin- |
| | 2-yl)methyl]-4-fluoro-6-[1-hydroxy-1-(1- |
| | methylpiperidin-4-yl)propyl]-3-[(3S)-oxolan- |
| | 3-yloxy]-2,3-dihydro-1H-isoindol-1-one |
| 450 | (3R)-3-(4-chlorophenyl)-2-[(5-chloropyrimidin- |
| | 2-yl)methyl]-4-fluoro-6-[1-hydroxy-1-(piperidin-4- |
| | yl)propyl]-3-methoxy-2,3-dihydro-1H-isoindol-1-one |
| 451 | 2-[(1R)-1-(4-chlorophenyl)-2-[(5-chloropyridin- |
| and | 2-yl)methyl]-1-methoxy-3-oxo-2,3-dihydro-1H- |
| 452 | isoindol-5-yl]-2-hydroxy-N-(1-methylpiperidin- |
| | 4-yl)propanamide |
| 453 | 2-[(1R)-1-(4-Chlorophenyl)-2-[(5-chloropyridin- |
| and | 2-yl)methyl]-1-methoxy-3-oxo-2,3-dihydro-1H- |
| 454 | isoindol-5-yl]-2-hydroxy-N-(1-methyl-1H-pyrazol- |
| | 4-yl)propanamide |
| 455 | 2-[(1R)-1-(4-Chlorophenyl)-2-[(5-chloropyridin- |
| and | 2-yl)methyl]-1-methoxy-3-oxo-2,3-dihydro-1H- |
| 456 | isoindol-5-yl]-2-hydroxy-N-(1-methylazetidin- |
| | 3-yl)propanamide |
| 457 | tert-Butyl 3-(4-{1-[(1R)-1-(4-chlorophenyl)-2-[(5- |
| and | chloropyridin-2-yl)methyl]-7-fluoro-1-methoxy-3- |
| 458 | oxo-2,3-dihydro-1H-isoindol-5-yl]-1-hydroxyethyl}- |
| | 1H-pyrazol-1-yl)azetidine-1-carboxylate |
| 459 | 2-(4-{1-[(1R)-1-(4-chlorophenyl)-2-[(5- |
| | chloropyridin-2-yl)methyl]-7-fluoro-1-methoxy-3-oxo-2,3- |
| | dihydro-1H-isoindol-5-yl]-1-hydroxyethyl}piperidin- |
| | 1-yl)acetic acid |
| 460 | (3R)-3-(4-Chlorophenyl)-3-{[1-(hydroxymethyl)cyclo- |
| | propyl]methoxy}-6-(2-hydroxypropan-2-yl)-2-[(5- |
| | methylpyrazin-2-yl)methyl]-2,3-dihydro-1H-isoindol-1-one |
| 461 | 2-{[(1R)-1-(4-chlorophenyl)-7-fluoro-5-[1-hydroxy- |
| | 1-(1-methyl-1H-imidazol-4-yl)propyl]-1-[(trans-3- |
| | hydroxycyclopentyl)oxy]-3-oxo-2,3-dihydro-1H- |
| | isoindol-2-yl]methyl}pyrimidine-5-carbonitrile |
| 462 | 2-{[(1R)-1-(4-chlorophenyl)-1-[(1-cyanocyclopro- |
| | pyl)methoxy]-7-fluoro-5-[1-hydroxy-1-(1-methyl- |
| | 1H-imidazol-4-yl)propyl]-3-oxo-2,3-dihydro-1H- |
| | isoindol-2-yl]methyl}pyrimidine-5-carbonitrile |
| 463 | 2-{[(1R)-1-(4-chlorophenyl)-1-[(1-cyanocyclo- |
| | propyl)methoxy]-7-fluoro-5-[1-hydroxy-1-(1-methyl- |
| | 1H-imidazol-4-yl)propyl]-3-oxo-2,3-dihydro-1H- |
| | isoindol-2-yl]methyl}pyrimidine-5-carbonitrile |
| 464 | (3R)-3-(4-chlorophenyl)-2-[(5-chloropyrimidin- |
| | 2-yl)methyl]-4-fluoro-6-[1-hydroxy-1-(1-methyl-1H- |
| | imidazol-4-yl)propyl]-3-[cis-3-hydroxycyclobutoxy]- |
| | 2,3-dihydro-1H-isoindol-1-one |
| 465 | (3R)-3-(4-chlorophenyl)-2-[(5-chloropyrimidin-2- |
| | yl)methyl]-4-fluoro-6-[1-hydroxy-1-(1-methyl-1H- |
| | imidazol-4-yl)propyl]-3-[cis-3-hydroxycyclobutoxy]- |
| | 2,3-dihydro-1H-isoindol-1-one |
| 466 | 6-{[(1R)-1-(4-chlorophenyl)-7-fluoro-5-[1-hydroxy- |
| | 1-(1-methyl-1H-imidazol-4-yl)propyl]-1-(3- |
| | hydroxy-2-methylidenepropoxy)-3-oxo-2,3-dihydro- |
| | 1H-isoindol-2-yl]methyl}pyridine-3-carbonitrile |
| 467 | 6-{[(1R)-1-(4-chlorophenyl)-7-fluoro-5-[1-hydroxy- |
| | 1-(1-methyl-1H-imidazol-4-yl)propyl]-1-(3- |
| | hydroxy-2-methylidenepropoxy)-3-oxo-2,3-dihydro- |
| | 1H-isoindol-2-yl]methyl}pyridine-3-carbonitrile |
| 468 | 2-{[(1R)-1-(4-chlorophenyl)-7-fluoro-1-[(3- |
| | fluorooxetan-3-yl)methoxy]-5-[1-hydroxy-1-(1-methyl- |
| | 1H-imidazol-4-yl)ethyl]-3-oxo-2,3-dihydro- |
| | 1H-isoindol-2-yl]methyl}pyrimidine-5-carbonitrile |
| 469 | 2-{[(1R)-1-(4-chlorophenyl)-7-fluoro-1-[(3- |
| | fluorooxetan-3-yl)methoxy]-5-[1-hydroxy-1-(1-methyl- |
| | 1H-imidazol-4-yl)ethyl]-3-oxo-2,3-dihydro- |
| | 1H-isoindol-2-yl]methyl}pyrimidine-5-carbonitrile |
| 470 | 2-{[(1R)-1-(4-chlorophenyl)-7-fluoro-1-[(3-fluorooxetan- |
| | 3-yl)methoxy]-5-[1-hydroxy-1-(1-methyl- |
| | 1H-imidazol-4-yl)propyl]-3-oxo-2,3-dihydro-1H- |
| | isoindol-2-yl]methyl}pyrimidine-5-carbonitrile |
| 471 | 2-{[(1R)-1-(4-chlorophenyl)-7-fluoro-1-[(3- |
| | fluorooxetan-3-yl)methoxy]-5-[1-hydroxy-1-(1-methyl- |
| | 1H-imidazol-4-yl)propyl]-3-oxo-2,3-dihydro- |
| | 1H-isoindol-2-yl]methyl}pyrimidine-5-carbonitrile |
| 472 | 6-{[(1R)-1-(4-chlorophenyl)-7-fluoro-5-[1-hydroxy- |
| | 1-(1-methyl-1H-imidazol-4-yl)butyl]-3-oxo-1- |
| | [(3S)-oxolan-3-yloxy]-2,3-dihydro-1H-isoindol- |
| | 2-yl]methyl}pyridine-3-carbonitrile |
| 473 | 6-{[(1R)-1-(4-chlorophenyl)-7-fluoro-5-[1-hydroxy- |
| | 1-(1-methyl-1H-imidazol-4-yl)butyl]-3-oxo-1- |
| | [(3S)-oxolan-3-yloxy]-2,3-dihydro-1H-isoindol- |
| | 2-yl]methyl}pyridine-3-carbonitrile |
| 474 | (3R)-3-(4-chlorophenyl)-2-[(5-chloropyrimidin-2- |
| | yl)methyl]-4-fluoro-6-[1-hydroxy-1-(1-methyl-1H- |
| | imidazol-4-yl)propyl]-3-[(3S)-oxolan-3-yloxy]- |
| | 2,3-dihydro-1H-isoindol-1-one |
| 475 | (3R)-3-(4-chlorophenyl)-2-[(5-chloropyrimidin-2- |
| | yl)methyl]-4-fluoro-6-[1-hydroxy-1-(1-methyl-1H- |
| | imidazol-4-yl)propyl]-3-[(3S)-oxolan-3-yloxy]- |
| | 2,3-dihydro-1H-isoindol-1-one |
| 476 | (3R)-3-(4-chlorophenyl)-2-[(5-chloropyrimidin-2- |
| | yl)methyl]-4-fluoro-6-[1-hydroxy-1-(1-methyl-1H- |
| | imidazol-4-yl)propyl]-3-{[1-(hydroxymethyl)cyclo- |
| | propyl]methoxy}-2,3-dihydro-1H-isoindol-1-one |
| 477 | (3R)-3-(4-chlorophenyl)-2-[(5-chloropyrimidin-2- |
| | yl)methyl]-4-fluoro-6-[1-hydroxy-1-(1-methyl-1H- |
| | imidazol-4-yl)propyl]-3-{[1-(hydroxymethyl)cyclo- |
| | propyl]methoxy}-2,3-dihydro-1H-isoindol-1-one |
| 478 | 2-{[(1R)-1-(4-chlorophenyl)-7-fluoro-5-[1-hydroxy-1- |
| | (1-methyl-1H-imidazol-4-yl)propyl]-1-[(trans-3- |
| | hydroxycyclopentyl)oxy]-3-oxo-2,3-dihydro-1H- |
| | isoindol-2-yl]methyl}pyrimidine-5-carbonitrile |
| 479 | 2-{[(1R)-1-(4-chlorophenyl)-7-fluoro-5-[1-hydroxy- |
| | 1-(1-methyl-1H-imidazol-4-yl)propyl]-1-[(trans-3- |
| | hydroxycyclopentyl)oxy]-3-oxo-2,3-dihydro-1H- |
| | isoindol-2-yl]methyl}pyrimidine-5-carbonitrile |
| 480 | 2-{[(1R)-1-(4-chlorophenyl)-7-fluoro-5-[1-hydroxy- |
| | 1-(1-methyl-1H-imidazol-4-yl)propyl]-1-[(trans-3- |
| | hydroxycyclopentyl)oxy]-3-oxo-2,3-dihydro-1H- |
| | isoindol-2-yl]methyl}pyrimidine-5-carbonitrile |
| 481 | 2-{[(1R)-1-(4-chlorophenyl)-7-fluoro-5-[1-hydroxy- |
| | 1-(1-methyl-1H-imidazol-4-yl)propyl]-3-oxo-1- |
| | [trans-3-(hydroxymethyl)cyclobutoxy]-2,3-dihydro- |
| | 1H-isoindol-2-yl]methyl}pyrimidine-5-carbonitrile |
| 482 | 2-{[(1R)-1-(4-chlorophenyl)-7-fluoro-5-[1-hydroxy- |
| | 1-(1-methyl-1H-imidazol-4-yl)propyl]-3-oxo-1- |
| | [trans-3-(hydroxymethyl)cyclobutoxy]-2,3-dihydro- |
| | 1H-isoindol-2-yl]methyl}pyrimidine-5-carbonitrile |
| 483 | 2-{[(1R)-1-(4-chlorophenyl)-7-fluoro-5-[1-hydroxy- |
| | 1-(1-methyl-1H-imidazol-4-yl)propyl]-3-oxo-1- |
| | {[trans-3-hydroxycyclobutyl]methoxy}-2,3-dihydro- |
| | 1H-isoindol-2-yl]methyl}pyrimidine-5-carbonitrile |
| 484 | 2-{[(1R)-1-(4-chlorophenyl)-7-fluoro-5-[1-hydroxy- |
| | 1-(1-methyl-1H-imidazol-4-yl)propyl]-3-oxo-1- |
| | {[trans-3-hydroxycyclobutyl]methoxy}-2,3-dihydro- |
| | 1H-isoindol-2-yl]methyl}pyrimidine-5-carbonitrile |
| 485 | 1-({[(1R)-1-(4-chlorophenyl)-2-[(5-chloropyrimidin- |
| | 2-yl)methyl]-7-fluoro-5-[1-hydroxy-1-(1-methyl- |
| | 1H-imidazol-4-yl)propyl]-3-oxo-2,3-dihydro-1H- |
| | isoindol-1-yl]oxy}methyl)cyclopropane-1- |
| | carboxamide |
| 486 | 1-({[(1R)-1-(4-chlorophenyl)-2-[(5-chloropyrimidin- |
| | 2-yl)methyl]-7-fluoro-5-[1-hydroxy-1-(1-methyl- |
| | 1H-imidazol-4-yl)propyl]-3-oxo-2,3-dihydro- |
| | 1H-isoindol-1-yl]oxy}methyl)cyclopropane-1- |
| | carboxamide |
| 487 | (3R)-2-[(5-chloro-3-hydroxypyridin-2-yl)methyl]- |
| | 3-(4-chlorophenyl)-4-fluoro-6-[1-hydroxy-1-(1- |
| | methyl-1H-imidazol-4-yl)propyl]-3-[cis-3- |
| | hydroxycyclobutoxy]-2,3-dihydro-1H-isoindol-1-one |
| 488 | (3R)-2-[(5-chloro-3-hydroxypyridin-2-yl)methyl]- |
| | 3-(4-chlorophenyl)-4-fluoro-6-[1-hydroxy-1-(1- |
| | methyl-1H-imidazol-4-yl)propyl]-3-[cis-3- |
| | hydroxycyclobutoxy]-2,3-dihydro-1H-isoindol-1-one |
| 489 | 2-{[(1R)-1-(4-chlorophenyl)-7-fluoro-5-[1-hydroxy- |
| | 1-(1-methyl-1H-pyrazol-3-yl)propyl]-3-oxo-1- |
| | [(3S)-oxolan-3-yloxy]-2,3-dihydro-1H-isoindol- |
| | 2-yl]methyl}pyrimidine-5-carbonitrile |
| 490 | 2-{[(1R)-1-(4-chlorophenyl)-7-fluoro-5-[1- |
| | hydroxy-1-(1-methyl-1H-pyrazol-3-yl)propyl]-3-oxo-1- |
| | [(3S)-oxolan-3-yloxy]-2,3-dihydro-1H-isoindol- |
| | 2-yl]methyl}pyrimidine-5-carbonitrile |
| 491 | (3R)-2-[(5-chloro-3-methoxypyridin-2-yl)methyl]- |
| | 3-(4-chlorophenyl)-4-fluoro-6-[1-hydroxy-1-(1- |
| | methyl-1H-imidazol-4-yl)propyl]-3-(2-hydroxyethoxy)- |
| | 2,3-dihydro-1H-isoindol-1-one |
| 492 | (3R)-2-[(5-chloro-3-methoxypyridin-2-yl)methyl]- |
| | 3-(4-chlorophenyl)-4-fluoro-6-[1-hydroxy-1-(1- |
| | methyl-1H-imidazol-4-yl)propyl]-3-(2-hydroxyethoxy)- |
| | 2,3-dihydro-1H-isoindol-1-one |
| 493 | (3R)-3-(4-chlorophenyl)-2-[(5-chloropyrimidin- |
| | 2-yl)methyl]-4-fluoro-6-[1-(4-fluorooxan-4-yl)-1- |
| | hydroxyethyl]-3-[(3S)-oxolan-3-yloxy]-2,3-dihydro- |
| | 1H-isoindol-1-one |
| 494 | (3R)-3-(4-chlorophenyl)-2-[(5-chloropyrimidin- |
| | 2-yl)methyl]-4-fluoro-6-[1-(4-fluorooxan-4-yl)-1- |
| | hydroxyethyl]-3-[(3S)-oxolan-3-yloxy]-2,3- |
| | dihydro-1H-isoindol-1-one |
| 495 | (3R)-3-(4-chlorophenyl)-2-[(5-chloropyrimidin-2- |
| | yl)methyl]-4-fluoro-6-{1-hydroxy-1-[trans-4- |
| | hydroxycyclohexyl}-3-[cis-3-hydroxycyclobutoxy]- |
| | 2,3-dihydro-1H-isoindol-1-one |
| 496 | (3R)-3-(4-chlorophenyl)-2-[(5-chloropyrimidin- |
| | 2-yl)methyl]-4-fluoro-6-{1-hydroxy-1-[trans-4- |
| | hydroxycyclohexyl}-3-[ cis-3-hydroxycyclobutoxy]- |
| | 2,3-dihydro-1H-isoindol-1-one: *fast eluting |
| | isomer |
| 497 | 6-{[(1R)-1-(4-chlorophenyl)-7-fluoro-5-[1-(4- |
| | fluorooxan-4-yl)-1-hydroxypropyl]-1-[(2R)-2- |
| | hydroxypropoxy]-3-oxo-2,3-dihydro-1H-isoindol- |
| | 2-yl]methyl}pyridine-3-carbonitrile |
| 498 | 6-{[(1R)-1-(4-chlorophenyl)-7-fluoro-5-[1-(4- |
| | fluorooxan-4-yl)-1-hydroxypropyl]-1-[(2R)-2- |
| | hydroxypropoxy]-3-oxo-2,3-dihydro-1H-isoindol- |
| | 2-yl]methyl}pyridine-3-carbonitrile |
| 499 | 6-{[(1R)-1-(4-chlorophenyl)-7-fluoro-5-[1- |
| | (4-fluorooxan-4-yl)-1-hydroxypropyl]-1-(2- |
| | hydroxyethoxy)-3-oxo-2,3-dihydro-1H-isoindol- |
| | 2-yl]methyl}pyridine-3-carbonitrile |
| 500 | 6-{[(1R)-1-(4-chlorophenyl)-7-fluoro-5-[1- |
| | (4-fluorooxan-4-yl)-1-hydroxypropyl]-1-(2- |
| | hydroxyethoxy)-3-oxo-2,3-dihydro-1H- |
| | isoindol-2-yl]methyl}pyridine-3-carbonitrile |
| 501 | (3R)-3-(4-chlorophenyl)-2-[(5-chloropyrimidin-2- |
| | yl)methyl]-4-fluoro-6-[(1R)-1-(4-fluorooxan-4-yl)-1- |
| | hydroxypropyl]-3-(2-hydroxyethoxy)-2,3-dihydro- |
| | 1H-isoindol-1-one |
| 502 | (3R)-3-(4-chlorophenyl)-2-[(5-chloropyrimidin-2- |
| | yl)methyl]-4-fluoro-6-[(1S)-1-(4-fluorooxan-4-yl)-1- |
| | hydroxypropyl]-3-(2-hydroxyethoxy)-2,3-dihydro- |
| | 1H-isoindol-1-one |
| 503 | 2-{[(1R)-1-(4-chlorophenyl)-7-fluoro-5-{1-hydroxy- |
| | 1-[trans-4-hydroxycyclohexyl]propyl}-3-oxo-1- |
| | [(3S)-oxolan-3-yloxy]-2,3-dihydro-1H-isoindol- |
| | 2-yl]methyl}pyrimidine-5-carbonitrile |
| 504 | 2-{[(1R)-1-(4-chlorophenyl)-7-fluoro-5-{1- |
| | hydroxy-1-[trans-4-hydroxycyclohexyl]propyl}-1- |
| | methoxy-3-oxo-2,3-dihydro-1H-isoindol-2- |
| | yl]methyl}pyrimidine-5-carbonitrile |
| 505 | 2-{[(1R)-1-(4-chlorophenyl)-7-fluoro-5-[(1R)-1- |
| | (4-fluorooxan-4-yl)-1-hydroxypropyl]-3-oxo-1-[cis-3- |
| | hydroxycyclobutoxy]-2,3-dihydro-1H-isoindol- |
| | 2-yl]methyl}pyrimidine-5-carbonitrile |
| 506 | 2-{[(1R)-1-(4-chlorophenyl)-7-fluoro-5-[(1S)-1- |
| | (4-fluorooxan-4-yl)-1-hydroxypropyl]-3-oxo-1-[cis-3- |
| | hydroxycyclobutoxy]-2,3-dihydro-1H-isoindol-2- |
| | yl]methyl}pyrimidine-5-carbonitrile |
| 507 | 2-{[(1R)-1-(4-chlorophenyl)-7-fluoro-5-[(1R)-1- |
| | (4-fluorooxan-4-yl)-1-hydroxypropyl]-3-oxo-1-[(3S)- |
| | oxolan-3-yloxy]-2,3-dihydro-1H-isoindol-2- |
| | yl]methyl}pyrimidine-5-carbonitrile |
| 508 | 2-{[(1R)-1-(4-chlorophenyl)-7-fluoro-5-[(1S)-1- |
| | (4-fluorooxan-4-yl)-1-hydroxypropyl]-3-oxo-1-[(3S)- |
| | oxolan-3-yloxy]-2,3-dihydro-1H-isoindol-2- |
| | yl]methyl}pyrimidine-5-carbonitrile |
| 509 | 2-{[(1R)-1-(4-chlorophenyl)-7-fluoro-5-[(1R)-1- |
| | (4-fluorooxan-4-yl)-1-hydroxypropyl]-1-(2- |
| | hydroxyethoxy)-3-oxo-2,3-dihydro-1H-isoindol- |
| | 2-yl]methyl}pyrimidine-5-carbonitrile |
| 510 | 2-{[(1R)-1-(4-chlorophenyl)-7-fluoro-5-{1-hydroxy- |
| | 1-[trans-4-hydroxycyclohexyl]propyl}-1-(2- |
| | hydroxyethoxy)-3-oxo-2,3-dihydro-1H-isoindol- |
| | 2-yl]methyl}pyrimidine-5-carbonitrile |
| 511 | (3R)-3-(4-chlorophenyl)-2-[(5-chloropyrimidin-2- |
| | yl)methyl]-4-fluoro-6-{1-hydroxy-1-[trans-4- |
| | hydroxycyclohexyl]propyl}-3-methoxy-2,3- |
| | dihydro-1H-isoindol-1-one |
| 512 | 2-{[(1R)-1-(4-chlorophenyl)-7-fluoro-5-{1-hydroxy- |
| | 1-[trans-4-hydroxycyclohexyl]propyl}-1-[(2R)-2- |
| | hydroxypropoxy]-3-oxo-2,3-dihydro-1H-isoindol- |
| | 2-yl]methyl}pyrimidine-5-carbonitrile |
| 513 | 2-{[(1R)-1-(4-chlorophenyl)-7-fluoro-1-({1- |
| | [hydroxydideuteromethyl]cyclopropyl}dideuteromethoxy)- |
| | 5-(2-hydroxypropan-2-yl)-3-oxo-2,3- |
| | dihydro-1H-isoindol-2-yl]methyl}pyrimidine-5-carbonitrile |
| 514 | 5-chloro-2-{[(1R)-1-(4-chlorophenyl)-7-fluoro-5- |
| | [(1R)-1-(4-fluorooxan-4-yl)-1-hydroxypropyl]-1- |
| | methoxy-3-oxo-2,3-dihydro-1H-isoindol- |
| | 2-yl]methyl}pyridine-3-carboxylic acid |
| | (tris(hydroxymethyl)aminomethane salt) |
| 515 | 3-{[(1R)-1-(4-chlorophenyl)-7-fluoro-5-[(1R)-1- |
| | (4-fluorooxan-4-yl)-1-hydroxypropyl]-1-methoxy-3- |
| | oxo-2,3-dihydro-1H-isoindol-2-yl]methyl}-6- |
| | methylpyridine-2-carboxylic acid |
| 516 | (3R)-3-(4-chlorophenyl)-2-[(5-chloropyrimidin-2- |
| | yl)dideuteromethyl]-4-fluoro-6-[1-(4-fluorooxan-4- |
| | yl)-1-hydroxyethyl]-3-[(3S)-oxolan-3-yloxy]-2,3- |
| | dihydro-1H-isoindol-1-one |
| 517 | (3R)-3-(4-chlorophenyl)-2-[(5-chloropyrimidin- |
| | 2-yl)methyl]-4-fluoro-6-{1-hydroxy-1-[trans-4- |
| | hydroxycyclohexyl]propyl}-3-(2-hydroxyethoxy)- |
| | 2,3-dihydro-1H-isoindol-1-one |
| 518 | 6-{[(1R)-1-(4-chlorophenyl)-7-fluoro-5-{1-hydroxy- |
| | 1-[trans-4-hydroxycyclohexyl]propyl}-3-oxo-1- |
| | [cis-3-hydroxycyclobutoxy]-2,3-dihydro-1H-isoindol- |
| | 2-yl]methyl}pyridine-3-carbonitrile |
| 519 | (3R)-3-(4-chlorophenyl)-2-[(5-chloropyrimidin- |
| | 2-yl)methyl]-4-fluoro-6-{1-hydroxy-1-[trans-4- |
| | hydroxycyclohexyl]propyl}-3-(2-hydroxyethoxy)- |
| | 2,3-dihydro-1H-isoindol-1-one |
| 520 | 6-{[(1R)-1-(4-chlorophenyl)-7-fluoro-5-{1-hydroxy- |
| | 1-[trans-4-hydroxycyclohexyl]propyl}-3-oxo-1- |
| | [cis-3-hydroxycyclobutoxy]-2,3-dihydro-1H-isoindol- |
| | 2-yl]methyl}pyridine-3-carbonitrile |
| 521 | 6-{[(1R)-1-(4-chlorophenyl)-7-fluoro-5-{1-hydroxy- |
| | 1-[trans-4-hydroxycyclohexyl]propyl}-1- |
| | methoxy-3-oxo-2,3-dihydro-1H-isoindol-2- |
| | yl]methyl}pyridine-3-carbonitrile |
| 522 | (3R)-3-(4-chlorophenyl)-2-[(5-chloropyrimidin- |
| and | 2-yl)methyl]-4-fluoro-6-[2-hydroxy-1-(4- |
| 523 | methylpiperazin-1-yl)butan-2-yl]-3-[(3S)-oxolan- |
| | 3-yloxy]-2,3-dihydro-1H-isoindol-1-one |
| 524 | (3R)-3-(4-chlorophenyl)-2-[(5-chloropyrimidin-2- |
| | yl)methyl]-4-fluoro-6-[2-hydroxy-1-(piperazin-1- |
| | yl)butan-2-yl]-3-[(3S)-oxolan-3-yloxy]-2,3-dihydro- |
| | 1H-isoindol-1-one |
| 525 | (3R)-3-(4-chlorophenyl)-2-[(5-chloropyrimidin-2- |
| | yl)methyl]-4-fluoro-6-[2-hydroxy-1-(piperazin-1- |
| | yl)butan-2-yl]-3-[(3S)-oxolan-3-yloxy]-2,3-dihydro- |
| | 1H-isoindol-1-one |
| 526 | (3R)-3-(4-chlorophenyl)-2-[(5-chloropyrimidin-2- |
| | yl)methyl]-4-fluoro-6-{2-hydroxy-1-[(3R)-3- |
| | hydroxypyrrolidin-1-yl]butan-2-yl}-3-[(3S)- |
| | oxolan-3-yloxy]-2,3-dihydro-1H-isoindol-1-one |
| 527 | (3R)-3-(4-chlorophenyl)-2-[(5-chloropyrimidin- |
| | 2-yl)methyl]-4-fluoro-6-{2-hydroxy-1-[(3R)-3- |
| | hydroxypyrrolidin-1-yl]butan-2-yl}-3-[(3S)-oxolan- |
| | 3-yloxy]-2,3-dihydro-1H-isoindol-1-one |
| 528 | (3R)-3-(4-chlorophenyl)-2-[(5-chloropyrimidin-2- |
| | yl)methyl]-6-[1-(dimethylamino)-2-hydroxybutan- |
| | 2-yl]-4-fluoro-3-[(3S)-oxolan-3-yloxy]-2,3-dihydro- |
| | 1H-isoindol-1-one |
| 529 | (3R)-3-(4-chlorophenyl)-2-[(5-chloropyrimidin-2- |
| | yl)methyl]-6-[1-(dimethylamino)-2-hydroxybutan- |
| | 2-yl]-4-fluoro-3-[(3S)-oxolan-3-yloxy]-2,3-dihydro- |
| | 1H-isoindol-1-one |
| 530 | (3R)-3-(4-chlorophenyl)-2-[(5-chloropyrimidin-2- |
| | yl)methyl]-4-fluoro-6-[2-hydroxy-1-(piperazin-1- |
| | yl)butan-2-yl]-3-(2-hydroxyethoxy)-2,3-dihydro- |
| | 1H-isoindol-1-one |
| 531 | (3R)-3-(4-chlorophenyl)-2-[(5-chloropyrimidin- |
| | 2-yl)methyl]-4-fluoro-6-[2-hydroxy-1-(piperazin-1- |
| | yl)butan-2-yl]-3-(2-hydroxyethoxy)-2,3-dihydro- |
| | 1H-isoindol-1-one |
| 532 | (3R)-3-(4-chlorophenyl)-2-[(5-chloropyrimidin- |
| | 2-yl)methyl]-4-fluoro-6-[2-hydroxy-1-(4- |
| | methylpiperazin-1-yl)butan-2-yl]-3-(2-hydroxyethoxy)- |
| | 2,3-dihydro-1H-isoindol-1-one |
| 533 | (3R)-3-(4-chlorophenyl)-2-[(5-chloropyrimidin- |
| | 2-yl)methyl]-4-fluoro-6-[2-hydroxy-1-(4- |
| | methylpiperazin-1-yl)butan-2-yl]-3-(2-hydroxyethoxy)- |
| | 2,3-dihydro-1H-isoindol-1-one |
| 534 | 1-({[(1R)-1-(4-chlorophenyl)-2-[(5-chloropyrimidin- |
| | 2-yl)methyl]-7-fluoro-5-[2-hydroxy-1-(4- |
| | methylpiperazin-1-yl)butan-2-yl]-3-oxo-2,3-dihydro- |
| | 1H-isoindol-1-yl]oxy}methyl)cyclopropane-1- |
| | carbonitrile |
| 535 | 1-({[(1R)-1-(4-chlorophenyl)-2-[(5-chloropyrimidin- |
| | 2-yl)methyl]-7-fluoro-5-[2-hydroxy-1-(4- |
| | methylpiperazin-1-yl)butan-2-yl]-3-oxo-2,3-dihydro- |
| | 1H-isoindol-1-yl]oxy}methyl)cyclopropane-1- |
| | carbonitrile |
| 536 | 1-({[(1R)-1-(4-chlorophenyl)-2-[(5-chloropyrimidin- |
| | 2-yl)methyl]-7-fluoro-5-[2-hydroxy-1-(piperazin- |
| | 1-yl)butan-2-yl]-3-oxo-2,3-dihydro-1H-isoindol- |
| | 1-yl]oxy}methyl)cyclopropane-1-carbonitrile |
| 537 | 1-({[(1R)-1-(4-chlorophenyl)-2-[(5-chloropyrimidin- |
| | 2-yl)methyl]-7-fluoro-5-[2-hydroxy-1-(piperazin- |
| | 1-yl)butan-2-yl]-3-oxo-2,3-dihydro-1H-isoindol- |
| | 1-yl]oxy}methyl)cyclopropane-1-carbonitrile |
| 538 | (3R)-3-(4-chlorophenyl)-2-[(5-chloropyrimidin- |
| | 2-yl)methyl]-4-fluoro-3-[(3-fluorooxetan-3- |
| | yl)methoxy]-6-[2-hydroxy-1-(piperazin-1-yl)butan- |
| | 2-yl]-2,3-dihydro-1H-isoindol-1-one |
| 539 | (3R)-3-(4-chlorophenyl)-2-[(5-chloropyrimidin- |
| | 2-yl)methyl]-4-fluoro-3-[(3-fluorooxetan-3- |
| | yl)methoxy]-6-[2-hydroxy-1-(piperazin-1- |
| | yl)butan-2-yl]-2,3-dihydro-1H-isoindol-1-one |
| 540 | (3R)-3-(4-chlorophenyl)-2-[(5-chloropyrimidin-2- |
| | yl)methyl]-4-fluoro-6-[1-(4-fluoropiperidin-4-yl)-1- |
| | hydroxypropyl]-3-[(3S)-oxolan-3-yloxy]- |
| | 2,3-dihydro-1H-isoindol-1-one |
| 541 | (3R)-3-(4-chlorophenyl)-2-[(5-chloropyrimidin-2- |
| | yl)methyl]-4-fluoro-6-[1-(4-fluoropiperidin-4-yl)-1- |
| | hydroxypropyl]-3-[(3S)-oxolan-3-yloxy]-2,3- |
| | dihydro-1H-isoindol-1-one |
| 541 | (3R)-3-(4-chlorophenyl)-2-[(5-chloropyrimidin-2- |
| a | yl)methyl]-4-fluoro-6-[1-(4-fluoropiperidin-4-yl)-1- |
| | hydroxypropyl]-3-[(3S)-oxolan-3-yloxy]-2,3-dihydro- |
| | 1H-isoindol-1-one-L-(+)-lactic acid salt |
| 542 | (3R)-3-(4-chlorophenyl)-2-[(5-chloropyrimidin-2- |
| | yl)methyl]-4-fluoro-6-[1-(4-fluoropiperidin-4-yl)-1- |
| | hydroxypropyl]-3-[cis-3-hydroxycyclobutoxy]- |
| | 2,3-dihydro-1H-isoindol-1-one |
| 543 | (3R)-3-(4-chlorophenyl)-2-[(5-chloropyrimidin-2- |
| | yl)methyl]-4-fluoro-6-[1-(4-fluoropiperidin-4-yl)-1- |
| | hydroxypropyl]-3-[(2R)-2-hydroxypropoxy]- |
| | 2,3-dihydro-1H-isoindol-1-one |
| 544 | (3R)-3-(4-chlorophenyl)-2-[(5-chloropyrimidin-2- |
| | yl)methyl]-4-fluoro-6-[1-(4-fluoropiperidin-4-yl)-1- |
| | hydroxypropyl]-3-methoxy-2,3-dihydro-1H-isoindol-1-one |
| 545 | 1-({[(1R)-1-(4-chlorophenyl)-2-[(5-chloropyrimidin- |
| | 2-yl)methyl]-7-fluoro-5-[1-(4-fluoropiperidin-4- |
| | yl)-1-hydroxypropyl]-3-oxo-2,3-dihydro-1H-isoindol- |
| | 1-yl]oxy}methyl)cyclopropane-1-carbonitrile |
| 546 | 6-{[(1R)-1-(4-chlorophenyl)-7-fluoro-5-[1-(4- |
| | fluoropiperidin-4-yl)-1-hydroxypropyl]-3-oxo-1-[(3S)- |
| | oxolan-3-yloxy]-2,3-dihydro-1H-isoindol-2- |
| | yl]methyl}pyridine-3-carbonitrile |
| 547 | 2-{[(1R)-1-(4-chlorophenyl)-7-fluoro-5-[1-(4- |
| | fluoropiperidin-4-yl)-1-hydroxypropyl]-3-oxo-1-[(3S)- |
| | oxolan-3-yloxy]-2,3-dihydro-1H-isoindol-2- |
| | yl]methyl}pyrimidine-5-carbonitrile |
| 548 | (3R)-3-(4-chlorophenyl)-2-[(5-chloropyridin-2- |
| | yl)methyl]-4-fluoro-6-[1-(4-fluoropiperidin-4-yl)-1- |
| | hydroxypropyl]-3-[(3S)-oxolan-3-yloxy]-2,3- |
| | dihydro-1H-isoindol-1-one |
| 549 | (3R)-3-(4-chlorophenyl)-2-[(5-chloropyridin-2- |
| | yl)methyl]-4-fluoro-6-[1-(4-fluoropiperidin-4-yl)-1- |
| | hydroxypropyl]-3-[(2R)-2-hydroxypropoxy]- |
| | 2,3-dihydro-1H-isoindol-1-one |
| 550 | (3R)-3-(4-chlorophenyl)-2-[(5-chloropyrimidin- |
| | 2-yl)methyl]-4-fluoro-6-[(1S)-1-hydroxy-1-[1-(2- |
| | hydroxyethyl)piperidin-4-yl]propyl]-3-methoxy- |
| | 2,3-dihydro-1H-isoindol-1-one |
| 551 | (3R)-3-(4-chlorophenyl)-2-[(5-chloropyrimidin-2- |
| | yl)methyl]-4-fluoro-6-[(1S)-1-hydroxy-1-[1-(oxetan- |
| | 3-yl)piperidin-4-yl]propyl]-3-methoxy-2,3-dihydro- |
| | 1H-isoindol-1-one |
| 552 | (3R)-3-(4-chlorophenyl)-2-[(5-chloropyrimidin-2- |
| | yl)methyl]-4-fluoro-6-[1-[4-fluoro-1-(oxetan-3- |
| | yl)piperidin-4-yl]-1-hydroxypropyl]-3-methoxy- |
| | 2,3-dihydro-1H-isoindol-1-one |
| 553 | (3R)-3-(4-chlorophenyl)-2-[(5-chloropyrimidin-2- |
| | yl)methyl]-4-fluoro-6-[(1S)-1-hydroxy-1-(1- |
| | methylpiperidin-4-yl)propyl]-3-methoxy-2,3- |
| | dihydro-1H-isoindol-1-one |
| 554 | (3R)-3-(4-chlorophenyl)-2-[(5-chloropyrimidin- |
| | 2-yl)methyl]-4-fluoro-6-[1-hydroxy-1-(1- |
| | methylpiperidin-4-yl)ethyl]-3-{[1-(hydroxymethyl)cyclo- |
| | propyl]methoxy}-2,3-dihydro-1H-isoindol-1- |
| | one |
| 555 | (3R)-3-(4-chlorophenyl)-2-[(5-chloropyridin- |
| | 2-yl)methyl]-4-fluoro-6-[1-hydroxy-1-(1- |
| | methylpiperidin-4-yl)ethyl]-3-{[1-(hydroxymethyl)cyclo- |
| | propyl]methoxy}-2,3-dihydro-1H-isoindol-1- |
| | one |
| 556 | (3R)-3-(4-chlorophenyl)-2-[(5-chloropyrimidin- |
| | 2-yl)methyl]-4-fluoro-6-[(1S)-1-hydroxy-1-(1- |
| | methylpiperidin-4-yl)propyl]-3-{[1-(hydroxymethyl)cyclo- |
| | propyl]methoxy}-2,3-dihydro-1H-isoindol-1- |
| | one |
| 557 | 2-{[(1R)-1-(4-chlorophenyl)-7-fluoro-5-[(1S)-1- |
| | hydroxy-1-(1-methylpiperidin-4-yl)propyl]-3-oxo-1- |
| | [(3S)-oxolan-3-yloxy]-2,3-dihydro-1H-isoindol- |
| | 2-yl]methyl}pyrimidine-5-carbonitrile |
| 558 | (3R)-3-(4-chlorophenyl)-2-[(5-chloropyrimidin- |
| | 2-yl)methyl]-4-fluoro-3-[(3-fluorooxetan-3- |
| | yl)methoxy]-6-[1-hydroxy-1-(1-methylpiperidin- |
| | 4-yl)ethyl]-2,3-dihydro-1H-isoindol-1-one |
| 559 | (3R)-3-(4-chlorophenyl)-2-[(5-chloropyrimidin- |
| | 2-yl)methyl]-4-fluoro-6-[(1S)-1-hydroxy-1-(1- |
| | methylpiperidin-4-yl)propyl]-3-(2-hydroxyethoxy)- |
| | 2,3-dihydro-1H-isoindol-1-one |
| 560 | (3R)-3-(4-chlorophenyl)-2-[(5-chloropyrimidin- |
| | 2-yl)methyl]-4-fluoro-6-[(1S)-1-hydroxy-1-(1- |
| | methylpiperidin-4-yl)propyl]-3-[cis-3- |
| | hydroxycyclobutoxy]-2,3-dihydro-1H-isoindol-1-one |
| 561 | 2-{[(1R)-1-(4-chlorophenyl)-7-fluoro-5-[1-(4-fluoro- |
| | 1-methylpiperidin-4-yl)-1-hydroxypropyl]-3-oxo- |
| | 1-[(3S)-oxolan-3-yloxy]-2,3-dihydro-1H-isoindol- |
| | 2-yl]methyl}pyrimidine-5-carbonitrile |
| 562 | (3R)-3-(4-chlorophenyl)-2-[(5-chloropyrimidin- |
| | 2-yl)methyl]-4-fluoro-6-[(1S)-1-hydroxy-1-(1- |
| | methylpiperidin-4-yl)propyl]-3-[(2R)-2- |
| | hydroxypropoxy]-2,3-dihydro-1H-isoindol-1-one |
| 563 | (3R)-3-(4-chlorophenyl)-2-[(5-chloropyrimidin- |
| | 2-yl)methyl]-4-fluoro-6-[1-(4-fluoro-1- |
| | methylpiperidin-4-yl)-1-hydroxypropyl]-3- |
| | methoxy-2,3-dihydro-1H-isoindol-1-one |
| 563 | (3R)-3-(4-chlorophenyl)-2-[(5-chloropyrimidin- |
| a | 2-yl)methyl]-4-fluoro-6-[1-(4-fluoro-1- |
| | methylpiperidin-4-yl)-1-hydroxypropyl]-3-methoxy- |
| | 2,3-dihydro-1H-isoindol-1-one-L-(+)-lactic acid |
| | salt |
| 563 | (3R)-3-(4-chlorophenyl)-2-[(5-chloropyrimidin- |
| b | 2-yl)methyl]-4-fluoro-6-[1-(4-fluoro-1- |
| | methylpiperidin-4-yl)-1-hydroxypropyl]-3- |
| | methoxy-2,3-dihydro-1H-isoindol-1-one-hydrochloride |
| | salt |
| 564 | 1-({[(1R)-1-(4-chlorophenyl)-2-[(5-chloropyrimidin- |
| | 2-yl)methyl]-7-fluoro-5-[1-(4-fluoro-1- |
| | methylpiperidin-4-yl)-1-hydroxypropyl]-3- |
| | oxo-2,3-dihydro-1H-isoindol-1- |
| | yl]oxy}methyl)cyclopropane-1-carbonitrile |
| 564 | 1-({[(1R)-1-(4-chlorophenyl)-2-[(5-chloropyrimidin- |
| a | 2-yl)methyl]-7-fluoro-5-[1-(4-fluoro-1- |
| | methylpiperidin-4-yl)-1-hydroxypropyl]- |
| | 3-oxo-2,3-dihydro-1H-isoindol-1- |
| | yl]oxy}methyl)cyclopropane-1-carbonitrile-L-(+)- |
| | lactic acid salt |
| 564 | 1-({[(1R)-1-(4-chlorophenyl)-2-[(5-chloropyrimidin- |
| b | 2-yl)methyl]-7-fluoro-5-[1-(4-fluoro-1- |
| | methylpiperidin-4-yl)-1-hydroxypropyl]-3- |
| | oxo-2,3-dihydro-1H-isoindol-1- |
| | yl]oxy}methyl)cyclopropane-1-carbonitrile- |
| | hydrochloride salt |
| 565 | (3R)-3-(4-chlorophenyl)-2-[(5-chloropyrimidin- |
| | 2-yl)methyl]-4-fluoro-6-[1-(4-fluoro-1- |
| | methylpiperidin-4-yl)-1-hydroxypropyl]-3-(2- |
| | hydroxyethoxy)-2,3-dihydro-1H-isoindol-1-one |
| 566 | (3R)-3-(4-chlorophenyl)-2-[(5-chloropyrimidin- |
| | 2-yl)methyl]-4-fluoro-6-[(1R)-1-hydroxy-1-(1- |
| | methylpiperidin-4-yl)propyl]-3-methoxy- |
| | 2,3-dihydro-1H-isoindol-1-one |
| 567 | (3R)-3-(4-chlorophenyl)-2-[(5-chloropyrimidin- |
| | 2-yl)methyl]-4-fluoro-6-[(1R)-1-hydroxy-1-(1- |
| | methylpiperidin-4-yl)propyl]-3-[(3S)-oxolan- |
| | 3-yloxy]-2,3-dihydro-1H-isoindol-1-one |
| 568 | 2-[(5-chloro-3-hydroxypyridin-2-yl)methyl]-3- |
| | (4-chlorophenyl)-4-fluoro-6-[(1S)-1-hydroxy-1-(1- |
| | methylpiperidin-4-yl)propyl]-3-[(3S)-oxolan- |
| | 3-yloxy]-2,3-dihydro-1H-isoindol-1-one |
| 570 | (3R)-3-(4-chlorophenyl)-2-[(5-chloropyrimidin- |
| | 2-yl)methyl]-4-fluoro-6-[1-(4-fluoro-1- |
| | methylpiperidin-4-yl)-1-hydroxypropyl]-3-[cis-3- |
| | hydroxycyclobutoxy]-2,3-dihydro-1H-isoindol-1-one |
| 571 | (3R)-3-(4-chlorophenyl)-2-[(5-chloropyrimidin- |
| | 2-yl)methyl]-4-fluoro-6-[1-(4-fluoro-1- |
| | methylpiperidin-4-yl)-1-hydroxypropyl]-3-[(3S)- |
| | oxolan-3-yloxy]-2,3-dihydro-1H-isoindol-1-one |
| 572 | (3R)-3-(4-chlorophenyl)-2-[(5-chloropyrimidin- |
| | 2-yl)methyl]-4-fluoro-6-[1-(4-fluoro-1- |
| | methylpiperidin-4-yl)-1-hydroxypropyl]-3-[(2R)- |
| | 2-hydroxypropoxy]-2,3-dihydro-1H-isoindol-1-one |
| 574 | 2-{[(1R)-1-(4-chlorophenyl)-1-[(1-cyanocyclo- |
| | propyl)methoxy]-7-fluoro-5-[1-(4-fluoro-1- |
| | methylpiperidin-4-yl)-1-hydroxypropyl]-3-oxo-2 |
| | ,3-dihydro-1H-isoindol-2-yl]methyl}pyrimidine-5- |
| | carbonitrile |
| 575 | 2-{[(1R)-1-(4-chlorophenyl)-7-fluoro-5-[1-(4-fluoro- |
| | 1-methylpiperidin-4-yl)-1-hydroxypropyl]-1- |
| | methoxy-3-oxo-2,3-dihydro-1H-isoindol-2- |
| | yl]methyl}pyrimidine-5-carbonitrile |
| 576 | 2-{[(1R)-1-(4-chlorophenyl)-7-fluoro-1-({1- |
| | [hydroxydideuteromethyl]cyclopropyl}dideutero- |
| | methoxy)-5-(2-hydroxybutan-2-yl)-3-oxo-2,3- |
| | dihydro-1H-isoindol-2-yl]methyl}pyrimidine-5-carbonitrile |
| 577 | 2-{[(1R)-1-(4-chlorophenyl)-7-fluoro-1-({1- |
| | [hydroxydideuteromethyl]cyclopropyl}dideuteromethoxy)- |
| | 5-(2-hydroxybutan-2-yl)-3-oxo-2,3- |
| | dihydro-1H-isoindol-2-yl]methyl}pyrimidine-5-carbonitrile |
| 578 | (3R)-3-(4-chlorophenyl)-2-[(5-chloropyrimidin- |
| | 2-yl)methyl]-4-fluoro-6-[1-(4-fluorooxan-4-yl)-1- |
| | hydroxyethyl]-3-(2-hydroxyethoxy)-2,3-dihydro- |
| | 1H-isoindol-1-one |
| 579 | 6-{[(1R)-1-(4-chlorophenyl)-7-fluoro-1-[(2S)-3-fluoro- |
| | 2-hydroxypropoxy]-5-[1-(4-fluorooxan-4-yl)-1- |
| | hydroxyethyl]-3-oxo-2,3-dihydro-1H-isoindol-2- |
| | yl]methyl}pyridine-3-carbonitrile |
| 580 | (3R)-3-(4-chlorophenyl)-2-[(5-chloropyrimidin- |
| | 2-yl)methyl]-4-fluoro-6-[1-(4-fluoro-1- |
| | methylpiperidin-4-yl)-1-hydroxypropyl]-3-[2- |
| | hydroxy(1,1,2,2-tetradeutero)ethoxy]-2,3-dihydro-1H- |
| | isoindol-1-one |

Preparation 1 of (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid (“Compound 1”)

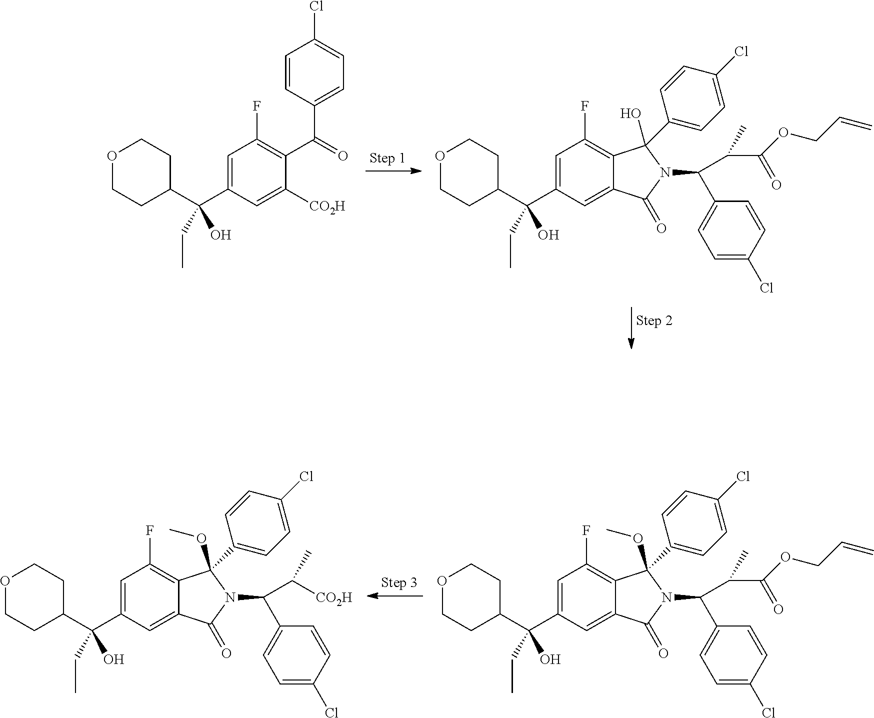 [SMILES: CC[C@@](O)(c1cc(F)c(C(=O)c2ccc(Cl)cc2)c(C(=O)O)c1)C1CCOCC1 ; C=CCOC(=O)[C@@H](C)[C@@H](c1ccc(Cl)cc1)N1C(=O)c2cc([C@](O)(CC)C3CCOCC3)cc(F)c2C1(O)c1ccc(Cl)cc1 ; CC[C@@](O)(c1cc(F)c2c(c1)C(=O)N([C@H](c1ccc(Cl)cc1)[C@H](C)C(=O)O)[C@@]2(OC)c1ccc(Cl)cc1)C1CCOCC1 ; C=CCOC(=O)[C@@H](C)[C@@H](c1ccc(Cl)cc1)N1C(=O)c2cc([C@](O)(CC)C3CCOCC3)cc(F)c2[C@]1(OC)c1ccc(Cl)cc1]

Step 1: Prop-2-en-1-yl (2S,3S)-3-(4-chlorophenyl)-3-[1-(4-chlorophenyl)-7-fluoro-1-hydroxy-5-[(1S)-1-hydroxy-1-(oxan-4-yl)propyl]-3-oxo-2,3-dihydro-1H-isoindol-2-yl]-2-methylpropanoate

To a solution of (S)-2-(4-chlorobenzoyl)-3-fluoro-5-(1-hydroxy-1-(tetrahydro-2H-pyran-4-yl)propyl)benzoic acid (Preparation 52) (0.686 g, 1.6 mmol), prop-2-en-1-yl (2S,3S)-3-amino-3-(4-chlorophenyl)-2-methylpropanoate (Preparation 62) (0.54 g, 2.12 mmol) and diisopropylethylamine (0.83 mL, 4.8 mmol) in DMF (15 mL) was added HATU (0.91 g, 2.4 mmol) and the reaction mixture was stirred for 2 hrs. Water was added and extracted with ethyl acetate. The organic phase was washed with saturated NaHCO₃, brine, dried and the solvent evaporated. The crude product was purified by chromatography to afford the title compound (0.75 g, 72%). MS: [M−H]⁻=654.

Step 2: Prop-2-en-1-yl (2S,3S)-3-(4-chlorophenyl)-3-[(1R)-1-(4-chlorophenyl)-7-fluoro-5-[(15)-1-hydroxy-1-(oxan-4-yl)propyl]-1-methoxy-3-oxo-2,3-dihydro-1H-isoindol-2-yl]-2-methylpropanoate

The title compound was prepared from ethyl (2S,3S)-3-(4-chlorophenyl)-3-[1-(4-chlorophenyl)-7-fluoro-1-hydroxy-5-[(1S)-1-hydroxy-1-(oxan-4-yl)propyl]-3-oxo-2,3-dihydro-1 H-isoindol-2-yl]-2-methylpropanoate and methanol in a similar manner as described in Preparation 10, but using MeOH instead of 1,1-bis(hydroxymethyl)cyclopropane. The diastereoisomers were separated by chiral SFC, the title compound was the faster eluting isomer. MS: [M+H]⁺=670.

Step 3: (2S,3S)-3-(4-Chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid

The title compound was prepared from prop-2-en-1-yl (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1 H-isoindol-2-yl]-2-methylpropanoate in an analogous fashion as described in Example 90, step 4. 1H NMR (400 MHz, DMSO-d6): 12.56-12.00 (1H, m), 7.71 (1H, s), 7.42 (1H, d), 7.02 (4H, d), 6.88 (3H, d), 4.91 (1H, s), 4.23 (1H, d), 3.99-3.85 (2H, m), 3.75 (1H, dd), 3.25-3.10 (5H, m), 2.02-1.90 (1H, m), 1.90-1.78 (2H, m), 1.67 (1H, d), 1.43-1.17 (6H, m), 0.95 (1H, d), 0.58 (3H, t). MS:[M+H]⁺=630.

(2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid (tris(hydroxymethyl)aminomethane salt)

Compound above was dissolved in EtOH and 1 mol. eq. of tris(hydroxymethyl)aminomethane was added. The solvent was removed in vacuo to give a colourless solid. ¹H NMR (500 MHz, DMSO-d6) δ 7.69 (s, 1H), 7.39 (d, J=10.7 Hz, 1H), 7.01 (broad s, 4H), 6.96-6.88 (m, 4H), 4.92 (broad s, 1H), 4.34-4.22 (m, 1H), 3.88 (dd, J=10.9, 4.2 Hz, 1H), 3.74 (dd, J=11.1, 4.2 Hz, 1H), 3.71-3.61 (m, 1H), 3.29 (s, 6H), 3.33-3.22 (m, 1H), 3.21-3.14 (m, 1H), 3.13 (s, 3H), 1.94 (tt, J=12.2, 3.6 Hz, 1H), 1.89-1.78 (m, 2H), 1.66 (d, J=12.8 Hz, 1H), 1.41-1.24 (m, 2H), 1.19 (d, J=6.8 Hz, 3H), 0.93 (d, J=13.2 Hz, 1H), 0.57 (t, J=7.3 Hz, 3H). MS:[M+H]⁺=630.

Preparation 2 of (2S,3S)-3-(4-chlorophenyl)-3-[(1R)-1-(4-chlorophenyl)-7-fluoro-5-[(15)-1-hydroxy-1-(oxan-4-yl)propyl]-1-methoxy-3-oxo-2,3-dihydro-1H-isoindol-2-yl]-2-methylpropanoic acid (“Compound 1”)

Stage 1: tert-butyl 3-bromo-5-fluorobenzoate

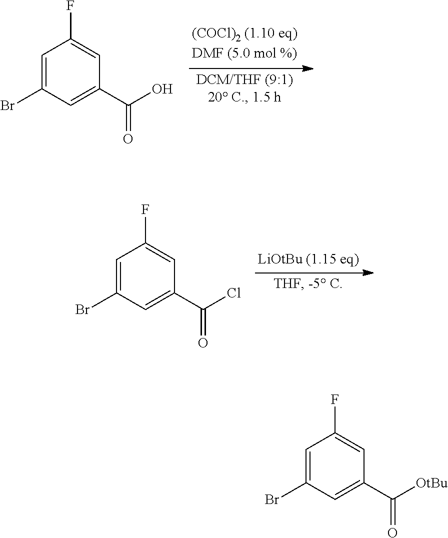 [SMILES: O=C(O)c1cc(F)cc(Br)c1 ; O=C(Cl)c1cc(F)cc(Br)c1 ; CC(C)(C)OC(=O)c1cc(F)cc(Br)c1]

3-bromo-5-fluorobenzoic acid (32.0 g, 1.0 equiv) was stirred in a mixture of DCM (288 mL, 9 vol) and THF (32 mL, 1 vol) until the majority of the solid dissolved. DMF (0.57 mL, 5 mol %) was added, and the flask placed in an ambient temperature water bath. Oxalyl chloride (13.7 mL, 1.10 equiv) was added over 1 h via syringe pump; 30 minute after the end of addition the reaction was complete by HPLC (sample quenched into MeOH to form methyl ester prior to analysis). The resulting thin slurry was aged overnight, concentrated to 100 mL volume, diluted with THF (160 mL, 5 vol) and again concentrated to 100 mL. The resulting thin slurry of acid chloride was diluted to 160 mL total volume with THF. A solution of LiOtBu in THF (20 wt %, 67.3 g, 77 mL, 1.15 equiv) was diluted with THF (243 mL), then this solution was cooled to an internal temperature of −9° C. with an ice/salt bath. To this was added the slurry containing acid chloride over 55 min, while the internal temperature remained below −3° C. The reaction was complete 15 min following the end of addition. The solution was aged overnight as it warmed to ambient temperature, diluted with heptane (320 mL, 10 vol), and washed with water (160 mL, 5 vol).

The aqueous layer was removed to the insoluble rag at the interface, then the organic layer was filtered through a pad of solka-floc. The pad was rinsed with heptane (10 mL), then the combined organic layer was washed 2× with water (2×80 mL, 2.5 vol). The resulting organic layer was distilled under reduced pressure to a 100 mL final volume, diluted with heptane (160 mL, 5 vol), and concentrated again to 100 mL total volume. The solution of tert-butyl 3-bromo-5-fluorobenzoate was used directly in the next step. NMR ¹H (400 MHz; CDCl₃): 7.89-7.88 (1H, m), 7.60-7.57 (1H, m), 7.40-7.37 (1H, m), 1.57 (9H, s).

Stage 2: 3-fluoro-5-[1-hydroxy-1-(oxan-4-yl)propyl]benzoic acid

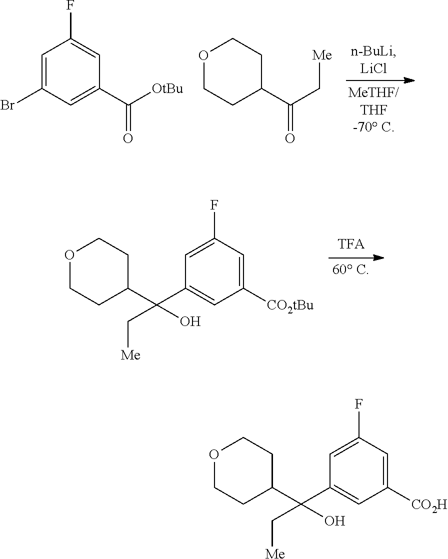

A solution of tert-butyl 3-bromo-5-fluorobenzoate (20.0 g, 1.0 equiv) and 1-(oxan-4-yl)propan-1-one (10.85 g, 1.05 equiv) in 2-MeTHF (200 mL, 10 vol) was treated with a 0.5 M solution of LiCl in THE (72.7 mL, 0.5 equiv) and cooled to −70° C. A solution of n-butyllithium in hexanes (2.2 M, 39.0 mL, 1.1 equiv) was added dropwise over 1 h; the reaction was complete upon end of addition. The mixture was warmed to −20° C., quenched with half-saturated aq. NH₄Cl solution (200 mL) and agitated for 10 minutes. The mixture was allowed to settle and the layers were separated. The organic phase was washed with water (50 mL, 2.5 vol). The solution assayed by HPLC for 20.6 g tert-butyl 3-fluoro-5-[1-hydroxy-1-(oxan-4-yl)propyl]benzoate (84% assay yield). LCMS (M−H)⁻; m/z=337.2. The organic solution was concentrated to ca 40 mL total volume (˜2 vol) by distillation under reduced pressure. The concentrated solution of tert-butyl 3-fluoro-5-[1-hydroxy-1-(oxan-4-yl)propyl]benzoate was treated with TFA (28.0 mL, 6.0 equiv) at 20° C. and the solution warmed to 60° C. and aged for 2 hours when HPLC analysis showed the reaction was 98% complete; the mixture was cooled to 20° C. then diluted with MTBE (40 mL, 2 vol) and heptane (80 mL, 4 vol). The solution was seeded with authentic tert-butyl 3-fluoro-5-[1-hydroxy-1-(oxan-4-yl)propyl]benzoate and aged for 30 min while a seed bed grew. The slurry was diluted over 1 h by addition of heptane (120 mL), filtered, and the cake washed with heptane (40 mL) to give the title compound as an off-white solid (14.89 g, 87% yield). NMR ¹H (400 MHz; DMSO): 13.23 (1H, s), 7.79 (1H, t), 7.50-7.47 (1H, m), 7.43-7.39 (1H, m), 4.79 (1H, s, broad), 3.79 (2H, ddd), 3.18 (2H, dt), 1.86-1.79 (3H, m), 1.64 (1 H, d), 1.36-1.09 (2H, m), 0.93 (1 H, d), 0.58 (3H, t); LCMS (M+H)+: m/z=283.1

Stage 3: 3-fluoro-5-[1-(oxan-4-yl)-1-[(trimethylsilyl)oxy]propyl]benzoic acid

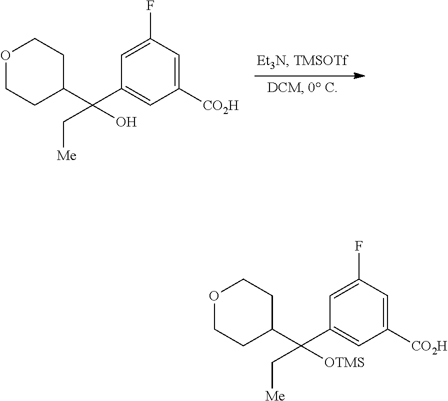

To a suspension of 3-fluoro-5-[1-hydroxy-1-(oxan-4-yl)propyl]benzoic acid (7.06 g, 1.0 equiv) in DCM (40 mL) at 0° C. was added Et₃N (7.08 g, 2.6 equiv) over 30 mins (maintaining a temperature below 5° C.). The resulting clear solution was treated with a solution of TMSOTf (13.34 g, 2.4 equiv) in DCM (40 mL) over 60 mins (maintaining a temperature below 5° C.). The reaction mixture was stirred for a further 1 h at 0° C. Water (88 mL) was added to the cold reaction mixture over 15 mins and the phases were separated. The organic phase was washed with 0.2M KHSO₄ solution (53 mL) and water (2×88 mL). The solution was dried over Na₂SO₄ and concentrated in vacuo. The crude product (an oil) was crystallized from DCM/heptane to afford the titled compound (8.24g, 93%) as an off-white solid. NMR ¹H (400 MHz; DMSO): 7.79 (1H, t), 7.65-8.62 (1H, m), 7.35-7.31 (1H, m), 3.98 (2H, ddd), 3.33 (2H, dtd), 2.04-1.84 (3H, m), 1.75 (1H, d), 1.37 (1h, qd), 1.26-1.20 (2H, m), 0.72 (3H, t), 0.25 (9H, s); LCMS (M+H)⁺: m/z=355.2

Stage 4: 2-(4-chlorobenzoyl)-3-fluoro-5-[1-hydroxy-1-(oxan-4-yl)propyl]benzoic acid

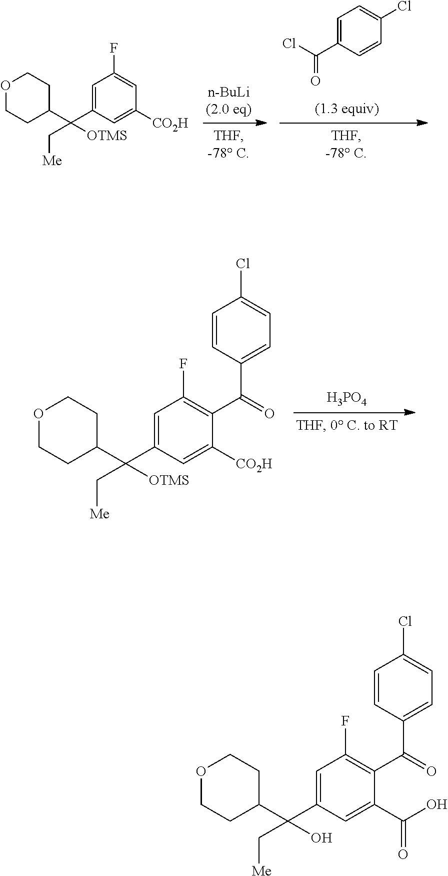

To THF (60 mL, 15 vol) at −70° C. internal temp was added n-BuLi (9.8 mL, 2.0 equiv, 2.3M solution in hexanes). A solution of 3-fluoro-5-[1-(oxan-4-yl)-1-[(trimethylsilyl)oxy]propyl]benzoic acid (4.0 g, 1.0 equiv) in THF (20.0 mL, 5 vol) was added dropwise over 60 min while the internal temperature was kept below −65° C. The resulting pale red solution was stirred for 30 min after the end of addition, and 4-chlorobenzoyl chloride (1.6 mL, 1.15 equiv) in THF (2 vol, 8.0 mL) was added over 10 min while the internal temperature was kept below −60° C.—the reaction is complete at the end of addition; this solution was warmed to 0° C. to give 2-(4-chlorobenzoyl)-3-fluoro-5-[1-(oxan-4-yl)-1-[(trimethylsilyl)oxy]propyl]benzoic acid as a solution in THF. LCMS (M+H)⁺: m/z=493.2 To the solution was added conc. H₃PO₄ (3.8 mL, 5.0 equiv) and the mixture was stirred at 50° C. for 18 h. The mixture was diluted with toluene (40 mL, 10 vol) and 4% aq. NaCl (20 mL, 5 vol). The phases were separated, and the top organic layer was washed with 4% aq. NaCl (20 mL) and water (10 mL).

The organic layer was concentrated to ˜1/3 volume, then diluted with toluene (60 mL, 15 vol). The solution was concentrated to −35 mL total volume (˜9 vol, 50° C. bath temp, 80 mbar pressure), over which time a while solid precipitated. The slurry was aged at 50° C. for 1 h, then cooled to ambient temperature and aged for 3 h. The slurry was filtered, and the cake washed with 2×8 mL (2×2 vol) toluene before being dried in a vacuum oven (50° C. oven temp) to a constant mass. The title compound was obtained as a white solid in 81% corr. yield (4.04 g, 95 wt %). LCMS (M+H)⁺: m/z=421.1

Stage 5: 2-(4-chlorobenzoyl)-3-fluoro-5-[(1S)-1-hydroxy-1-(oxan-4-yl)propyl]benzoic acid-bis[(1S)-1-phenylethyl]amine salt

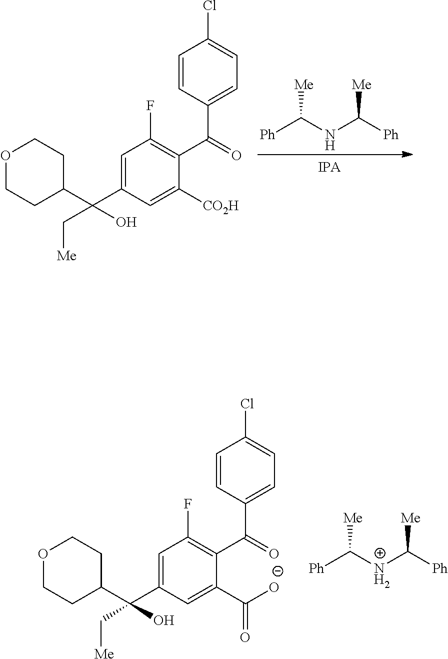

2-(4-chlorobenzoyl)-3-fluoro-5-[1-hydroxy-1-(oxan-4-yl)propyl]benzoic acid (racemate, 300g, 85 wt %, 255 g 6, 1.0 equiv) was dissolved in Isopropanol (4000 mL) by stirring at 55° C. for 10 min to give a homogeneous solution before cooling to 25° C. To the solution was added bis[(1S)-1-phenylethyl]amine (136.52 g; 1.0 equiv) in IPA (300 ml) over 2 minutes followed by an IPA rinse (200 mL). The solution was stirred at ambient temperature (22-23°) for 15 minutes and then seeded with authentic sample of the title compound (0.50 g); a solid crystallized readily and a slight endotherm (ca −0.4°) was observed. The suspension was stirred at an internal temperature of 19° C. for 20 h, filtered, and the cake washed with IPA (450 mL). The solid was dried under vacuum aspiration for 2 h then in a vacuum oven at 50° C. for 20 h to give a beige solid; 175.5 g (41% yield as IPA solvate)—by HPLC, the mixture is 95:5 e.r.

### Chiral HPLC Conditions:

Column: ChiralPak IC-3 3 pcolumn 4.6×150 mm
Column Temp: 270
Eluent: Heptane/IPA 80: 20 with 0.1% TFA
Flow rate: 1.0 mL/min @ 254 nm
Retention Desired (S) enantiomer; RT=4.60 mins. Undesired (R) enantiomer), RT=5.83 mins

The material (250 g, 1.0 equiv, 95:5 e.r.) was dissolved in IPA (4000 mL, 16 vol) by warming to 800 and stirring at this temperature for 15 min until a homogeneous solution formed. The solution was cooled over ˜1 h to 52° C., seeded with an authentic sample of the title compound (0.50 g) and the suspension was cooled to 20° C. over 4 hours and then stirred at ambient temperature this temperature overnight (total 24 h). The solid was isolated by filtration under vacuum, the filter cake washed with IPA (2×450 mL) and the filter cake sucked dry for 5 mins before further drying in a 50° C. vacuum oven. 2-(4-chlorobenzoyl)-3-fluoro-5-[(1S)-1-hydroxy-1-(oxan-4-yl)propyl]benzoic acid—bis[(1S)-1-phenylethyl]amine salt was obtained as a beige solid (219.2 g; 88% recovery); by HPLC the e.r. was 99.6: 0.4. NMR ¹H (400 MHz; DMSO): 7.84 (1H, d), 7.67 (1H, t), 7.65 (1H, t), 7.58 (1H, t), 7.56 (1H, t), 7.47 (1H, dd), 7.34-7.30 (4H, m), 7.28-7.20 (6H, m), 4.90 (1H, s), 3.90 (1H, dd), 3.80-3.72 (1H, m), 3.51-3.46 (1H, m), 3.30-3.15 (1H, m), 1.93-1.83 (3H, m), 1.68 (1H, d), 1.41-1.28 (1H, m), 1.26 (3H, s), 1.24 (3H, s), 1.04 (3H, s), 1.03 (3H, s), 0.65 (3H, t)

Stage 6: 2-(trimethylsilyl)ethyl (2S,3S)-3-amino-3-(4-chlorophenyl)-2-methylpropanoate-hydrochloride salt

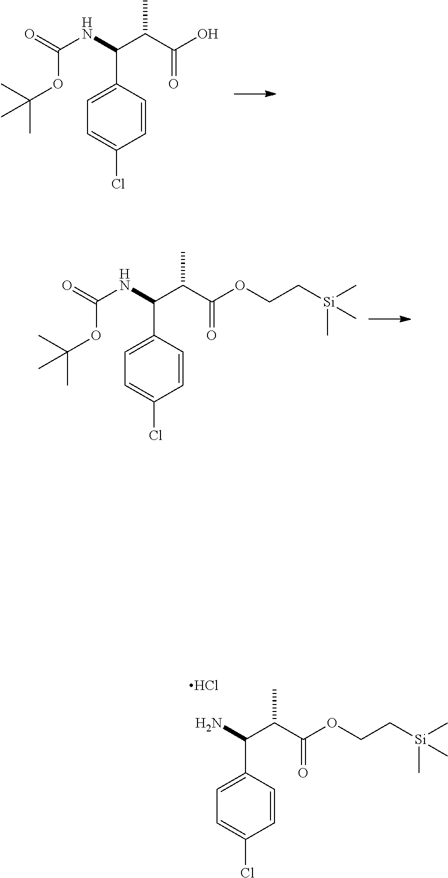

To a suspension of (2S,3S)-3-{[(tert-butoxy)carbonyl]amino}-3-(4-chlorophenyl)-2-methylpropanoic acid (109.82 g, 1.0 equiv), 2-trimethylsilylethanol (49.66g, 1.2 equiv) and DMAP (4.28 g, 0.05 mol %) in DCM (1100 mL, 10 vol) at −10° C. was added EDCHCl (100.65g, 1.5 equiv) in five equal portions over 75 mins (maintaining a temperature below 0° C.). The resulting clear solution was slowly allowed to warm to room temperature and stirred for 16h. 1N HCl solution (1000 mL) was slowly added to the reaction mixture over 15 mins and the phases were separated. The organic phase was washed with 5% NaHCO₃solution (500 mL) and water (2×500 mL). The organic phase was concentrated in vacuo to give a 2-(trimethylsilyl)ethyl (2S,3S)-3-{[(tert-butoxy)carbonyl]amino}-3-(4-chlorophenyl)-2-methylpropanoate, which was used directly in the next step. LCMS (M+H)⁺: m/z=414.2

The crude material (a waxy white solid) was redissolved into DCM (200 mL)/heptane (1500 mL) and a 4N solution of HCl in dioxane (350 mL, 4.0 equiv) was added dropwise to the heptane solution over 2 hrs. During this addition HCl salt begins to precipitate and the suspension gradually thickens as the reaction is aged at ambient temperature for 24 h. The suspension was diluted with MTBE (800 mL), filtered and the filter cake washed with MTBE (2×200 mL) to afford the title compound as a white flaky solid (108.22g, 88%) after drying in a vacuum oven at 50° C. to a constant weight. NMR ¹H (400 MHz; CDCl₃): 8.93 (3H, bs), 7.39-7.29 (4H, m), 4.3 (1H, bd), 4.06-3.92 (2H, m), 3.17-3.08 (1H, m), 1.32 (3H, d), 0.80-0.71 (2H, m), −0.02 (9H, s); LCMS (M+H)⁺: m/z=314.1 Stage 7: 2-(trimethylsilyl)ethyl (2S,3S)-3-(4-chlorophenyl)-3-[(1R)-1-(4-chlorophenyl)-7-fluoro-1-hydroxy-5-[(1S)-1-hydroxy-1-(oxan-4-yl)propyl]-3-oxo-2,3-dihydro-1H-isoindol-2-yl]-2-methylpropanoate

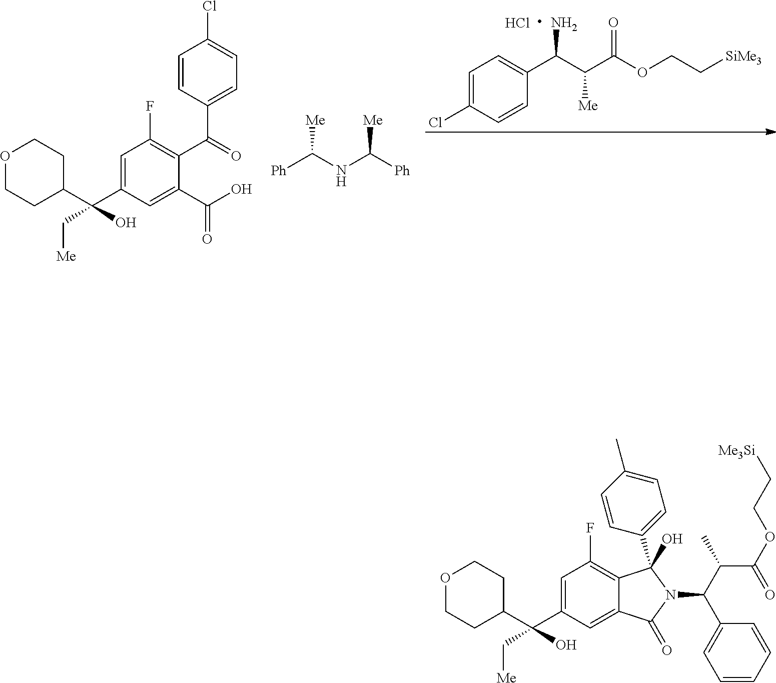

Dichloromethane (150 mL, 10 vol) was added to a mixture of 2-(4-chlorobenzoyl)-3-fluoro-5-[(1S)-1-hydroxy-1-(oxan-4-yl)propyl]benzoic acid—bis[(1S)-1-phenylethyl]amine salt (15.0 g, 1.0 equiv), 2-(trimethylsilyl)ethyl (2S,3S)-3-amino-3-(4-chlorophenyl)-2-methylpropanoate—hydrochloride salt (8.2 g, 1.1 equiv), EDC hydrochloride (4.7 g, 1.15 equiv), DMAP (260 mg, 0.1 equiv), and 2-hydroxypyridine-N-oxide (230 mg, 0.1 equiv). The mixture was stirred for 18h, then quenched by addition of aq. NaHCO₃(4.5 g, 2.5 equiv in 60 mL H₂O). The layers were separated and the DCM phase concentrated to 30 mL (2 vol). MTBE (150 mL, 10 vol) was added, and the organic layer washed sequentially with 2× aq. H₃PO₄ (3.5 mL, 2.5 equiv in 60 mL water), aq. NaHCO₃(4.5 g, 2.5 equiv in 60 mL H₂O), and water (60 mL).

The organic layer was concentrated to 60 mL (2 vol), diluted with MeOH (300 mL, 20 vol), and concentrated to 150 mL (10 vol). The MeOH solution was diluted with water (15 mL), seeded with authentic sample (15 mg, 0.1 wt %), and aged at ambient temperature for 30 min while a seed bed grew.

The slurry was diluted with water (45 mL) added over 2 h, aged for 1 h, then filtered. The cake was washed with 2.5/1 MeOH:H₂O (45 mL) and water (45 mL), and dried in a vacuum oven at 50° C. for 18 h to give the title compound as a white solid (13.5 g, 89% yield, d.r. >99:1 by 19F NMR). NMR ¹H (400 MHz; CDCl₃): 7.80 (1H, s), 7.15 (1H, d), 7.01-6.99 (4H, m), 6.97-6.92 (4H, m), 4.77 (1H, s), 4.36 (1H, d), 4.16-4.08 (1H, m), 3.94-3.90 (1H, m), 3.89-3.79 (2H, m), 3.47 (1H, d), 3.31 (1H, t), 3.08 (1H, t), 2.55 (1H, s), 1.91 (1H, sep), 1.86-1.77 (2H, m), 1.74-1.71 (1H, m), 1.41-1.22 (5H, m), 0.94 (1H, d), 0.68-0.54 (5H, m), 0.10 (9H, s), NMR ¹⁹F (376 MHz, CDCl₃) δ:-119.1 and LCMS (M+H)⁺: m/z=716.2

Stage 8: 2-(trimethylsilyl)ethyl (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoate

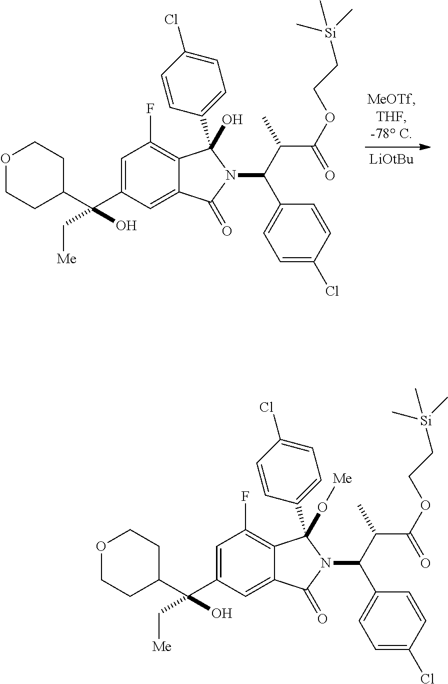

Solid 2-(trimethylsilyl)ethyl (2S,3S)-3-(4-chlorophenyl)-3-[(1R)-1-(4-chlorophenyl)-7-fluoro-1-hydroxy-5-[(1S)-1-hydroxy-1-(oxan-4-yl)propyl]-3-oxo-2,3-dihydro-1H-isoindol-2-yl]-2-methylpropanoate (2.5 g, 1.0 equiv) was dissolved in anhydrous THF (12.5 mL, 5 vol) in a 100 mL 3-neck flask at room temperature. The solution was cooled to −70° C. internal temperature, and MeOTf (methyl trifluoromethanesulfonate) (0.46 mL, 1.2 equiv) was added. The resulting clear solution was held at internal temperature of −70° C. LiOtBu (20 wt % in THF, 1.9 mL, 1.2 equiv) was added dropwise over a period of 1 h by syringe pump. The mixture was held at −70° C. for 18 h then warmed to −15° C. over 2 h at which point conversion was >98%. The reaction mixture was diluted with IPA (12.5 mL) and then water (12.5 mL). The solution was seeded with product 10, and stirred at ambient temperature for 30 minutes while a seed bed formed. Additional water (25 mL) was added slowly via a syringe pump over 1.5 h and the slurry aged for 1h at ambient temperature before being filtered. The cake was washed with 1:1 IPA/water (20 mL) and dried in a vacuum oven at 50° C. to give the title compound (2.4 g) (94% uncorrected yield, 100:0.5 d.r by 19F NMR). NMR ¹H (400 MHz; CDCl₃): 7.67 (1H, d), 7.28 (1H, dd), 6.93-6.88 (8H, m), 4.30-4.19 (m, 2H), 4.01 (dd, 1H), 3.92-3.77 (m, 3H), 3.40-3.26 (m, 2H), 3.22 (s, 3H), 1.97-1.84 (m, 4H), 1.72 (bs, 3H), 1.49-1.38 (m, 2H), 1.36 (d, 3H), 1.07 (bd, 1H), 0.69 (t, 3H), 0.61-0.52 (m, 2H), −0.08 (s, 9H); NMR ¹⁹F (376 MHz, CDCl₃) δ:-118.8 and LCMS (M+H)⁺: m/z=730.3

Stage 9: (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid

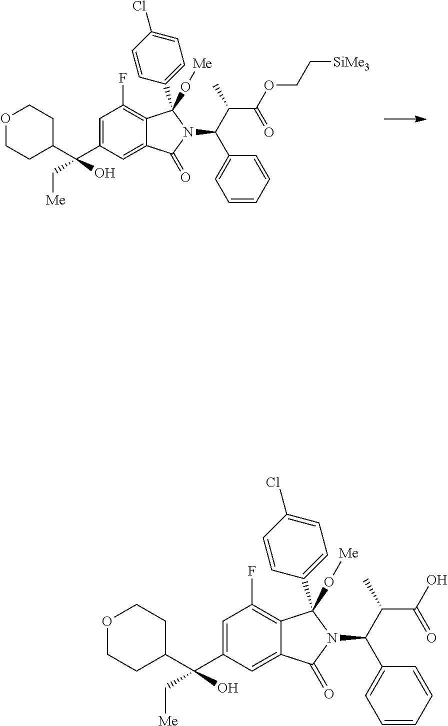 [SMILES: CC[C@@](O)(c1cc(F)c2c(c1)C(=O)N([C@H](c1ccccc1)[C@H](C)C(=O)OCC[Si](C)(C)C)[C@@]2(OC)c1ccc(Cl)cc1)C1CCOCC1 ; CC[C@@](O)(c1cc(F)c2c(c1)C(=O)N([C@H](c1ccccc1)[C@H](C)C(=O)O)[C@@]2(OC)c1ccc(Cl)cc1)C1CCOCC1]

2-(trimethylsilyl)ethyl (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoate (170.0 g, 1.0 equiv) and CsF (70.7 g, 2.0 equiv) were charged to a 5L fixed vessel and DMF (510 mL, 3 vol) was added at ambient temperature. The mixture was warmed to 60° C. and aged for 7 h at this temperature at which point the reaction was complete. The mixture was cooled to 20° C. and stirred overnight. The DMF was diluted with EtOAc (1700 ml-, 10 mL) and 1 M HCl (510 ml-, 3 vol). The layers were separated, and the organic layer was washed sequentially with 5% aq. LiCl (4×680 ml-, 4 vol) and water (2×680 ml-, 4 vol) before being concentrated. The resulting oil was concentrated twice from EtOAc (250 mL each time) to give the title compound as a pale yellow foam (141 g corr., 92 wt %., 96% yield). The solid was suspended in EtOAc (684 ml-, 4 vol) and heated to 70 00, held at this temperature for 1 h, then cooled to 20 00 over 2 h. Heptane (1370 ml-, 8 vol) was added over 70 min and the slurry aged overnight.

The solid was filtered, washed with EtOAc/heptane 1:2 (2×300 mL), and dried to a constant weight in a vacuum oven at 50 00 to give 133 g (86% yield).

The product was isolated in stable anhydrous crystalline form. This has been designated as free acid ‘Form F’ and is a stable crystalline polymorph.

The XRPD has peaks at the following resonances (Table 6):

TABLE 6

| Pos. | Height | FWHM | d-spacing | Rel. Int. |
|---|---|---|---|---|
| [°2Th.] | [cts] | [°2Th.] | [A] | [%] |
| 5.5324 | 119.60 | 0.4093 | 15.97459 | 3.19 |
| 8.0939 | 363.89 | 0.0768 | 10.92389 | 9.72 |
| 8.7670 | 654.55 | 0.0768 | 10.08658 | 17.48 |
| 10.0983 | 1123.93 | 0.0768 | 8.75963 | 30.02 |
| 11.0597 | 867.75 | 0.0768 | 8.00021 | 23.18 |
| 11.2706 | 1141.77 | 0.1023 | 7.85102 | 30.50 |
| 11.7674 | 80.69 | 0.1535 | 7.52066 | 2.16 |
| 13.5705 | 1039.22 | 0.1023 | 6.52514 | 27.76 |
| 14.2250 | 333.44 | 0.0768 | 6.22639 | 8.91 |
| 15.1034 | 2704.30 | 0.1279 | 5.86616 | 72.24 |
| 15.5082 | 3743.65 | 0.1279 | 5.71395 | 100.00 |
| 15.7699 | 2649.88 | 0.1023 | 5.61973 | 70.78 |
| 16.1290 | 684.97 | 0.1023 | 5.49539 | 18.30 |
| 16.5503 | 413.16 | 0.1023 | 5.35644 | 11.04 |
| 17.1682 | 1577.31 | 0.1279 | 5.16504 | 42.13 |
| 17.6278 | 246.51 | 0.1023 | 5.03138 | 6.58 |
| 18.1385 | 279.01 | 0.1023 | 4.89085 | 7.45 |
| 18.8833 | 723.33 | 0.1279 | 4.69961 | 19.32 |
| 19.1793 | 179.94 | 0.0768 | 4.62773 | 4.81 |
| 19.6727 | 256.37 | 0.1279 | 4.51276 | 6.85 |
| 20.3698 | 132.83 | 0.1023 | 4.35988 | 3.55 |
| 20.8132 | 2330.35 | 0.1279 | 4.26799 | 62.25 |
| 21.4724 | 496.23 | 0.1279 | 4.13844 | 13.26 |
| 22.2644 | 2823.66 | 0.2303 | 3.99297 | 75.43 |
| 23.2042 | 254.87 | 0.1023 | 3.83333 | 6.81 |
| 23.9443 | 465.26 | 0.1279 | 3.71650 | 12.43 |
| 24.5109 | 196.57 | 0.1023 | 3.63186 | 5.25 |
| 24.9654 | 105.69 | 0.1279 | 3.56676 | 2.82 |
| 25.4394 | 438.68 | 0.1023 | 3.50137 | 11.72 |
| 25.8370 | 351.04 | 0.1023 | 3.44839 | 9.38 |
| 26.5691 | 327.59 | 0.1535 | 3.35500 | 8.75 |
| 26.9367 | 637.86 | 0.1791 | 3.31004 | 17.04 |
| 27.3570 | 1012.15 | 0.1279 | 3.26015 | 27.04 |
| 28.2316 | 985.61 | 0.1535 | 3.16110 | 26.33 |
| 28.6372 | 1599.45 | 0.1535 | 3.11725 | 42.72 |
| 29.2407 | 315.65 | 0.1535 | 3.05427 | 8.43 |
| 29.9430 | 289.99 | 0.1791 | 2.98422 | 7.75 |
| 30.6433 | 463.31 | 0.1535 | 2.91759 | 12.38 |
| 31.2365 | 165.53 | 0.1279 | 2.86353 | 4.42 |
| 31.5627 | 201.49 | 0.1279 | 2.83467 | 5.38 |
| 32.1380 | 66.90 | 0.1535 | 2.78523 | 1.79 |
| 33.5238 | 129.51 | 0.2047 | 2.67320 | 3.46 |
| 33.7620 | 120.56 | 0.1535 | 2.65488 | 3.22 |
| 34.4905 | 171.78 | 0.1279 | 2.60045 | 4.59 |

Step 10a: (2S,3S)-3-(4-Chlorophenyl)-3-[(1R)-1-(4-chlorophenyl)-7-fluoro-5-[(15)-I-hydroxy-1-(oxan-4-yl)propyl]-1-methoxy-3-oxo-2,3-dihydro-1H-isoindol-2-yl]-2-methylpropanoic acid tris(hydroxymethyl)aminomethane salt

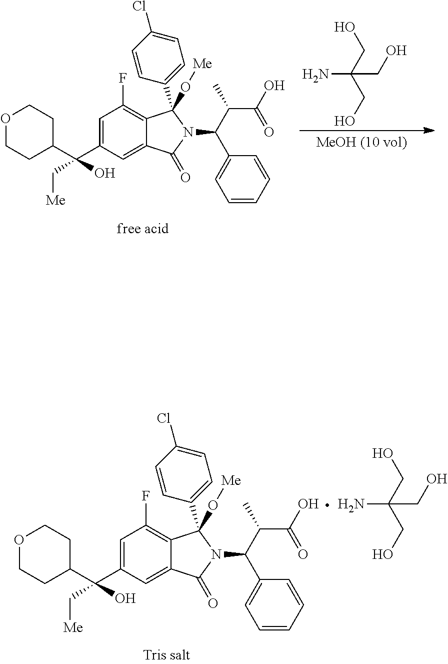 [SMILES: CC[C@@](O)(c1cc(F)c2c(c1)C(=O)N([C@H](c1ccccc1)[C@H](C)C(=O)O)[C@@]2(OC)c1ccc(Cl)cc1)C1CCOCC1 ; NC(CO)(CO)CO ; CC[C@@](O)(c1cc(F)c2c(c1)C(=O)N([C@H](c1ccccc1)[C@H](C)C(=O)O)[C@@]2(OC)c1ccc(Cl)cc1)C1CCOCC1 ; NC(CO)(CO)CO]

(2S,3S)-3-(4-chlorophenyl)-3-[(1R)-1-(4-chlorophenyl)-7-fluoro-5-[(1 S)-1-hydroxy-1-(oxan-4-yl)propyl]-1-methoxy-3-oxo-2,3-dihydro-1 H-isoindol-2-yl]-2-methylpropanoic acid (11 3.0g, 1.0 equiv) and tris(hydroxymethyl)aminomethane (21.95g, 1.01 equiv) were charged as solids to a 2L vessel. Methanol (1130 mL) was added with stirring under nitrogen to give a mobile suspension. The solids were dissolved by warming to 38-40° over 30 mins to give a clear solution. This was cooled to 20-22° and then concentrated under reduced pressure on a Buchi rotavapor to give a white foam. The foam was transferred to a crystallization dish and dried under vacuum (ca 20 mmHg) at 600 over a weekend (60h) to give the title compound as a crisp white foam (134.1 g; 99.5).

Other methods for the preparation of compound 1 can be found in international patent application no PCT/GB2018/050845 which was published as WO 2018/178691 on 04.10.2018.

### Biological Assays

Example 1—Compounds of Formula (I°)

### MDM2-p53 Interaction Using a 96-Well Plate Binding Assay (ELISA)

The ELISA assay was performed in streptavidin coated plates which were preincubated with 200 μl per well of 1 μg ml⁻¹ biotinylated IP3 peptide; The plates were ready to use for MDM2 binding after washing the plate with PBS.

Compounds and control solutions in DMSO aliquoted in 96-well plates were pre-incubated in a final 2.5-5% (v/v) DMSO concentration at room temperature (for example 20° C.) for 20 min with 190 μl aliquots of optimized concentrations of in vitro translated MDM2, before transfer of the MDM2-compound mixture to the b-IP3 streptavidin plates, and incubation at 4° C. for 90 min. After washing three times with PBS to remove unbound MDM2, each well was incubated at 20° C. for 1 hour with a TBS-Tween (50 mM Tris pH7.5; 150 mM NaCl; 0.05% Tween 20 nonionic detergent) buffered solution of primary mouse monoclonal anti-MDM2 antibody (Ab-5, Calbiochem, used at a 1/10000 or 1/200 dilution depending on the antibody stock solution used), then washed three times with TBS-Tween before incubation for 45 mins at 20° C. with a TBS-Tween buffered solution of a goat-anti-mouse horseradish peroxidase (HRP) conjugated secondary antibody (used at 1/20000 or 1/2000 depending on the antibody stock solution). The unbound secondary antibody was removed by washing three times with TBS-Tween. The bound HRP activity was measured by enhanced chemiluminesence (ECL™, Amersham Biosciences) using the oxidation of the diacylhydrazide substrate, luminol, to generate a quantifiable light signal. The percentage of MDM2 inhibition at a given concentration is calculated as the [1—(RLU detected in the compound treated sample—RLU negative DMSO control)_(RLU of DMSO positive and negative controls)]×100 or as the (RLU detected in the compound treated sample=RLU of DMSO controls)×100. The IC₅₀ was calculated using a plot of % MDM2 inhibition vs concentration and is the average of two or three independent experiments.

### Western Blot Analysis

SJSA cells were treated for 6 hours with 5, 10 and 20 μM of compounds in 0.5% DMSO. The cells together with 0.5% DMSO only controls were washed with ice-cold phosphate buffered saline (PBS) and protein extracts prepared by lysing the cells in SDS buffer (62.5 mM Tris pH 6.8; 2% sodium dodecyl sulphate(SDS); 10% glycerol) with sonication for 2×5 seconds (Soniprep 150ME) to break down high molecular weight DNA and reduce the viscosity of the samples. The protein concentration of the samples was estimated using the Pierce BCA assay system (Pierce, Rockford, IL) and 50 μg aliquots of protein analysed using standard SDS-polyacrylamide gel electrophoresis (SDS-PAGE) and Western immunoblotting procedures. β-mercaptoethanol (5%) and bromophenol blue (0.05%) were added and the samples, which were then boiled for 5 minutes, followed by brief centrifugation, before loading onto a pre-cast 4-20% gradient Tris-Glycine buffered SDS-polyacrylamide gel (Invitrogen). Molecular weight standards (SeeBlue™, Invitrogen) were included on every gel and electrophoresis was carried out in a Novex XL tank (Invitrogen) at 180 volts for 90 minutes. The separated proteins were transferred electrophoretically overnight from the gel onto a Hybond C nitrocellulose membrane (Amersham) using a BioRad electrophoresis tank and 25 mM Tris, 190 mM glycine and 20% methanol transfer buffer at 30 volts or two hours at 70 volts. Primary antibodies used for immunodetection of the transferred proteins were: mouse monoclonal NCL-p53DO-7 (Novocastra) at 1:1000; MDM2(Ab-1, clone IF2) (Oncogene) at 1:500; WAF1 (Ab-1, clone 4D10) (Oncogene) at 1:100; Actin (AC40) (Sigma) at 1:1000. The secondary antibody used was peroxidase conjugated, affinity purified, goat anti-mouse (Dako) at 1:1000. Protein detection and visualisation was performed by enhanced chemiluminescence (ECL™ Amersham) with light detection by exposure to blue-sensitive autoradiography film (Super RX, Fuji).

### Protocol A: SJSA-1 and SN40R2 Assays

The MDM2 amplified cell lines tested were an isogenic matched pair of p53 wild-type and mutated osteosarcoma (SJSA-1 and SN40R2, respectively). All cell cultures were grown in RPMI 1640 medium (Gibco, Paisley, UK) supplemented with 10% fetal calf serum and routinely tested and confirmed negative for mycoplasma infection. The growth of cells and its inhibition was measured using the sulphorhodamine B (SRB) method as previously outlined. 100 μl of 3×10⁴/ml and 2×10⁴/ml SJSA-1 and SN40R2 cells, respectively, were seeded into 96-well tissue culture plates and incubated at 37° C. in a 5% CO₂ humidified incubator for 24 hrs, after which the medium was replaced with 100 μl of test medium containing a range of MDM2-p53 antagonist concentrations and incubated for a further 72 hrs to allow cell growth before adding 25 μL of 50% trichloroacetic acid (TCA) to fix the cells for 1 h at 4° C. The TCA was washed off with distilled water and 100 μL of SRB dye (0.4% w/v in 1% acetic acid) (Sigma-Aldrich, Poole, Dorset) added to each well of the plate. Following incubation with the SRB dye at room temperature for 30 min, the plates were washed with 1% acetic acid and left to dry. The SRB stained protein, which is a measure of the number of cells in a well, was then resuspended in 100 μL of 10 mM Tris-HCl (pH 10.5) and the absorbance at A=570 nm measured in each well using a FluoStar Omega Plate reader. The G150 was calculated by non-linear regression analysis of the data using Prism v4.0 statistical software.

### Protocol B: SJSA-1 and SN40R2 Assays

The CellTiter-Glo® Luminescent Cell Viability Assay is a homogeneous method to determine the number of viable cells in culture based on quantitation of the ATP present, which signals the presence of metabolically active cells. Both SJSA-1 and SN40R2 were grown in RPMI 1640 (Life Technologies #61870) supplemented with 10% FBS (PAA #A15-204) and 10 U/ml penicillin/streptomycin. 2000 cells in 75 μl were seeded in each well of a 96 well plate and left at 37° C. in a 5% CO₂ humidified incubator for 24 hrs. A range of MDM2-p53 antagonist concentrations in DMSO was then added to the cells to a final DMSO concentration of 0.3%, and incubated for a further 72 hrs to allow cell growth. 100 μl of CTG reagent (Promega #G7573) was added to all wells and luminescence was measured on the topcount.

The EC₅₀ values were determined from a sigmoidal 4 parameter curve lit using XLfit in conjunction with Activity Base (IMBS; Guildford, Surrey, UK).

Anti-proliferative Activity Inhibition of cell growth is measured using the Alamar Blue assay (Nociari, M. M, Shalev, A., Benias, P., Russo, C. Journal of Immunological Methods 1998, 213, 157-167). The method is based on the ability of viable cells to reduce resazurin to its fluorescent product resorufin. For each proliferation assay cells are plated onto 96 well plates and allowed to recover for 16 hours prior to the addition of inhibitor compounds (in 0.1% DMSO v/v) for a further 72 hours. At the end of the incubation period 10% (v/v) Alamar Blue is added and incubated for a further 6 hours prior to determination of fluorescent product at 535 nM ex/590 nM em. The anti-proliferative activities of compounds of the invention can be determined by measuring the ability of the compounds to inhibit growth in cancer cell lines for example as available from DSMZ, ECACC or ATCC.

### Results: First Set of Examples Wherein Cyc is Phenyl

TABLE 7
biological data obtained from assays as described herein

| | | SJSA-1 | SJSA1 | SN40R2 | SN40R2 |
|---|---|---|---|---|---|
| Patent | MDM2 | IC50 (μM) | IC50 (μM) | IC50 (μM) | IC50 (μM) |
| Example | IC50 (μM) | (Protocol A) | (Protocol B) | (Protocol A) | (Protocol B) |
| 1 | 0.012 | 0.49 | 0.55 | 18 | 10% at 10 |
| 2 | 0.0046 | 0.33 | 0.46 | 17 | 22% at 10 |
| 3 | 0.093 | | | | |
| 4 | 0.043 | | | | |
| 5 | 0.14 | | | | |
| 6 | 0.12 | | | | |
| 7 | 0.0066 | | | | |
| 8 | 0.0047 | 0.33 | | 18 | |
| 9 | 0.011 | | | | |
| 10 | 0.0037 | 0.14 | | 7.5 | |
| 11 | 0.033 | | | | |
| 12 | 0.0058 | 0.51 | 0.69 | 5.9 | |
| 13 | 0.12 | 4.6 | | 5.9 | |
| 14 | 0.0050 | 0.83 | 0.49 | 10% at 30 | 9% at 10 |
| 15 | 0.019 | | | | |
| 16 | 0.14 | 2.1 | | 13 | |
| 17 | 0.063 | 0.95 | | 8.1 | |
| 18 | 0.045 | 0.80 | | 18 | |
| 19 | 0.022 | 0.62 | 2.0 | 13 | 13 |
| 20 | 0.011 | 0.33 | | 11 | |
| 21 | 0.0078 | 0.23 | 0.39 | 15 | 51% at 10 |
| 22 | 0.0052 | 0.21 | | 18 | |
| 24 | 0.0075 | 0.37 | 0.63 | 21 | 19% at 10 |
| 25 | 0.0072 | 0.71 | 1.1 | 25 | 14% at 10 |
| 26 | 0.032 | 1.7 | | 17 | |
| 27 | 0.065 | 2.1 | | 29% at 30 | |
| 28 | 0.026 | 0.93 | | 26% at 30 | |
| 29 | 0.11 | 1.4 | | 17 | |
| 30 | 0.086 | 2.4 | | 27 | |
| 31 | 0.038 | 1.2 | | 18 | |
| 32 | | 0.87 | | 15 | |
| 33 | 0.0019 | 9.1 | | 7% at 30 | |
| 34 | 0.0046 | 0.093 | | 9.9 | |
| 35 | 0.0018 | 0.16 | 0.69 | 23 | 13 |
| 36 | 0.0019 | 0.078 | | 17 | |
| 37 | 0.041 | 1.2 | | 13 | |
| 38 | 0.026 | 0.67 | | 17 | |
| 39 | 0.068 | 2.0 | | 18 | |
| 40 | 0.063 | 1.5 | | 17 | |
| 41 | 0.0016 | 0.14 | | 13 | |
| 42 | 34%@0.00030 | 0.011 | 0.03 | 12 | 10 |
| 43 | 47%@0.0010 | 0.57 | | 12 | |
| 44 | 0.0058 | 0.83 | | 6.8 | |
| 45 | 0.23 | | | | |
| 46 | 10.78 | | | | |
| 47 | 0.43 | | | | |
| 48 | 0.0073 | 0.46 | 0.97 | 17 | 24% at 10 |
| 49 | 0.082 | 1.6 | | 18 | |
| 50 | 0.00080 | 0.079 | 0.032 | 17 | 22% at 10 |
| 51 | 0.13 | | | | |
| 52 | 0.15 | 1.8 | | | |
| 53 | 0.12 | 1.9 | | | |
| 54 | 0.15 | | | | |
| 55 | | 1.7 | | 11 | |
| 56 | 0.12 | | | | |
| 57 | 0.061 | 1.4 | | 16 | |
| 58 | 0.018 | 0.59 | | 15 | |
| 59 | 0.0041 | 0.25 | | 19 | |
| 60 | 0.014 | | | | |
| 61 | 0.016 | 0.69 | | 44% at 30 | |
| 62 | 0.0023 | 0.055 | | 55% at 30 | |
| 63 | 71%@0.0010 | | 0.096 | | 19% at 10 |
| 64 | 0.0021 | | | | |
| 65 | 0.0018 | | 0.26 | | |
| 66 | 0.0030 | | | | |
| 67 | 60%@0.0010 | | 0.53 | | 9.4 |
| 68 | 0.0070 | | 1.8 | | 13 |
| 69 | 0.00070 | 0.081 | 0.16 | 15 | 6.6 |
| 70 | 0.0057 | | 0.68 | | 4.9 |
| 71 | 0.0020 | 0.66 | 0.7 | 44 | 3% at 10 |
| 72 | 0.0015 | 0.14 | 0.17 | 16 | 45% at 10 |
| 73 | 0.012 | | 3.6 | | 39% at 30 |
| 74 | 0.00050 | 0.28 | 1.0 | 28 | 13 |
| 75 | 73%@0.0010 | 0.12 | 0.35 | 22 | 12 |
| 76 | 0.0095 | | 1.0 | | 13 |
| 77 | 61%@0.00030 | | 0.46 | | 3.7 |
| 78 | 0.0046 | 0.41 | 1.4 | 5.9 | 4.2 |
| 79 | 0.0022 | | 8.1 | | 10% at 30 |
| 80 | 73%@0.0010 | | 0.83 | | 13 |
| 81 | 0.0026 | | | | |
| 82 | 0.0025 | 0.21 | | 51% at 30 | |
| 83 | 0.0010 | | 0.53 | | 11 |
| 84 | 39%@0.00030 | 0.065 | | 18 | |
| 85 | 0.00049 | | 0.049 | | 13 |
| 86 | 56%@0.10 | | | | |
| 87 | 82%@0.0030 | | 37% at 10 | | 1% at 10 |
| 88 | 0.00079 | 0.15 | 0.23 | 39 | 11% at 10 |
| 89 | 0.012 | | 3.6 | | 3% at 10 |
| 90 | 39%@0.030 | | 97% at 10 | | 6% at 10 |
| 91 | 78%@0.0010 | 0.080 | 0.059 | 26 | 13% at 10 |
| 92 | 76%@0.0010 | 0.080 | 0.084 | 36 | 12% at 10 |
| 93 | 49%@0.030 | | 3.3 | | 12% at 10 |
| 94 | 64%@0.10 | | | | |
| 95 | 87%@0.0010 | 0.036 | 0.022 | 16 | 21% at 10 |
| 96 | 0.00064 | 0.071 | 0.075 | 19 | 17% at 10 |
| 97 | 45%@0.10 | | | | |
| 98 | 0.0008 | 0.081 | 0.13 | 33 | 11% at 10 |
| 99 | 0.012 | | 3.2 | | 4% at 10 |
| 100 | 0.0063 | | 1.7 | | 7% at 10 |
| 101 | 55%@0.00030 | 0.026 | 0.026 | 18 | 11% at 3 |
| 102 | 0.017 | | 1.4 | | 26% at 10 |
| 103 | 55%@0.030 | | 0.8 | | 18% at 10 |
| 104 | 70%@0.10 | | 42% at 10 | | 5% at 10 |
| 105 | 92%@0.0010 | 0.022 | 0.05 | 33 | 20% at 10 |
| 106 | 57%@0.030 | | 3.2 | | 8% at 10 |
| 107 | 78%@0.0010 | 0.021 | 0.038 | 24 | 18% at 10 |
| 108 | 0.0061 | | 27% at 10 | | 29% at 10 |
| 109 | 92%@0.0010 | 0.012 | 0.02 | 26 | 75% at 10 |
| 110 | 76%@0.0010 | 0.026 | 0.013 | 17 | 30% at 10 |
| 111 | 61%@0.0010 | 0.024 | 0.037 | 9 | 51% at 10 |
| 113 | 57%@0.0010 | | 0.02 | | 10% at 10 |
| 114 | 81%@0.0010 | 0.029 | 0.063 | 20 | 15% at 10 |
| 115 | 73%@0.0010 | | 0.22 | | 2% at 10 |
| 116 | 88%@0.0010 | 0.08 | 0.14 | 44 | 12% at 10 |
| 117 | 45% at 0.03 | | 30% at 10 | | 19% at 10 |
| 118 | 87%@0.0010 | | 0.36 | | 8% at 10 |
| 119 | 54%@0.0010 | 0.06 | 0.2 | 39 | 7% at 10 |
| 120 | 76%@0.0010 | 0.063 | 0.095 | 40% at 50 | 4% at 10 |
| 121 | 93%@0.0010 | 0.015 | 0.015 | 26 | 18% at 10 |
| 122 | 88%@0.0010 | | 0.024 | | 20% at 10 |
| 123 | 42%@0.030 | | 107% at 10 | | 16% at 10 |
| 124 | 80%@0.0010 | 0.023 | 0.027 | 23 | 55% at 10 |
| 125 | 18%@0.10 | | | | |
| 126 | 0.0019 | 0.6 | 0.61 | 30 | 7% at 10 |
| 127 | 0.0045 | | 1.4 | | 14% at 10 |
| 128 | 39%@0.10 | | | | |
| 129 | 90%@0.0010 | 0.047 | 0.048 | 6 | 112% at 10 |
| 130 | 98%@0.0010 | | 0.23 | | 87% at 10 |
| 131 | 89%@0.0010 | 0.044 | 0.093 | 22 | −3% at 10 |
| 132 | 43%@0.030 | | 0.75 | | 34% at 10 |
| 133 | 6%@0.10 | | 37% at 10 | | 89% at 10 |
| 134 | 0.0011 | | 0.78 | | 2% at 10 |
| 135 | 40%@0.10 | | 20% at 10 | | 7% at 10 |
| 136 | 0.0013 | | 0.056 | | 86% at 10 |
| 137 | 0.00057 | | 0.15 | | 12% at 10 |

Where more than one data point has been obtained, the table above shows an average (e.g. geometric or arithmetic mean) of these data points.

It is of course to be understood that the invention is not intended to be restricted to the details of the above embodiments which are described by way of example only.

### Results: Second Set of Examples Wherein Cyc is Het

### Results

TABLE 8
biological data obtained from assays as described herein

| | | SJSA-1 | SJSA1 | SN40R2 | SN40R2 |
|---|---|---|---|---|---|
| | MDM2 | IC50 (μM) | IC50 (μM) | IC50 (μM) | IC50 (μM) |
| E.g. | IC50 (μM) | (Protocol A) | (Protocol B) | (Protocol A) | (Protocol B) |
| 7 | 0.0036 | 0.11 | 0.34 | 26 | 8% at 10 |
| 8 | 0.053 | 2.0 | 3.8 | 34% at 30 | 11% at 10 |
| 6 | 0.023 | 1.7 | 2.5 | 13% at 30 | 4% at 10 |
| 9 | 0.015 | 0.82 | | 32% at 30 | |
| 93 | 0.030 | 1.0 | | 40% at 30 | |
| 31 | 0.017 | 0.55 | 0.76 | 20% at 30 | 0% at 10 |
| 1 | 0.0020 | 0.088 | 0.2 | 24 | 14% at 10 |
| 94 | 0.10 | 2.4 | | 15% at 30 | |
| 2 | 0.026 | 1.7 | 3.4 | 38% at 30 | 10% at 10 |
| 47 | 0.12 | 1.6 | | 24 | |
| 46 | 0.016 | 0.59 | 0.76 | 42% at 30 | 13% at 10 |
| 10 | 0.016 | 0.32 | | 6% at 30 | |
| 44 | 0.015 | 0.28 | | 24 | |
| 61 | 0.11 | 0.86 | | 26% at 30 | |
| 62 | 0.041 | 0.75 | | 29% at 30 | |
| 5 | 0.0038 | 0.20 | 0.28 | 20% at 30 | 7% at 10 |
| 38 | 0.0094 | 0.64 | | 15% at 30 | |
| 39 | 0.0044 | 0.17 | | 3% at 30 | |
| 45 | 0.0084 | 0.23 | | 27 | |
| 63 | 0.032 | 0.57 | | 27 | |
| 11 | 0.0087 | 0.23 | 0.46 | 15% at 30 | |
| 32 | 0.0012 | 0.089 | 0.14 | 27 | 9% at 10 |
| 12 | 0.046 | 1.5 | | 0% at 30 | |
| 33 | 0.010 | 0.61 | | 31% at 30 | |
| 13 | 0.0077 | 0.52 | 0.73 | 24 | 21% at 10 |
| 48 | 0.018 | 0.60 | 0.56 | 4% at 30 | −3% at 10 |
| 64 | 0.040 | 0.75 | | 3% at 30 | |
| 95 | 0.060 | 2.0 | | 19 | |
| 34 | 0.085 | 0.97 | 2.2 | 14% at 30 | 20% at 30 |
| 16 | 0.030 | 0.27 | | 24% at 30 | |
| 17 | 0.0038 | 0.10 | 0.21 | 29% at 30 | |
| 3 | 0.12 | 0.96 | | 7% at 30 | 9% at 30 |
| 14 | 0.029 | 0.55 | | 13% at 30 | |
| 15 | 0.0068 | 0.21 | | 22% at 30 | |
| 54 | 0.10 | 2.2 | | 25 | |
| 59 | 0.034 | 0.70 | | 34% at 30 | |
| 4 | 0.010 | 0.23 | 0.24 | 24% at 30 | 9% at 10 |
| 49 | 0.040 | 0.38 | | 18% at 30 | |
| 60 | 0.020 | 0.55 | | 27 | |
| 18 | 0.020 | 0.51 | | 20% at 30 | |
| 19 | 0.0027 | 0.069 | | 20% at 30 | |
| 65 | 0.021 | 0.41 | | 24 | |
| 35 | 0.010 | 0.45 | 0.8 | 8% at 30 | 6% at 10 |
| 42 | 0.010 | 0.45 | 0.60 | 29% at 30 | 14% at 10 |
| 43 | 0.026 | 0.49 | 0.48 | 16% at 30 | 13% at 10 |
| 40 | 0.046 | 0.81 | 1.0 | 2% at 30 | 9% at 10 |
| 41 | 0.013 | 0.32 | 0.47 | 15% at 30 | 8% at 10 |
| 37 | 0.035 | 0.26 | | 25 | 12 |
| 50 | 0.0088 | 0.23 | | 24 | |
| 96 | 0.14 | | | | |
| 51 | 0.69 | | | | |
| 22 | 0.0018 | 0.16 | 0.059 | 19 | 13 |
| 23 | 0.0074 | 0.55 | | 17 | |
| 36 | 0.0051 | 0.21 | 0.18 | 13% at 30 | |
| 74 | 0.015 | 0.31 | | 24 | |
| 28 | 0.014 | 0.19 | | 44% at 30 | |
| 55 | 0.49 | | | | |
| 56 | 0.021 | 0.33 | | 24 | |
| 30 | 0.017 | 0.30 | | 0% at 30 | |
| 24 | 0.0077 | 0.24 | | 42% at 30 | |
| 25 | 0.0018 | 0.054 | 0.090 | 26 | 13% at 10 |
| 26 | 0.027 | 0.58 | | 28 | |
| 27 | 42%@0.0030 | 0.24 | 0.71 | 23 | 11% at 10 |
| 52 | 0.031 | 0.25 | | 15% at 30 | |
| 87 | 0.031 | 0.71 | | 19 | |
| 77 | 0.076 | 2.2 | | 48% at 30 | |
| 78 | 0.026 | 0.77 | | 26 | |
| 53 | 0.12 | | | | |
| 29 | 0.012 | 0.39 | 0.52 | 16% at 30 | 25% at 30 |
| 20 | 0.026 | 1.6 | | 4% at 30 | |
| 21 | 0.0052 | 0.27 | | 10% at 30 | |
| 119 | 0.018 | 0.53 | | 47% at 30 | |
| 118 | 0.034 | 0.67 | | 39% at 30 | |
| 79 | 0.0046 | 0.12 | 0.38 | 25 | 2% at 10 |
| 97 | 0.013 | 0.37 | | 24 | |
| 98 | 0.018 | 0.43 | | 23 | |
| 73 | 0.082 | 1.8 | | 0% at 30 | |
| 75 | 0.0045 | 0.14 | 0.47 | 29 | 15% at 10 |
| 70 | 0.0032 | 0.21 | | 48% at 30 | |
| 76 | 0.0065 | 0.54 | | 20 | |
| 71 | 0.082 | 5.3 | | 33% at 30 | |
| 124 | 0.093 | 1.9 | | 2% at 30 | |
| 122 | 0.033 | 0.68 | | 9.9 | |
| 123 | 0.0098 | 0.23 | | 21 | |
| 120 | 0.085 | 1.9 | | 39% at 30 | |
| 121 | 0.023 | 0.55 | | 28% at 30 | |
| 104 | 52%@1.0 | | | | |
| 105 | 0.015 | 0.40 | | 9.8 | |
| 67 | 0.029 | 0.71 | | 36% at 30 | |
| 85 | 0.0017 | 0.10 | | 34% at 30 | |
| 86 | 0.15 | | | | |
| 110 | 55%@1.0 | | | | |
| 111 | 0.059 | 2.0 | | 29 | |
| 106 | 0.52 | 5.2 | | 24 | |
| 107 | 0.016 | 0.38 | | 29 | |
| 108 | 0.79 | 8.2 | | 16 | |
| 109 | 0.11 | 1.7 | 3.5 | 18 | 24% at 10 |
| 114 | 0.12 | 2.0 | | 10% at 30 | |
| 115 | 81%@0.10 | 5.2 | | 13% at 30 | |
| 82 | 0.027 | 0.62 | | 32% at 30 | |
| 83 | 41%@0.0010 | 0.038 | | 36% at 30 | |
| 66 | 0.0099 | 0.51 | 0.73 | 18 | 13 |
| 89 | 0.011 | 0.45 | | 23% at 30 | |
| 90 | 0.00064 | 0.046 | | 24% at 30 | |
| 112 | 0.18 | 5.4 | | 15% at 30 | |
| 113 | 0.0069 | 0.50 | | 13% at 30 | |
| 84 | 0.022 | 1.3 | | 23% at 30 | |
| 99 | 35%@1.0 | | | | |
| 100 | 0.016 | 0.47 | | 38% at 20 | |
| 101 | 0.013 | 0.28 | | 25% at 30 | |
| 72 | 0.0086 | 0.36 | | 35% at 30 | |
| 81 | 0.11 | | | | |
| 91 | 41%@0.15 | | | | |
| 92 | 0.0059 | 0.24 | | 21 | |
| 102 | 37%@0.30 | | | | |
| 103 | 0.022 | 0.43 | | 9.5 | |
| 68 | 0.0016 | 3.2 | | 19% at 30 | |
| 69 | 0.0081 | 7.6 | | 32% at 30 | |
| 57 | 44%@0.30 | | | | |
| 58 | 0.0053 | 0.23 | | 24 | |
| 88 | 0.028 | 1.5 | | 12% at 30 | |
| 125 | 0.10 | | | | |
| 126 | 0.015 | | | | |
| 116 | 48%@0.10 | | | | |
| 117 | 0.0078 | 0.26 | | 26 | |
| 419 | 0.018 | 0.70 | | 21% at 30 | |
| 318 | 11%@0.025 | | | | |
| 319 | 0.0076 | 0.16 | | 14 | |
| 327 | 42%@0.30 | 9.3 | | 9% at 30 | |
| 328 | 42%@0.10 | 2.3 | | 25 | |
| 329 | 61%@0.30 | 3.0 | | 8% at 30 | |
| 330 | 36%@0.30 | 7.1 | | 18% at 30 | |
| 381 | 33%@0.30 | 46% at 10 | | 17 | |
| 382 | 0.036 | 0.82 | | 18 | |
| 383 | 31%@0.30 | 6.6 | | 18 | |
| 384 | 39%@0.030 | 0.36 | | 16 | |
| 157 | 39%@0.30 | 6.4 | | 30% at 30 | |
| 158 | 57%@0.10 | 1.2 | | 30% at 30 | |
| 242 | 35%@0.30 | 6.3 | | 6.2 | |
| 243 | 0.018 | 0.63 | | 5.8 | |
| 245 | 51%@0.30 | 7.4 | | 16 | |
| 241 | 0.012 | 0.58 | | 8.9 | |
| 239 | 0.015 | | | | |
| 248 | 37%@0.30 | | | | |
| 247 | 0.022 | 0.76 | | 18 | |
| 238 | 41%@0.30 | 52% at 10 | | 19 | |
| 246 | 37%@0.30 | 7.6 | | 18 | |
| 237 | 0.013 | 0.55 | | 16 | |
| 244 | 36%@0.30 | 40% at 10 | | 18 | |
| 240 | 0.032 | 0.91 | | 17 | |
| 159 | 0.031 | 1.2 | | 24 | |
| 160 | 43%@0.30 | | | | |
| 167 | 0.011 | 0.64 | | 19% at 30 | |
| 253 | 53%@1.0 | | | | |
| 253a | 0.035 | 0.85 | | 29 | |
| 252 | 41%@0.30 | | | | |
| 249 | 0.013 | 0.51 | | 34% at 30 | |
| 251 | 61%@1.0 | | | | |
| 250 | 0.0072 | 1.6 | | 18 | |
| 320 | 0.0060 | 2.7 | | 24 | |
| 321 | 0.0027 | 1.4 | | 22 | |
| 258 | 50%@1.0 | | | | |
| 257 | 0.12 | 1.9 | | 29% at 30 | |
| 256 | 59%@1.0 | | | | |
| 255 | 0.0032 | 0.51 | | 16 | 7.1 |
| 254 | 45%@0.30 | | | | |
| 259 | 0.0097 | 0.96 | | 5.5 | |
| 127 | 45%@0.0033 | 0.38 | | 20% at 30 | |
| 134 | 47%@0.30 | | | | |
| 135 | 0.049 | 1.6 | | 38% at 30 | |
| 323 | 46%@0.64 | | | | |
| 324 | 0.028 | 0.57 | | 23 | |
| 260 | 39%@1.0 | | | | |
| 261 | 0.048 | 0.65 | | 19 | |
| 169 | 0.0046 | 0.26 | | 43 | |
| 170 | 4%@0.10 | | | | |
| 275 | 43%@0.30 | | | | |
| 262 | 0.033 | 0.47 | | 22 | |
| 233 | 37%@1.0 | | | | |
| 234 | 0.015 | 0.38 | | 21 | |
| 128 | 0.11 | 2.4 | | 23 | |
| 129 | 0.0047 | 0.24 | | 21 | |
| 263 | 45%@1.0 | | | | |
| 264 | 0.043 | 0.67 | | 8.0 | |
| 235 | 40%@1.0 | | | | |
| 236 | 0.041 | 0.72 | | 28 | |
| 316 | 0.0072 | 0.86 | | 17% at 30 | |
| 317 | 0.0016 | 0.19 | | 36% at 50 | |
| 377 | 0.11 | | | | |
| 378 | 52%@0.30 | | | | |
| 376 | 0.0040 | | 0.32 | | 18% at 10 |
| 302 | 63%@0.10 | 5.3 | | 9% at 30 | |
| 303 | 0.0016 | 0.55 | | 13% at 30 | |
| 268 | 45%@1.0 | | | | |
| 266 | 0.015 | | | | |
| 267 | 42%@1.0 | | | | |
| 265 | 0.044 | | | | |
| 289 | 0.012 | | | | |
| 291 | 45%@0.0010 | | | | |
| 292 | 0.021 | | | | |
| 172 | 0.13 | | | | |
| 171 | 0.14 | | | | |
| 270 | 33%@1.0 | | | | |
| 269 | 0.16 | | | | |
| 290 | 0.0025 | | | | |
| 168 | 0.039 | | | | |
| 175 | 0.0061 | | | | |
| 176 | 0.0010 | | | | |
| 379 | 52%@1.0 | | | | |
| 271 | 0.014 | | | | |
| 380 | 59%@1.0 | | | | |
| 274 | 0.0097 | | | | |
| 309 | 0.0023 | | | | |
| 273 | 47%@1.0 | | | | |
| 272 | 0.0088 | | | | |
| 177 | 47%@0.030 | | | | |
| 178 | 0.00079 | 0.16 | 0.10 | 43% at 50 | 18% at 30 |
| 145 | 0.21 | | | | |
| 147 | 44%@0.10 | | | | |
| 310 | 53%@0.0010 | | | | |
| 173 | 0.025 | | | | |
| 146 | 0.081 | | | | |
| 148 | 0.035 | | | | |
| 153 | 0.015 | | | | |
| 154 | 0.014 | | | | |
| 287 | 0.0031 | | | | |
| 151 | 32%@0.30 | | | | |
| 152 | 0.30 | | | | |
| 149 | 53%@1.0 | | | | |
| 150 | 49%@0.10 | | | | |
| 345 | 0.0037 | | | | |
| 346 | 46%@0.00030 | 0.031 | 0.012 | 20 | 12 |
| 288 | 0.046 | | | | |
| 281 | 58%@0.10 | | | | |
| 280 | 0.0063 | | | | |
| 131 | 0.092 | | | | |
| 130 | 0.0057 | | 0.17 | | 8% at 10 |
| 285 | 41%@0.10 | | | | |
| 284 | 0.0025 | | 0.017 | | 12% at 30 |
| 132 | 51%@0.30 | | | | |
| 133 | 33%@1.0 | | | | |
| 305 | 0.0015 | | 1.9 | | 32% at 30 |
| 282 | 0.0021 | | 0.061 | | 5% at 30 |
| 283 | 57%@0.10 | | | | |
| 304 | 0.0022 | | 0.36 | | 9.0 |
| 161 | 0.016 | | 4.3 | | 28% at 30 |
| 162 | 0.0022 | | 0.22 | | 13 |
| 308 | 0.044 | | | | |
| 136 | 0.037 | | 1.4 | | 13 |
| 137 | 0.0016 | 0.20 | 0.27 | 21 | 11 |
| 306 | 55%@0.030 | | 6.6 | | 13 |
| 199 | 49%@0.030 | | 0.83 | | 16% at 30 |
| 200 | 0.00071 | 0.066 | 0.099 | 40 | 21% at 30 |
| 189 | 0.013 | | 1.0 | | 47% at 30 |
| 190 | 0.037 | | 5.0 | | 19% at 30 |
| 205 | 0.0012 | 0.18 | 0.22 | 34 | 12 |
| 206 | 0.0015 | | 0.45 | | 13 |
| 207 | 56%@0.10 | | 2.2 | | 13 |
| 307 | 62%@0.030 | | 1.9 | | 11 |
| 315 | 0.46 | | 12 | | 13 |
| 163 | 0.042 | | 3.1 | | 13 |
| 164 | 0.034 | | 2.2 | | 28% at 30 |
| 165 | 0.017 | | 1.4 | | 13 |
| 166 | 48%@0.010 | | 3.8 | | 33% at 30 |
| 208 | 0.027 | | 2.3 | | 24% at 30 |
| 298 | 0.00066 | 0.22 | 0.55 | 7.6 | 8.9 |
| 299 | 0.0096 | | 2.0 | | 8.0 |
| 191 | 0.048 | | 2.5 | | 11 |
| 192 | 0.0021 | | 1.4 | | 11 |
| 420 | 68%@0.0010 | 0.49 | 0.87 | 17 | 4.4 |
| 301 | 75%@0.0010 | 0.070 | 0.036 | 28 | 12 |
| 286 | 0.0041 | | 0.68 | | 12 |
| 293 | 0.0011 | 0.11 | 0.37 | 35 | 45% at 30 |
| 209 | 0.0041 | 0.59 | 0.45 | 22% at 50 | 17% at 30 |
| 210 | 46%@0.030 | | | | 17% at 30 |
| 187 | 0.0055 | | 0.89 | | 5% at 10 |
| 188 | 49%@0.10 | | | | |
| 294 | 0.00093 | | 0.077 | | 23% at 10 |
| 197 | 0.00062 | | 0.21 | | 2% at 10 |
| 198 | 0.0050 | | | | |
| 211 | 0.0010 | 0.28 | 0.36 | 41% at 50 | 5% at 10 |
| 212 | 54%@0.030 | | | | |
| 202 | 72%@0.0010 | 0.064 | 0.11 | 24 | 13% at 10 |
| 201 | 0.0029 | | 0.76 | | 9% at 10 |
| 194 | 0.0033 | | | | |
| 193 | 0.00077 | 0.13 | 0.14 | 34 | 11% at 10 |
| 144 | 0.0021 | | 2.9 | | 3% at 10 |
| 300 | 60%@0.0010 | | | | |
| 179 | 48%@0.030 | | | | |
| 180 | 0.00095 | | 0.16 | | 14% at 10 |
| 295 | 0.00093 | | | | |
| 138 | 0.0044 | | 0.56 | | 18% at 10 |
| 139 | 42%@0.030 | | | | |
| 156 | 0.0011 | | 0.25 | | 9% at 10 |
| 213 | 0.0021 | 0.26 | 0.25 | 42% at 50 | 3% at 10 |
| 343 | 49%@0.10 | | | | |
| 203 | 0.0012 | 0.10 | 0.080 | 19% at 50 | 7% at 10 |
| 204 | 0.012 | | 0.94 | | −1% at 10 |
| 214 | 0.0014 | 0.26 | 0.22 | 12% at 50 | 0% at 10 |
| 215 | 51%@0.030 | | | | |
| 311 | 0.0026 | | 0.25 | | 11% at 10 |
| 312 | 57%@0.030 | | | | |
| 216 | 0.0032 | | 0.28 | | 1% at 10 |
| 217 | 52%@0.10 | | | | |
| 181 | 42%@0.010 | | | | |
| 182 | 0.0013 | | 0.88 | | 6% at 10 |
| 140 | 0.00070 | | 0.23 | | |
| 141 | 0.017 | | 1.8 | | |
| 142 | 0.00073 | | 0.23 | | |
| 143 | 0.0043 | | 0.86 | | |
| 277 | 0.0012 | | 1.2 | | 5% at 10 |
| 276 | 0.0036 | | 2.5 | | 33% at 10 |
| 279 | 0.00097 | | 0.57 | | 17% at 10 |
| 278 | 0.0034 | | 2.6 | | 18% at 10 |
| 196 | 0.0013 | 0.11 | 0.15 | 27% at 50 | 7% at 10 |
| 218 | 0.00086 | 0.22 | 0.43 | 31% at 50 | 4% at 10 |
| 219 | 0.00095 | 0.087 | 0.11 | 33 | 8% at 10 |
| 220 | 0.0081 | | 0.60 | | 14% at 10 |
| 296 | 62%@0.0010 | 0.11 | 0.16 | 32 | 12% at 10 |
| 195 | 0.0055 | | 0.61 | | 4% at 10 |
| 221 | 0.033 | | 2.2 | | 3% at 10 |
| 222 | 80%@0.0010 | 0.064 | 0.099 | 36 | 14% at 10 |
| 223 | 0.0026 | | 0.52 | | 8% at 10 |
| 324 | 0.0048 | | 2.2 | | 2% at 10 |
| 224 | 0.00070 | 0.078 | 0.12 | 47 | 6% at 10 |
| 226 | 0.0095 | | 0.57 | | 3% at 10 |
| 225 | 48%@0.030 | | 1.8 | | 9% at 10 |
| 347 | 63%@0.10 | | 1.7 | | |
| 325 | 0.0013 | | 0.24 | | |
| 227 | 0.0048 | | 0.29 | | 8% at 10 |
| 228 | 61%@0.010 | | 0.21 | | |
| 174 | 0.0038 | | | | |
| 183 | 0.0042 | | 0.43 | | 4% at 10 |
| 184 | 0.00092 | | 0.14 | | 8% at 10 |
| 372 | 52%@0.10 | | 2.6 | | |
| 373 | 0.0023 | | 0.26 | | 12% at 10 |
| 297 | 0.0026 | | 0.76 | | 5% at 10 |
| 229 | 51%@0.10 | | 1.4 | | |
| 230 | 0.0055 | | 0.22 | | 0% at 10 |
| 344 | 0.010 | | 1.9 | | 0% at 10 |
| 231 | 0.0028 | | 0.33 | | 5% at 10 |
| 232 | 50%@0.10 | | 2.3 | | 5% at 10 |
| 185 | 47%@0.010 | | 2.2 | | 8% at 10 |
| 186 | 0.0089 | | 0.42 | | 2% at 10 |
| 313 | 0.0028 | | 0.95 | | 2% at 10 |
| 314 | 52%@0.010 | | 66% at 10 | | 9% at 10 |
| 155 | 0.0094 | | 0.31 | | |
| 353 | 69%@0.0010 | 0.19 | 0.27 | 44% at 50 | 6% at 10 |
| 352 | 0.0055 | | 1.1 | | 13% at 10 |
| 385 | 0.0061 | | 0.45 | | 5% at 10 |
| 354 | 0.0013 | 0.16 | 0.34 | 36 | 8% at 10 |
| 421 | 0.00084 | | 0.59 | | 5% at 10 |
| 357 | 0.0015 | | 0.30 | | 10% at 10 |
| 360 | 0.0032 | | 0.74 | | 9% at 10 |
| 358 | 74%@0.0010 | | 0.039 | | 9% at 10 |
| 359 | 50%@0.10 | | 3.9 | | 6% at 10 |
| 389 | 41%@0.10 | | 4.2 | | 9% at 10 |
| 390 | 0.0035 | | 0.63 | | 11% at 10 |
| 391 | 0.0066 | | 0.66 | | 2% at 10 |
| 350 | 54%@0.030 | | 0.51 | | 2% at 10 |
| 351 | 25%@0.10 | | 4.3 | | 5% at 10 |
| 405 | 0.010 | | 0.63 | | 6% at 10 |
| 406 | 54%@0.10 | | 3.9 | | 11% at 10 |
| 418 | 0.00081 | 0.12 | 0.28 | 25 | 8% at 10 |
| 326 | 50%@0.030 | | 2.0 | | 16% at 10 |
| 407 | 62%@0.0010 | | 0.58 | | −9% at 10 |
| 408 | 0.0011 | | 0.75 | | 5% at 10 |
| 409 | 0.0019 | 0.28 | 0.41 | 12% at 50 | 5% at 10 |
| 395 | 44%@0.030 | | | | |
| 396 | 0.0044 | | 0.28 | | 9% at 10 |
| 392 | 45%@0.10 | | | | |
| 340 | 56%@0.10 | | 2.0 | | |
| 348 | 0.0026 | | 0.27 | | 5% at 10 |
| 349 | 79%@0.0010 | 0.042 | 0.028 | 44 | 6% at 10 |
| 341 | 0.0023 | 0.53 | 0.50 | 15% at 30 | −0% at 10 |
| 386 | 0.00065 | 0.034 | 0.019 | 45 | 6% at 10 |
| 331 | 49%@0.030 | | | | |
| 403 | 52%@0.030 | | 1.2 | | 2% at 10 |
| 397 | 49%@0.030 | | 2.5 | | 7% at 10 |
| 422 | 0.0032 | | 1.1 | | |
| 404 | 0.0018 | 0.16 | 0.095 | 25 | 12% at 10 |
| 355 | 16%@0.10 | | 4.7 | | |
| 356 | 0.0059 | 1.1 | 0.96 | 17% at 50 | 5% at 10 |
| 410 | 0.0022 | | 0.19 | | 4% at 10 |
| 411 | 0.00093 | 0.11 | 0.077 | 40 | 6% at 10 |
| 412 | 0.0023 | | 0.27 | | 8% at 10 |
| 413 | 0.0020 | 0.22 | 0.24 | 41 | 12% at 10 |
| 398 | 0.0023 | 0.58 | 0.58 | 16% at 50 | 4% at 10 |
| 423 | 0.0020 | 0.24 | 0.25 | 34 | |
| 416 | 0.0011 | 0.17 | 0.15 | 19 | 25% at 10 |
| 417 | 0.0052 | | 0.41 | | 16% at 10 |
| 332 | 60%@0.030 | | 2.6 | | 5% at 10 |
| 414 | 60%@0.0030 | | 0.11 | | |
| 415 | 0.00084 | | 0.24 | | |
| 393 | 0.0065 | | 0.79 | | |
| 394 | 18%@0.10 | | | | |
| 424 | 45%@0.0030 | | 0.69 | | |
| 338 | 50%@0.030 | | 1.5 | | 6% at 10 |
| 337 | 48%@0.00010 | 0.051 | 0.058 | 46 | 7% at 10 |
| 361 | 0.0039 | | 0.78 | | 8% at 10 |
| 362 | 52%@0.10 | | | | |
| 425 | 0.0019 | | 0.74 | | 7% at 10 |
| 399 | 50%@0.00030 | | 11% at 10 | | 2% at 10 |
| 400 | 0.0031 | | 1.1 | | 4% at 10 |
| 363 | 48%@0.10 | | | | |
| 364 | 0.0055 | | 0.47 | | 9% at 10 |
| 333 | 0.0044 | | 0.22 | | 5% at 10 |
| 334 | 42%@0.10 | | 73% at 10 | | 4% at 10 |
| 365 | 0.0011 | 0.067 | 0.10 | 33% at 50 | −0% at 10 |
| 366 | 0.19 | | 2.7 | | 2% at 10 |
| 335 | 51%@0.10 | | 2.8 | | 7% at 10 |
| 336 | 56%@0.0010 | 0.10 | 0.16 | 42 | 4% at 10 |
| 401 | 37%@0.00030 | 0.0089 | 0.0098 | 36 | 6% at 10 |
| 402 | 0.0043 | | 0.21 | | 5% at 10 |
| 367 | 0.0026 | 0.22 | 0.11 | 46 | 1% at 10 |
| 368 | 27%@0.10 | | 52% at 10 | | 6% at 10 |
| 371 | 43%@0.030 | | 2.3 | | 3% at 10 |
| 374 | 0.00090 | 0.64 | 0.85 | 26% at 50 | 2% at 10 |
| 375 | 37%@0.10 | | | | |
| 387 | 42%@0.10 | | | | |
| 388 | 0.0021 | 0.066 | 0.23 | 39 | 4% at 10 |
| 369 | 0.00061 | 0.058 | 0.062 | 18% at 50 | 5% at 10 |
| 370 | 51%@0.10 | | 3.5 | | 0% at 10 |
| 339 | 0.00051 | | 0.21 | | 3% at 10 |
| 342 | 25%@0.10 | | 21% at 10 | | 4% at 10 |
| 428 | 65%@0.0010 | 0.11 | 0.19 | 22 | 12% at 10 |
| 429 | 65%@0.10 | | | | |
| 430 | 59%@0.0010 | 0.11 | 0.18 | 33% at 50 | 4% at 10 |
| 431 | 48%@0.0010 | 0.11 | 0.16 | 39% at 50 | 5% at 10 |
| 432 | 45%@0.030 | | 6.2 | | 8% at 10 |
| 443 | 58%@0.0010 | 0.11 | 0.093 | 38 | 3% at 10 |
| 444 | 63%@0.10 | | | | |
| 433 | 0.011 | | 1.5 | | 20% at 30 |
| 434 | 0.0013 | | 0.47 | | 7% at 10 |
| 448 | 84%@0.0010 | | 0.86 | | 48% at 30 |
| 445 | 43%@0.0010 | | 3.3 | | 16% at 10 |
| | 56%@0.10 | | | | |
| 446 | 76%@0.0010 | 0.018 | 0.023 | 29 | 13% at 10 |
| 447 | 74%@0.0010 | | 0.011 | | 10% at 10 |
| 435 | 66%@0.0010 | | 0.015 | | 0% at 10 |
| 436 | 53%@0.10 | | | | |
| 426 | 0.00072 | 0.22 | 0.32 | 29% at 50 | 4% at 10 |
| 427 | 41%@0.10 | | | | |
| 437 | 51%@0.030 | | 2.5 | | −4% at 10 |
| 438 | 0.0016 | | 0.34 | | 2% at 10 |
| 439 | 85%@0.0010 | 0.045 | 0.030 | 46% at 50 | 3% at 10 |
| 440 | 0.012 | | 1.9 | | 7% at 10 |
| 449 | 78%@0.0010 | 0.041 | 0.012 | 18 | 19% at 10 |
| 450 | 76%@0.0010 | | 0.38 | | 15% at 10 |
| 441 | 0.012 | | 1.5 | | 1% at 10 |
| 442 | 0.0034 | 0.42 | 0.50 | 26% at 50 | 6% at 10 |
| 451 | 0.078 | | | | |
| 452 | 0.15 | | | | |
| 453 | 0.094 | | | | |
| 454 | 0.035 | | | | |
| 455 | 58%@1.0 | | | | |
| 456 | 0.21 | | | | |
| 457 | 53%@0.30 | | | | |
| 458 | 44%@0.010 | | | | 12 |
| 459 | 0.0020 | | 6.4 | | 7% at 10 |
| 460 | 0.48 | | | | |
| 461 | 0.00053 | 0.075 | 0.091 | 19% at 50 | 6% at 10 |
| 462 | 80%@0.0010 | 0.073 | 0.047 | 37% at 50 | 4% at 10 |
| 463 | 73%@0.10 | | | | |
| 464 | 0.0019 | | 0.18 | | 46% at 10 |
| 465 | 29%@0.10 | | | | |
| 466 | 0.00086 | 0.21 | 0.14 | 41% at 50 | 4% at 10 |
| 467 | 36%@0.10 | | | | |
| 468 | 0.0035 | | 0.48 | | 2% at 10 |
| 469 | 52%@0.030 | | | | |
| 470 | 0.001 | | 0.1 | | 3% at 10 |
| 471 | 38%@0.10 | | 3.5 | | 2% at 10 |
| 472 | 48%@0.0030 | | 0.39 | | 10% at 10 |
| 473 | 44%@0.10 | | 104% at | | 11% at 10 |
| | | | 10 | | |
| 474 | 38%@0.030 | | 1.3 | | 12% at 10 |
| 475 | 71%@0.0010 | 0.028 | 0.022 | 46% at 50 | 7% at 10 |
| 476 | 51%@0.030 | | 0.6 | | 4% at 10 |
| 477 | 86%@0.0010 | 0.013 | 0.0091 | | 5% at 10 |
| 478 | 58%@0.10 | | 81% at 10 | | −0% at 10 |
| 479 | 0.0006 | | 0.079 | | 2% at 10 |
| 480 | 67%@0.10 | | 3.7 | | 2% at 10 |
| 481 | 0.0014 | | 0.33 | | 8% at 10 |
| 483 | 58%@0.0010 | | 0.2 | | 9% at 10 |
| 485 | 58%@0.10 | | 4.2 | | 3% at 10 |
| 486 | 59%@0.0010 | 0.075 | 0.051 | 10% at 50 | 7% at 10 |
| 493 | 0.00054 | 0.038 | 0.02 | 25 | 11% at 10 |
| 494 | 0.013 | | | | |
| 495 | 0.0011 | 0.068 | 0.054 | 34% at 50 | 7% at 10 |
| 496 | 56%@0.10 | | 1.8 | | −0% at 10 |
| 500 | 21%@0.10 | | 16% at 10 | | 6% at 10 |
| 501 | 0.0031 | 0.015 | 0.022 | 6.4 | 8% at 3.0 |
| 502 | 18%@0.10 | | 4.9 | | 11% at 10 |
| 503 | 87%@0.0010 | | 0.011 | | 13% at 10 |
| 505 | 60%@0.0010 | | 0.027 | | 5% at 10 |
| 506 | 36%@0.030 | | 3.4 | | 2% at 10 |
| 507 | 65%@0.0030 | | 0.02 | | 5% at 3 |
| 508 | 17%@0.10 | | 11% at 10 | | 9% at 10 |
| 509 | 0.00094 | | 0.042 | | 6% at 10 |
| 510 | 79%@0.0010 | 0.084 | 0.073 | 5% at 50 | 9% at 10 |
| 511 | 72%@0.0010 | | 0.018 | | 10% at 10 |
| 512 | 0.00048 | 0.053 | 0.025 | 27% at 50 | 5% at 10 |
| 514 | | | 0.64 | | 3% at 10 |
| 516 | 0.0012 | | 0.038 | | 4% at 10 |
| 517 | 82%@0.0010 | 0.019 | 0.01 | 26% at 50 | 5% at 10 |
| 518 | 79%@0.0010 | 0.058 | 0.065 | 46% at 50 | 10% at 10 |
| 519 | 35%@0.030 | | 1.9 | | 12% at 10 |
| 520 | 32%@0.030 | | 3.9 | | 5% at 10 |
| 521 | 77%@0.0010 | 0.035 | 0.033 | 37 | 11% at 10 |
| 524 | 0.021 | | 1.1 | | 35% at 10 |
| 525 | 50%@0.0010 | 0.17 | 0.078 | 17 | 40% at 10 |
| 526 | 0.0013 | 0.11 | 0.11 | | 9% at 10 |
| 527 | 0.017 | | 1.4 | | 14% at 10 |
| 528 | 0.0029 | | 0.32 | | 10% at 10 |
| 530 | 0.0031 | | 0.39 | | 2% at 10 |
| 531 | 42%@0.10 | | 4.3 | | 8% at 10 |
| 532 | 0.0031 | | 0.14 | | 7% at 10 |
| 533 | 44%@0.10 | | 4 | | 20% at 10 |
| 534 | 0.0088 | | 0.7 | | 44% at 10 |
| 535 | 65%@0.0010 | 0.05 | 0.058 | 18 | 45% at 10 |
| 536 | 64%@0.0010 | | 0.13 | | 46% at 10 |
| 522 | 0.0007 | 0.053 | 0.048 | 19 | 24% at 10 |
| 523 | 50%@0.030 | | 1.5 | | 29% at 10 |
| 537 | | | 0.76 | | 45% at 10 |
| 538 | 44%@0.030 | | 2.2 | | 43% at 10 |
| 539 | 41%@0.0010 | | 0.092 | | 38% at 10 |
| 540 | 33%@0.030 | | 1.8 | | 62% at 10 |
| 541 | 64%@0.00030 | 0.19 | 0.014 | 13 | 10 |
| 542 | 57%@0.010 | | 3.1 | | −1% at 10 |
| 543 | 68%@0.0010 | | 0.69 | | −6% at 10 |
| 544 | 86%@0.0010 | 0.032 | 0.041 | 7.4 | 109% at 10 |
| 545 | 94%@0.0010 | | 0.094 | | 4% at 3 |
| 546 | 86%@0.0010 | | 0.7 | | 17% at 10 |
| 547 | 82%@0.0010 | | 0.96 | | 4% at 10 |
| 548 | 82%@0.0010 | 0.14 | 0.17 | 6.3 | 111% at 10 |
| 549 | 85%@0.0010 | | 0.27 | | 33% at 10 |
| 553 | 0.0006 | | 0.065 | | 24% at 10 |
| 554 | 74%@0.0010 | 0.21 | 0.07 | 46% at 50 | 11% at 10 |
| 555 | 55%@0.0010 | | 0.079 | | 58% at 10 |
| 556 | 91%@0.0010 | 0.056 | 0.0064 | 27 | 14% at 10 |
| 557 | 81%@0.0010 | 0.25 | 0.037 | 37% at 10 | 6% at 10 |
| 558 | 53%@0.0030 | 0.22 | 0.082 | 35 | 6% at 10 |
| 559 | 0.00062 | 0.46 | 0.052 | 22% at 50 | 1% at 10 |
| 560 | 30%@0.0010 | | 0.13 | | 3% at 10 |
| 561 | 47%@0.0010 | 0.021 | 0.065 | 26 | 12% at 10 |
| 562 | 0.0013 | | 0.25 | | 1% at 10 |
| 563 | 76%@0.0010 | 0.025 | 0.027 | 15 | 72% at 10 |
| 564 | 86%@0.0010 | 0.018 | 0.00038 | 16 | 10% at 3 |
| 565 | 65%@0.0010 | 0.045 | 0.035 | 33 | 11% at 10 |
| 566 | 47%@0.030 | | 1.9 | | 46% at 10 |
| 567 | 57%@0.10 | | | | |
| 568 | 76%@0.0010 | | 0.094 | | 36% at 10 |
| 570 | 83%@0.0010 | 0.034 | 0.046 | | 26% at 10 |
| 571 | 77%@0.0010 | 0.023 | 0.0085 | 17 | 38% at 10 |
| 572 | 53%@0.00075 | 0.019 | 0.022 | 18 | 23% at 10 |
| 550 | 0.00098 | 0.077 | 0.056 | 17 | 33% at 10 |
| 551 | 70%@0.0010 | 0.031 | 0.026 | 36 | 13% at 10 |
| 552 | 79%@0.0010 | 0.028 | 0.02 | 21 | 7% at 10 |
| 513 | 0.0031 | 0.26 | 0.38 | 19% at 50 | 4% at 10 |
| 576 | 42%@0.0030 | | 0.14 | | 5% at 10 |
| 577 | 0.00093 | | 0.079 | | 7% at 10 |
| 491 | 0.0089 | | 0.81 | | 5% at 10 |
| 578 | 0.002 | | 0.12 | | 4% at 10 |
| 499 | 68%@0.0010 | | 0.053 | | 2% at 10 |
| 498 | 32%@0.10 | | 96% at 10 | | 4% at 10 |
| 497 | 83%@0.0010 | 0.039 | 0.046 | 45 | 10% at 10 |
| 487 | 59%@0.0010 | 0.12 | 0.025 | 27 | 12% at 10 |
| 488 | 56%@0.10 | | | | |
| 529 | 0.028 | | 2.2 | | 17% at 10 |
| 482 | 58%@0.10 | | 5 | | 3% at 10 |
| 484 | | | 4.9 | | 2% at 10 |
| 574 | 45%@0.0010 | 0.021 | 0.016 | 17 | 27% at 10 |
| 575 | 38%@0.0010 | 0.028 | 0.066 | 21 | 14% at 10 |
| 504 | 71%@0.0010 | 0.039 | 0.034 | 48 | 0% at 10 |
| 492 | 31%@0.10 | | 25% at 10 | | 19% at 10 |
| 579 | 41%@0.030 | | 19% at 10 | | 4% at 10 |
| 515 | 0.0014 | | 1.0 | | 8% at 10 |
| 489 | | | 3.3 | | −9% at 10 |
| 490 | 0.0023 | | 0.27 | | −0% at 10 |
| 580 | | | 0.014 | | 13% at 10 |

Where more than one data point has been obtained, the table above shows an average (e.g. geometric or arithmetic mean) of these data points.

It is of course to be understood that the invention is not intended to be restricted to the details of the above embodiments which are described by way of example only.

Example 2—DNA Damage Response (DDR) is the Top Sensitizinq Pathway to Compound 1, Identified by Loss of Function CRISPR Screen and Confirmed by Further Experiments and Data

A dual CRISPR screen (CRISPR knock-out and CRISPRi) was carried out in a panel of three P53-wild lung cancer cell lines in the presence or absence of Compound 1, to identify novel predictive biomarkers of MDM2 antagonist sensitivity.

Several DNA-damage-response (DDR) related genes were identified as the top hits (FIG. 1A-B). Interestingly, these genes are involved in several DDR pathways, such as Homologous Recombination, Fanconi Anemia (FA), Base Excision Repair (BER) and Replication Stress pathways. FIG. 1A shows enrichment of the Fanconi Anemia pathway in the CRISPR hits.

Replication stress is a readout of genomic instability and is caused by multiple defects in DDR pathways leading to high levels of DNA damage. High levels of DNA damage in turn affect the process of DNA replication. The CRISPR screen data indicate that tumours with defects in their DDR machinery are generally sensitive to Compound 1 treatment.

These data therefore demonstrate the link between MDM2 antagonist sensitivity and defects in multiple DDR pathways, including the Fanconi Anemia (FA) and Base Excision Repair (BER) pathways. Loss of function in a DDR pathway is therefore a biomarker for MDM2 antagonist sensitivity.

To confirm the results of the CRISPR screen, early passages human mesothelioma cell lines, previously characterized for their sensitivity to Compound 1, were used to identify transcriptomic signatures differentially expressed between apoptotic and non-apoptotic samples. A “Replication Stress” signature was strongly enriched in mesothelioma apoptotic cell lines, confirming the link between Compound 1 sensitivity and activated DDR pathways (FIG. 1C).

The following Examples, below, describe bioinformatic and wet lab analysis on multiple systems that validate specific biomarkers in DDR pathways as biomarkers for MDM2 antagonist sensitivity. These additionally validated biomarker pathways are summarized in FIG. 5. In addition to providing further evidence on the exemplified biomarker pathways, these data re-confirm the reliability of the CRISPR screen data more generally.

### Bioinformatics Analysis of Genome-Wide CRISPR Screen Data

Whole-genome Dual LoF screens (CRISPRko and CRISPRi) in the presence of compound 1 were conducted by Horizon Discovery (https://horizondiscovery.com/) in a panel of three P53-wild type lung cancer cell lines (A549, NCI-H460, NCI-H292). CRISPRko and CRISPRi were run in parallel and analysed together to identify potential hits and pathways of sensitisation to compound 1.

NGS analysis showed excellent QC with a mean Quality score of 35 and all samples sequenced with >97% of reads with a quality score more than 30. Overall, there was an excellent correlation between replicates for all samples. Control sgRNAs (positive, negative & non-targeting) performed as expected with a distinct drop-out of essential genes in control-treated samples versus the initial library plasmid.

Data were analysed by Horizon as well as in-house using two different computational methods: DrugZ and MAGeCK. CRISPR hits were ranked by fold-change and significant p-values. There was a good overlap of significant hits between CRISPRko and CRISPRi. Both unique and overlapping sensitising genes were identified, particularly genes associated with DNA damage repair (DDR) showed strong drop-outs in the screens. Further, network analysis of CRISPR hits showed strong enrichment of genes from the Fanconi Anemia Pathway suggesting a role in interstrand crosslink repair (FANCA, FANCB, FANCD2) (FIG. 1A). Gene set enrichment analysis (GSEA) was performed on CRISPR hits ranked by fold-change values using different gene set signatures: Hallmark, Reactome, KEGG & Biocarta pathways. GSEA results revealed significant enrichment of DNA repair associated pathways such as Base Excision Repair pathway and Homologous Recombination (FIG. 1B) in the top depleted hits.

Based on our findings from the CRISPR screen, we also investigated DDR gene expression signatures in our in-house RNA-seq data of apoptotic and non-apoptotic mesothelioma cell lines.

Early passages human mesothelioma cell lines were purchased from the UK Mesobank (vyw.e sb c). Gene expression profiling of mesobank cell lines was performed by paired-end, stranded RNA-sequencing using the Illumina HiSeq platform and 3 biological replicates for each sample.

Sequencing was done by GATC Biotech (now Eurofins Genomics) and the bioinformatics analysis of RNA-seq data was done in-house. On average around 37 million reads were produced per sample. The RNA-seq reads were aligned to the human genome hg38/GRCh38 using the STAR aligner (v2.5.4b).

On average, 94% of the reads were aligned uniquely to the genome. Aligned BAM files were used for transcript and gene quantification using the htseq-count tool of the HTSeq software suite (version 0.11.1) based on the GENCODE v27 annotations. Variance stabilizing transformation function from the DESeq2R package (v1.20.0) was used to normalize the raw count data and unsupervised hierarchical clustering was performed. Biological replicates were highly correlated (R²=0.98). Differential gene expression was performed using the DESeq2R package. Genes with more than 2-fold expression and adjusted β-values <le-7 were considered as significantly differentially expressed between apoptotic and non-apoptotic samples. Interestingly, we found a significant up-regulation of genes associated with replication stress in the apoptotic mesothelioma cell lines (FIG. 1C), which was consistent with the CRISPR data screen output.

### HR Pathway: BRCA1, BRCA2 and ATM Alterations

Genes involved in the homologous recombination pathway were identified in the CRISPR screen. Homologous recombination (HR) is an error-free DSB repair pathway that is largely restricted to the S—and G2-phases of the cell cycle. The enormous importance of the HR pathway for genome maintenance is shown by the identification of several cancer disabling mutations in BRCA1, BRCA2, ATM, CHEK2, RAD50, RAD51C in numerous cancers.

One of the central components of the HR is the serine-threonine kinase Ataxia Telangiectasia Mutated protein (ATM), which phosphorylates numerous key players in various branches of the DDR. Somatic ATM mutations or deletions are commonly found in lymphoid malignancies, as well as several solid tumors leading to loss of protein expression and impairment of DNA double strand breaks repair in the genome.

Bioinformatics analysis of publicly available DepMAP RNAi data (version 20Q4) for MDM2 predicted that ATM mutant cell lines are significantly more dependent on MDM2 as compared to ATM wild cell lines (FIG. 2a). In addition, a strong enrichment of ATM mutation was detected in apoptotic patient-derived mesothelioma lines (6/9) compared to non-apoptotic lines which are all ATM wild-type (6/6) (FIG. 2B).

Further, in vitro validation on four ATMmut cell lines from different indications (HCC1500—breast, LNCap—prostate, HT-144—melanoma, HepG2—liver) showed sensitivity to Compound 1 as measured by reduction in cell proliferation (FIG. 2C), while data on LNCap—prostate and HepG2—liver showed sensitivity to Compound 1 as measured by increased apoptosis (FIG. 2D). Furthermore, western blot analysis showed a clear modulation of the DDR signalling pathway upon Compound 1 treatment (FIG. 2E).

Together with the identification of ATM mutations as a biomarker for MDM2 antagonist sensitivity additional bioinformatic analysis indicate that loss or mutation of other HR pathway genes may act as biomarkers for MDM2 antagonist sensitivity. HR pathway genes that may act as biomarkers for MDM2 antagonist therapy include but are not limited to BRCA1 and/or BRCA2.

To confirm if ATM, BRCA1 and/or BRCA2 mutations (or loss of expression) could be linked to MDM2 antagonist sensitivity further in vitro validation on patient-derived organoids (PDOs) was carried out. 4 PDOs from various indications with alterations in either ATM, BRCA1 and/or BRCA2 showed sensitivity to Compound 1 as measured by reduction in cell proliferation (FIG. 2F-G). Of note 4 additional PDOs from the same indications but with no alterations in ATM, BRCA1 and/or BRCA2 were resistant to Compound 1.

FA pathway genes are also identified as MDM2 antagonist therapy biomarkers.

### Bioinformatics Analysis

We interrogated publicly available MDM2 RNAi dependency data (version 20Q4) of cancer cell lines from DepMAP (www.depmap.org). Genomic features for these cancer cell lines such as somatic mutations and copy number alterations were obtained from the DepMAP datasets. We found that the cell lines differentially dependent on MDM2 were enriched for mutations in ATM (FIG. 2A). Based on our findings on ATM mutations from publicly available RNAi dependency datasets, we further investigated status of the ATM gene in our proprietary panel of apoptotic and non-apoptotic mesothelioma cell lines (FIG. 2B).

The DNA isolation and exome sequencing of mesothelioma cell lines were performed by GATC Biotech (now Eurofins) according to their guidelines. Genomic DNA was extracted and exome sequencing was performed using Agilent SureSelect Human All Exon V6 kit. The sequencing library was constructed and analysed by the Illumina HiSeq using the 101-bp paired-end sequencing. Low quality calls (average Phred score below 15) were removed before proceeding with any further processing and only mate pairs (forward and reverse read) were used for the analysis. Mapping to the reference human genome assembly hg19 was done using BWA v0.7.15 with default parameters. On average, 96% of reads mapped uniquely to the reference genome and the mean target coverage was 94.02±18.66-fold. Exome data was used to call Single Nucleotide Polymorphisms (SNPs) and Insertion-deletion (InDels) which was done by GATC Biotech using GATK and the Ingenuity software. Data was also analysed in-house using VarScan2. We considered only SNPs and InDels called commonly by at least two methods to obtain high-confidence results. For GATK, sequence alignments were refined by performing local alignment and PCR duplicates were removed using PICARD (http://picard.sourceforge.net/). The SNP and InDel calling was done using GATK's Haplotype Caller and were annotated using snpEff. For varScan2, SNPs and InDels were called using SAMtools, mpileup data as input and running varScan2 in somatic mode. ATM mutations predicted as deleterious loss of function mutations by at least two methods were considered for further analysis.

### Proliferation Assays on Cancer Cell Lines

Cancer cells were cultured in appropriate medium. Cells were harvested, counted, adjusted to the appropriate density and seeded in a volume of 100 μL into 96-well opaque-walled clear bottom plates and incubated overnight at 37° C. in a humidified atmosphere of 5% CO₂. A 10 mM stock solution of Compound 1 was prepared in DMSO. The stock solution was further diluted in DMSO, before addition to duplicate wells of the 96-well plates containing the cells, to give a 0.1% DMSO final concentration. Plates were then incubated at 37° C. in a humidified atmosphere of 5% CO₂ for 3 days. Each cell line was tested in triplicates. 100 μL of CellTiter-Glo reagent was added to each well of the assay plate. Plates were mixed on an orbital shaker for 10 minutes, before undergoing a 10-minute incubation at room temperature. The plate was then read (for luminescence) in an EnSpire plate reader. Each well was calculated, minus medium only control (no cells) as percentage of the mean DMSO control minus medium only control. Sigmoidal dose-response (variable slope) curves and IC₅₀ values were calculated using GraphPad Prism (GraphPad Software, La Jolla California USA).

### Apoptosis Assays

Cells were seeded into 6-well plates at a density of 2×10⁵ per well and incubated overnight at 37° C. in a humidified atmosphere of 5% CO₂ in air. Compound 1 was prepared in DMSO and added to the cells at the indicated concentrations. After 72-hour incubation of the cells with compound, cells were trypsinised, washed with PBS and immediately used for flow cytometry analysis.

The samples were stained with eBioscience™ Annexin V-FITC Apoptosis Detection Kit (#BMS500FI-100, Thermo Fisher Scientific, Waltham, MA, USA) according to manufacturer's recommendations. Briefly, the samples were washed in PBS and resuspended in 200 μl of 1× binding buffer containing 5 μl of Annexin V-FITC for 10 min at room temperature. Following the incubation, samples were washed with 1× binding buffer and resuspended in 200 μl 1× binding buffer and 10 μl of the 20 μg/ml PI.

The stained samples were then immediately analysed by flow cytometry on Guava easyCyte HT cytometer (Merck-Millipore). The cell populations were separated into three groups: live cells showing only a low level of fluorescence (Annexin V-/PI-), apoptotic cells showing green fluorescence (Annexin V+/PI−) and dead cells showing both red and green fluorescence (Annexin V+/PI+). Data analyses were performed using Microsoft excel and results plotted on Prism version 7 (GraphPad Software, California, USA).

Western Blotting Cell lysates were prepared by taking the cell pellets and adding ice-cold 1× complete Tris lysis buffer (1% Triton X-100, 150 mM NaCl, 20 mM Tris.HCl pH 7.5, plus protease inhibitors (complete mini, 1 tablet/10 ml, Roche, Welwyn Garden City, Herts, UK), 50 mM NaF and 1 mM Na3V04). Samples were vortexed and left on ice for 30 min. Lysates were cleared by centrifugation for 15 minutes at 14,000 rpm in a cooled microfuge and a sample of the supernatant removed for protein determination (BCA assay—Pierce, Paisley, UK).

The cell lysates were then analysed by Western blotting. Equivalent amounts of protein lysate were mixed with SDS sample buffer (Novex, Paisley, UK) and DTT before being boiled for 10 min. Samples were resolved by SDS PAGE (4-12% Nu-PAGE gels—Novex, Paisley, Scotland), blotted onto nitrocellulose filters, blocked with Odyssey Blocking Buffer (LI-COR Bioscience, Lincoln, USA) and incubated overnight at 4° C. with the specific primary antibodies, diluted in Odyssey blocking buffer. After washing, blots were incubated for 1 hour with infrared dye labelled anti-rabbit IR800 or anti-goat IR800 secondary antibodies at a dilution of 1: 10,000 in Odyssey blocking buffer (LiCor Biosciences, Lincoln, USA). Blots were then scanned to detect infrared fluorescence on the Odyssey infrared imaging system (LiCOR Biosciences, Lincoln, USA).

### Proliferation Assays on Patient-Derived Organoids (PDOs)

Organoids were retrieved from the CrownBIO organoid bank and expanded before getting enough quantity according to standard operation procedure. One day before organoids seeding, the needed number of organoids were passaged 1:1 using 50% Matrigel and 50% corresponding culture medium. On Day 0 organoids were seeded and compound added: organoids were collected by adding 20 μl 100× Dispase solution to each well from a 6-well plate, and incubating at 37C for 30 min. After incubation, organoids were collected from all wells and pipetted through a pre-wet 100 μm filter into a 50 ml plastic tube, then filtered the flow through over a pre-wet 20 μm filter, inverted the 20 μm filter and recovered the organoids in a new 50 ml tube, Collect organoids were then resuspended in the corresponding culture media, and counted. Organoids cell suspensions were added to 384-well plate by Multidrop dispenser. 2-4 hours after organoids seeding, compounds were added by Tecan D300e, then plates were placed back into incubator for 5 days' incubation. On day 5, proliferation was assayed by CellTiter-Glo assay. CTG reagent was added to each well of the assay plate. Plates were mixed on an orbital shaker for 10 minutes, before undergoing a 10-minute incubation at room temperature. The plate was then read (for luminescence) in an Envision plate reader.

### NHEJ Pathway: ATRX Loss

Additionally, bioinformatics analysis of the cell panel data predicted loss of ATRX as a significant biomarker of sensitivity to MDM2 Compound 1 (FIG. 3). ATRX is also been implicated in the regulation of DDR both by non-homologous end joining (NHEJ) and homologous recombination repair (HRR).

The identification of ATRX loss as a biomarker for MDM2 antagonist therapy and bioinformatics analysis demonstrate that loss or mutation of other NHEJ or HRR pathway genes may act as biomarkers for MDM2 antagonist sensitivity.

### Bioinformatics Analysis

Compound 1 was screened in a panel of 237 cancer cell lines. IC50s and activity areas were calculated from the raw dose-response curves. Genomic features for these cell lines such as somatic mutations, copy number alterations and hypermethylation were obtained from the list of Cancer Functional Events as mentioned in Garnett et al (2016) ANOVA method was used to identify significant associations of genomics features to drug response. We identified ATRX loss as statistically significant (adjusted p-value <0.20) biomarker of compound 1 sensitivity (FIGS. 3a and 3b).

### MMR Pathway: Microsatellite Instability (MSI)

Microsatellites are regions that contain multiple repeats of 1 to 5 base pair motifs which are widely dispersed throughout the human genome. In normal cells, repeat count of microsatellites is verified and maintained during cell division by the mismatch repair (MMR). Impairment of the MMR system can render cells unable to regulate the lengths of their microsatellites during cell division, termed MSI (for microsatellite instability). MSI has been frequently observed within several types of cancer (colorectal, endometrial, and gastric adenocarcinomas) and MSI-High colorectal tumors have been shown to be more susceptible to immune-enhancing therapies.

In the cell panel data, MSI-H colorectal cell lines were sensitive to Compound 1. Information on microsatellite stability and tumour mutation burden for cell lines was obtained from Sanger Cell Models Passport database. We found that MSI-H cell lines exhibited a high tumour mutation burden (mutations/Mb) and were enriched in mutations related to DNA mismatch repair pathway (eg. MSH2, MSH3, MSH6, MLH1, MLH3, PMS2). Further, MSI-H cell lines showed strong enrichment of mutational signatures associated with defects in DNA mismatch repair and POLD1 and/or POLE mutations (FIG. 4A). Together, these findings were consistent and suggested that MSI tumours such as colorectal, endometroid and gastric would be sensitive to MDM2 antagonists. Further, in vitro validation on eight MSI-H cell lines from different indications showed sensitivity to Compound 1 as measured by reduction in cell proliferation (FIG. 4B). To confirm if MSI-H status could be linked to MDM2 antagonist sensitivity further in vitro validation on patient-derived organoids (PDOs) was carried out. 6 MSI-H colorectal cancer PDOs showed sensitivity to Compound 1 as measured by reduction in cell proliferation (FIG. 4C).In vivo efficacy data confirmed that Compound 1 significantly inhibits tumour growth in a xenograft model of MSI-H colorectal cancer (HCT-116) (FIG. 4D).

### Proliferation Assays on Cancer Cell Lines

Cancer cells were cultured in appropriate medium. Cells were harvested, counted, adjusted to the appropriate density and seeded in a volume of 100 μL into 96-well opaque-walled clear bottom plates and incubated overnight at 37° C. in a humidified atmosphere of 5% CO₂. A 10 mM stock solution of Compound 1 was prepared in DMSO. The stock solution was further diluted in DMSO, before addition to duplicate wells of the 96-well plates containing the cells, to give a 0.1% DMSO final concentration. Plates were then incubated at 37° C. in a humidified atmosphere of 5% CO₂ for 3 days. Each cell line was tested in triplicates. 100 μL of CellTiter-Glo reagent was added to each well of the assay plate. Plates were mixed on an orbital shaker for 10 minutes, before undergoing a 10-minute incubation at room temperature. The plate was then read (for luminescence) in an EnSpire plate reader. Each well was calculated, minus medium only control (no cells) as percentage of the mean DMSO control minus medium only control. Sigmoidal dose-response (variable slope) curves and IC₅₀ values were calculated using GraphPad Prism (GraphPad Software, La Jolla California USA).

### Proliferation Assays on Patient-Derived Organoids (PDOs)

Organoids were retrieved from the CrownBIO organoid bank and expanded before getting enough quantity according to standard operation procedure. One day before organoids seeding, the needed number of organoids were passaged 1:1 using 50% Matrigel and 50% corresponding culture medium. On Day 0 organoids were seeded and compound added: organoids were collected by adding 20 μl 100× Dispase solution to each well from a 6-well plate, and incubating at 37C for 30 min. After incubation, organoids were collected from all wells and pipetted through a pre-wet 100 μm filter into a 50 ml plastic tube, then filtered the flow through over a pre-wet 20 μm filter, inverted the 20 μm filter and recovered the organoids in a new 50 ml tube, Collect organoids were then resuspended in the corresponding culture media, and counted. Organoids cell suspensions were added to 384-well plate by Multidrop dispenser. 2-4 hours after organoids seeding, compounds were added by Tecan D300e, then plates were placed back into incubator for 5 days' incubation. On day 5, proliferation was assayed by CellTiter-Glo assay. CTG reagent was added to each well of the assay plate. Plates were mixed on an orbital shaker for 10 minutes, before undergoing a 10-minute incubation at room temperature. The plate was then read (for luminescence) in an Envision plate reader.

### In Vivo Efficacy

5×10⁶ HCT116 cells (in 100 μl of PBS) were injected subcutaneously into the right hind flank of groups of BALB/c nude mice. Tumours were measured externally by using a pair of digital callipers and volumes calculated as length×width²×0.523. Before starting the study, the mice were arranged into groups of 8 according to tumour volume; mean volume should be circa 100 mm³ with a normal range of 50 to 150 mm³. Mice were dosed daily with 50 mg/kg of Compound 1 and body weights recorded daily during the experimental period. Dosing was stopped if the animals showed any signs of toxicity or if the body weight fell below 85% of the initial body weight. Tumour volumes were measured every 2 to 3 days; animals were sacrificed when tumour volume was >1000 mm³ or if the tumours started to show any abnormalities, such as ulceration.

Summary of DDR Pathway Data The dual loss of function CRISPR screen identified DDR pathways as a top sensitizing hit to MDM2 antagonism. These analyses are in general agreement with HORIZON (HR, replication stress, NER, FA pathways).

Further analyses support the link between DDR deficiency and MDM2 antagonist sensitivity:
DDR/replication stress signature present in apoptotic patient-derived mesothelioma
ATRX mutations are associated with IFN cluster in sarcoma
All apoptotic patient-derived mesothelioma lines except 1 carry ATM mutations. Non-apoptotic lines are all ATM wild-type.
ATM mutations are linked to MDM2 dependency in the RNAi dataset; consistent with anecdotal observations in the clinic
ATM mutant cell lines (HCC1500, HT-144, LNCap, HepG2) show increased sensitivity to Compound 1 BRCA1, BRCA2 and/or ATM mutant patient derived organoids show increased sensitivity to Compound 1 compared to PDOs carrying no alterations in those genes
MSI-H cell lines from various indications and patient-derived colorectal cancer models show sensitivity to Compound 1
Compound 1 significantly inhibits tumour growth in MSI-H xenograft model (HCT-116)

Example 3—Combination effect of Compound 1 with PARP inhibitors on cancer cell viability Objective

The objective of this study is to investigate the potential combination effect of Compound 1 with 2 compounds on cancer cell viability. Firstly, the 50% inhibition concentration (IC₅₀) of 3 compounds will be determined using CellTiter-Glo (CTG) luminescent cell viability assays after incubation with different test article concentrations. Then, the synergy effect of Compound 1 in combination with 1 compound (Olaparib, Talazoparib) each will be evaluated by Combination matrix respectively.

### Experimental Design

The cell line will be treated with test articles alone, matrix combinations with test articles and culture medium as vehicle control.

Materials and Methods

### 1. Cell Line

| | | | Incubation |
|---|---|---|---|
| Cell Line Name | Tissue Origin | Reference Control | Time |
| CAL-51 | Breast | Cisplatin | 72 h |
| A2780 | Ovary | Cisplatin | 72 h |

Cells will be cultured in the media supplemented with 10% FBS (fetal Bovine serum), at a temperature of 37° C., 5% CO₂ and 95% humidity.

### 2. Material and Reagents

### General Cell Culture Reagents and Plastic.

FBS, (Cat #FND500, ExCell Bio)
96-Well Flat Clear Bottom Black Polystyrene TC-Treated Microplates (Cat #3603, Corning).
CellTiter-Glo® Luminescent Cell Viability Assay (Cat #G7572, Promega)

### Reagent Preparation

a. Thaw the CellTiter-Glo Buffer, and equilibrate to room temperature (RT) prior to use.
b. Equilibrate the lyophilized CellTiter-Glo Substrate to room temperature prior to use.
c. Transfer the appropriate volume (100 mL) of CellTiter-Glo Buffer into the amber bottle containing CellTiter-Glo Substrate to reconstitute the lyophilized enzyme/substrate mixture. This forms the CellTiter-Glo Reagent.

### Substrate Vial.

d. Mix by gently vortexing, swirling or by inverting the contents to obtain a homogeneous solution. The CellTiter-Glo Substrate should go into solution easily in less than one minute.

### 3. Test Articles and Reference Control

### 3.1 Test Articles:

| Item | Sample | Sample | Quantity | Storage | Molecular | |
|---|---|---|---|---|---|---|
| No. | Label | Description | (vial, mg) | Conditions | Weight | Solvent |
| 1 | Compound 1 | Powder | 31.41 mg + | RT | 751.74 | DMSO |
| | | | 30.75 mg | | | |
| 2 | Olaparib | Powder | 10.74 mg + | RT | 434.51 | DMSO |
| | | | 12.54 mg | | | |
| 3 | Talazoparib | Powder | 11 mg + | RT | 380.39 | DMSO |
| | | | 12.12 mg | | | |

### 3.2 Reference Control:

| | Molecular | | | | |
|---|---|---|---|---|---|
| Subject | Weight | Package | Supplier | Properties | Storage |
| Cisplatin | 300.05 | 20 mg | Qilu Pharm, | Powder | RT |
| | | | China | | |

### Determination of the Half Maximal Inhibition Concentration IC₅₀

### Part 1: IC₅₀ Determination Using CellTiter-Glo™ Cell Viability Assay

1. Harvest cells during the logarithmic growth period and count cell number.
2. Adjust cell concentrations to 4.0×10⁴cells/mL with respective culture medium.
3. Add 100 μL cell suspensions to three 96-well plates (plates A, B and C) with the final cell density of 4×10³ cells/well.
   4a. Next Day: For the Plates of TO Reading:
1) Equilibrate the plate A and its content at RT for approximately 30 min.
2) Add 50 μL CellTiter-Glo reagent to each well.
3) Mix content for 5 min on an orbital shaker to induce cell lysis.
4) Allow the plate to incubate at RT for 20 min to stabilize luminescent signal.
5) Record luminescence (TO) using EnVision Multi Label Reader.
   4b. For the Plates of Test Reading:
1) Prepare 500× solution of test articles (Top working concentration: 10 μM/100 μM of test articles in media with 3-fold serial dilutions to achieve 9 dose levels.
2) Prepare 10× reference control solutions (Top working concentration: 100 μM in media with 3.16-fold serial dilutions.
3) Dispense 500× drug solutions in each well (triplicate for each drug concentration) of the plate B using digital dispenser. (DMSO final concentration in culture medium: 0.2% [v/v])
4) Remove 10 μL culture media of reference control plate C with liquid handling Biomek FXP
5) Dispense 10 μL (10x) drug solution of reference control in each well (triplicate for each drug concentration) of the plate C.
6) Incubate the test plate B and C for 72 h in the humidified incubator at 37° C. with 5% CO₂ and then measured by means of CTG assay.
5. Equilibrate the plate and its content at RT for approximately 30 min.
6. Add 50 μL CellTiter-Glo reagent to each well.
7. Mix contents for 5 min on an orbital shaker to induce cell lysis.
8. Allow the plates to incubate at RT for 20 min to stabilize luminescent signal.
9. Record luminescence (T3) using EnVision Multi Label Reader.

### Part 2: Synergism or Antagonism Determination

1. Harvest cells during the logarithmic growth period and count cell number.
2. Adjust cell concentrations to 4×10⁴ cells/mL with respective culture medium.
3. Add 100 μL cell suspensions to three 96-well plates (plates A, B, C, D) with the final cell density of 4×10³ cells/well.
   4a. Next Day: For the Plates of TO Reading:
1) Equilibrate the plate A and its content at RT for approximately 30 min.
2) Add 50 μL CellTiter-Glo reagent to each well.
3) Mix content for 5 min on an orbital shaker to induce cell lysis.
4) Allow the plate to incubate at RT for 20 min to stabilize luminescent signal.
5) Record luminescence (TO) using EnVision Multi Label Reader.
   4b. For the Plates of Test Reading:
1) Prepare the drug solution of each test article Dose range: 10 μM, 3.33 μM, 1.11 μM, 0.37 μM, 0.12 μM, 0.041 μM.
2) Dispense 1000× drug solution of each test article simultaneously in each well (triplicate for each drug concentration) of test plates B, C, D using digital dispenser to obtain following final concentrations: 10 μM, 3.33 μM, 1.11 μM, 0.37 μM, 0.12 μM, 0.041 μM.
3) Incubate the test plates B, C, D for 72 h in the humidified incubator at 37° C. with 5% CO₂ and then measured by means of CTG assay.
4) Equilibrate the plate and its contents at room temperature for approximately 30 min.
5) Add 50 μL CellTiter-Glo to each well.
6) Mix contents for 5 min on an orbital shaker to induce cell lysis.
7) Allow the plate to incubate at room temperature for 20 min to stabilize luminescent signal. Note: Uneven luminescent signal within standard plates can be caused by temperature gradients, uneven seeding of cells or edge effects in multiwall plates.
8) Record luminescence (T3) using EnVision Multi Label Reader.

### Data Analysis

### Part 1: IC₅₀ Determination

The data will be displayed graphically using GraphPad Prism 5.0.

In order to calculate absolute IC50 (EC50), a dose-response curve will be fitted using nonlinear regression model with a sigmoidal dose response. The formula for calculating surviving rate is shown below and the absolute IC₅₀ (EC50) will be calculated according to the dose-response curve generated by GraphPad Prism 5.0.

The surviving rate (%)=(Lum_{Test article}−Lum_{Medium control})/(Lum_{Non-treated}−Lum_{Medium control})×100%.

### Part 2: Synergism or Antagonism Determination

Synergy is calculated based on Combenefit software [Bioinformatics 2016, 32 (18), 2866-2868.] using one of the three built-in algorithms for calculating synergy—highest single agent (HSA) [Nature biotechnology 2012, 30 (11), 1125-30].

An average delta score higher than 5 (5% response beyond expectation) is considered as significant.

The delta score reported here indicates the percentage of response beyond expectation of drug combination at some specific dose levels. A delta score higher than 5 (5% response beyond expectation) is considered as significant.

### Results

| | | | HSA_Average <−5 | | |
|---|---|---|---|---|---|
| | | | Antagonism >+5 | | |
| Cell Line | DrugA | DrugB | Synergy | TP53 Status | DDR status |
| CAL-51 | Olaparib | Compound 1 | 8.0 | WT | BRCA2 Mutant |
| CAL-51 | Talazoparib | Compound 1 | 15.7 | WT | BRCA2 Mutant |
| A2780 | Olaparib | Compound 1 | 6.2 | WT | DDR |
| | | | | | defective* |
| A2780 | Talazoparib | Compound 1 | 12.3 | WT | DDR |
| | | | | | defective* |

*A2780 is reported to carry DDR defects (The Journal of Molecular Diagnostics Volume 21, Issue 2, March 2019, Pages 198-213).

The results show a clear synergy between compound 1 and PARP inhibitors. In particular the results show a clear synergy between compound 1 and PARP inhibitors in DDR depleted cell lines.

Pharmaceutical Formulation Examples

### (i) Tablet Formulation

A tablet composition containing a compound of formula (I°) is prepared by mixing an appropriate amount of the compound (for example 50-250 mg) with an appropriate diluent, disintegrant, compression agent and/or glidant. One possible tablet comprises 50 mg of the compound with 197 mg of lactose (BP) as diluent, and 3 mg magnesium stearate as a lubricant and compressing to form a tablet in known manner. The compressed tablet may be optionally film coated.

### (ii) Capsule Formulation

A capsule formulation is prepared by mixing 100-250 mg of a compound of formula (I°) with an equivalent amount of lactose and filling the resulting mixture into standard hard gelatin capsules. An appropriate disintegrant and/or glidant can be included in appropriate amounts as required.

(iii) Injectable Formulation I

A parenteral composition for administration by injection can be prepared by dissolving a compound of formula (I°) (e.g. in a salt form) in water containing 10% propylene glycol to give a concentration of active compound of 1.5% by weight. The solution is then made isotonic, sterilised by filtration or by terminal sterilisation, filled into an ampoule or vial or pre-filled syringe, and sealed.

### (iv) Injectable Formulation II

A parenteral composition for injection is prepared by dissolving in water a compound of formula (I°) (e.g. in salt form) (2 mg/ml) and mannitol (50 mg/ml), sterile filtering the solution or by terminal sterilisation, and filling into sealable 1 ml vials or ampoules or pre-filled syringe.

(v) Injectable formulation Ill

A formulation for i.v. delivery by injection or infusion can be prepared by dissolving the compound of formula (I°) (e.g. in a salt form) in water at 20 mg/ml and then adjusted for isotonicity. The vial is then sealed and sterilised by autoclaving or filled into an ampoule or vial or pre-filled syringe, sterilised by filtration and sealed.

(vi) Injectable formulation IV

A formulation for i.v. delivery by injection or infusion can be prepared by dissolving the compound of

formula (I°) (e.g. in a salt form) in water containing a buffer (e.g. 0.2 M acetate pH 4.6) at 20 mg/ml. The vial, ampoule or pre-filled syringe is then sealed and sterilised by autoclaving or sterilized by filtration and sealed.

(vii) Subcutaneous or Intramuscular Injection Formulation

A composition for sub-cutaneous or intramuscular administration is prepared by mixing a compound of formula (I°) with pharmaceutical grade corn oil to give a concentration of 5-50 mg/ml. The composition is sterilised and filled into a suitable container.

(viii) Lyophilised Formulation I
Aliquots of formulated compound of formula (I°) are put into 50 ml vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.
(ix) Lyophilised Formulation II Aliquots of formulated compound of formula (I°) or a salt thereof as defined herein are put into 50 mL vials and lyophilized. During lyophilisation, the compositions are frozen using a one-step freezing protocol at (−45° C.). The temperature is raised to −10° C. for annealing, then lowered to freezing at −45° C., followed by primary drying at +25° C. for approximately 3400 minutes, followed by a secondary drying with increased steps if temperature to 50° C. The pressure during primary and secondary drying is set at 80 millitor.
(x) Lyophilised Formulation for Use in i.v. Administration III

An aqueous buffered solution is prepared by dissolving a compound of formula (I°) in a buffer. The buffered solution is filled, with filtration to remove particulate matter, into a container (such as a Type 1 glass vial) which is then partially sealed (e.g. by means of a Fluorotec stopper). If the compound and formulation are sufficiently stable, the formulation is sterilised by autoclaving at 1210C for a suitable period of time. If the formulation is not stable to autoclaving, it can be sterilised using a suitable filter and filled under sterile conditions into sterile vials. The solution is freeze dried using a suitable cycle.

On completion of the freeze drying cycle the vials are back filled with nitrogen to atmospheric pressure, stoppered and secured (e.g. with an aluminium crimp). For intravenous administration, the freeze dried solid can be reconstituted with a pharmaceutically acceptable diluent, such as 0.9% saline or 5% dextrose. The solution can be dosed as is, or can be diluted further into an infusion bag (containing a pharmaceutically acceptable diluent, such as 0.9% saline or 5% dextrose), before administration.

(xii) Powder in a Bottle

A composition for oral administration is prepared by filling a bottle or vial with a compound of formula (I°). The composition is then reconstituted with a suitable diluent for example water, fruit juice, or commercially available vehicle such as OraSweet or Syrspend. The reconstituted solution may be dispensed into dosing cups or oral syringes for administration.

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

1-57. (canceled)

58. A method of treating cancer in a patient, comprising a treatment step that comprises administering to the patient a therapeutically effective amount of an MDM2 antagonist, wherein the cancer is depleted of one or more genes or gene products in one or more DNA damage repair (DDR) pathways, or wherein the cancer has at least one loss of function mutation in at least one DDR pathway gene, wherein the one or more DDR pathway genes or gene products comprise BRCA1 and/or BRCA2.

59. The method according to claim 58, wherein the one or more DDR pathways are:
a. the homologous recombination repair (HRR) pathway;
b. the non-homologous end joining (NHEJ) pathway;
c. the mismatch repair (MMR) pathway;
d. the Fanconi Anemia (FA) pathway; and/or
e. the base excision repair (BER) pathway.

60. The method according to claim 58, wherein:
the one or more genes or gene products comprise or consist of HRR pathway genes or gene products other than ATM; or
the one or more genes or gene products comprise or consist of BRCA1 and/or BRCA2, and ATM.

61. The method according to claim 58, wherein:
(i) the one or more genes or gene products comprise ATRX; and/or
(ii) the one or more genes or gene products comprise MSH2, MSH3, MSH6, MLH1, MLH3, PMS2, POLE and/or POLD1; or the cancer comprises mutational signature SBS6 or SBS26 associated with defects in DNA mismatch repair, and/or the POLD1 mutational signature SBS20; and/or
(iii) the one or more genes or gene products comprise FANCA, FANCB, FANCC, FANCD1, FANCD2, FANCE, FANCF, FANCG, FANCI, FANCJ, FANCL, FANCM, FANCN, FANCO, FANCP, FANCQ, FANCR, FANCS, FANCT, FANCU, FANCV and/or FANCW.

62. The method according to claim 58, comprising detecting the depletion or mutation in a DDR gene by assessing the microsatellite instability status and/or tumour mutational burden of the cancer, optionally wherein the cancer is MSI-high.

63. The method according to claim 58, comprising testing a sample of patient tissue to determine the cancer expression profile prior to treatment, optionally wherein the patient is selected for treatment based on the determined expression profile, optionally wherein
the sample comprises cancer DNA, ctDNA, or cancer cells; and/or
the testing comprises an assay to detect protein, mRNA and/or ctDNA, optionally wherein (i) protein is detected using an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, a radioimmunoassay, immunofluorescence, immunochemiluminescence, immunoelectrochemiluminescence, immunoelectrophoretic, a competitive immunoassay, or immunoprecipitation; and/or
(ii) wherein mRNA is detected using RT-PCR or a quantitative gene expression assay; and/or (iii) wherein DNA or RNA is detected by Next Generation Sequencing; and/or
(iv) wherein protein is detected by immunohistochemistry.

64. The method according to claim 58, wherein:
(i) the cancer is
acute myeloid leukemia (AML), squamous cell carcinoma or tumors of the head, neck, skin, gastrointestinal system or genital tract; or
prostate, ovarian, breast or gynaecological cancers; or
colorectal, gastric or gynaecological cancers;
and/or
(ii) the cancer is P53 wild-type; and/or
(iii) the cancer cells undergo apoptosis following the treatment step; and/or
(iv) activated caspase-3 is induced by the MDM2 antagonist in at least a proportion of the cancer cells; and/or
(v) activated caspase-3 is induced by the MDM2 antagonist in at least 40% of the cancer cells or at least 60% of the cancer cells.

65. The method according to claim 58, wherein the cancer shows:
decreased expression, relative to a control, of one, two or three of CDKN2 Å, BAP1 and SKP2; and/or
increased expression, relative to a control, of one, two, three, four, five or more of the interferon signature genes, optionally wherein the interferon signature genes are CXCL10, CXCL11, RSAD2, MX1, BATF2, IFI44L, IFITM1, ISG15, CMPK2, IFI27, CD74, IFIH1, CCRL2, IFI44, HERC6, ISG20, IFIT3, HLA-C, OAS1, IFI35, IRF9, EPSTI1, USP18, BST2, CSF1, CIS, DHX58, TRIM14, OASL, IRF7, LGALS3BP, DDX60, LAP3, LAMP3, PARP12, PARP9, SP 110, PLSCR1, WARS, STAT1, IRF3, IRF5, MSC, JUN, SPI1, IRF1, COMMD3-BMI1, STAT2, RUNX3, SREBF1 and FLI1; or
increased expression of CXCL10 or CXCL11.

66. The method according to claim 58, wherein the cancer shows increased expression of one, two, three, four, five or more of IRF7, STAT1, IRF3, IRF5, MSC, JUN, SPI1, IRF1, COMMD3-BMI1, STAT2, RUNX3, SREBF1, IRF9 and FLI1.

67. The method according to claim 58, wherein the MDM2 antagonist:
is a compound of formula (I°) or a tautomer, N-oxide, pharmaceutically acceptable salt or solvate thereof as defined herein, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid or a tautomer, N-oxide, pharmaceutically acceptable salt or solvate thereof; or
is selected from the group consisting of Compound 1, idasanutlin (RG-7388), HDM-201, KRT-232 (AMG-232), ALRN-6924, MI-773 (SAR405838), CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and 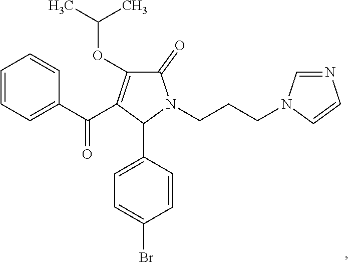 [SMILES: CC(C)OC1=C(C(=O)c2ccccc2)C(c2ccc(Br)cc2)N(CCCn2ccnc2)C1=O]
or a tautomer or a solvate or a pharmaceutically acceptable salt thereof.

68. The method according to claim 58, comprising, prior to the treatment step:
prognosing or assessing the responsiveness of a human cancer patient to treatment with an MDM2 antagonist, comprising assessing the expression or activity level in a sample from a cancer patient of one or more DDR pathway genes, wherein the one or more DDR pathway genes comprise BRCA1 and/or BRCA2, and determining whether the tested expression or activity level indicates that the cancer should be treated with an MDM2 antagonist,
optionally wherein
the assessment step comprises comparing the expression or activity level with the expression or activity level (i) associated with responsiveness or non-responsiveness to treatment with an MDM2 antagonist or (ii) from a healthy non-cancer cell of the same type; and/or
the patient is classified into a group based on the biomarker profile, optionally wherein the groups comprise or consist of:
(i) responders and non-responders; or
(ii) strong responders;
and/or
a patient is identified as particularly suitable for treatment when 1, 2, 3, 4, 5, 6, 7, 8, 9, 10 or more DDR pathway genes are expressed at a lower level than in a patient identified as not suitable for treatment; and/or
the patient is identified for treatment with the MDM2 antagonist when decreased expression of one or DDR pathway genes is detected, relative to the expression level (i) associated with non-responsiveness to treatment with an MDM2 antagonist or (ii) from a healthy non-cancer cell of the same type.

69. The method according to claim 68, comprising the step of detecting the expression or activity level of the biomarkers in a sample of cancer cells from said human patient, optionally wherein the detection is carried out using an in vitro detection assay.

70. The method according to claim 68, wherein:
the MDM2 antagonist is a compound of formula (I°) or a tautomer, N-oxide, pharmaceutically acceptable salt or solvate thereof as defined herein, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid or a tautomer, N-oxide, pharmaceutically acceptable salt or solvate thereof, or
the MDM2 antagonist is selected from the group consisting of Compound 1, idasanutlin, HDM-201, KRT-232, ALRN-6924, ALRN-6924, CGM-097, milademetan tosylate, APG-115, BI-907828, LE-004, DS-5272, SJ-0211, BI-0252, AM-7209, SP-141, SCH-1450206, NXN-6, ADO-21, CTX-50—CTX-1, ISA-27, RO-8994, RO-6839921, ATSP-7041, SAH-p53-8, PM-2, K-178, MMRi-64 and 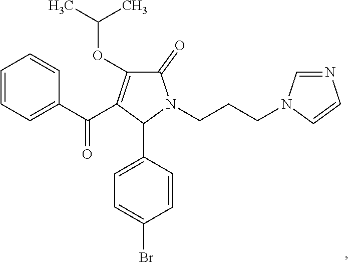 [SMILES: CC(C)OC1=C(C(=O)c2ccccc2)C(c2ccc(Br)cc2)N(CCCn2ccnc2)C1=O]
or a tautomer or a solvate or a pharmaceutically acceptable salt thereof,
and/or
further comprising providing an MDM2 antagonist for use in a method of treating the cancer in the patient, optionally wherein the treatment is selected for the patient based on the outcome of the method.

71. A kit or device, or a system, selected from:
a kit or device for detecting the expression or activity level of at least one biomarker for sensitivity to MDM2 inhibition in a sample from a human patient, comprising detection reagents for detecting one or more DDR pathway genes or gene products, wherein the one or more DDR pathway genes or gene products comprise BRCA1 and/or BRCA2; or
a system for determining the suitability of a human cancer patient for treatment with an MDM2 antagonist, comprising a storage memory for storing data associated with a sample from the patient comprising data associated with a panel of biomarkers indicating biomarker expression or activity levels in the sample from the subject, the panel of biomarkers comprising one or more DDR pathway genes or gene products wherein the one or more DDR pathway genes or gene products comprise BRCA1 and/or BRCA2; and
a processor communicatively coupled to the storage memory for classifying the patient.

72. The method according to claim 58, wherein the cancer shows loss of one or more DDR pathway genes, gene products or activities, wherein the one or more DDR pathway genes, gene products or activities comprise BRCA1 and/or BRCA2.

73. The method according to claim 58, wherein the MDM2 antagonist is part of a combination therapy with a second therapeutic agent, optionally a PARP inhibitor.

74. The method according to claim 58, wherein:
(i) the cancer has normal or high levels of one or more genes or gene products in one or more DNA damage repair (DDR) pathways, wherein the one or more DDR genes or gene products comprise BRCA1 and/or BRCA2, or wherein the cancer has no detectable loss of function mutation in any DDR pathway gene,
and wherein the MDM2 antagonist is in combination with an agent to induce sensitivity to an MDM2 antagonist for example to lower the levels of one or more genes or gene products in a DNA damage repair (DDR) pathway; or
(ii) said method comprises the steps of selecting a patient:
(a) having normal or high levels of DDR pathway genes or gene products within a biological sample obtained from said patient, wherein the one or more DDR pathway genes or gene products comprise BRCA1 and/or BRCA2; and
(b) administering a therapeutically effective amount of an MDM2 antagonist and an agent to induce sensitivity to an MDM2 antagonist for example by lowering the levels of one or more genes or gene products in a DNA damage repair (DDR) pathway, to said patient selected in step (a); optionally wherein the agent to induce sensitivity to an MDM2 antagonist is a DNA-damaging agent or a DNA repair inhibitor.

75. The method according to claim 58, comprising administering to the patient an additional therapeutic agent, e.g. ASTX660 or a tautomer or a solvate or a pharmaceutically acceptable salt thereof.

76. A packaged pharmaceutical product comprising:
(i) an MDM2 antagonist; and
(ii) a patient insert detailing instructions for administering the MDM2 antagonist in the treatment of a patient having cancer that is depleted of one or more genes or gene products in one or more DNA damage repair (DDR) pathways, or wherein the cancer has at least one loss of function mutation in at least one DDR pathway gene, wherein the one or more DDR pathway genes or gene products comprise BRCA1 and/or BRCA2.

77. The packaged pharmaceutical product according to claim 76, wherein the MDM2 antagonist is a compound of formula (I°) or a tautomer, N-oxide, pharmaceutically acceptable salt or solvate thereof, for example (2S,3S)-3-(4-chlorophenyl)-3-[(1R)-1-(4-chlorophenyl)-7-fluoro-5-[(1S)-1-hydroxy-1-(oxan-4-yl)propyl]-1-methoxy-3-oxo-2,3-dihydro-1H-isoindol-2-yl]-2-methylpropanoic acid or a tautomer, N-oxide, pharmaceutically acceptable salt or solvate thereof, for use in the treatment of cancer in a patient.
